(19) United States
(12) Patent Application Publication
(10) Pub. No.: US 2012/0189670 A1
(43) Pub. Date: Jul. 26, 2012

(54) PHARMACEUTICAL COMPOSITIONS AND FORMULATIONS INCLUDING INHIBITORS OF THE PLECKSTRIN HOMOLOGY DOMAIN AND METHODS FOR USING SAME

(76) Inventors: D. Lynn Kirkpatrick, Houston, TX (US); Jennifer L. H. Johnson, Tucson, AZ (US)

(21) Appl. No.: 13/394,470

(22) PCT Filed: Sep. 14, 2010
(86) PCT No.: PCT/US10/48813

§ 371 Date: Apr. 6, 2012

Related U.S. Application Data
(60) Provisional application No. 61/242,120, filed on Sep. 14, 2009.
(60) Provisional application No. 61/242,147, filed on Sep. 14, 2009.

(51) Int. Cl. A61K 31/635; A61P 35/00; A61K 9/00; A61K 31/433

(52) U.S. Cl. 424/400; 514/158; 514/363

(57) ABSTRACT

Pleckstrin homology domain binding compounds, pharmaceutical compositions including such compounds, and methods for their use are described herein.

### CROSS REFERENCE

This application claims priority from U.S. Provisional Application No. 61/242,120 entitled “Pharmaceutical Compositions and Formulations Including Inhibitors or Plackstrin Homology Domain and Methods for Using Same” filed Sep. 14, 2009 and U.S. Provisional Application No. 61/242,147 entitled “Pharmaceutical Compositions and Formulations Including Inhibitors or Plackstrin Homology Domain and Methods for Using Same” filed Sep. 14, 2009, the contents of each of which are hereby incorporated by reference in their entireties.

### GOVERNMENT INTERESTS

Not Applicable

### PARTIES TO A JOINT RESEARCH AGREEMENT

Not Applicable

### INCORPORATION BY REFERENCE OF MATERIAL SUBMITTED ON A COMPACT DISC

Not Applicable

### BACKGROUND

Pleckstrin homology (PH) domains contain 100-120 amino acids and are found in over 250 human proteins (1). About 40 PH domains are known to bind phosphorylated phosphatidylinositide (PtdIns) lipids held in cell membranes. PtdIns phosphorylation and the subsequent binding of PH domain-containing proteins are vital components of signal transduction pathways that regulate cell growth and survival. For example, phosphorylation of PtdIns(4,5)P₂ to produce PtdIns(3,4,5)P₃ by PtdIns 3-K signals the recruitment and binding of AKT to the inner leaflet of the plasma membrane via recognition of the PH domain (5,6). The phosphatidylinositol-3-kinase (PtdIns-3-kinase)/Akt pathway is a survival signaling pathway that is activated in many types of human cancer. Cancer cells are resistant to the mechanisms that cause programmed cell death (apoptosis) in normal cells because they contain these activated survival signaling pathways. The PH domains of proteins, and specifically in this case in Akt, provide novel molecular targets for new types of drugs to prevent and treat cancer.

The PtdIns 3-kinase (PtdIns 3-K)/AKT pathway is of critically importance for cell proliferation and survival. Phosphorylation of PtdIns(4,5)P2 to produce PtdIns(3,4,5)P3 by PtdIns 3-K signals the recruitment and docking of AKT to the inner leaflet of the plasma membrane via its pleckstrin homology (PH) domain. AKT is then phosphorylated at Thr308 by the plasma membrane bound PtdIns dependent kinase-1 (PDK1) and on Ser473 by either intergrin linked kinase (ILK), by the kinase activity of AKT itself or by mammalian target of rapamycin (mTOR)-rictor (TORC2). Once fully phosphorylated, AKT translocates back to the cytosol and nucleus, where it phosphorylates a variety of downstream targets including pro-apoptotic promoters such as forkhead transcription factors FKHR and AFX, as well as the Bcl-2 family member Bad, which is directly inhibited by phosphorylation via AKT. AKT promotes cell survival by activating CREB, and promotes proliferation by activating p70S6kinase and GSK-3β which contributes to cyclin D accumulation of cell cycle entry. AKT also acts as a mediator for VEGF production and angiogenesis by phosphorylation of mTOR, and defects in the PtdIns 3-K/AKT pathway are found in a variety of cancers, with most abnormalities occurring with mutation events in PTEN. Given the importance of AKT in proliferation and survival signaling, it has the potential to be an important target for cancer drug discovery.

Three genes encode AKT within the mammalian species to produce AKT-1/α, AKT-2/β, and AKT-3/γ isoforms of AKT of which AKT-1 and AKT-2 are expressed throughout the organism while AKT-3 is predominantly expressed in the brain, heart, and kidney. The three isoforms share a high degree of sequence homology within their PH domains but diverge within other regions. However, despite these differences they appear to have similar effects on cellular growth and apoptosis, and these similarities in biological and physiological properties between isoforms coupled with the similarities between their PH domains offers a fortuitous advantage in designing drugs that inhibit all AKT activity.

### SUMMARY OF THE INVENTION

In one embodiment, a pharmaceutical composition is provided comprising a pharmaceutically effective amount of a small molecule that binds a Pleckstrin Homology domain (PH) of AKT protein kinases and inhibits AKT protein kinase activity. The composition in this embodiment includes and one or more pharmaceutically acceptable carriers, excipients, or combinations thereof; and an enteric coating formulated to release the small molecule at a pH of from about 7.0 to about 11.

In another embodiment, a pharmacaeutical composition for topical administration is provided. In this embodiment, the composition includes a pharmaceutically effective amount of a small molecule that binds to the Pleckstrin Homology domain (PH) of AKT protein kinases and inhibits AKT protein kinase activity; and one or more of pharmaceutically acceptable lipophilic bases, cosolvents, cosurfactants, or combinations thereof.

Other embodiments are included and described herein.

### DESCRIPTION OF THE DRAWINGS

For a fuller understanding of the nature and advantages of the present invention, reference should be made to the following detailed description taken in connection with the accompanying drawings, in which:

FIG. 1 is a graphical representation of an in vitro screen.

FIGS. 2A-2B illustrate the biological activity of compound 100 in Panc-1 cells.

FIGS. 3A-3D illustrate the modeling of interactions of compounds 100, 101, 103b, 104 and 137 to AKT.

FIGS. 4A-4C illustrate the biological properties of compounds 100, 101, 102, 103 and 104.

FIGS. 5A-5C illustrate inhibition of AKT and downstream proteins by compound 104.

FIGS. 6A-6C illustrate anti-tumor activity and inhibition of AKT by compound 104.

FIG. 7 is a graphical representation that shows the relative binding of compounds 104, 155, 154, 153, 156, 157 and 158 to the expressed PH domain of AKT.

FIG. 8 is a graphical representation that shows the effects of R¹ alkyl chain length on calculated logP and CaCo-2 permeability of compound 104 like compounds.

FIG. 9 is a graphical representation that shows the antitumor activity of compounds 104, 155, 154 and 153.

FIG. 10 is a graphical representation that shows tumor growth inhibition of compound 104 in different carcinogenic cell lines.

FIG. 11 is a graphical representation that shows anti-tumor activity of compound 104 alone or incombination with paclitaxel in MCF-7 human breast cancer xenografts.

FIGS. 12A-12C illustrate the induction of apoptosis in HaCaT cells by compound 104.

FIG. 13A-13B illustrate the localization of compound 137 in HaCaT cells and a comparison of inhibition of AKT phosphorylation for compound 104 and compound 137.

FIGS. 14A-14C illustrate inhibition of UVB-induced AKT phosphorylation in HaCaT cells by compound 104.

FIGS. 15A-15C illustrate the effects of compound 104 on total AKT in scid mouse skin.

FIGS. 16A-16D illustrate the interactions of compound 316 with the human AKT1 and PDK1 PH domain.

FIGS. 17A-17B illustrate the binding of the compounds 316 and 331 to the PH domain of AKT1 and IRS1.

FIGS. 18A-18B illustrate a graphical representation of ELISA competitive binding assays for compounds 316 and 331.

FIGS. 19A-19D illustrates inhibition of AKT in cancer cells for compounds 316, 331, 332, 333, 360 and 335.

FIGS. 20A-20C shows graphical representation of the in vivo activity of compound 316.

FIGS. 21A-21C illustrates a time course (A) and concentration dependent (B) inhibition of AKT and PDK1 in cultured cells.

FIG. 22A-22B illustrates in vivo inhibition of AKT and PDK1 by Western blot (A) and a bar graph of the results (B).

FIG. 23 shows graphical representation of a pH-solubility profile of compound 104.

FIG. 24 shows graphical representation of the stability profile of compound 104 in Vehicle #3 at 4° C.

FIG. 25 shows graphical representation of the stability profile of compound 104 in Vehicle #3 at 25° C.

FIG. 26 shows graphical representation of shows the stability profile of compound 104 in Vehicle #3 at 40° C.

FIG. 27 shows graphical representation of stability of compound in Vehicle #3 stored at 40° C. (yellow), 60° C. (orange), 75° C. (red) and 100° C. (pink).

FIG. 28 shows graphical representation of a modified Arrhenius plot showing the log of the number of weeks to 90% on the y-axis and the reciprocal temperature on the x-axis. The colored diamonds represent the storage conditions: 40° C. (yellow), 60° C. (orange), 75° C. (red) and 100° C. (pink).

FIG. 29 shows graphical representation of the change in tumor size (fold) in mice with intra dermal tumors at day 10 versus day 0. Mice were treated twice daily with 100 μL compound 104 in vehicle #3 containing 50 μg/mL.

FIG. 30 shows graphical representation of the stability/compatibility of compound 104 with certain pharmaceutically acceptable oral excipients: MgStear: starch, MCC, magnesium stearate and compound 104; and StearAc: starch, MCC, stearic acid and compound 104. Error bars represent 1 standard deviation.

### DETAILED DESCRIPTION

Before the compositions and methods of the invention are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims.

It must be noted that, as used herein, and in the appended claims, the singular forms “a”, “an” and “the” include plural reference unless the context clearly dictates otherwise. Unless defined otherwise, all technical and scientific terms used herein have the same meanings as commonly understood by one of ordinary skill in the art. Although any methods similar or equivalent to those described herein can be used in the practice or testing of embodiments of the present invention, the preferred methods are now described. All publications and references mentioned herein are incorporated by reference. Nothing herein is to be construed as an admission that the invention is not entitled to antedate such disclosure by virtue of prior invention.

As used herein, the term “about” means plus or minus 10% of the numerical value of the number with which it is being used. Therefore, about 50% means in the range of 45%-55%.

The term “alkyl” as employed herein by itself or as part of another group refers to both straight and branched chain radicals of up to 25 carbons, unless the chain length is otherwise limited, such as methyl, ethyl, propyl, isopropyl, butyl, s-butyl, t-butyl, isobutyl, pentyl, hexyl, isohexyl, heptyl, 4,4-dimethylpentyl, octyl, 2,2,4-trimethylpentyl, nonyl, or decyl.

The term “alkenyl” is used herein to mean a straight or branched chain radical of 2-10 carbon atoms, unless the chain length is otherwise limited, wherein there is at least one double bond between two of the carbon atoms in the chain, including, but not limited to, ethenyl, 1-propenyl, 2-propenyl, 2-methyl-1-propenyl, 1-butenyl, 2-butenyl, and the like. Preferably, the alkenyl chain is 2 to 20 carbon atoms in length, most preferably from 2 to 12 carbon atoms in length.

The term “alkynyl” is used herein to mean a straight or branched chain radical of 2-10 carbon atoms, unless the chain length is otherwise limited, wherein there is at least one triple bond between two of the carbon atoms in the chain, including, but not limited to, ethynyl, 1-propynyl, 2-propynyl, and the like. Preferably, the alkynyl chain is 2 to 20 carbon atoms in length, most preferably from 2 to 12 carbon atoms in length.

In all instances herein where there is an alkenyl or alkynyl moiety as a substituent group, the unsaturated linkage, i.e., the vinyl or ethenyl linkage, is preferably not directly attached to a nitrogen, oxygen or sulfur moiety.

The term “alkoxy” or “alkyloxy” refers to any of the above alkyl groups linked to an oxygen atom. Typical examples are methoxy, ethoxy, isopropyloxy, sec-butyloxy, and t-butyloxy.

The term “aryl” as employed herein by itself or as part of another group refers to monocyclic or bicyclic aromatic groups containing from 6 to 12 carbons in the ring portion, preferably 6-10 carbons in the ring portion. Typical examples include phenyl, biphenyl, naphthyl or tetrahydronaphthyl.

The term “aralkyl” or “arylalkyl” as employed herein by itself or as part of another group refers to C₁₋₆ alkyl groups as discussed above having an aryl substituent, such as benzyl, phenylethyl or 2-naphthylmethyl.

The term “heterocycle” may refer to a “heteroaryl.” “Heteroaryl” as employed herein refers to groups having 5 to 14 ring atoms; 6, 10 or 14 pi electrons shared in a cyclic array; and containing carbon atoms and 1, 2, 3, or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazolyl, carbazolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl, and tetrazolyl groups).

The term “heterocycle” may also refer to a “heterocycloalkyl.” “Heterocycloalkyls” as used herein may refer to any saturated or partially unsaturated heterocycle. By itself or as part of another group, “heterocycle” may refer to a saturated or partially unsaturated ring system having 5 to 14 ring atoms selected from carbon atoms and 1, 2, 3, or 4 oxygen, nitrogen, or sulfur heteroatoms. Typical saturated examples include pyrrolidinyl, imidazolidinyl, pyrazolidinyl, tetrahydrofuranyl, tetrahydropyranyl, piperidyl, piperazinyl, quinuclidinyl, morpholinyl, and dioxacyclohexyl. Typical partially unsaturated examples include pyrrolinyl, imidazolinyl, pyrazolinyl, dihydropyridinyl, tetrahydropyridinyl, and dihydropyranyl. Either of these systems can be fused to a benzene ring. When a substituent is oxo (i.e., ═O), then 2 hydrogens on the atom are replaced. When aromatic moieties are substituted by an oxo group, the aromatic ring is replaced by the corresponding partially unsaturated ring. For example, a pyridyl group substituted by oxo results in a pyridone.

The terms “heteroarylalkyl” or “heteroaralkyl” as employed herein both refer to a heteroaryl group attached to an alkyl group. Typical examples include 2-(3-pyridyl)ethyl, 3-(2-furyl)-n-propyl, 3-(3-thienyl)-n-propyl, and 4-(1-isoquinolinyl)-n-butyl.

The term “cycloalkyl” as employed herein by itself or as part of another group refers to cycloalkyl groups containing 3 to 9 carbon atoms. Typical examples are cyclopropyl, cyclobutyl, cyclopentyl, cyclohexyl, cycloheptyl, cyclooctyl and cyclononyl.

The term “cycloalkylalkyl” or “cycloalkyl(alkyl)” as employed herein, by itself or as part of another group, refers to a cycloalkyl group attached to an alkyl group. Typical examples are 2-cyclopentylethyl, cyclohexylmethyl, cyclopentylmethyl, 3-cyclohexyl-n-propyl, and 5-cyclobutyl-n-pentyl.

The term “cycloalkenyl” as employed herein, by itself or as part of another group, refers to cycloalkenyl groups containing 3 to 9 carbon atoms and 1 to 3 carbon-carbon double bonds. Typical examples include cyclopropenyl, cyclobutenyl, cyclopentenyl, cyclohexenyl, cyclohexadienyl, cycloheptenyl, cycloheptadienyl, cyclooctenyl, cyclooctadienyl, cyclooctatrienyl, cyclononenyl, and cyclononadienyl.

The term “halogen” or “halo” as employed herein by itself or as part of another group refers to chlorine, bromine, fluorine or iodine.

The term “monoalkylamine” or “monoalkylamino” as employed herein by itself or as part of another group refers to the group NH₂ wherein one hydrogen has been replaced by an alkyl group, as defined above.

The term “dialkylamine” or “dialkylamino” as employed herein by itself or as part of another group refers to the group NH₂ wherein both hydrogens have been replaced by alkyl groups, as defined above.

The term “hydroxyalkyl” as employed herein refers to any of the above alkyl groups wherein one or more hydrogens thereof are substituted by one or more hydroxyl moieties.

The term “haloalkyl” as employed herein refers to any of the above alkyl groups wherein one or more hydrogens thereof are substituted by one or more halo moieties. Typical examples include fluoromethyl, difluoromethyl, trifluoromethyl, trichloroethyl, trifluoroethyl, fluoropropyl, and bromobutyl.

The term “carboxyalkyl” as employed herein refers to any of the above alkyl groups wherein one or more hydrogens thereof are substituted by one or more carboxylic acid moieties.

The term “heteroatom” is used herein to mean an oxygen atom (“O”), a sulfur atom (“S”) or a nitrogen atom (“N”). It will be recognized that when the heteroatom is nitrogen, it may form an NR^{a}R^{b} moiety, wherein R^{a} and R^{b} are, independently from one another, hydrogen or C₁ to C₈ alkyl, or together with the nitrogen to which they are bound form a saturated or unsaturated 5-, 6-, or 7-membered ring.

The terms “hydroxy” and “hydroxyl” are used interchangeably to refer to the radical —OH. The terms “pyridyl” and “pyridinyl” are used interchangeably to refer to a monovalent radical of pyridine. The terms “carbamoyl” and “aminocarbonyl” are used interchangeably to refer to the radical NH₂—C(O)—. The terms “ureido” and “aminocarbonylamino” are used interchangeably to refer to the radical NH₂—C(O)—NH—.

“Optional” or “optionally” may be taken to mean that the subsequently described structure, event or circumstance may or may not occur, and that the description includes instances where the event occurs and instances where it does not.

The phrase “optionally substituted” when not explicitly defined refers to a group or groups being optionally substituted with one or more substituents independently selected from the group consisting of hydroxy, nitro, trifluoromethyl, halogen, C₁₋₆ alkyl, C₁₋₆ haloalkyl, C₁₋₆ alkoxy, C₁₋₆ alkylenedioxy, C₁₋₆ aminoalkyl, C₁₋₆ hydroxyalkyl, C₂₋₄ alkenyl, C₂₋₄ alkynyl, C₆₋₁₀ aryl, phenoxy, benzyloxy, 5-10 membered heteroaryl, C₁₋₆ aminoalkoxy, amino, mono(C₁₋₄)alkylamino, di(C₁₋₄)alkylamino, C₂₋₆ alkylcarbonylamino, C₂₋₆ alkoxycarbonylamino, C₂₋₆ alkoxycarbonyl, C₂₋₆ alkoxycarbonylalkyl, carboxy, C₂₋₆ hydroxyalkoxy, (C₁₋₆)alkoxy(C₂₋₆)alkoxy C₂₋₁₀ mono(C₄alkylamino(C₂₋₆)alkoxy, di(C₁₋₄)alkylamino(C₂₋₆)alkoxy C₂₋₁₀ mono(carboxyalkyl)amino, bis(C₂₋₁₀ carboxyalkyl)amino, C₂₋₆ carboxyalkoxy, C₂₋₆ carboxyalkyl, carboxyalkylamino, guanidinoalkyl, hydroxyguanidinoalkyl, cyano, trifluoromethoxy, perfluoroethoxy, aminocarbonylamino, mono(C₁₋₄alkylaminocarbonylamino, di(C₁₋₄alkylaminocarbonylamino, N—(C₁₋₄)alkyl-N-aminocarbonyl-amino, N—(C₁₋₄)alkyl-N-mono(C₁₋₄)alkylaminocarbonyl-amino or N—(C₁₋₄)alkyl-N-di(C₁₋₄alkylaminocarbonyl-amino.

“Administering” when used in conjunction with a therapeutic means to administer a therapeutic directly into or onto a target tissue or to administer a therapeutic to a patient whereby the therapeutic positively impacts the tissue to which it is targeted. “Administering” a composition may be accomplished by oral administration, injection, infusion, absorption or by any method in combination with other known techniques.

The term “target”, as used herein, refers to the material for which either deactivation, rupture, disruption or destruction or preservation, maintenance, restoration or improvement of function or state is desired. For example, diseased cells, pathogens, or infectious material may be considered undesirable material in a diseased subject and may be a target for therapy.

Generally speaking, the term “tissue” refers to any aggregation of similarly specialized cells, which are united in the performance of a particular function.

The term “improves” is used to convey that the present invention changes the appearance, form, characteristics and/or physical attributes of the tissue to which it is being provided, applied or administered. “Improves” may also refer to the overall physical state of an individual to whom an active agent has been administered. For example, the overall physical state of an individual may “improve” if one or more symptoms of cancer or other proliferative disorder are alleviated by administration of an active agent. For example, in various embodiments, improvement may be embodied by a reduction in size or density of diseased tissue and/or a reduction in the rate of proliferation of diseased tissue.

As used herein, the term “therapeutic” means an agent utilized to treat, combat, ameliorate or prevent an unwanted condition or disease of a patient.

The terms “therapeutically effective amount” or “therapeutic dose” as used herein are interchangeable and may refer to the amount of an active agent or pharmaceutical compound or composition that elicits a biological or medicinal response in a tissue, system, animal, individual or human that is being sought by a researcher, veterinarian, medical doctor or other clinician. A biological or medicinal response may include, for example, one or more of the following: (1) preventing a disease, condition or disorder in an individual that may be predisposed to the disease, condition or disorder but does not yet experience or display pathology or symptoms of the disease, condition or disorder, (2) inhibiting a disease, condition or disorder in an individual that is experiencing or displaying the pathology or pathology and/or symptoms of the disease, condition or disorder, and (3) ameliorating a disease, condition or disorder in an individual that is experiencing or exhibiting the pathology or symptoms of the disease, condition or disorder or reversing the pathology and/or symptoms experienced or exhibited by the individual.

The term “treating” may be taken to mean prophylaxis of a specific disorder, disease or condition, alleviation of the symptoms associated with a specific disorder, disease or condition and/or prevention of the symptoms associated with a specific disorder, disease or condition. In some embodiments, the term refers to slowing the progression of the disorder, disease or condition or alleviating the symptoms associated with the specific disorder, disease or condition. In some embodiments, the term refers to slowing the progression of the disorder, disease or condition. In some embodiments, the term refers to alleviating the symptoms associated with the specific disorder, disease or condition. In some embodiments, the term refers to restoring function, which was impaired or lost due to a specific disorder, disease or condition.

The term “patient” generally refers to any living organism to which to compounds described herein are administered and may include, but is not limited to, any non-human mammal, primate or human. Such “patients” may or may not be exhibiting the signs, symptoms or pathology of the particular diseased state.

The term “pharmaceutical composition” shall mean a composition including at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan. A pharmaceutical composition may, for example, contain an AKT inhibitor or a pharmaceutically acceptable salt of AKT inhibitor as the active ingredient.

For the purposes of this disclosure, a “salt” is any acid addition salt, preferably a pharmaceutically acceptable acid addition salt, including but not limited to, halogenic acid salts such as hydrobromic, hydrochloric, hydrofluoric and hydroiodic acid salt; an inorganic acid salt such as, for example, nitric, perchloric, sulfuric and phosphoric acid salt; an organic acid salt such as, for example, sulfonic acid salts (methanesulfonic, trifluoromethan sulfonic, ethanesulfonic, benzenesulfonic or p-toluenesulfonic), acetic, malic, fumaric, succinic, citric, benzoic, gluconic, lactic, mandelic, mucic, pamoic, pantothenic, oxalic and maleic acid salts; and an amino acid salt such as aspartic or glutamic acid salt. The acid addition salt may be a mono- or di-acid addition salt, such as a di-hydrohalogenic, di-sulfuric, di-phosphoric or di-organic acid salt. In all cases, the acid addition salt is used as an achiral reagent which is not selected on the basis of any expected or known preference for interaction with or precipitation of a specific optical isomer of the products of this disclosure.

“Pharmaceutically acceptable salt” is meant to indicate those salts which are, within the scope of sound medical judgment, suitable for use in contact with the tissues of a patient without undue toxicity, irritation, allergic response and the like, and are commensurate with a reasonable benefit/risk ratio. Pharmaceutically acceptable salts are well known in the art. For example, Berge et al. (1977) J. Pharm. Sciences, Vol 6. 1-19, which is hereby incorporated by reference in its entirety describes pharmaceutically acceptable salts in detail.

As used herein, the term “daily dose amount” refers to the amount of pramipexole per day that is administered or prescribed to a patient. This amount can be administered in multiple unit doses or in a single unit dose, in a single time during the day or at multiple times during the day.

A “dose amount” as used herein, is generally equal to the dosage of the active ingredient, which may be administered per day. For example, a non-effective dose amount of 10 mg/day to 10,000 mg/day of an AKT inhibitor.

The term “unit dose” as used herein may be taken to indicate a discrete amount of the therapeutic composition that contains a predetermined amount of the active compound. The amount of the active compound is generally equal to the dosage of the active ingredient, which may be administered on or more times per day. For example, the unit dose may be a fraction of the desired daily dose which may be given in fractional increments, such as, for example, one-half or one-third the dosage.

Various embodiments of the invention are directed to small molecules that bind to the Pleckstrin Homology domain (PH) of AKT protein kinases and inhibit their activity, pharmaceutical compositions including such small molecules, and methods for using such small molecules to treat proliferative diseases such as, for example, cancer. Various other embodiments are directed to small molecules that may bind to and inhibitor PDK1, pharmaceutical compositions including such small molecules, and methods of using such small molecules to treat proliferative diseases, such as, for example cancer. Certain embodiments are directed to molecules that include two or more susbstituted or unsubstituted 5- or 6 membered rings having 0-3 ring forming heteroatoms connected by flexible linkers. For example, various embodiments may include compounds of general formula I:

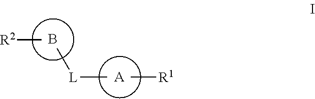

or pharmaceutically acceptable salts or solvates thereof, wherein:

L may be —S—, —S(O)₂—, —C(O)—, —P(O)(OH)—, —NH—, —N(R³)—, —CH₂—, —C(R³)₂—, —(CH₂)—OC(O)—(CH₂)₂—CH(C(O)OH)—NHC(O)O—(CH₂)—, or —(CH₂)—OC(O)—(CH₂)—CH(C(O)OH)—NHC(O)O—(CH₂)—;

L¹ and L² may each, independently, be —O—, —S—, —S(O)₂—, —C(O)—, —P(O)(OH)—, —NH—, —NR³, —CH₂—, —C(R³)₂—, or piperazinyl;

n may be 1 or 2;

each R³ may, independently, be —H, —CH₃, —CH₂CH₃, —CH₂CH₂CH₃, —NH₂, —C₆H₅ heteroarylalkyl, or C(O)R^{3a};

R^{3a} may be C₁₋₆ alkyl or aryl, each substituted with 0, 1, or 2 substituents independently selected from halogen and CN;

ring A may be a substituted or unsubstituted, 5- or 6-membered ring having 1-3 ring-forming heteroatoms or substituted or unsubstituted phenyl, and in some embodiments, ring A may be be substituted with one or more methyl, methoxy, sulfonyl, sulfonic acid ester group in addition to R¹;

R¹ may be —H, —CH₃, —CH₂CH₃, —CH₂(CH₂)ₘCH₃, —C(CH₃)₃, —CH₂CH₂R⁴, —OH, —OCH₃, —CH₂OH, —C(O)OH, —CH₂C(O)OH, —CH₂CH₂C(O)OH, —C(O)R⁴, —C(O)OR⁴, —CH₂C(O)OR⁴, —CH₂CH₂C(O)OR⁴, —NH₂, CH₂NH₂, —S(O)₂R⁴, —CH₂S(O)₂R⁴, C₆H₅, —C₆H₄R⁴, —CH₂C₆H₅, —S(O₂)C₆H₅, —CH₂S(O)₂C₆H₅, heteroaryl, heteroarylalkyl, morpholino, or halogen;

R⁴ may be —H, —OH, —NH₂, —CH₃, —CH₂CH₃, —CH₂CH₂CH₃, —OCH₃, —C(O)OH, —C₆H₅, —C₆H₄R⁵, —CH₂C₆H₅, —CH₂C₆H₄R⁵, halogen, heteroaryl, heteroarylalkyl, or piperazinyl;

R⁵ may be —H, —OH, —NH₂, —CH₃, —CH₂CH₃, —CH₂CH₂CH₃, —C(O)OH, or halogen;

ring B may be a substituted or unsubstituted, 5-14 membered aromatic or polyaromatic ring having 1 to 2 ring-forming heteroatoms, and in particular embodiments, ring B may be a substituted or unsubstituted phenyl;

R² may be —H, —CH₃, —C(CH₃)₃, C₁-C₂₀ alkyl, —OH, —NH₂, —OR⁶, —NHC(O)R⁶, —NR^{6a}R^{6b}, —NHS(O)₂R⁶, —S(O)₂OH, —CH(O), —C(O)OH, —C(O)OR⁶, —CH₂OH, —CH₂C(O)OH, —S(O)₂NH₂, —CH₂(CH₂)ₚR⁶—, CH₂(CH₂)ₚOR⁶, —CH₂—O—(CH₂)_{pOR}⁶, —CH₂(CH₂)ₚSO₂R⁶, —CH₂(CH₂)ₚNHR⁶, —C₆H₅, or —C₆H₄R⁶, wherein when R² is C₁-C₂₀ alkyl it may be optionally substituted with one or more substituents independently selected from halogen, OH, —NH₂, —NHC(O)R⁶, and —NR^{6a}R^{6b};

or R² may be

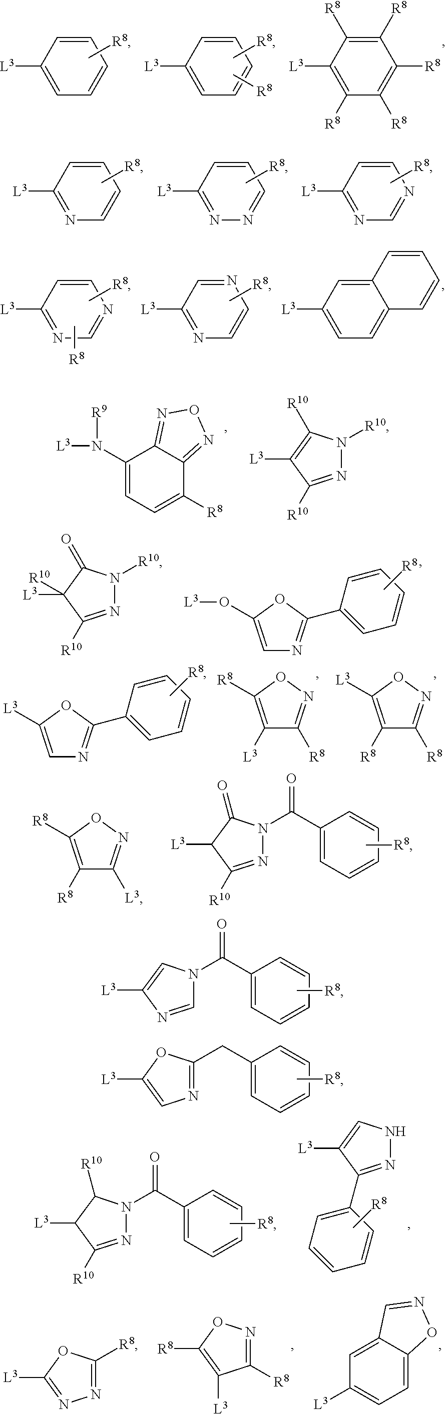

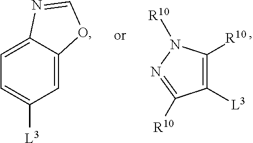

wherein R² is attached to ring B through L³;

R⁶ may be —H, —NH₂, —OH, —CH₃, —CH₂CH₃, —CH₂CH₂CH₃, —C₆H₅, —C₆H₄R⁷, —CH₂C₆H₅, —CH₂C₆H₄R⁷, halogen, aryl, heteroaryl, or C₁-C₂₀ alkyl, wherein each of the aryl, heteroaryl, or C₁-C₂₀ alkyl which may be optionally substituted with one or more substituents independently selected from —NH₂, —OH, —NH₂, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅;

L³ may be a bond, —CH₂—, —CH₂(CH₂)_{q}—, —CH(OH)—, —C(O)—, —O—, —NH—, —S—, —CH₂CH₂—, —CH═CH—, —OCH₂—, —OP(O)(OH)—, —NHS(O)₂—, —SCH₂—, —S(O)₂CH₂—, —S(O)₂O—, or —C(O)NH—;

each R⁷ and R⁸ may, independently, be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen;

R¹⁰ may be —H, —CH₃, —OH, —OCH₃, —C₆H₅, —C₆H₄R⁹,

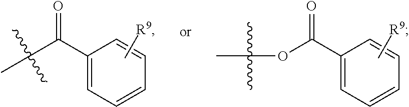

R⁹ may be —H, —CH₃, —C(CH₃), —OH, —NH₂, NO₂, —OCH₃, —C(O)OH, —C(O)NH₂, or halogen; and

m, p and q may each independently be an integer selected from 1 to 20.

In particular embodiments, the compounds of the invention may be of general formula II:

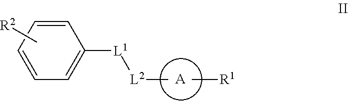

or pharmaceutically acceptable salt or solvate thereof, wherein:

L¹ and L² may each, independently, be —S—, —S(O)₂—, —C(O)—, —P(O)(OH)—, —NH—, —N(CH₃)—, —N(R³)—, —CH₂—, or —C(R³)₂—;

each R³ may, independently, be —H, —CH₃, CH₂CH₃, CH₂CH₂CH₃, NH₂, or —C₆H₅;

ring A may be a substituted or unsubstituted, 5- or 6-membered ring having 1-3 ring-forming heteroatoms and, in some embodiments, ring A may optionally be substituted with a methyl, methoxy, sulfonyl, or sulfonic acid ester group in addition to R¹;

R¹ may be —H, —CH₃, —CH₂CH₃, —CH₂(CH₂)ₘCH₃, —C(CH₃)₃, —CH₂CH₂R⁴, —OH, —OCH₃, —CH₂OH, —C(O)OH, —CH₂C(O)OH, —CH₂CH₂C(O)OH, —C(O)R⁴, —C(O)OR⁴, —CH₂C(O)OR⁴, —CH₂CH₂C(O)OR⁴, —NH₂, CH₂NH₂, —S(O)₂R⁴, —CH₂S(O)₂R⁴, C₆H₅, —C₆H₄R⁴, —CH₂C₆H₅, —S(O₂)C₆H₅, —CH₂S(O)₂C₆H₅, heteroaryl, heteroarylalkyl, morpholino, or halogen;

R⁴ may be —H, —OH, —NH₂, —CH₃, —CH₂CH₃, —CH₂CH₂CH₃, —OCH₃, —C(O)OH, —C₆H₅, —C₆H₄R⁵, —CH₂C₆H₅, —CH₂C₆H₄R⁵, halogen, heteroaryl, heteroarylalkyl, or piperazinyl;

R⁵ may be —H, —OH, —NH₂, —CH₃, —CH₂CH₃, —CH₂CH₂CH₃, —C(O)OH, or halogen;

R² may be —H, —CH₃, —C(CH₃)₃, C₁-C₂₀ alkyl, —OH, —NH₂, —OR⁶, —NHC(O)R⁶, —NR^{6a}R^{6b}, —NHS(O)₂R⁶, —S(O)₂OH, —CH(O), —C(O)OH, —C(O)OR⁶, —CH₂OH, —CH₂C(O)OH, —S(O₂)NH₂, —CH₂(CH₂)ₚR⁶—, CH₂(CH₂)ₚOR⁶, —CH₂—O—(CH₂)ₚOR⁶, —CH₂(CH₂)ₚSO₂R⁶, —CH₂(CH₂)ₚNHR⁶, —C₆H₅, or —C₆H₄R⁶, wherein when R² is C₁-C₂₀ alkyl, it may be optionally substituted with one or more substituents independently selected from halogen, OH, —NH₂, —NHC(O)R⁶, and —NR^{6a}R^{6b};

or R² may be

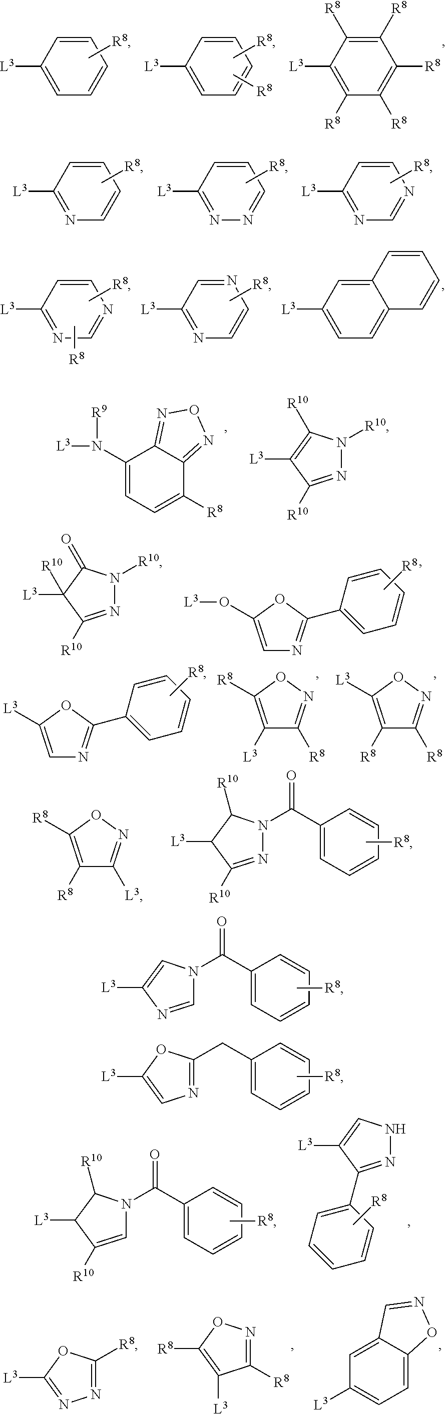

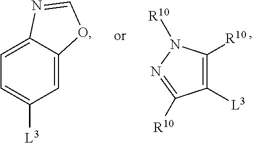

wherein R² is attached to the phenyl ring of Formula II through L³;

R⁶ may be —H, —NH₂, —OH, —CH₃, —CH₂CH₃, —CH₂CH₂CH₃, —C₆H₅, —C₆H₄R⁷, —CH₂C₆H₅, —CH₂C₆H₄R⁷, halogen, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —NH₂, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅;

L³ may be a bond, —CH₂—, —CH₂(CH₂)_{q}—, —CH(OH)—, —C(O)—, —O—, —NH—, —S—, —CH₂CH₂—, —CH═CH—, —N═N—, —OCH₂—, —OP(O)(OH)—, —NHS(O)₂—, —SCH₂—, —S(O)₂CH₂—, —S(O)₂O—, or —C(O)NH—;

each R⁷ and R⁸ may, independently, be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen;

R¹⁰ may be —H, —CH₃, —OH, —OCH₃, —C₆H₅, —C₆H₄R⁹,

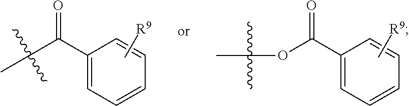

R⁹ may be —H, —CH₃, —C(CH₃), —OH, —NH₂, NO₂, —OCH₃, —C(O)OH, —C(O)NH₂, or halogen; and

m, p and q are each independently an integer selected from 1 to 20.

In some embodiments in the compound of general formula H or pharmaceutically acceptable salt or solvate thereof, L¹ may be —S—, —S(O)₂—, —C(O)—, or —P(O)(OH)—, and in other embodiments, L² may be —NH—, —NR³, —CH₂—, or —C(R³)₂—. In still other embodiments, L¹ may be —NH—, —NR³, —CH₂—, or —C(R³)₂—, and in yet other embodiments, L² may be —S—, —S(O)₂—, —C(O)—, or —P(O)(OH)—. In certain embodiments, L¹ may be —S(O)₂— and L² is —NH—.

In various embodiments, ring A of the compounds of general formula II or pharmaceutically acceptable salt or solvate thereof, may be a 5-membered heteroaryl ring. For example, in certain embodiments, the moiety of

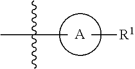

may be selected from:

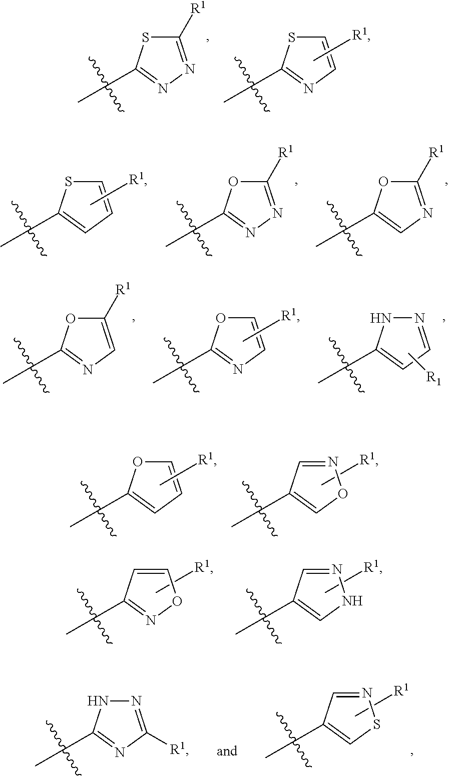

and in some embodiments ring A may be optionally substituted with one or more methyl, methoxy, sulfonyl, or sulfonic acid ester group in addition to R¹, and in particular embodiments, the moiety of

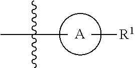

may be

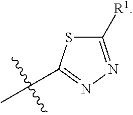

In still other embodiments, ring A may be a phenyl ring or a 6-membered heteroaryl ring. For example, in some embodiments, the moiety of

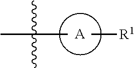

may be selected from:

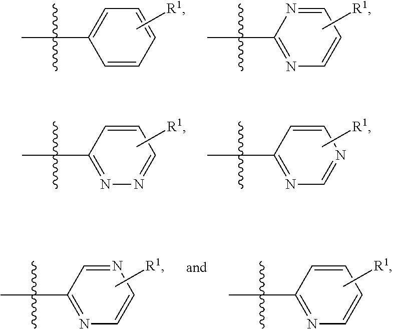

and in certain embodiments, ring A may be optionally substituted with one or more methyl, methoxy group, sulfonyl or sulfonic acid ester group in addition to R¹. In particular embodiments, the moiety of

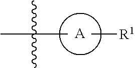

in compounds of general formula II may be

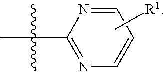

In some embodiments, in the compounds of general formula II or pharmaceutically acceptable salt or solvate thereof, R¹ may not be —S(O)₂NH₂ when R² is NH₂; L³ may not be —NHC(O)— or —NH— when the moiety of

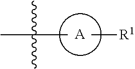

is

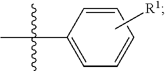

L³ may not be —NHS(O)₂— when the moiety of

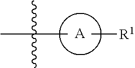

is

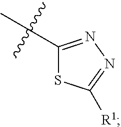

R¹ may not be —C(O)OR⁴ or —OR⁴ when the moiety of

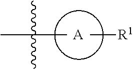

is

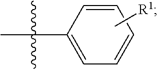

L³ may not be —NHC(O)— when the moiety of

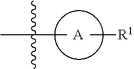

is

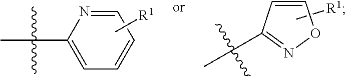

L³ may not be —S(O)₂NH— when the moiety of

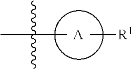

is

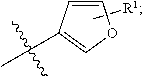

### or R² may not be

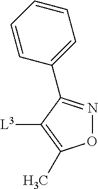

when the moiety of

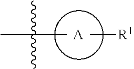

is

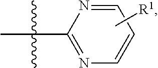

is or any combination thereof.

Particular embodiments of the invention include compounds of general formula III:

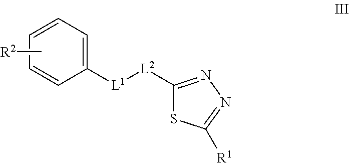

or pharmaceutically acceptable salt or solvate thereof, wherein:

L¹ and L² may each, independently, be —S—, —S(O)₂—, —C(O)—, —P(O)(OH)—, —NH—, —N(CH₃)—, —N(R³)—, —CH₂—, or —C(R³)₂—;

each R³ may, independently, be —H, —CH₃, CH₂CH₃, CH₂CH₂CH₃, NH₂, or —C₆H₅;

R¹ may be —H, —CH₃, —CH₂CH₃, —C(CH₃)₃, —C(O)OH, —CH₂C(O)OH, —CH₂C(O)OCH₃, —CH₂C(O)OCH₂CH₃, —OH, CH₂OH, —NH₂, —CH₂NH₂, —OCH₃, S(O)₂NH₂, S(O)₂C₆H₅, or S(O)₂CH₂C₆H₅;

R² may be —NH₂, —NHC(O)R⁶, —NR^{6a}R^{6b}, —NHS(O)₂R⁶, —OH, —OR⁶, C(O)OH, or C₁-C₂₀ alkyl, wherein each C₁-C₂₀ alkyl may be optionally substituted with one or more substituents independently selected from halogen, C₁₋₆ alkyl, OH, —NH₂, —NHC(O)R⁶, and —NR^{6a}R^{6b};

each R⁶ may, independently, be —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —NH₂, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In some embodiments in the compound of general formula III or pharmaceutically acceptable salt or solvate thereof, L¹ may be —S—, —S(O)₂—, —C(O)—, or —P(O)(OH)—, and in other embodiments, L² may be —NH—, —NR³, —CH₂—, or —C(R³)₂—. In still other embodiments, L¹ may be —NH—, —NR³, —CH₂—, or —C(R³)₂—, and in yet other embodiments, L² may be —S—, —S(O)₂—, —C(O)—, or —P(O)(OH)—. In certain embodiments, L1 may be —S—, —S(O)₂—, or —C(O)—, and L² may be —NH—, or —CH₂—, and in some embodiments, L¹ may be —S(O)₂— and L² is —NH—.

In particular embodiments, the compounds of general formula III or pharmaceutically acceptable salt or solvate thereof, wherein the compound is a compound of Formula III-a:

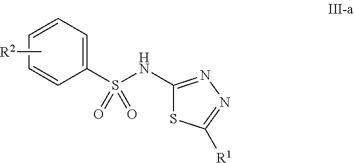

wherein:

R¹ may be —H or —CH₃;

R² may be —NH₂, —NHC(O)R⁶, —NHS(O)₂R⁶, or C₁-C₂₀ alkyl, wherein the C₁-C₂₀ alkyl may optionally be substituted with one or more substituents independently selected from halogen, C₁₋₆ alkyl, OH, —NH₂, —NHC(O)R⁶, and —NR^{6a}R^{6b};

R⁶ is —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may optionally be substituted with one or more substituents independently selected from —NH₂, —OH, —NH₂, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In some embodiments of the compound of general formula III-a or pharmaceutically acceptable salt or solvate thereof:

R¹ may be H;

R² may be C₁-C₂₀ alkyl optionally substituted with one or more substituents independently selected from halogen, OH, —NH₂, —NHC(O)R⁶, and —NR^{6a}R^{6b};

R⁶ may be —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —NH₂, —C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In other embodiments of the compounds of general formula III-a or pharmaceutically acceptable salt or solvate thereof:

R² may be —NH₂ or —NHS(O)₂R⁶;

R⁶ may be —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen; and

R⁷ may be —H, —CH₃, heteroaryl, —C(CH₃)₃, —OH, —NH₂, NHC(O)CH₃, S(O)₂OH, As(O)₂OH, NO₂, —OCH₃, —OCH₂CH₃, —C(O)OH, —C(O)NH₂, or halogen.

In still other embodiments of the compounds of general formula III-a or pharmaceutically acceptable salt or solvate thereof:

R² may be —NHS(O)₂R⁶;

R⁶ may be aryl or heteroaryl, each of which may be optionally substituted with one or more substituents independently selected from —NH₂, —OH, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In certain embodiments, R¹ may be H and R² may be —NH₂ in the compounds of general formula III-a.

In any of the embodiments of formulae III and III-a above, R² may be substituted on any carbon atom of the phenyl ring. For example, in some embodiments, R² may be positioned and arranged in the para configuration, and in other embodiments, R₂ may be positioned and arranged in the meta or ortho configuration.

Particular embodiments are directed to compounds of general formula IV:

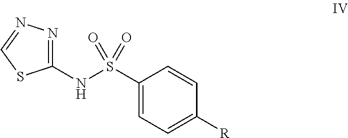

or pharmaceutically acceptable salt or solvate thereof wherein R may be an amine, methyl, alkyl, alkene, alkyne, aminoalkyl, alkyl carbamate, alkyl acetamide, alkyl sulfonyl, alkyl sulfonic acid ester, or alkyl sulfonamide such as, for example, a linear or branched C₂ to C₂₀ alkyl, linear or branched C₂ to C₂₀ alkene, linear or branched C₂ to C₂₀ alkyne, linear or branched C₂ to C₂₀ aminoalkyl, linear or branched C₂ to C₂₀ alkyl carbamate branched C₂ to C₂₀ alkyl acetamide, linear or branched C₂ to C₂₀ sulfonyl, linear or branched C₂ to C₂₀ sulfonic acid ester, or linear or branched C₂ to C₂₀ sulfonamide. In some embodiments, R may be a linear C₂-C₂₀ alkyl, and in other embodiments, R may be an alkyl acetamide of formula —NHC(O)CHₙCH₃ wherein n is 0 to 20. In particular embodiments, R may be —CH₁₁CH₃ or —NHC(O)CH₁₁CH₃, and in one exemplary embodiment, a compound of the invention may be:

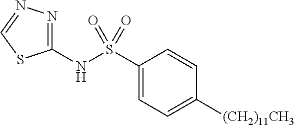

In still other embodiments, compounds encompassed by the invention may be of general formula V:

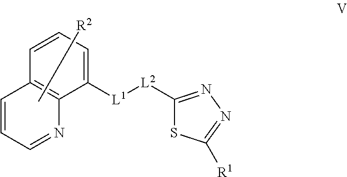

or pharmaceutically acceptable salt or solvate thereof, wherein:

L¹ and L² may each, independently, be —S—, —S(O)₂—, —C(O)—, —P(O)(OH)—, —NH—, —N(CH₃)—, —N(R³)—, —CH₂—, or —C(R³)₂—;

each R³ may, independently, be —H, —CH₃, CH₂CH₃, CH₂CH₂CH₃, NH₂, or —C₆H₅;

R¹ may be —H, —CH₃, —CH₂CH₃, —C(CH₃)₃, —C(O)OH, —CH₂C(O)OH, —CH₂C(O)OCH₃, —CH₂C(O)OCH₂CH₃, —OH, CH₂OH, —NH₂, —CH₂NH₂, —OCH₃, S(O)₂NH₂, S(O)₂C₆H₅, or S(O)₂CH₂C₆H₅;

R² may be —NH₂, —NHC(O)R⁶, —NR^{6a}R^{6b}, —NHS(O)₂R⁶, —OH, —OR⁶, C(O)OH, or C₁-C₂₀ alkyl, and wherein each C₁-C₂₀ alkyl may optionally be substituted with one or more substituents independently selected from halogen, C₁₋₆ alkyl, OH, —NH₂, —NHC(O)R⁶, and —NR^{6a}R^{6b};

R⁶ may be —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —NH₂, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In some embodiments in the compound of general formula V or pharmaceutically acceptable salt or solvate thereof, L′ may be —S—, —S(O)₂—, —C(O)—, or —P(O)(OH)—, and in other embodiments, L² may be —NH—, —NR³, —CH₂—, or —C(R³)₂—. In still other embodiments, L¹ may be —NH—, —NR³, —CH₂—, or —C(R³)₂—, and in yet other embodiments, L² may be —S—, —S(O)₂—, —C(O)—, or —P(O)(OH)—. In certain embodiments, L¹ may be —S—, —S(O)₂—, or —C(O)—, and L² may be —NH—, or —CH₂—, and in some embodiments, L¹ may be —S(O)₂— and L² is —NH—.

In other embodiments of compounds of general formula V or pharmaceutically acceptable salts or solvates thereof:

L¹ may be —S(O)₂—;

L² may be —NH—; and

R¹ may be S(O)₂NH₂.

Yet other embodiments of the invention are directed to compounds of general formula V:

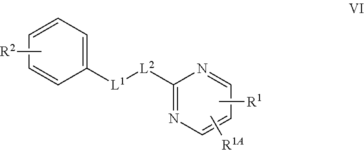

or pharmaceutically acceptable salt or solvate thereof, wherein:

L¹ and L² may each, independently, be —S—, —S(O)₂—, —C(O)—, —P(O)(OH)—, —NH—, —N(CH₃)—, —N(R³)—, —CH₂—, or —C(R³)₂—;

each R³ may, independently, be —H, —CH₃, CH₂CH₃, CH₂CH₂CH₃, NH₂, or —C₆H₅;

R¹ may be —H, —CH₃, or —OCH₃;

R^{1A} may be —H, —CH₃, or —OCH₃;

R² may be —NH₂, —NHC(O)R⁶, —NR^{6a}R^{6b}, —NHS(O)₂R⁶, —OH, —OR⁶, C(O)OH, or C₁-C₂₀ alkyl, and each C₁-C₂₀ alkyl may be optionally substituted with one or more substituents independently selected from halogen, C₁₋₆ alkyl, OH, —NH₂, —NHC(O)R⁶, and —NR^{6a}R^{6b};

R⁶ may be —CH₃, —CH₂CH₃, —CH₂CH₂CH₃, —C₆H₅, —C₆H₄R⁷, —CH₂C₆H₅, —CH₂C₆H₄R⁷, aryl, heteroaryl, or C₁-C₂₀ alkyl, wherein each of the aryl, heteroaryl, or C₁-C₂₀ alkyl may be optionally substituted with one or more substituents independently selected from —NH₂, —OH, —NH₂, —C₁₋₆ alkyl, —C₆H₅, —C₆H₄R⁷, —CH₂C₆H₅, —CH₂C₆H₄R⁷, and halogen;

R^{6a} may be H or methyl;

R^{6b} may be methyl, 7-nitrobenzo[c][1,2,5]oxadiazol-4-yl, or —C(O)C₆H₅;

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen;

or R² may be

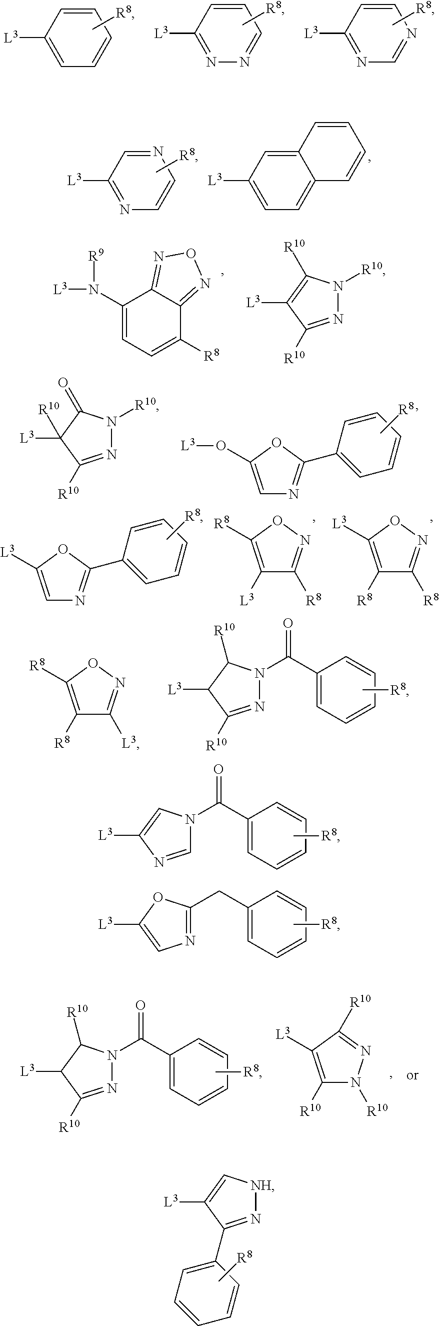

wherein R² is attached to the phenyl ring of Formula V through L³;

L³ may be a bond, —CH₂—, —CH₂(CH₂)ₙ—, —CH(OH)—, —C(O)—, —O—, —NH—, —S—, —CH₂CH₂—, —CH═CH—, —N═N—, —OCH₂—, —NHP(O)(OH)—, —NHS(O)₂—, —SCH₂—, —S(O)₂CH₂—, or —NHC(O)—;

R⁸ may be —H, —CH₃, —C(CH₃), —OH, —NH₂, NO₂, —OCH₃, —C(O)OH, —C(O)NH₂, or halogen;

R¹⁰ may be —H, —CH₃, —OH, —OCH₃, —C₆H₅,

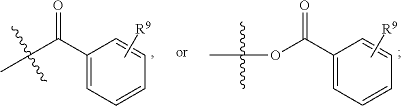

R⁹ may be —H, —CH₃, —C(CH₃), —OH, —NH₂, NO₂, —OCH₃, —C(O)OH, —C(O)NH₂, or halogen; and

s may be 1 to 20.

In some embodiments in the compound of general formula VI or pharmaceutically acceptable salt or solvate thereof, L¹ may be —S—, —S(O)₂—, —C(O)—, or —P(O)(OH)—, and in other embodiments, L² may be —NH—, —NR³, —CH₂—, or —C(R³)₂—. In still other embodiments, L¹ may be —NH—, —NR³, —CH₂—, or —C(R³)₂—, and in yet other embodiments, L² may be —S—, —S(O)₂—, —C(O)—, or —P(O)(OH)—. In certain embodiments, L′ may be —S—, —S(O)₂—, or —C(O)—, and L² may be —NH—, or —CH₂—, and in some embodiments, L¹ may be —S(O)₂— and L² is —NH—.

In other embodiments of compounds of formula VI or pharmaceutically acceptable salts or solvates thereof:

L¹ may be —S(O)₂—;

L² may be —NH—;

R² may be —NHS(O)₂R⁶;

R⁶ may be aryl, heteroaryl, or C₁-C₂₀ alkyl, wherein each of the aryl, heteroaryl, or C₁-C₂₀ alkyl, may be optionally substituted with one or more substituents independently selected from —NH₂, —OH, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen;

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

In some embodiments of formula VI, R² may be

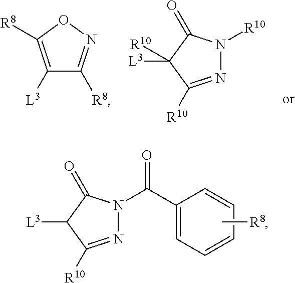

wherein R² is attached to the benzene ring of formula VI through L³, and L³ may be —NHS(O)₂— or —N═N—. In other embodiments, R² may be

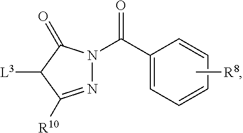

wherein R² is attached to the benzene ring of formula VI through L³ and L³ may be —N═N—. In yet other embodiments of formula VI or pharmaceutically acceptable salts or solvates thereof:

R² may be —NHS(O)₂R⁶;

R⁶ may be aryl or heteroaryl, each of which may optionally be substituted with one or more substituents independently selected from —NH₂, —OH, —CH₃, —CH₂CH₃, —CH₂CH₂CH₃, C₁₋₆ alkyl, —C₆H₅, —C₆H₄R⁷, —CH₂C₆H₅, —CH₂C₆H₄R⁷, and halogen; and

R⁷ may be —H, —CH₃, heteroaryl, —C(CH₃)₃, —OH, —NH₂, NHC(O)CH₃, S(O)₂OH, —P(O)₂OH, As(O)₂OH, NO₂, —OCH₃, —OCH₂CH₃, —C(O)OH, —C(O)NH₂, or halogen.

Still other embodiments of the invention are directed to compounds of general formula VII:

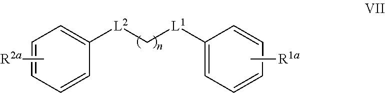

wherein:

L¹ and L² may be —S(O)₂—, —C(O)—, —CH₂—, —O—, or —S—;

n may be 1 or 2;

R^{1a} may be halogen, —C(O)OH, or

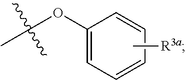

R^{1a} may be halogen, —H, —NH₂, C(CH₃)₃, or C(F)₃;

R^{2a} may be —NH₂, —NO₂, —C(O)OH, —CH₂C(O)OH, or

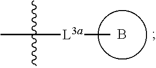

L^{3a} may be a bond, —NHC(O)—, —C(O)—, —NH—, or —O—; and

ring B may be an aryl or heteroaryl having one or two ring-forming N heteroatoms, each of which may optionally be substituted with one or more substituents independently selected from CH₃, —OH, —NH₂, —NO₂, —C(CH₃)₃, —C(O)OH, —S(O)₂OH, —P(O)₂OH, As(O)₃H, NHC(O)CH₃, —OH, —OCH₃, —OCH₂CH₃, and halogen.

In some embodiments of formula VII or pharmaceutically acceptable salts or solvates thereof, L¹ may be —S(O)₂—; L² may be —S—; and n may be 2, and in other embodiments:

R^{1a} may be halogen;

R^{2a} may be —NH₂, or

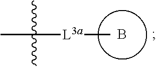

L^{3a} may be —NHC(O)— or —NH—; and

ring B may be an aryl or heteroaryl having one or two ring-forming N heteroatoms, each of which may be optionally substituted with one or more substituents independently selected from CH₃, —OH, —NH₂, —NO₂, —C(CH₃)₃, —C(O)OH, —S(O)₂OH, —P(O)₂OH, As(O)₃H, NHC(O)CH₃, —OH, —OCH₃, —OCH₂CH₃, and halogen.

Further embodiments of the invention are directed to compounds of general formula VIII:

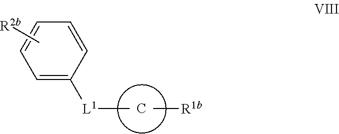

or pharmaceutically acceptable salts or solvates thereof wherein:

L¹ may be —S(O)₂— or —C(O)—;

ring C may be aryl, piperazine, or imidazole;

R^{1b} may be an aryl group substituted with one or more C(O)OH, CH₂C(O)OH, or imidazole;

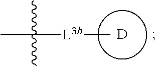

R^{2b} may be

L^{3b} may be a bond, —O—, or —S(O)₂—; and

ring D may be a substituted or unsubstituted, 5- to 9-membered cyclic of bicyclic ring having 0-3 ring-forming heteroatoms selected from N and O, wherein ring D may optionally be substituted with one or more substituents independently selected from —CH₃, —OCH₃, —NH₂, —NO₂, and halogen.

In particular embodiments of formula VII or pharmaceutically acceptable salts or solvates thereof, ring C may be a piperazine ring.

Still further embodiments of the invention include compound of formula IX:

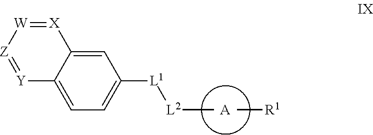

or pharmaceutically acceptable salts or solvates thereof wherein:

L¹ and L² may be —S—, —S(O)₂—, —C(O)—, —NH— or —CH₂—;

ring A may be a substituted or unsubstituted, 5- or 6-membered ring having 1-3 ring-forming heteroatoms or ring A may be a substituted or unsubstituted phenyl, wherein ring A may be optionally substituted with a methyl, methoxy group, sulfonyl, or sulfonic acid ester in addition to R¹;

R¹ may be —H, —CH₃, —CH₂CH₃, —C(CH₃)₃, —C(O)OH, —CH₂C(O)OH, —CH₂C(O)OCH₃, —CH₂C(O)OCH₂CH₃, —OH, CH₂OH, —NH₂, —CH₂NH₂, —OCH₃, S(O)₂NH₂, S(O)₂C₆H₅, or S(O)₂CH₂C₆H₅; and

W, X, Y, and Z may each independently be N or CH.

In some embodiments, L¹ may be —S—, —S(O)₂—, or —C(O)—, and L² may be —NH— or —CH₂—. In other embodiments, the bicylcic ring of formula VIII may be naphthalene, and in still other embodiment, at least one of W, X, Y, and Z of the bicyclic ring of formula VIII may be N.

Various embodiments of the invention are directed to specific compounds encompassed in general formulae I-VIII. For example, individual compounds that embody the present invention include, but are not limited to:

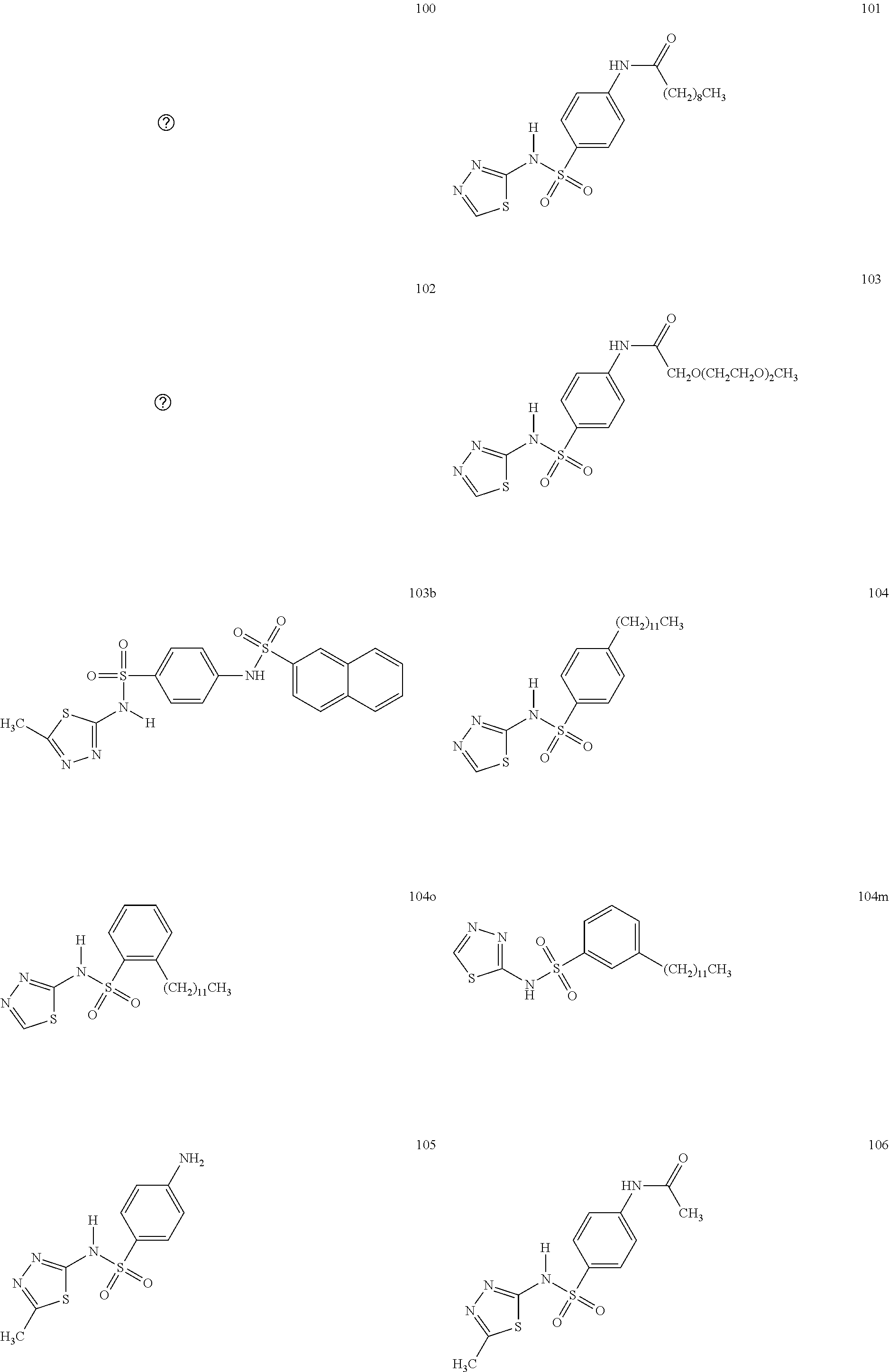

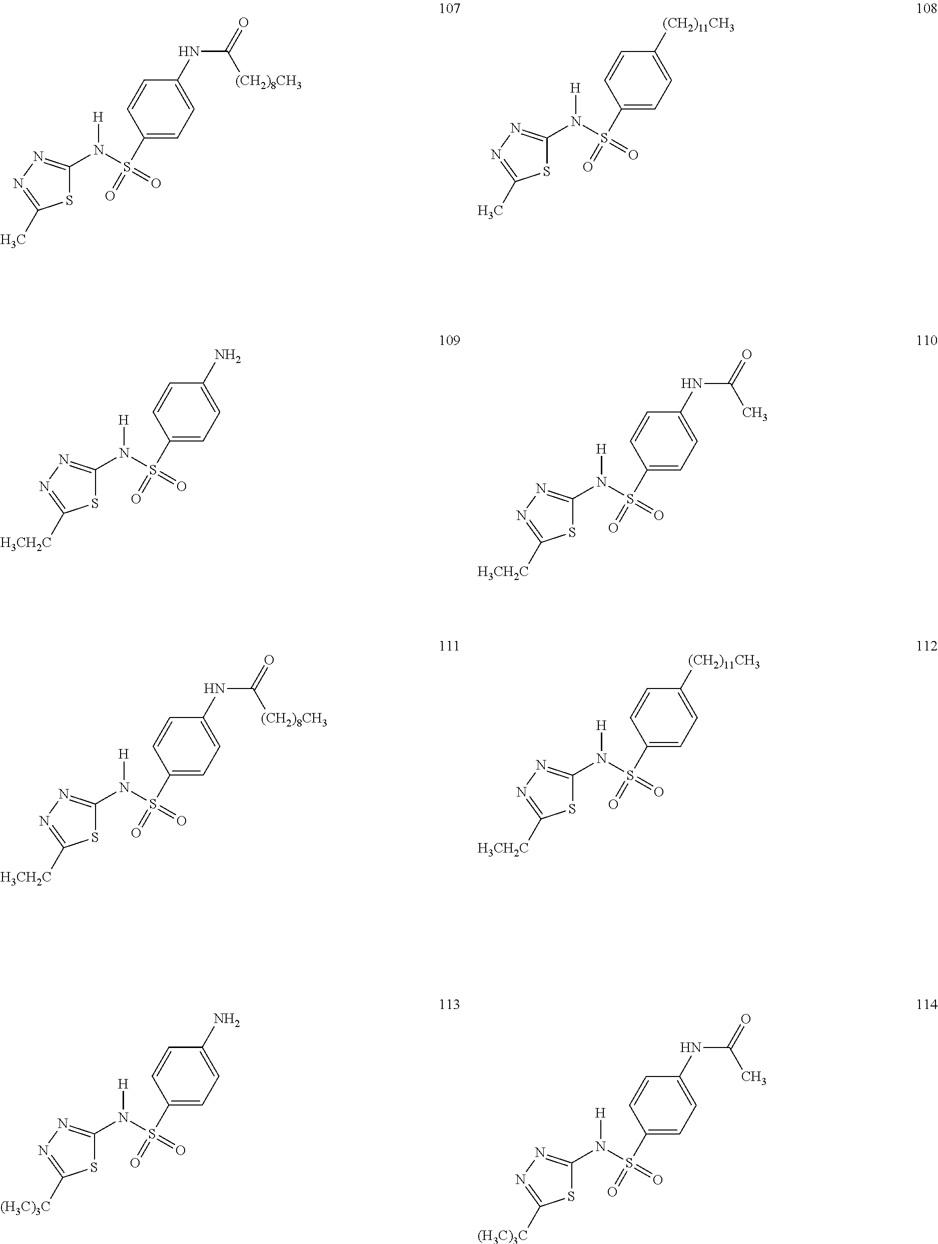

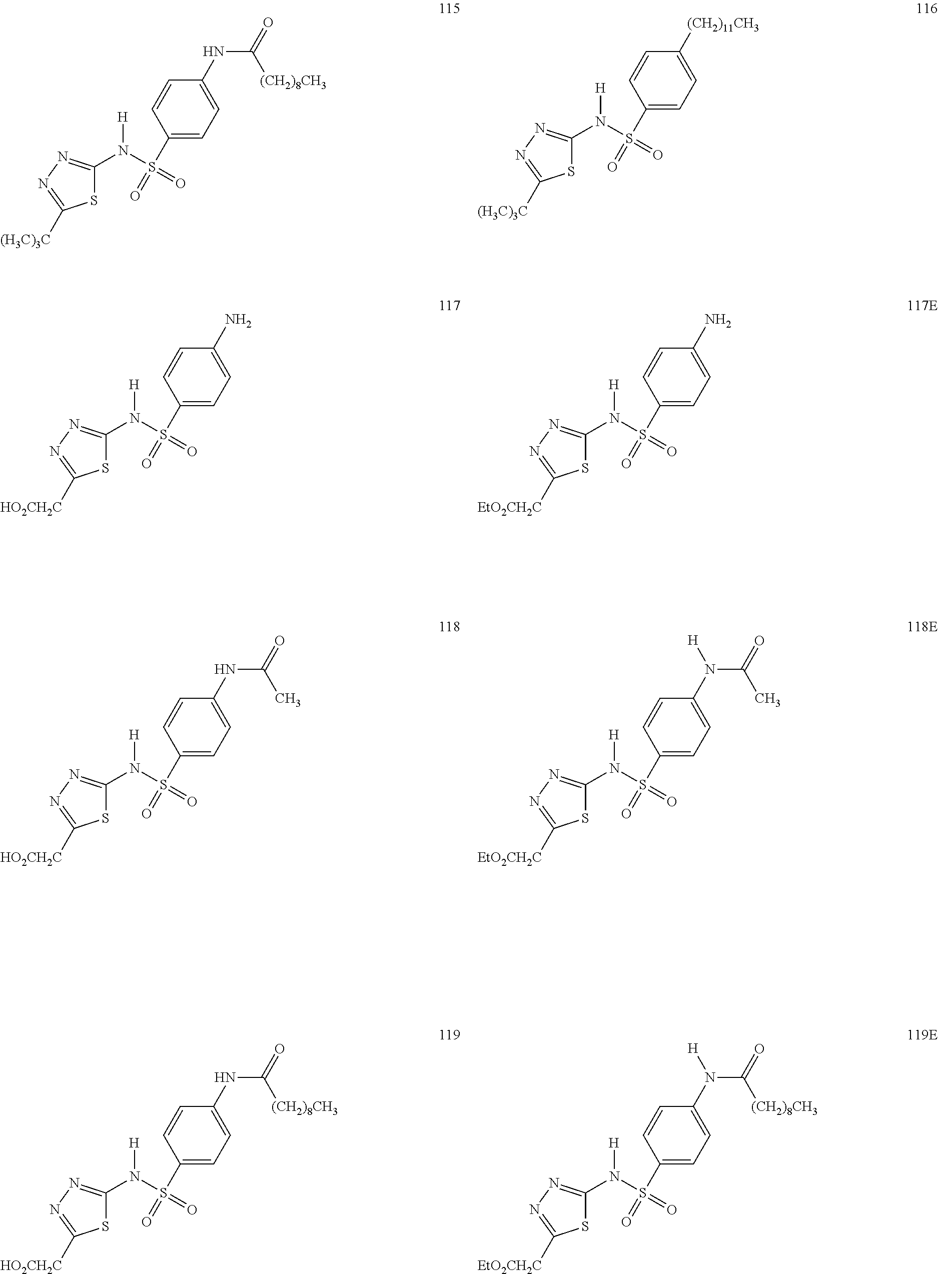

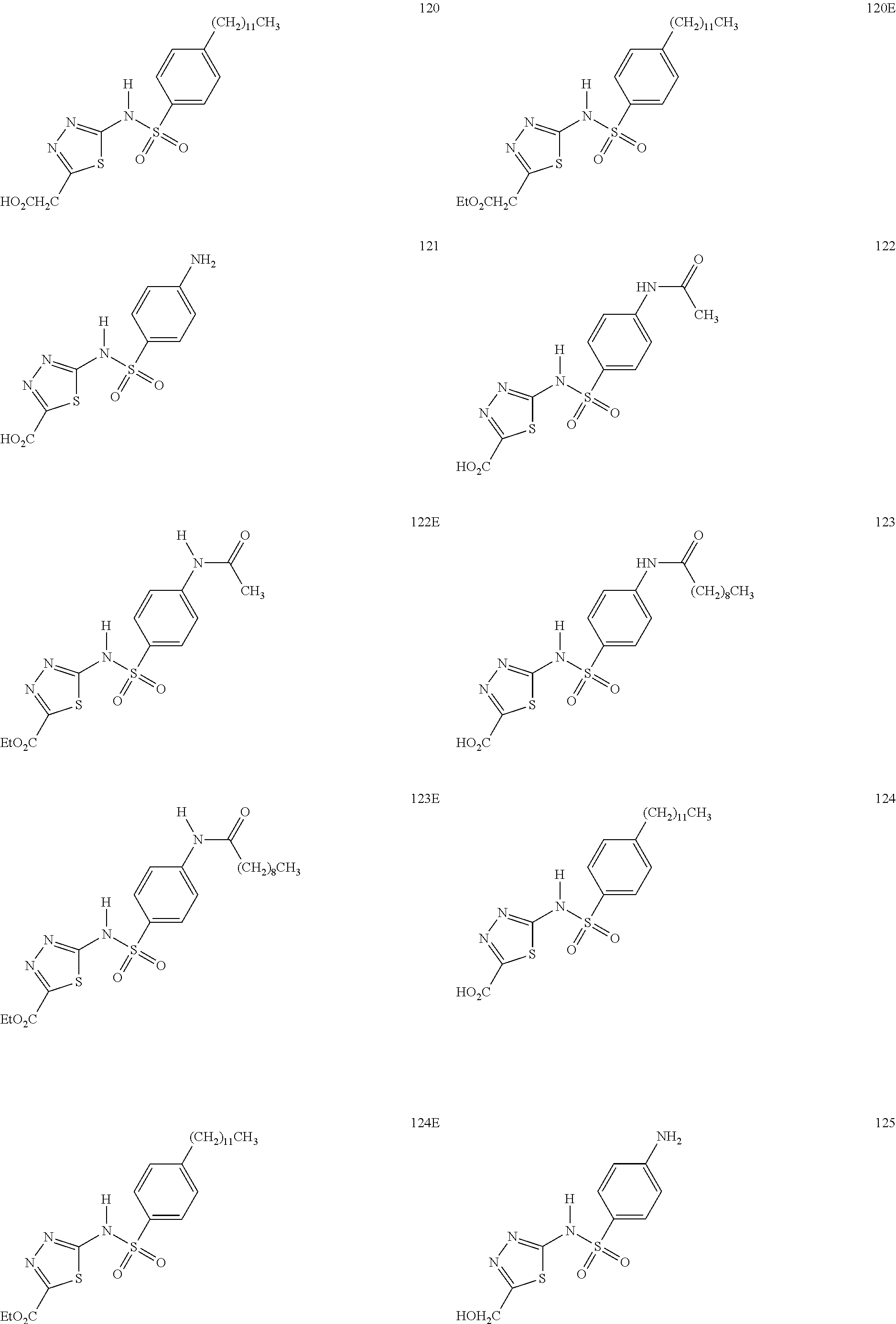

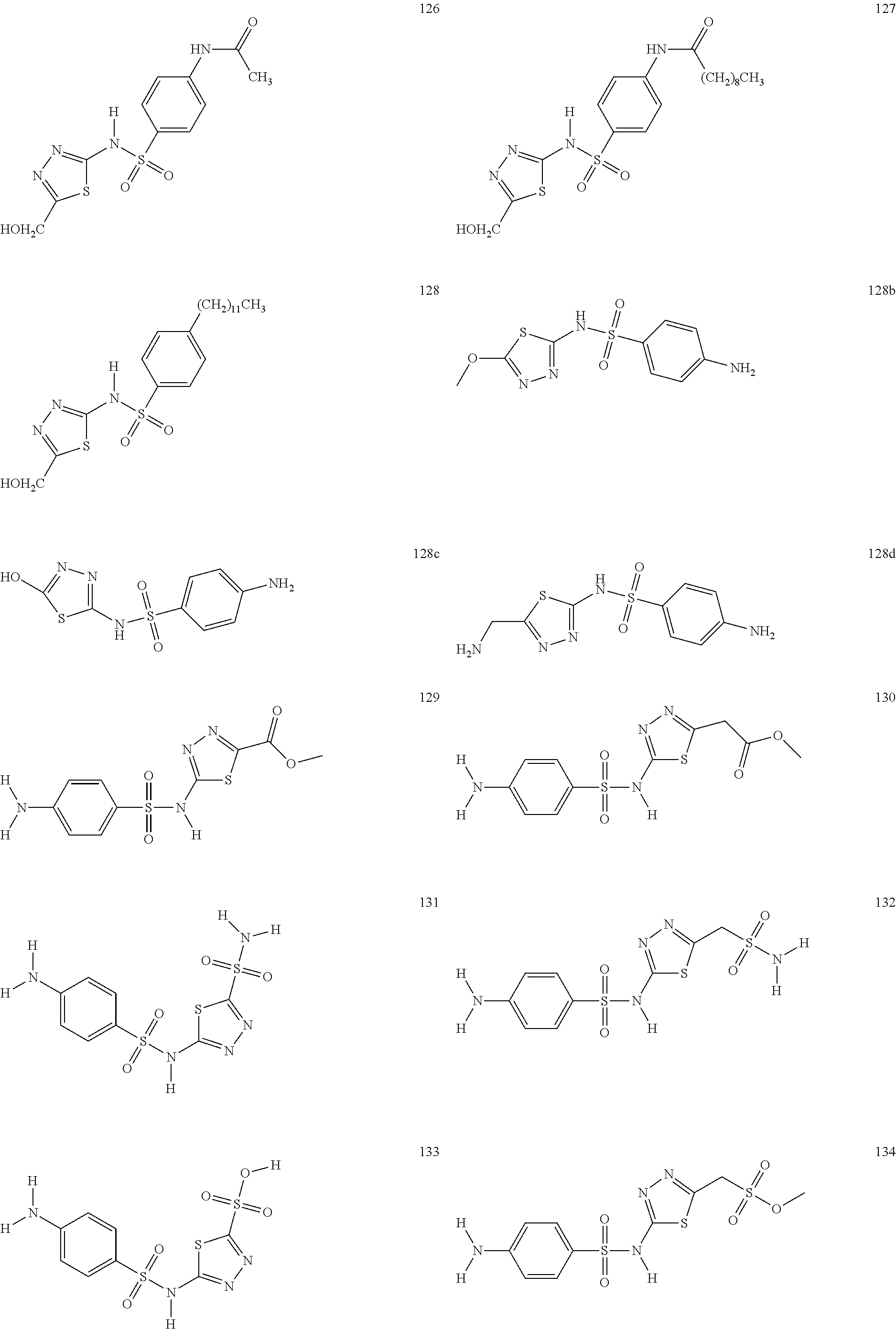

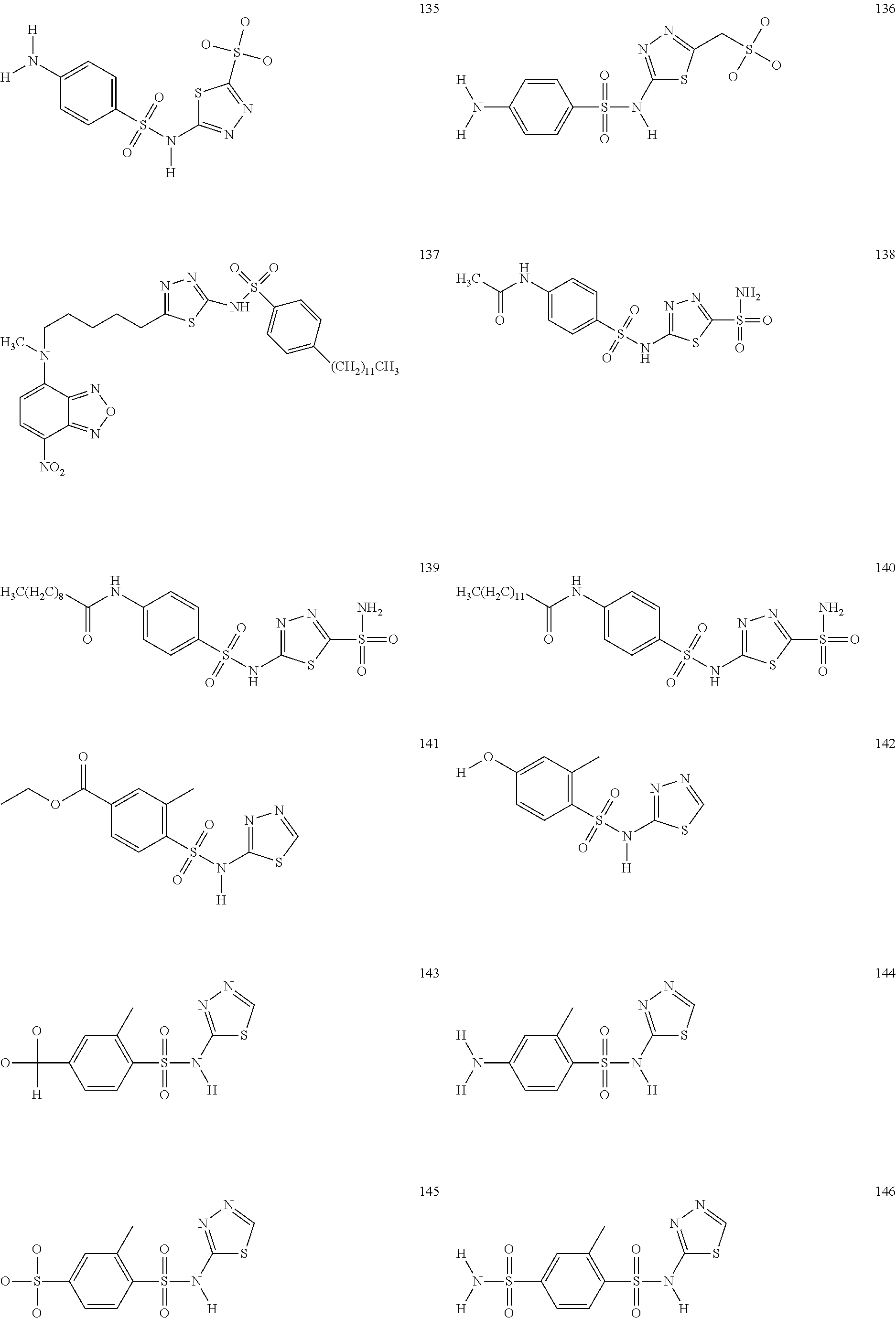

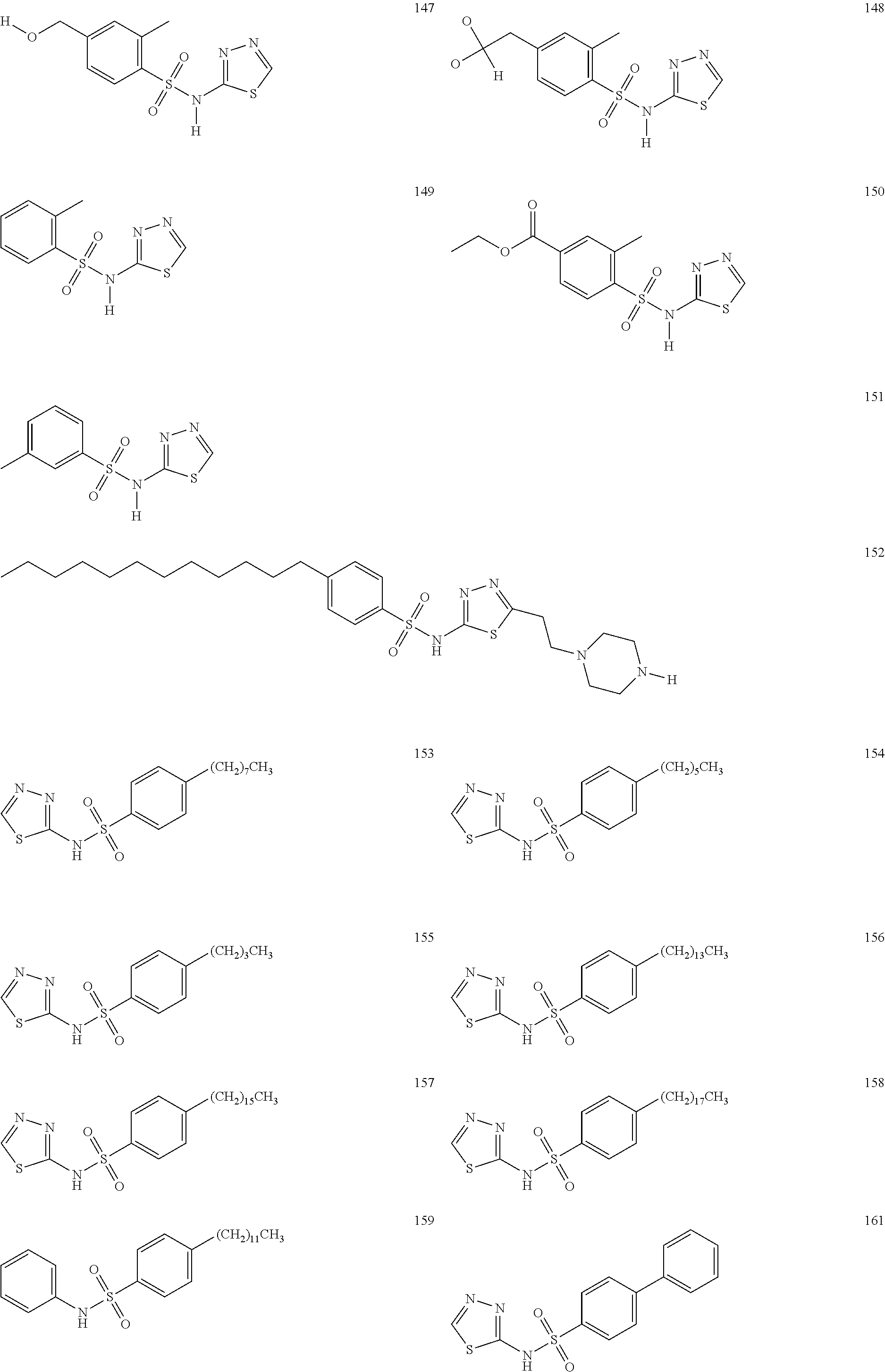

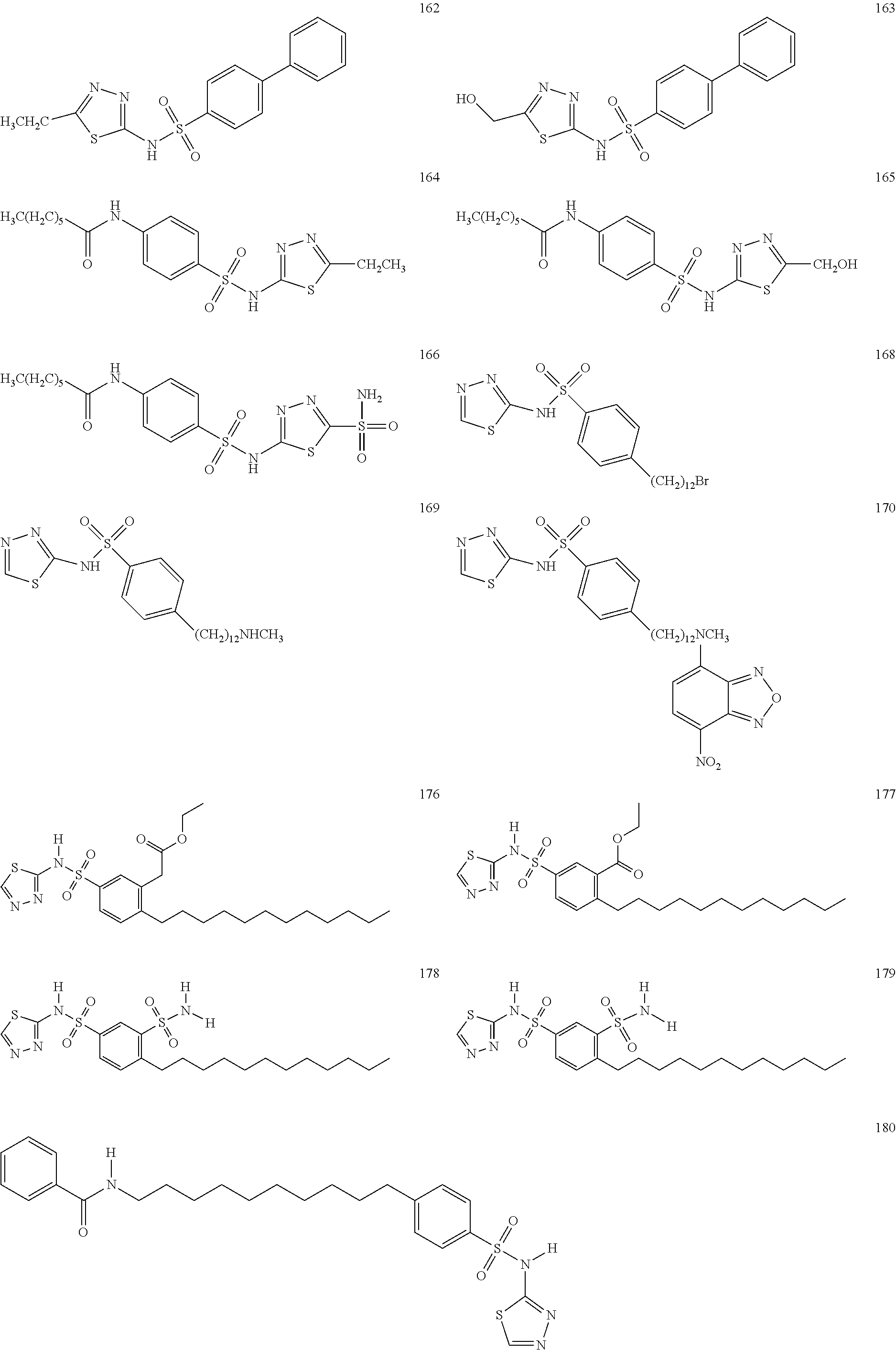

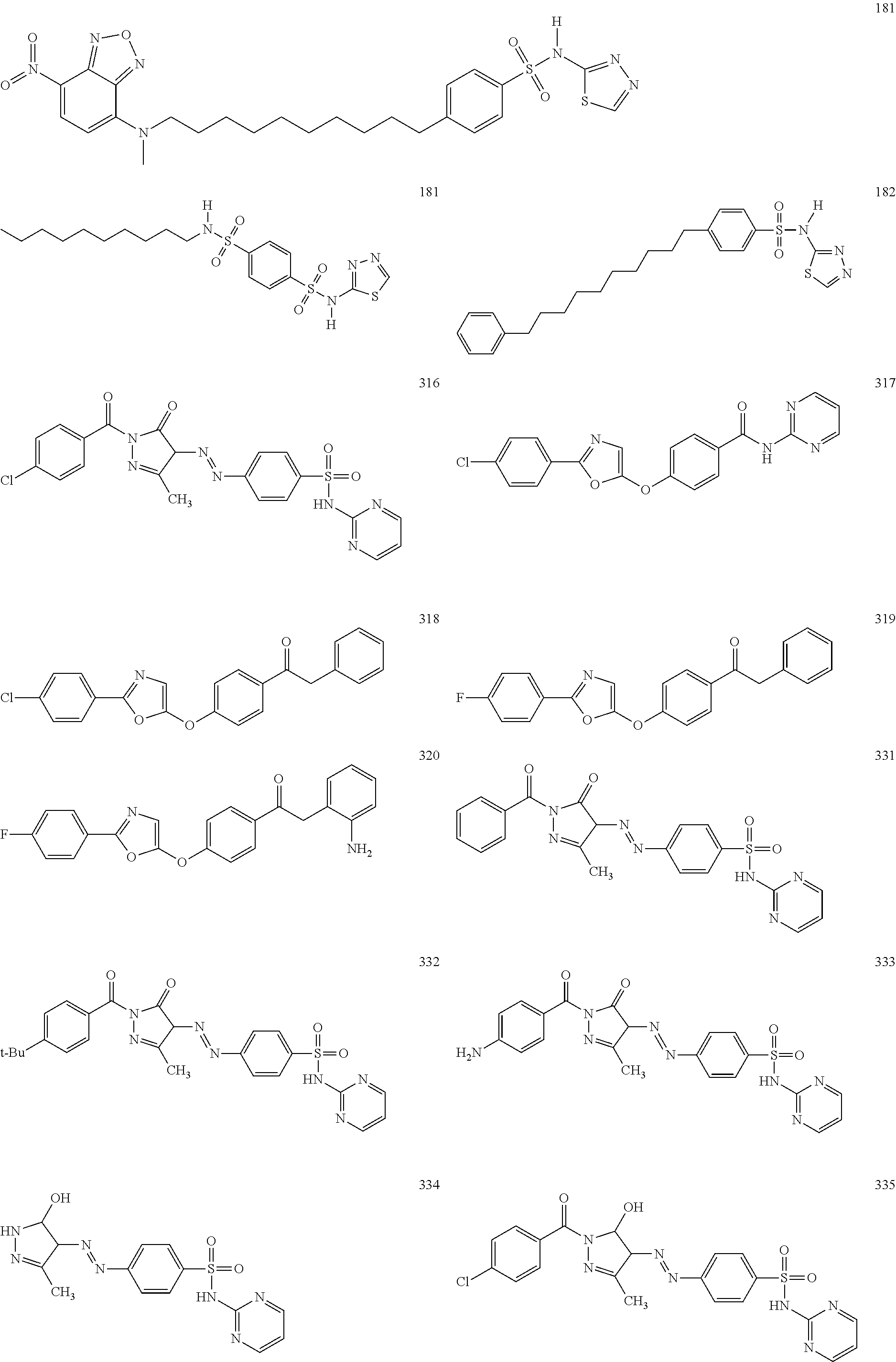

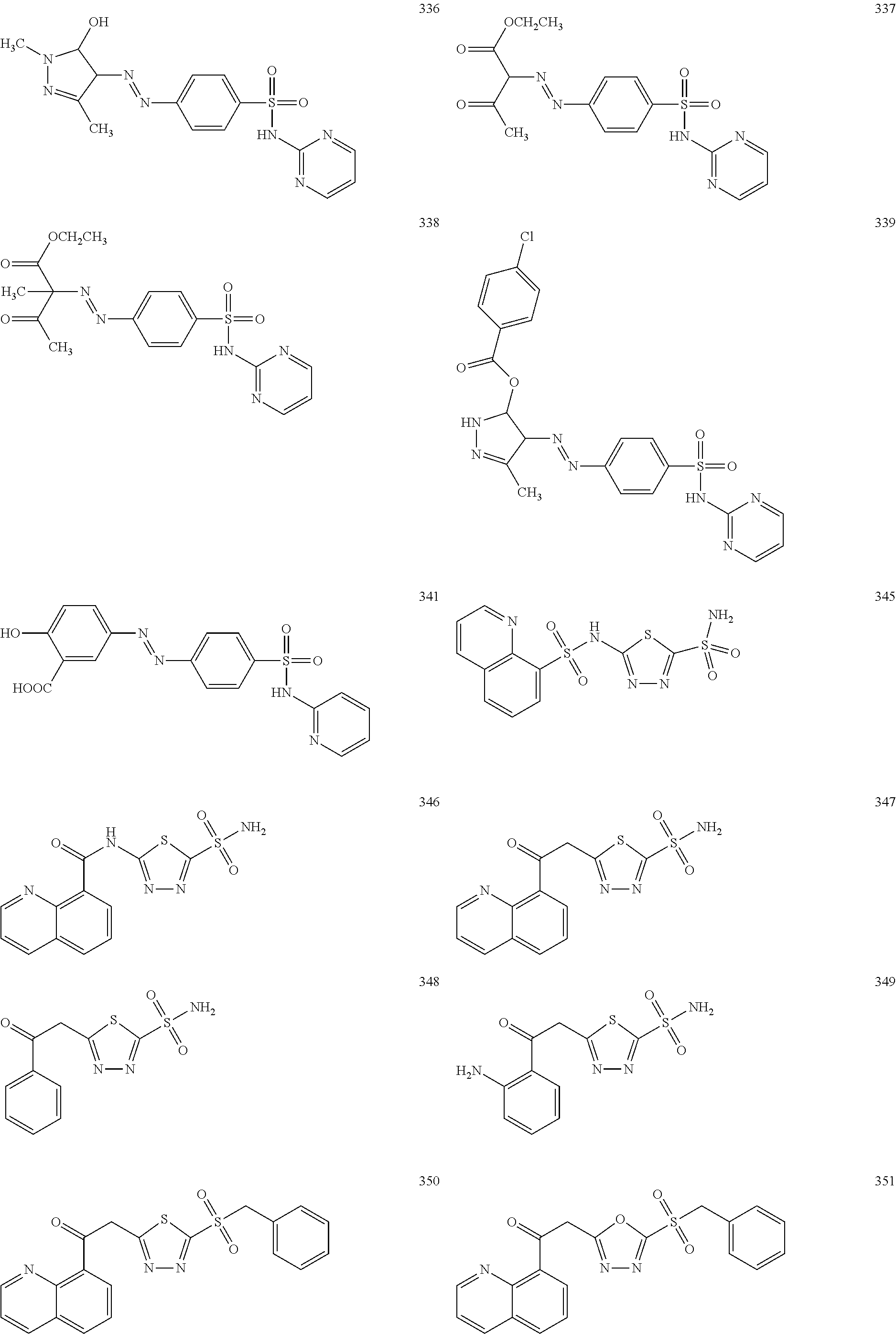

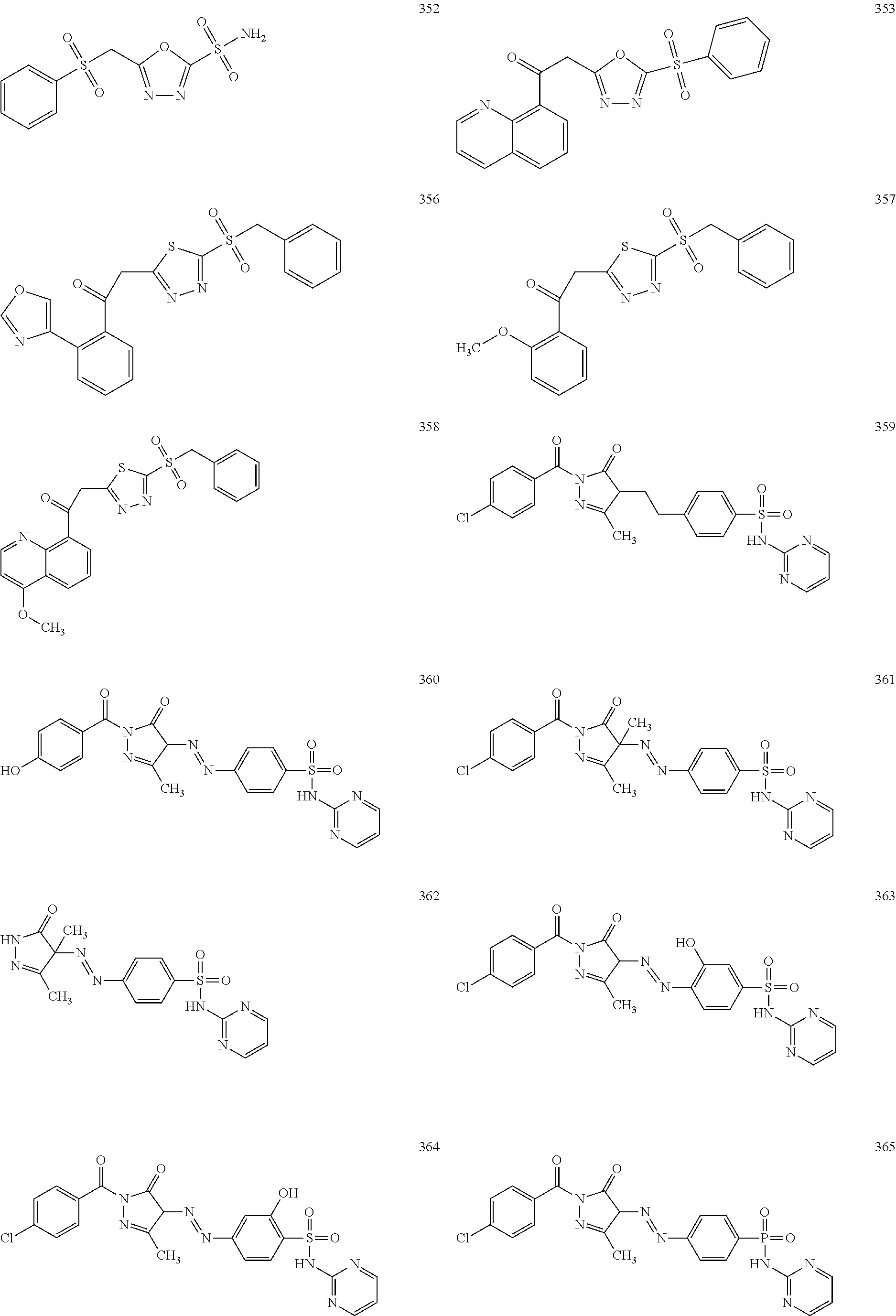

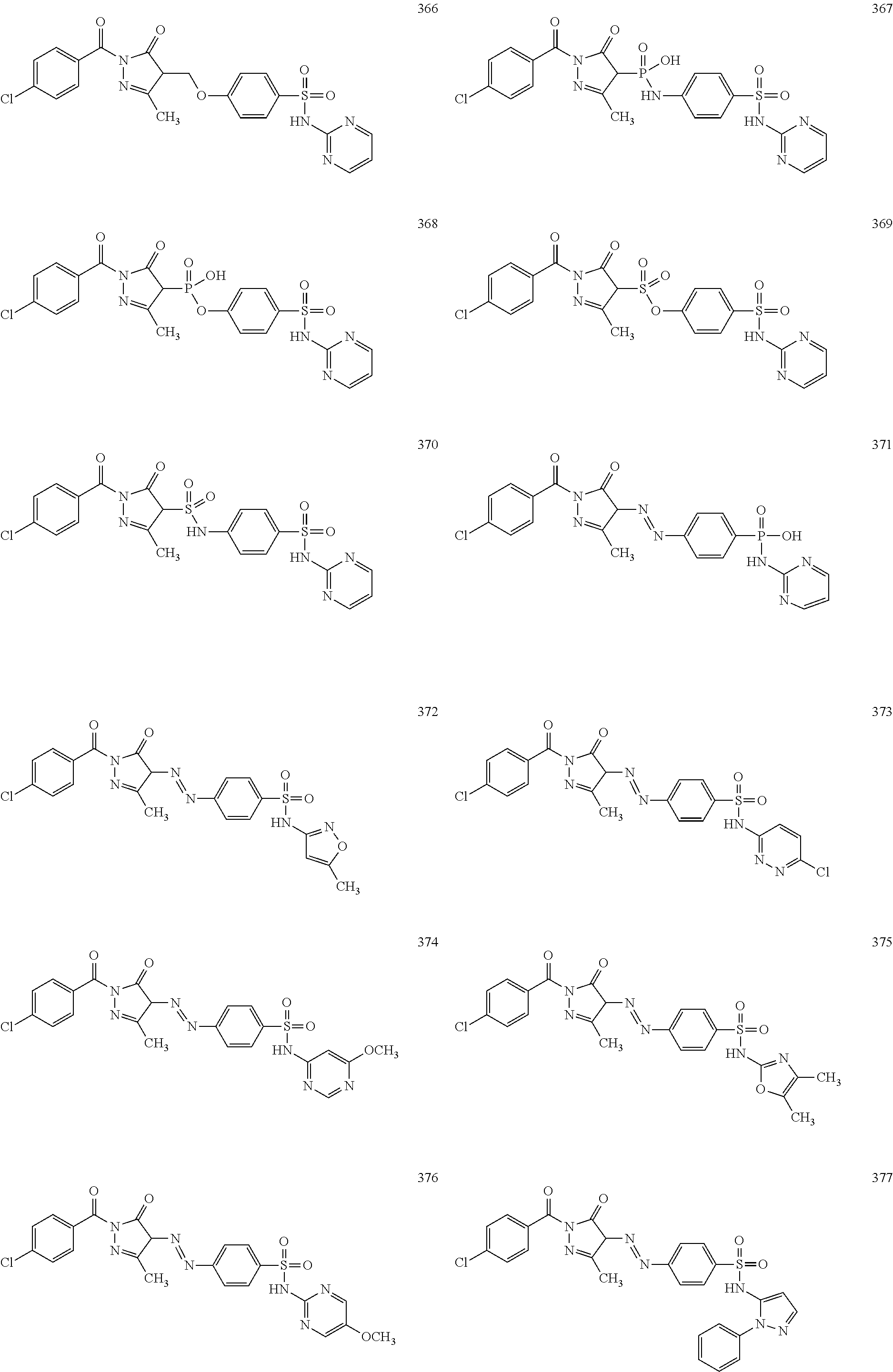

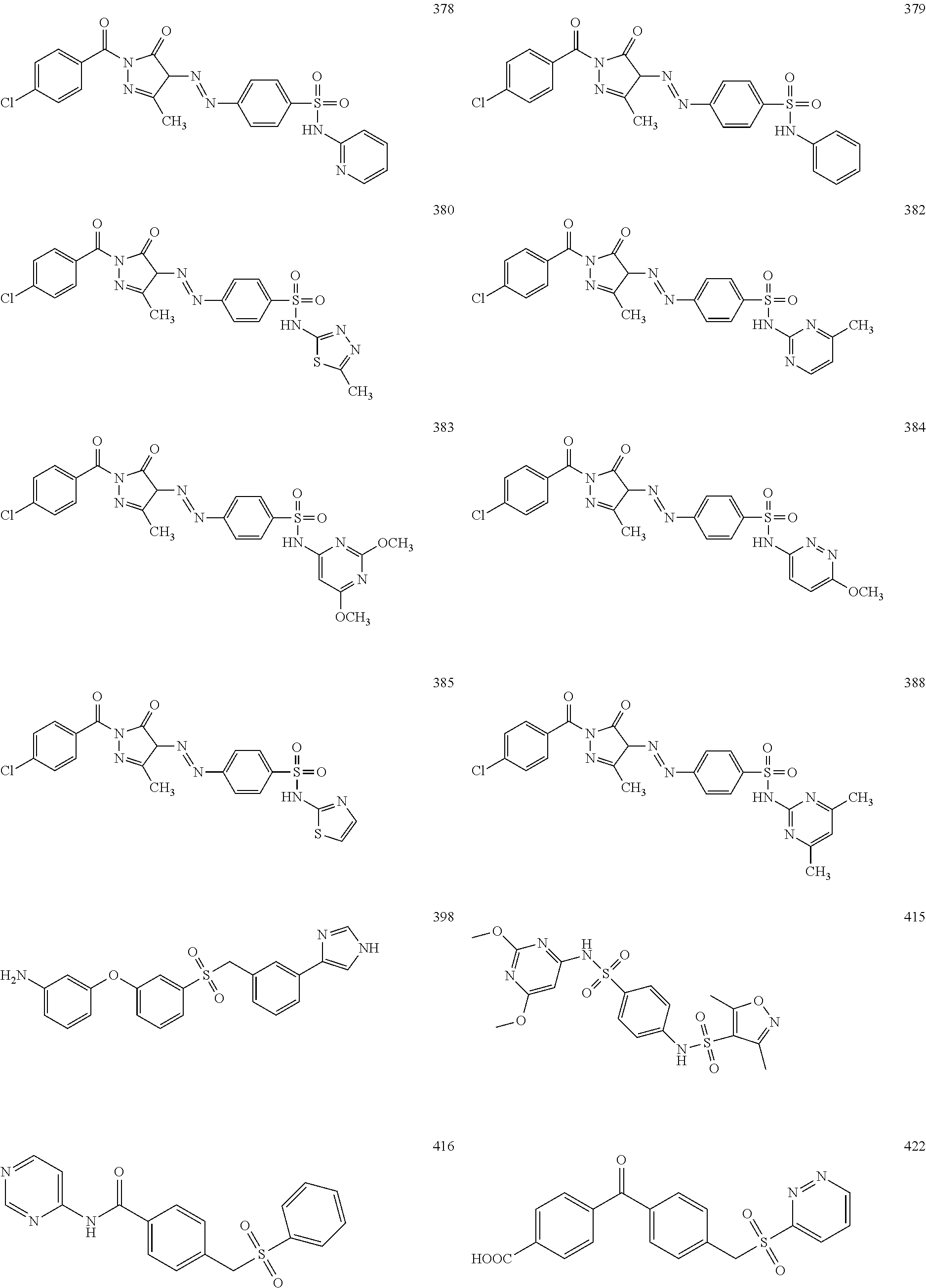

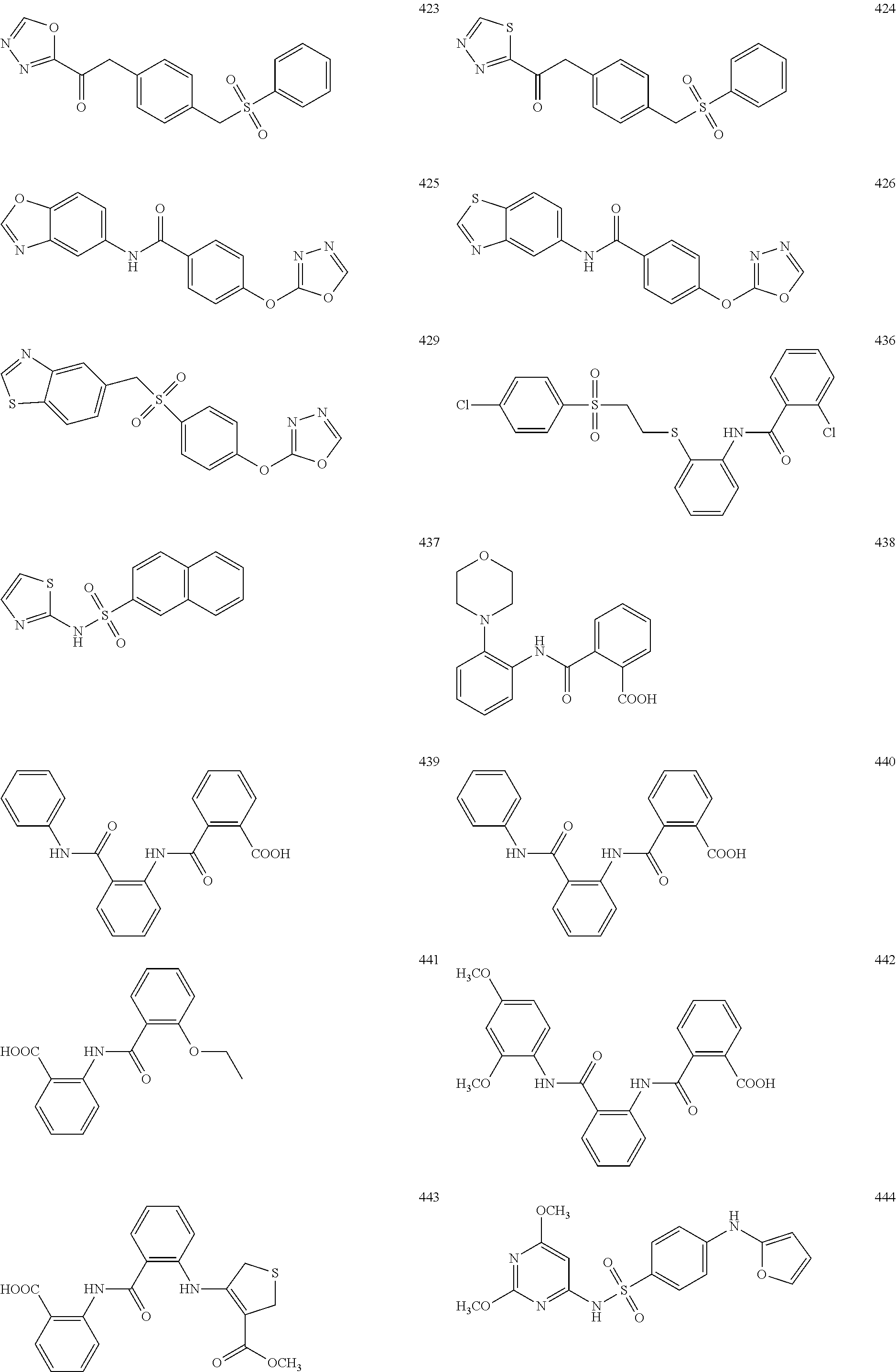

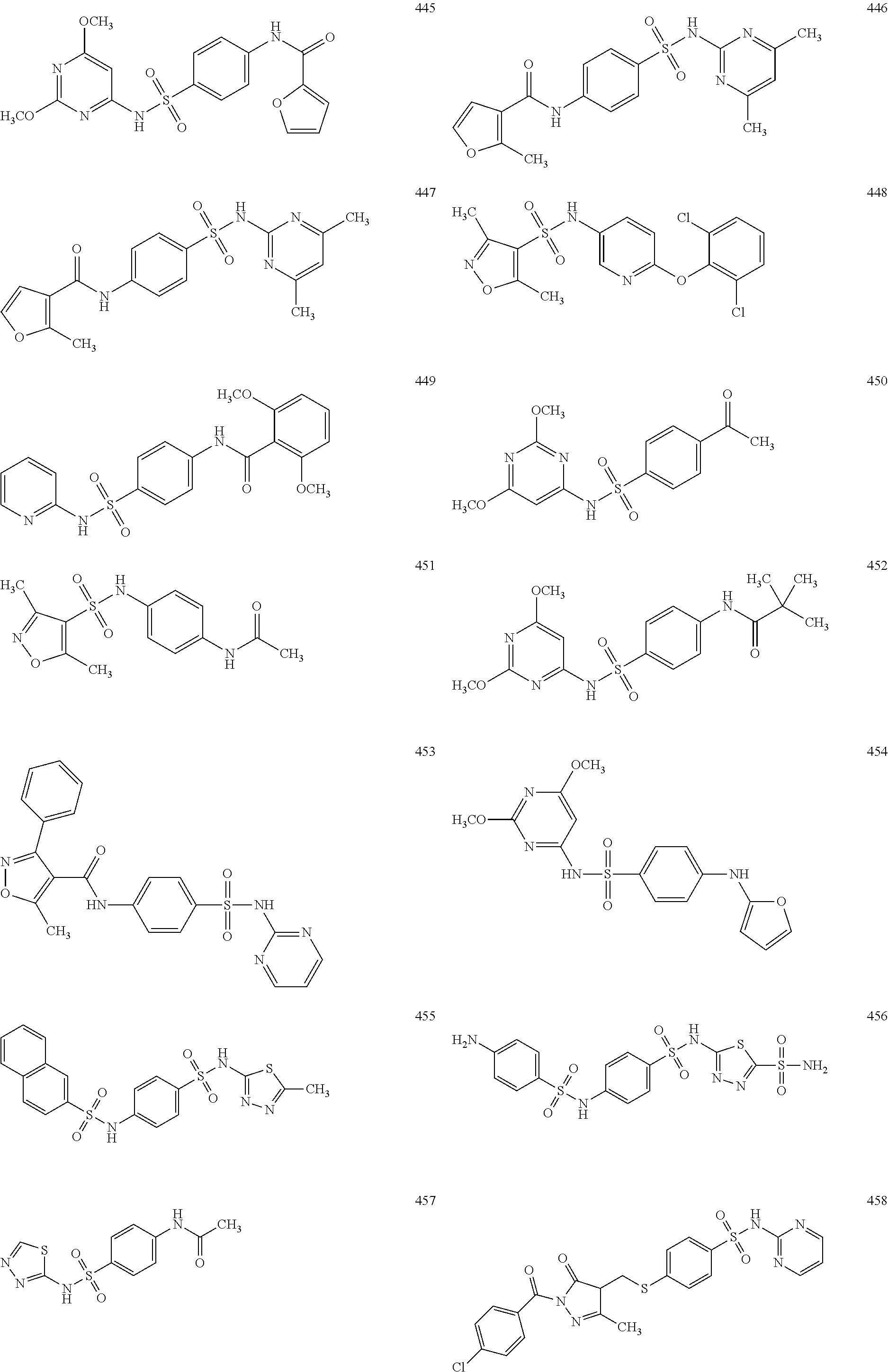

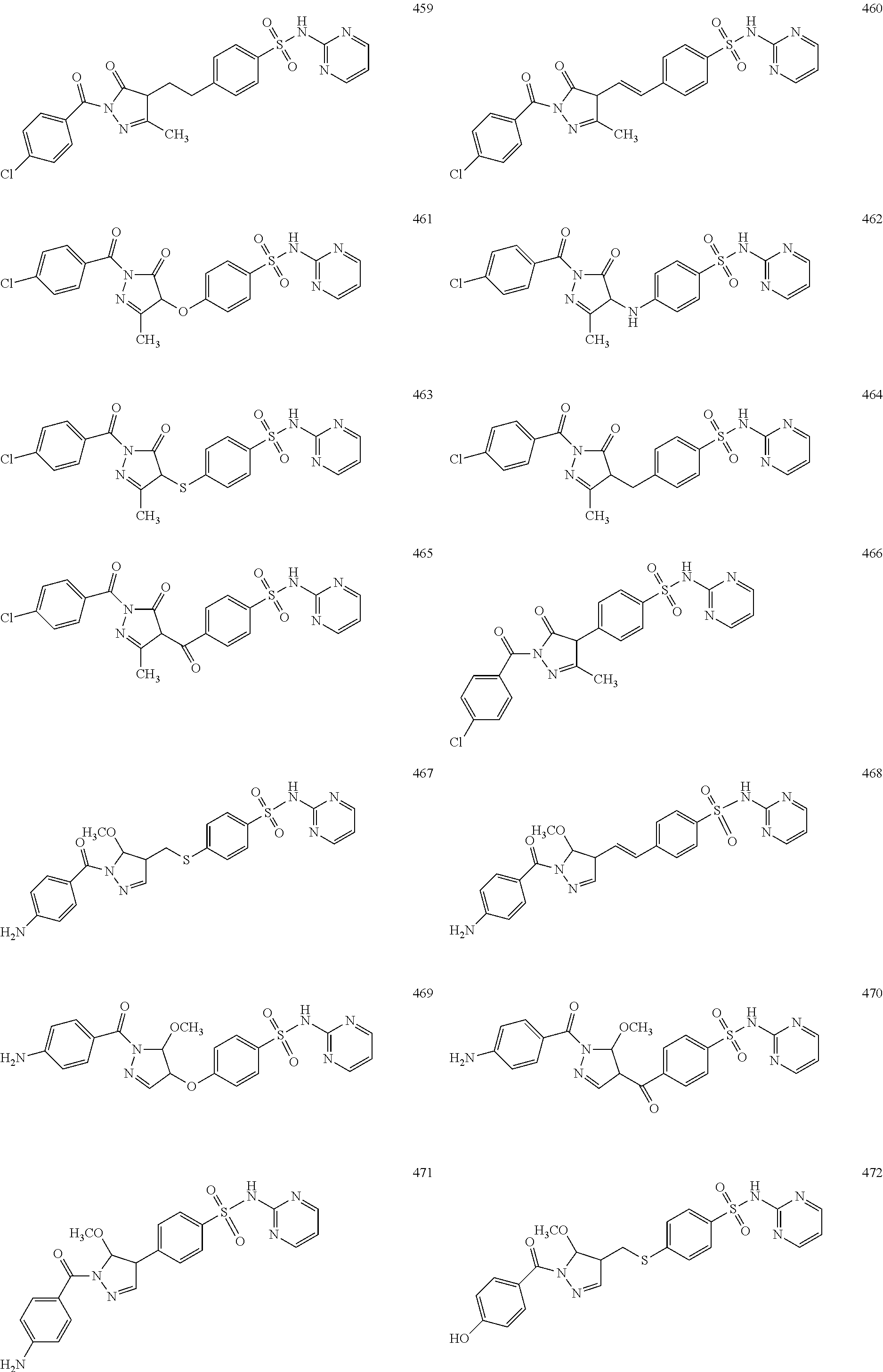

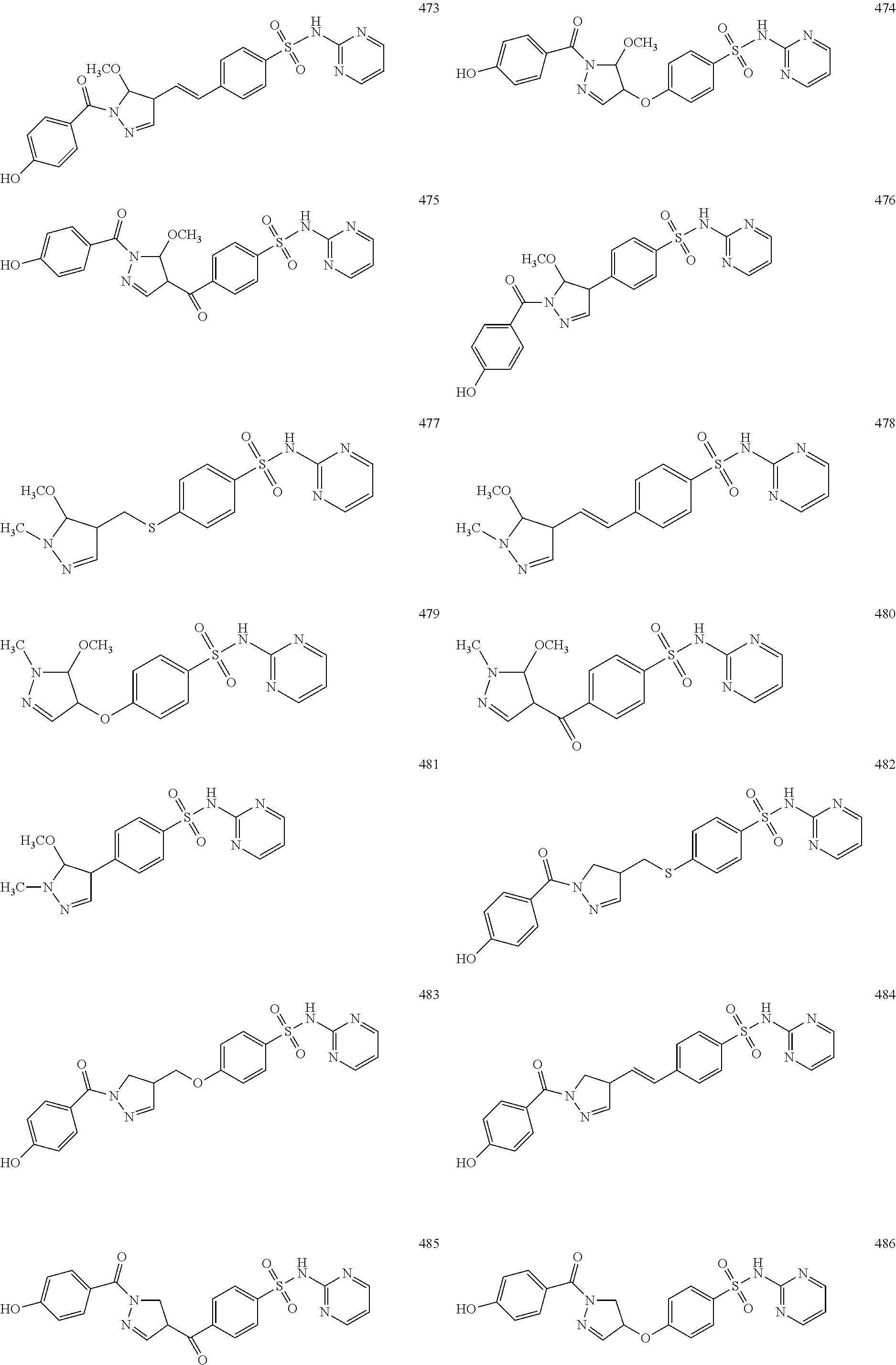

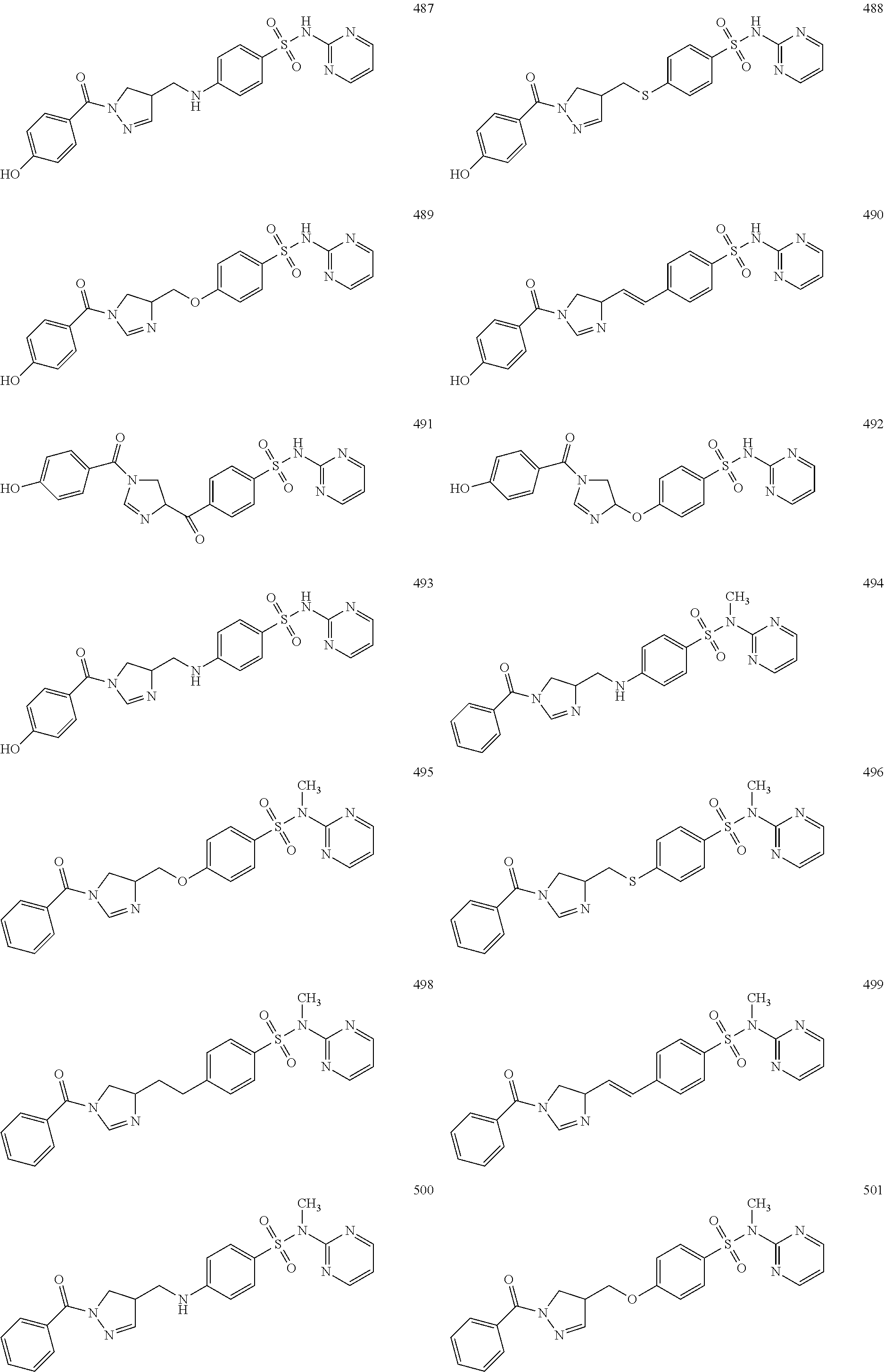

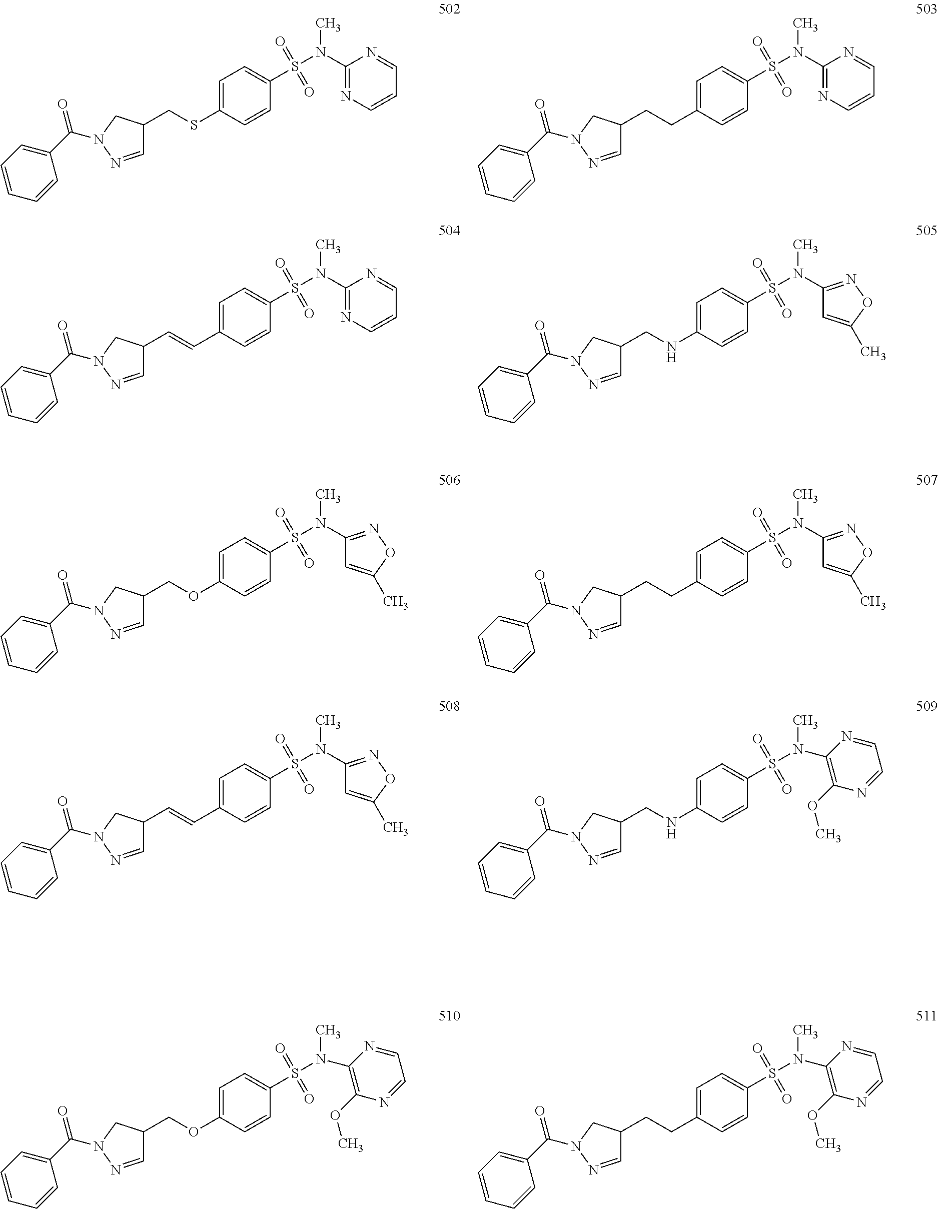

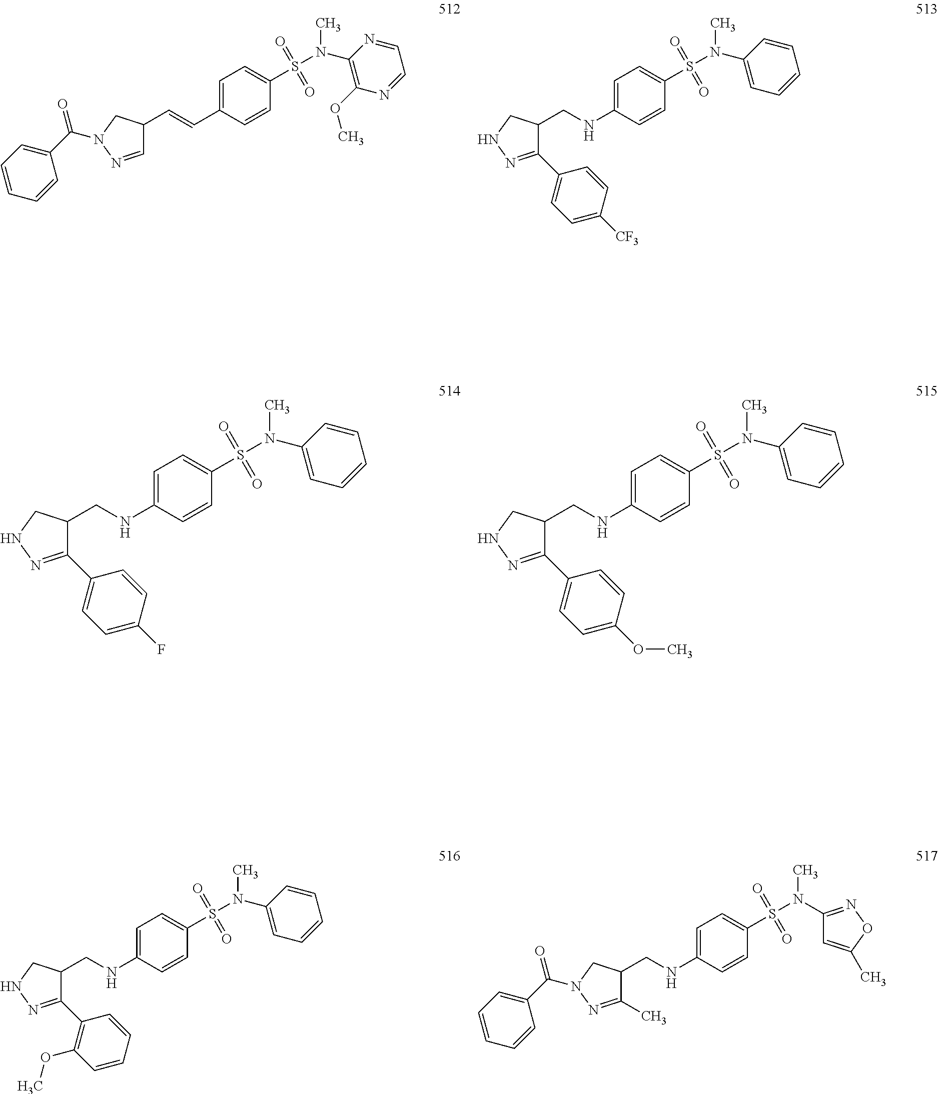

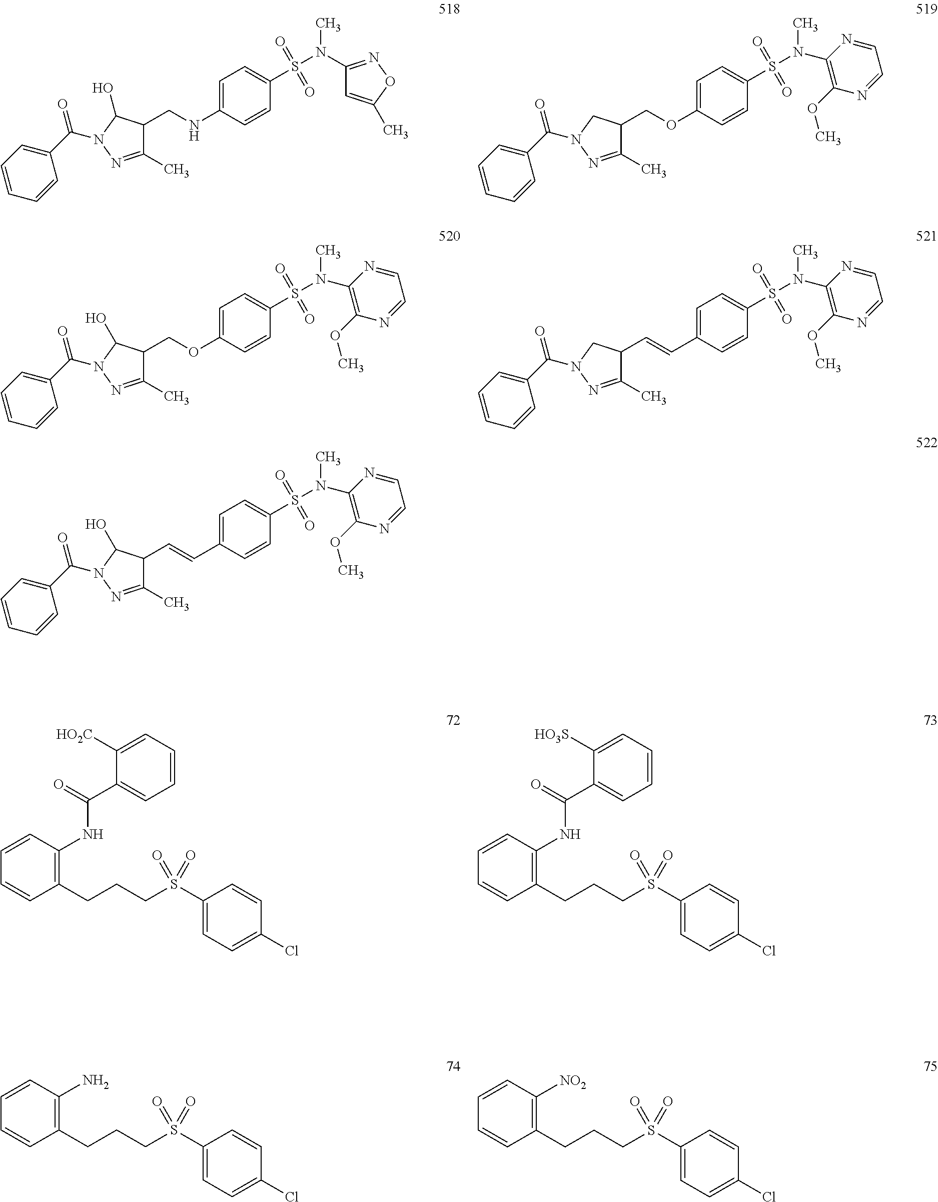

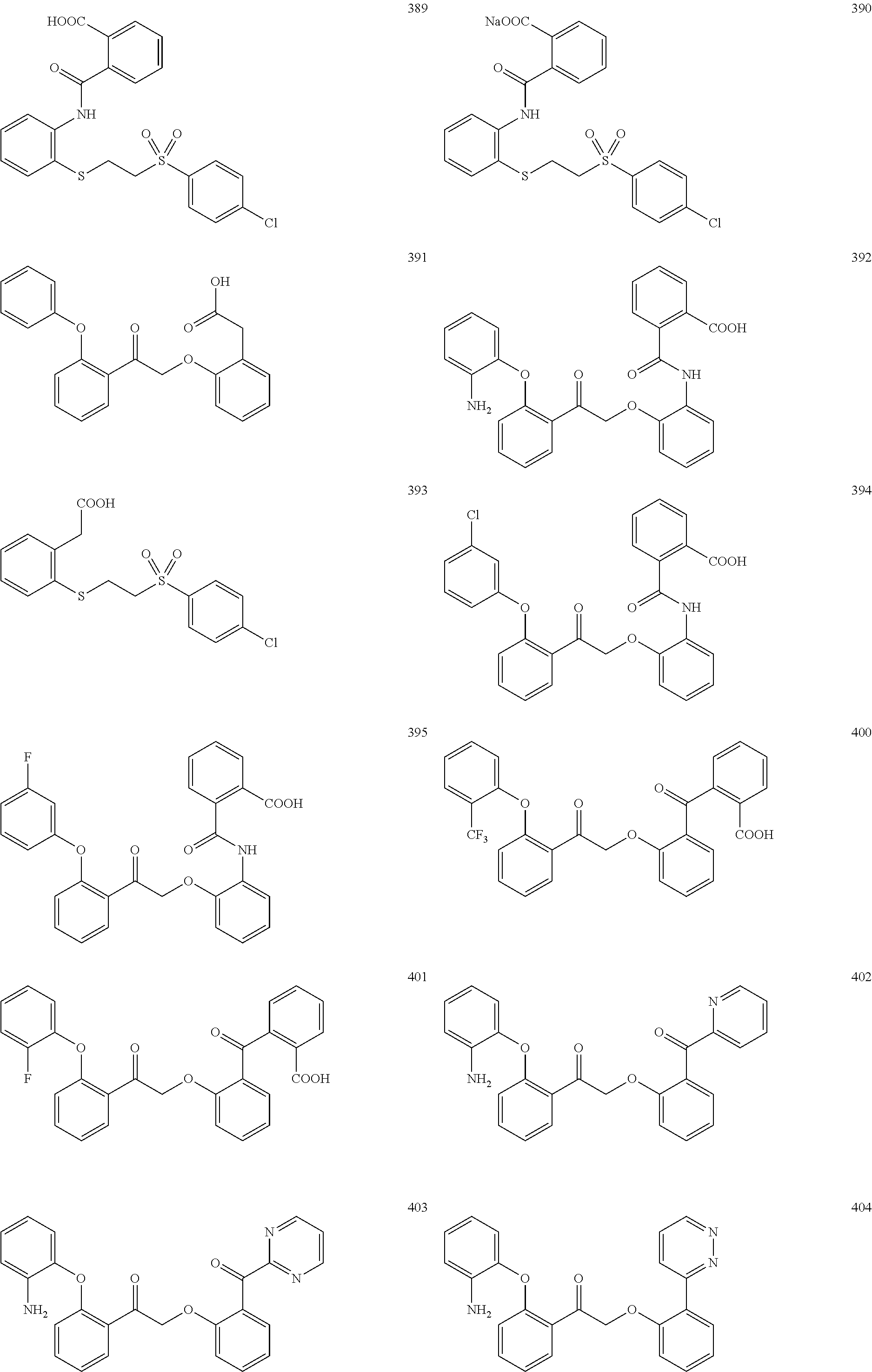

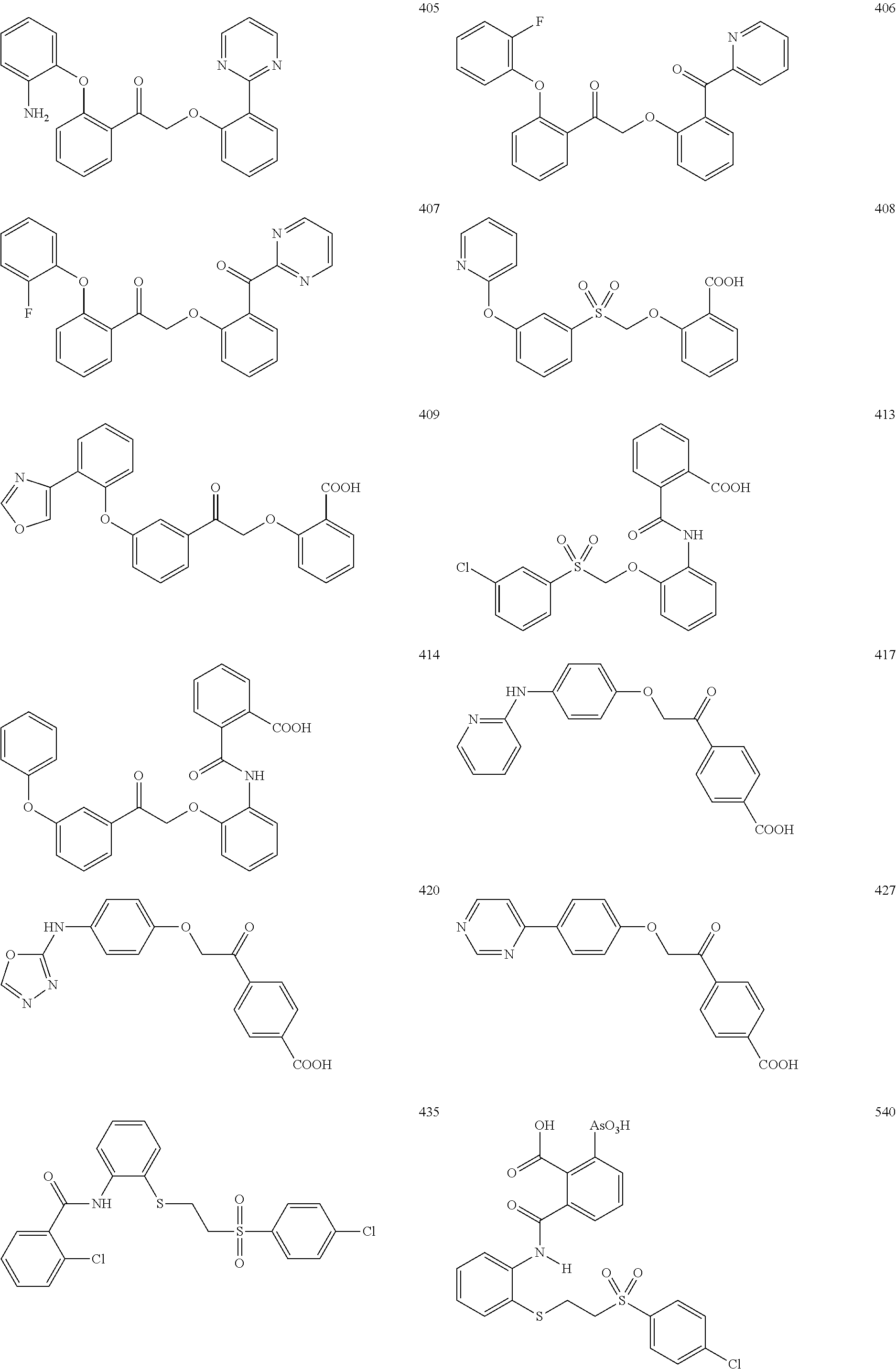

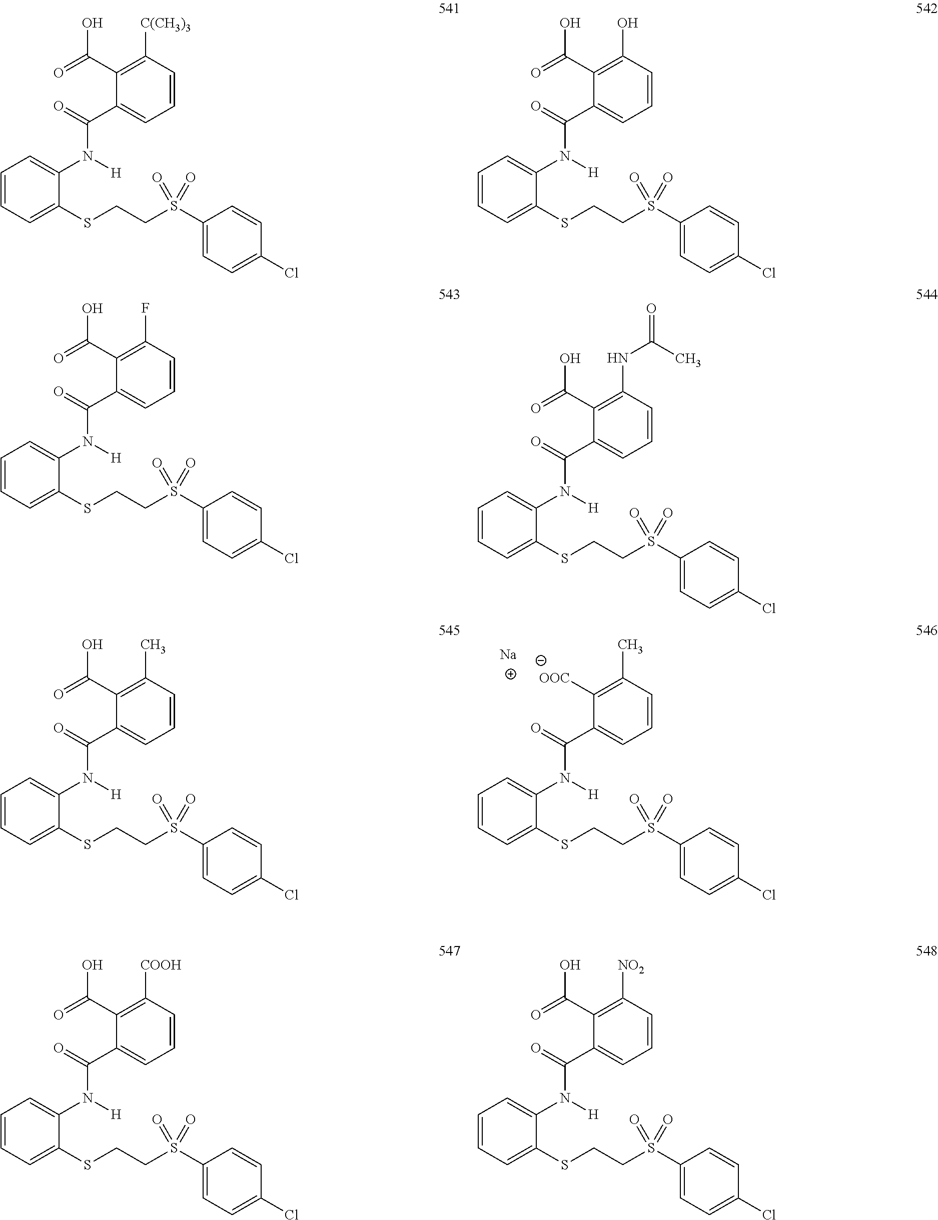

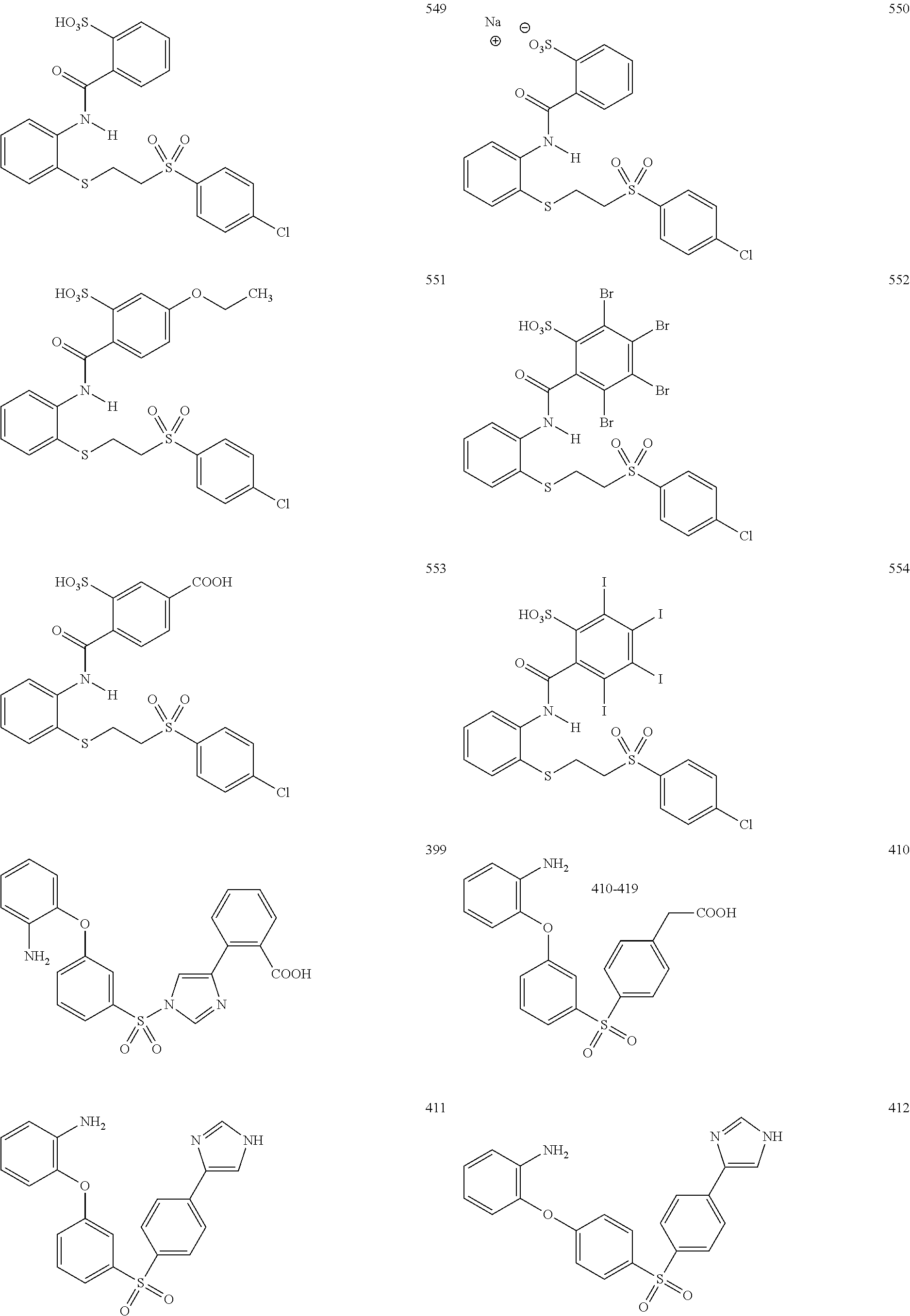

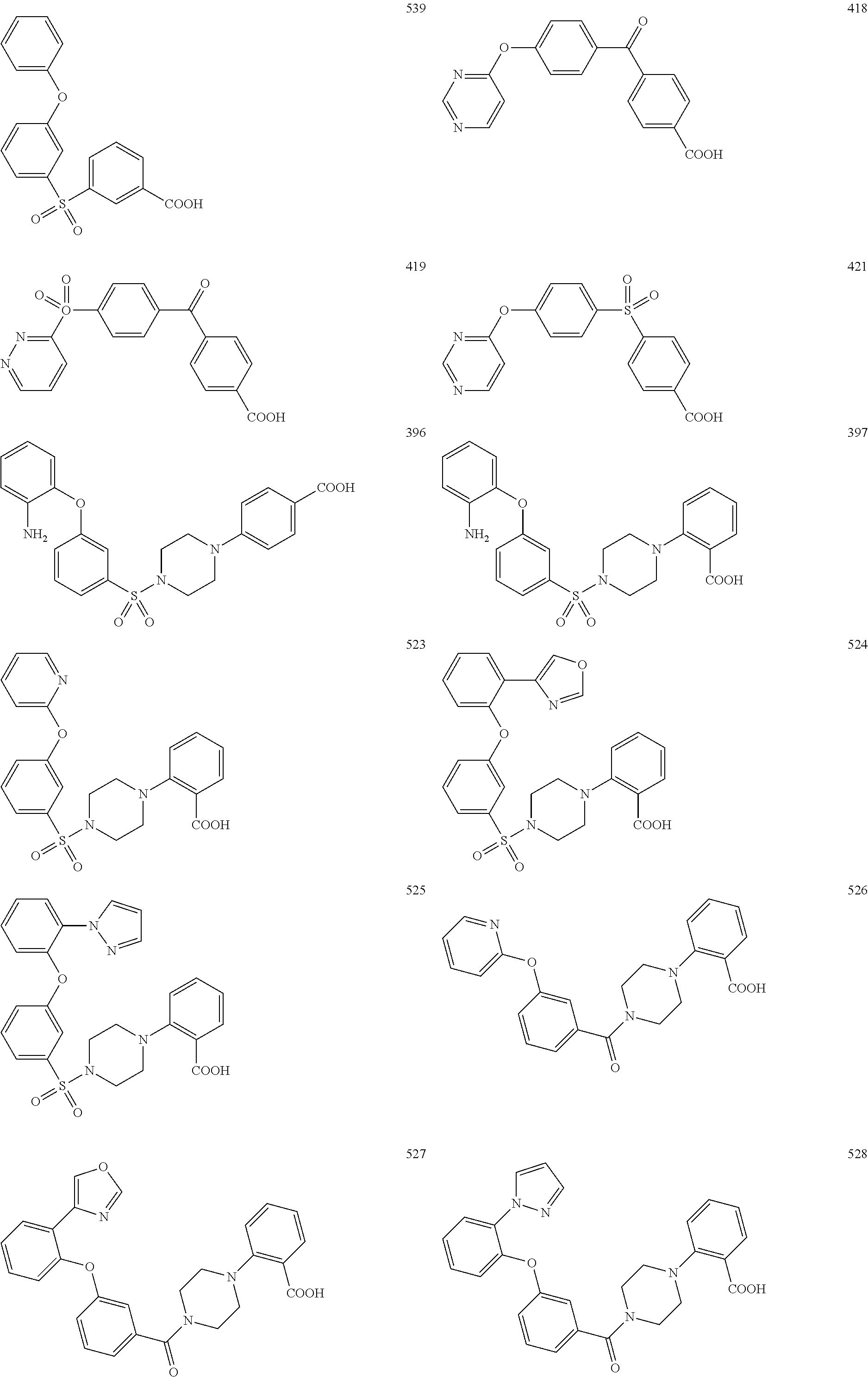

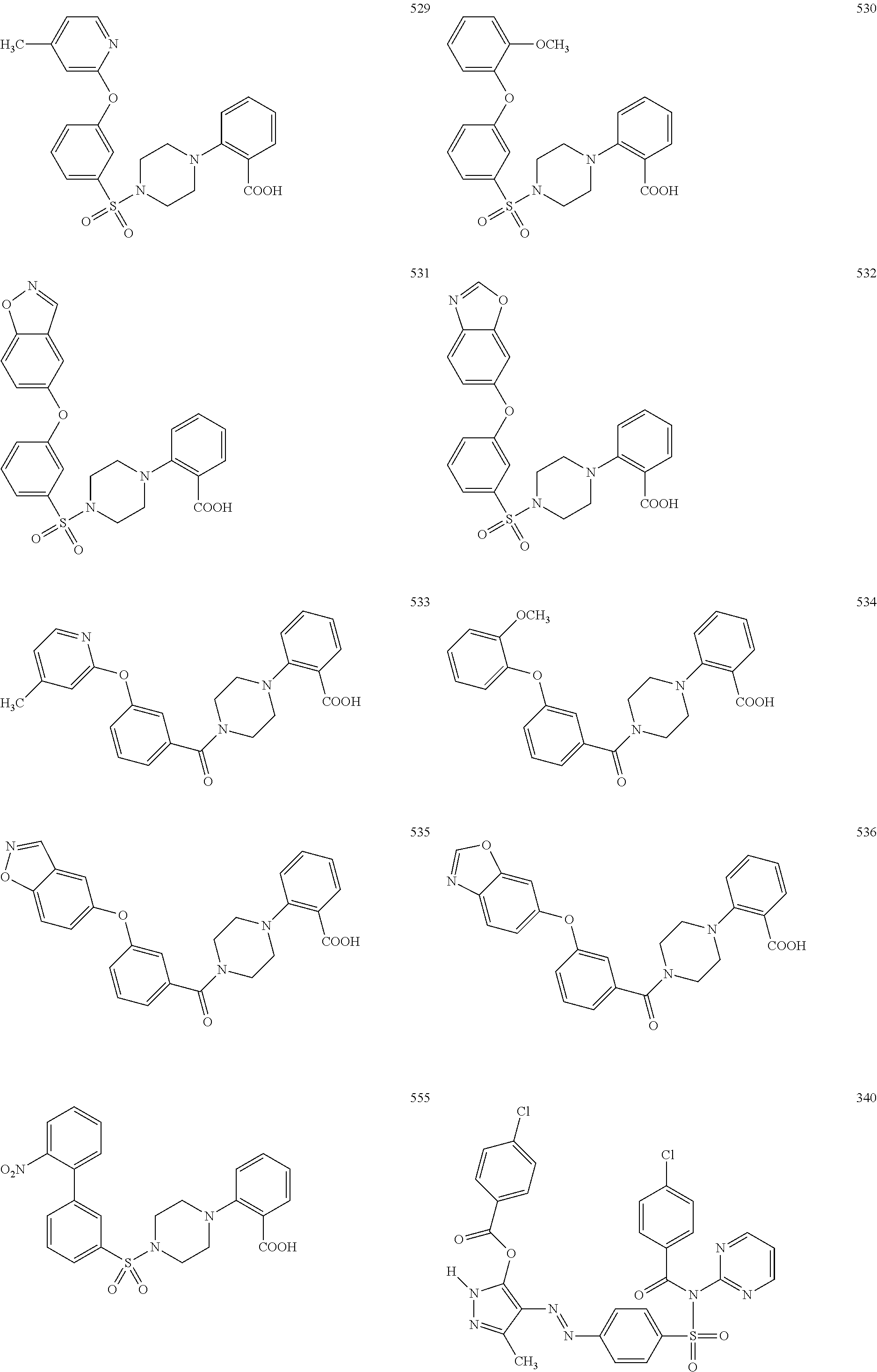

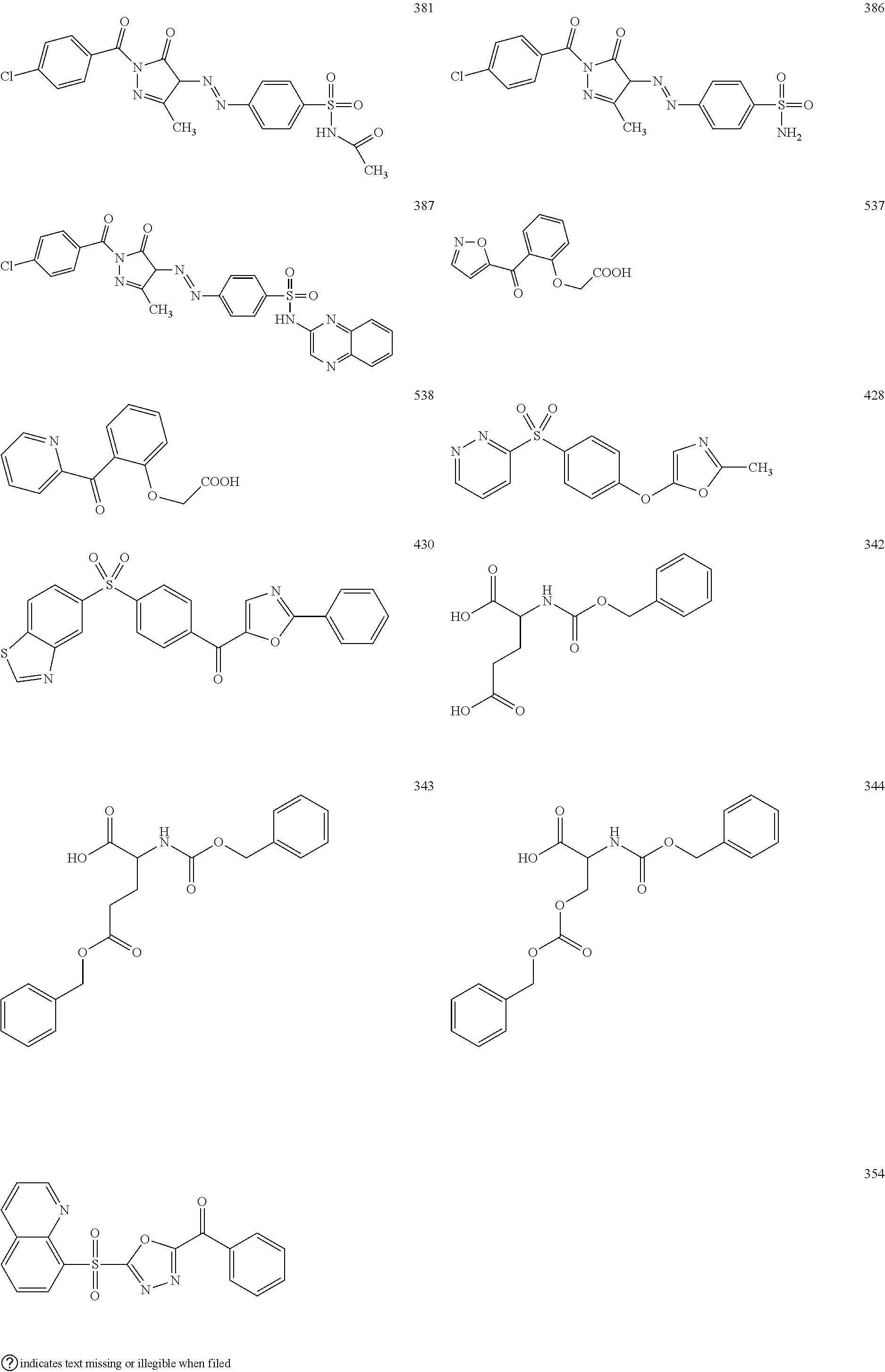

Embodiments of the invention encompass stereoisomers and optical isomers of the compounds described above including, e.g., mixtures of enantiomers, individual enantiomers and diastereomers, which can arise as a consequence of structural asymmetry of atoms in the compounds of the invention. Such embodiments further include the purified enantiomers, which may or may not contain trace amounts of a non-selected enantiomer or diastereomer.

In particular embodiments, the compounds described above may be modified to include a fluorescent label. Methods for fluorescently labeling organic molecules are well known and practiced in the art, and any such method may be used to fluorescently label the compounds of the invention. As such, numerous fluorescent labels may be applied to the molecules of the invention.

Some embodiments of the invention include salts of the compounds described above. In general, the term salt can refer to an acid and/or base addition salt of a compound. For example, an acid addition salt can be formed by adding an appropriate acid to a free base form of any of the compounds embodied above. Similarly, a base addition salts can be formed by adding an appropriate base to a free base form of any of the compounds described above. Examples of suitable salts include, but are not limited to, sodium, potassium, carbonate, methylamine, hydrochloride, hydrobromide, acetate, furmate, maleate, oxalate, and succinate salts. Methods for preparing free base forms of compounds such as those described herein and acid addition or base addition salts of such compounds are well known in the art, and any such method may be used to prepare the acid or base addition salts of embodiments of the invention.

Other embodiments of the invention include solvates or hydrates of the compounds of the invention. In some cases, hydration of a compound may occur during manufacture of the compounds or compositions including the compounds as a consequence of the method for preparing the compound or as a result of a specific step used to create a hydrate or solvate of the compound. In other cases, hydration may occur over time due to the hygroscopic nature of the compounds. Such hydrated compounds whether intentionally prepared or naturally produced are encompassed by the invention.

Embodiments of the invention also include derivatives of the compounds of the invention which may be referred to as “prodrugs.” The term “prodrug” as used herein denotes a derivative of a known drug that may have enhanced delivery characteristics, enhanced therapeutic value as compared to the active form of the drug, sustained release characteristics, reduced side-effects, or combinations thereof. For example, in some embodiments, a prodrug form of a compound of the invention may be administered in an inactive form or a form having reduced activity that is transformed into an active or more active form of the drug by an enzymatic or chemical process. For instance, in some embodiments, a prodrug form of a compound such as those described above may include one or more metabolically cleavable groups that are removed by solvolysis, hydrolysis or physiological metabolisms to release the pharmaceutically active form of the compound. In other embodiments, prodrugs may include acid derivatives of the compounds of the invention. Acid derivatives are well known in the art and include, but are not limited to, esters or double esters such as, for example, (acyloxy) alkyl esters or ((alkoxycarbonyl)oxy)alkyl esters prepared by reaction of an acid on the parent molecule with a suitable alcohol. Without wishing to be bound by theory, the compounds of the invention may have activity in both their acid and acid derivative forms. However, the acid derivative form may exhibit enhanced solubility, tissue compatibility or delayed release in the mammalian organism (see, e.g., Bundgard, H., Design of Prodrugs, pp. 7-9, 21-24, Elsevier, Amsterdam 1985). In still other embodiments, prodrugs that include an amide may be prepared by reacting a parent compound containing an acid with an amine, and in yet other embodiments, simple aliphatic or aromatic esters derived from acidic groups pendent on a compound of this invention may be prepared as prodrugs.

Embodiments of the invention also include pharmaceutical compositions or formulations including at least one compound embodied hereinabove, an acid or base addition salt, hydrate, solvate or prodrug of the at least one compound and one or more pharmaceutically acceptable carriers or excipients. Pharmaceutical formulations and pharmaceutical compositions are well known in the art, and can be found, for example, in Remington's Pharmaceutical Sciences, Mack Publishing Co., Easton, Pa., USA, which is hereby incorporated by reference in its entirety. Any formulations described therein or otherwise known in the art are embraced by embodiments of the invention. However, the endeavor of combining particular ingredients and components to yield formulations and associated delivery devices that have desirable properties (such as an acceptable bioavailability and pharmacokinetics, acceptable efficacy, low side effects, and long stability shelf life, for example) is not predictable in the art.

Without wishing to be bound by theory, the compounds of the invention may inhibit phosphorylation and subsequent activation of both AKT and PDKI by independently binding to these proteins and inhibiting their activation and/or activity. In some embodiments, the inhibition of phosphorylation associated with the reduction in AKT and/or PDK1 activity may occur simultaneously, but may result in differences in activation between the proteins. For example, in some embodiments, a relatively small dose of any one of the compounds described above may result in inhibition of AKT phosphosylation and activation, that may be relatively short lived. For example, in certain embodiments, a small does of compound 104 may result in a reduction in AKT phosphorylation for less than about 16 hours to less than about 24 hours. In other embodiments, a larger dose of any of the compounds described herein may be required to produce a discernable reduction in PDK1 phosphorylation; however, reduction in phosphorylation observed may be more prolonged than the observable inhibition AKT phosphorylation at equivalent concentrations. For example, in some embodiments, equivalent doses of compound 104 may produce a reduction in AKT phosphorylation within about 8 hours or less, while a reduction in PDK1 phosphorylation may not be observed until after about 12 hours of exposure to compound 104. During the same time course, a reduction in AKT phosphorylation inhibition may begin to be observed after about 16 hours, while PDK1 phosphorylation inhibition may be maintained for more than about 24 hours. Without wishing to be bound by theory, the prolonged reduction in PDK1 inhibition may provide an additional benefit to administration of the compounds of the invention.

Pharmaceutical excipients are well known in the art and include, but are not limited to, saccharides such as, for example, lactose or sucrose, mannitol or sorbitol, cellulose preparations, calcium phosphates such as tricalcium phosphate or calcium hydrogen phosphate, as well as binders, such as, starch paste such as, for example, maize starch, wheat starch, rice starch, potato starch, gelatin, tragacanth, methyl cellulose, hydroxypropylmethylcellulose, sodium carboxymethylcellulose, polyvinyl pyrrolidone or combinations thereof.

In particular embodiments, pharmaceutical formulations may include the active compound described and embodied above, a pharmaceutically acceptable carrier or excipient and any number of additional or auxiliary components known in the pharmaceutical arts such as, for example, binders, fillers, disintegrating agents, sweeteners, wetting agents, colorants, sustained release agents, and the like, and in certain embodiments, the pharmaceutical composition may include one or more secondary active agents. Disintegrating agents, such as starches as described above, carboxymethyl-starch, cross-linked polyvinyl pyrrolidone, agar, alginic acid or a salt thereof, such as sodium alginate and combinations thereof. Auxiliary agents may include, for example, flow-regulating agents and lubricants, such as silica, talc, stearic acid or salts thereof, such as magnesium stearate or calcium stearate, polyethylene glycol, and combinations thereof.

In certain embodiments, dragee cores may be prepared with suitable coatings that are resistant to gastric juices, such as concentrated saccharide solutions, which may contain, for example, gum arabic, talc, polyvinyl pyrrolidone, polyethylene glycol, titanium dioxide, lacquer solutions and suitable organic solvents or solvent mixtures, and combinations thereof. In order to produce coatings resistant to gastric juices, solutions of suitable cellulose preparations, such as acetylcellulose phthalate or hydroxypropylmethyl-cellulose phthalate may also be used. In still other embodiments, dye stuffs or pigments may be added to the tablets or dragee coatings, for example, for identification or in order to characterize combinations of active compound doses. In various embodiments, the carrier or excipient may not be dimethyl sulfoxide (DMSO). In certain embodiments, a carrier or excipient used in formulations may be ethanol at a concentration of greater than but not equal to 2.5%, such as greater than 5%, greater than 7.5%, or greater than 10%, and in some embodiments, a carrier or excipient used in formulations may be ethanol at a concentration of less than but not equal to 2.5%.

In some embodiments, the pharmaceutical compositions of the invention may include an enteric coating. Such enteric coatings may be stable at low (acidic) pH, as would be found in, for example, the stomach, but dissolve at higher (more basic) pH, as is found in the intestines. Without wishing to be bound by theory, some compounds embodied herein may by soluble at high pH such as, for example, from about 7 to about 11 or about 8 to about 10, or any individual pH there between. Therefore, such compounds may be more readily solubilized, and more easily absorbed by providing a pharmaceutical composition having an enteric coating that allows the compound to be delivered to a portion of the digestive tract where it is more soluble. In such embodiments, the compound may achieve improved (higher) blood concentrations at lower doses. For example, the pharmaceutical compositions of some embodiments may include an enteric coating that dissolves and releases the compound in the small intestine rather than the stomach. Enteric coatings are well known in the art and include include fatty acids, waxes, shellac and plastics, and plant fibers, and any such enteric coating may be used in the pharmaceutical compostions of embodiments. In particular embodiments, the enteric coating may be cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate, stearic acid, or combinations thereof.

Particular embodiments are directed to pharmaceutical formulations of any of the compounds described above for oral administration. In some embodiments, such fomulations may include solid doage formulations such as, for example, tablets and capsules, and in other embodiments, the formulations may include liquid dosage formulations. As may be recognized by the skill artisan, the compounds of some embodiments may include few readily ionizable functional groups, which may render such compounds insoluble in water. Therefore, in various embodiments, oral formulations may be prepared by complexation, with the use of cosolvents, or as emulsions. Embodiments are not limited by the type of system used to form the formulation. However, such systems may provide the a pharmaceutical composition including the compounds of embodiments described above that is sufficiently concentrated to administer orally and is sufficiently chemically stable. Chemical stability is important to provide long shelf life, as well as to avoid the degradation of desired pharmaceutical or biological activity, as well as to avoid the formation of toxic or other undesirable properties. For example, in some embodiments the system used may be provide a pharmaceutical composition that exhibits no more than about 5% to about 10% degradation of at least about 2 years under storage conditions such as, for example, storage at room temperature or refrigerated at about 4° C.

In certain embodiments, the oral formulation may be prepared as an emulsion. In some embodiments, pharmaceutical compositions including the compositions embodied hereinabove may be formulated as microemulsions. As used herein, the term “microemulsion” may refer to thermodynamically stable isotropically clear dispersions. The microemulsions of various embodiments may include any components useful for preparing emulsions and microemulsions known in the art and in some embodiments, may include a polar solvent, an oil, a surfactant, and a co-surfactant. For example, in some embodiments, the emulsion may be common oil-in-water emulsions including the compounds of various embodiments and a one or more common oils such as, for example, cottonseed, groundnut, corn, germ, olive, castor, soybean, mineral, and sesame oils. In other embodiments, emulsions may further include one or more solubilizers and emulsifiers such as, for example, ethyl alcohol, isopropyl alcohol, ethyl carbonate, ethyl acetate, benzyl alcohol, benzyl benzoate, propylene glycol, 1,3-butylene glycol, dimethyl formamide, glycerol, fatty acid derivatives of glycerol (for example, Labrasol® brand of caprylocaproyl macrogolglycerides (Polyoxylglycerides) by Callefossé Canada Inc.), tetrahydrofurfuryl alcohol, polyethylene glycols and fatty acid esters of sorbitan, and mixtures thereof. In still other embodiments, the emulsions may further contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof. In a particular embodiments, an oral formulation of a compound of the invention may include 2-(2-ethoxyethoxy)ethanol (for example, Transcutol® brand of 2-(2-ethoxyethoxy)ethanol by Gattefossé Canada Inc CG solution), and in some exemplary embodiments, a compound such as compound 104 may have a solubility of about 25 mg/ml in 80% Transcutol® GC at pH 4. In other embodiments, an oral formulation may include a compound of the invention and oleoyl macrogolglycerides such as polyoxylglycerides (for example, such as Labrofil® brand Oleoyl Macrogolglycerides (Polyoxylglycerides) by Cattefossé Canada Inc._solution), and in some exemplary embodiments, a compound such as compound 104 may have a solubility of about 40 mg/ml in 80% Labrofil® solution.

In some embodiments, such emulsions for oral administration may include cyclodextrins. As used, the term “cyclodextrin” can refer to any cyclic dextrin molecule, and in some embodiments, the cyclodextrin may be formed by an enzymatic conversion of starch. For example, enzymes such as various forms of cycloglycosyltransferase (CGTase) can break down helical starch and form specific cyclodextrin molecules having, for example, 6, 7, or 8 three-dimensional polyglucose rings. The cyclodextrins useful in embodiments of the invention include, but are not limited to, α-cyclodextrin having 6 glucose units, β-cyclodextrin having 7 glucose units, and γ-cyclodextrin having 8 glucose units, and combinations thereof. α-cyclodextrin, β-cyclodextrin, and γ-cyclodextrin are natural products and are generally regarded as safe in the U.S. and E.U., and β-cyclodextrin is FDA approved. The cyclodextrin molecules generally included a three-dimensional cavity of carbon atoms, hydrogen atoms and ether linkages, and which provide a hydrophobic cavity that can hold a variety of “guest” molecules that include a hydrophobic portion. A cyclodextrin molecule complexed with one or more guest molecules is generally referred to as “inclusion complex.” The term “guest” can refer to any molecule of which at least a portion can be held or captured within the three dimensional cavity present in the cyclodextrin molecule, and in various embodiments, the guest may be a compound of any of the general formulas described above. The cyclodextrins of embodiments may weigh up to 1,400 Daltons and may dissolve up to 40% by weight of the guest molecule in an aqueous media. Additionally, cyclodextrins may not precipitate upon dilution and may dilute at the same rate as the free compound.

Any of α-cyclodextrin, β-cyclodextrin, γ-cyclodextrin, or combinations thereof may be used in various embodiments of the invention. In some embodiments, the cyclodextrin used may be a modified cyclodextrin such as, for example, hydroxymethyl cyclodextrin, hydroxyethyl cyclodextrin, or hydroxypropyl cyclodextrin, where the modified cyclodextrin is α-cyclodextrin, β-cyclodextrin, γ-cyclodextrin or combination thereof. Without wishing to be bound by theory, modified cyclodextrin may provide enhanced solubility in aqueous media, increased physical stability of the compound, increased bioavailability, solubility, and dissolution rate, and improved permeability, gastrointestinal stability, buccal bioavailability, duration of therapeutic activity, and reduced toxicity. Such cyclodextrins may be provided at any concentration sufficient to solubilize the compound and may be provided at a concentration that is tolerable for oral administration to the subject. For example, in some embodiments, up to about 4 g of any of α-cyclodextrin, β-cyclodextrin, γ-cyclodextrin, or combination thereof may be combined with about 10 mL compound to produce a pharmaceutically acceptable formulation of the compound for oral administration. In certain embodiments, the cyclodextrin may not be 20% hydroxypropyl-β-cyclodextrin, but in some embodiments, the cyclodextrin may be less than but not equal to 20% hydroxypropyl-β-cyclodextrin such as, for example, less than or equal to 18%, less than 15%, or less than 10%. In other embodiments, the cyclodextrin may be greater than but not equal to 20% hydroxypropyl-β-cyclodextrin such as, for example, greater than or equal to 21%, greater than 25%, or greater than 30%. In other embodiments, the cyclodextrin may not be combined with ethanol or DMSO in water.

In yet other embodiments, the compound of the invention may be formulated for topical administration. As used herein, “topical” means application to the dermis, mucosa, or other external surfaces of a mammal, such as for example, application to skin, lips, nostrils, ear canals, genitals, or anus, for example. Topicals can be of low viscosity (such as a liquid) or of higher viscosity (such as, for example, a lotion). In such embodiments, the topical formulation may be an oil in water emulsion that may be prepared with a water or alcohol base, and in some embodiments, the water or alcohol concentration in the topical formulation may be sufficiently high to facilitate drying of the components of the formulation after application to the skin of the subject.

In other embodiments, the topical formulation of the invention may include a lipophilic base, which contain no, or substantially no, aqueous component or aqueous functional-equivalent. The lipophilic bases of various embodiments are not particularly limited, and any of those known in the pharmaceutical and cosmetic industries may be employed including lipophilic materials modified with thickeners, thinners, stabilizers, surfactants, etc. Non-limiting examples of lipophilic bases include oleaginous materials such as petrolatum, mineral oil thickened or gelled with polyethylene, high molecular weight paraffin waxes, mono and diglycerides of fatty acids gelled with high molecular weight fatty acids or polyamide complex of hydroxystearate, propylene glycol isostearate or isostearyl alcohol gelled with high molecular weight fatty acids, and mixtures thereof. In some embodiments, the lipophilic base may be a higher aliphatic alcohol having, for example, 8-18 carbon atoms, or an ester thereof. Examples of oleagenous (lipophilic) ointment bases include, but are not limited to, White Ointment USP, Yellow Ointment NF, Oleic Acid USP, Olive Oil USP, Paraffin USP, Petrolatum NF, White Petrolatum USP, Spermaceti Wax USP, Synthetic Spermaceti NF, Starch Glycerite NF, White Wax USP, Yellow Wax USP, and combinations, and in certain embodiments, the lipophilic base may be AQUAPHOR®.

Such topical formulations may include any components known in the art to be useful for the preparation of a topical formulation including, but not limited to, solubilizers, surfactants, coserfactants, penetration enhancers, and combinations thereof. In particular embodiments, such topical formulations may include solubilizers such as, for example, Capryol™ 90, Capryol™ Pgmc, Labrafil® M 1944 CS, Labrafil® M 2125 CS, Labrasol®, Labrafac™, Lipophile W1 1349, Labrafac™ PG, Lauroglycol™ 90, Lauroglycol™ FCC, Plurol® Oleique CC 497, Transcutol® P, and the like and combinations thereof, surfactants such as, for example, Labrasol, Plurol® Diisostearique, and the like and combinations thereof, cosurfactants, such as, for example, Capryol 90, Lauroglycol 90, and the like and combinations thereof. Penetration enhancers of various embodiments include solvents such as water; alcohols such as methanol, ethanol and 2-propanol, alkyl methyl sulfoxides such as dimethyl sulfoxide, decylmethyl sulfoxide, and tetradecylmethyl sulfoxide, pyrrolidones such as 2-pyrrolidone, N-methyl-2-pyrroloidone, and N-(2-hydroxyethyl)pyrrolidone, laurocapram, niacin, and niacinamide (and other vasodilators) and miscellaneous solvents such as acetone, dimethyl acetamide, dimethyl formamide, and tetrahyrdofurfuryl alcohol. Other penetration enhancers include amphiphiles such as L-amino acids, anionic surfactants, cationic surfactants, amphoteric surfactants, nonionic surfactants, fatty acids and alcohols, and still further penetration enhancers are disclosed in Remington: The Science and Practice of Pharmacy, 19.sup.th Edition (1995) on page 1583. In such embodiments, any of the solubilizers, surfactants, and cosurfactants listed may be used in separate topical formulations or may be combined in a single topical formulation.

Stable topical formulations of the compounds of various embodiments may effectively penetrate the skin of a patient to provide a biological effect. Thus, certain embodiments of the invention include topical formulations of one or more of the compounds described herein that provide a therapeutically effective amount of the compound to a target tissue below the skin, and other embodiments include methods for delivering a compound of the invention to a target tissue below the skin by topically applying the compound. The target tissues of various embodiments may include tissues exhibiting or susceptible to a proliferative disorder such as, for example, cancer. Without wishing to be bound by theory, delivery of an effective amount of the compounds of the invention to tissues below the skin is surprising and unexpected as most pharmaceutically active compounds are not capable of traversing the various layers of mammalian skin and producing a discernable biological effect.

Other embodiments include methods for preparing a topical formulation and the topical formulations prepared by such methods. For example, in some embodiments, a topical formulation may be prepared by combining any of the compounds described above with a solubilizer and providing the solubilizer at the highest concentration possible to provide a solution. In some embodiments, the method may further include identifying solubilizers having the best solubilizing properties, such as, highest MSA and using these solubilizers in further steps. Such methods may further include incorporating a surfactant, or emulsifier, into the solution, and in some embodiments, the surfactant may have a low hydrophile-lipophile balance (HLB) number. In some embodiments, the solubilized solution may be added to the surfactant very slowly, and in certain embodiments, the final concentration of solubilizer may be from about 60% to about 80% of the final solution. In other embodiments, an alcohol may be incorporated into the topical formulation and may provide improved drying times and may aid in preserving the compound or composition. In yet other embodiments, the method may include the addition of a costabilizer to produce a micro emulsion.

As indicated above, the oral formulations and topical formulations described in various embodiments may include any of the compounds described above including those encompassed by Formulae I, II, III, III-a, IV, V, VI, VII, VIII, and IX or combinations thereof. In certain embodiments, the compounds used in the oral formulation and topical formulations of may be any compound listed in the tables above and combinations thereof, and in particular embodiments, the compound may be compound 104.

Pharmaceutical compositions of the invention can be administered to any animal, and in particular, any mammal, that may experience a beneficial effect as a result of being administered a compound of the invention including, but not limited to, humans, canines, felines, livestock, horses, cattle, sheep, and the like. The dosage or amount of at least one compound according to the invention provided pharmaceutical compositions of embodiments may vary and may depend, for example, on the use of the pharmaceutical composition, the mode of administration or delivery of the pharmaceutical composition, the disease indication being treated, the age, health, weight, etc. of the recipient, concurrent treatment, if any, frequency of treatment, and the nature of the effect desired and so on. Various embodiments of the invention include pharmaceutical compositions that include one or more compounds of the invention in an amount sufficient to treat or prevent diseases such as, for example, cancer. An effective amount of the one or more compounds may vary and may be, for example, from about 0.001 mg to about 1000 mg or, in other embodiments, from about 0.01 mg to about 100 mg. In still other embodiments, the effective amount may be from about 0.1 mg to about 75 mg, about 0.5 mg to about 50 mg, or about 1 mg to about 25 mg. Of course, any individual dosage encompassed within these ranges are included in various embodiments of the invention.

The pharmaceutical compositions of the invention can be administered by any means that achieve their intended purpose. For example, routes of administration encompassed by the invention include, but are not limited to, subcutaneous, intravenous, intramuscular, intraperitoneal, buccal, or ocular routes, rectally, parenterally, intrasystemically, intravaginally, topically (as by powders, ointments, drops or transdermal patch), oral or nasal spray are contemplated in combination with the above described compositions.

Embodiments of the invention also include methods for preparing pharmaceutical compositions as described above by, for example, conventional mixing, granulating, dragee-making, dissolving, lyophilizing processes and the like. For example, pharmaceutical compositions for oral use can be obtained by combining the one or more active compounds with one or more solid excipients and, optionally, grinding the mixture. Suitable auxiliaries may then be added and the mixture may be processed to form granules which may be used to form tablets or dragee cores. Other pharmaceutical solid preparations include push-fit capsules containing granules of one or more compound of the invention that can, in some embodiments, be mixed, for example, with fillers, binders, lubricants, stearate, stabilizers or combinations thereof. Push-fit capsules are well known and may be made of gelatin alone or gelatin in combination with one or more plasticizer such as glycerol or sorbitol to form a soft capsule. In embodiments in which soft capsules are utilized, compounds of the invention may be dissolved or suspended in one or more suitable liquids, such as, fatty oils or liquid paraffin and, in some cases, one or more stabilizers.

Liquid dosage formulations suitable for oral administration are also encompassed by embodiments of the invention. Such embodiments, may include one or more compounds of the invention in pharmaceutically acceptable emulsions, solutions, suspensions, syrups, and elixirs that may contain, for example, one or more inert diluents commonly used in the art such as, but not limited to, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (for example, cottonseed, groundnut, corn, germ, olive, castor, and sesame oils), glycerol, fatty acid derivatives of glycerol (for example, labrasol), tetrahydrofurfuryl alcohol, polyethylene glycols and fatty acid esters of sorbitan, and mixtures thereof. Suspensions may further contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations for parenteral administration may include one or more compounds of the invention in water-soluble form, for example, water-soluble salts, alkaline solutions, and cyclodextrin inclusion complexes in a physiologically acceptable diluent which may be administered by injection. Physiologically acceptable diluent of such embodiments, may include, for example, sterile liquids such as water, saline, aqueous dextrose, other pharmaceutically acceptable sugar solutions; alcohols such as ethanol, isopropanol or hexadecyl alcohol; glycols such as propylene glycol or polyethylene glycol; glycerol ketals such as 2,2-dimethyl-1,3-dioxolane-4-methanol; ethers such as poly(ethyleneglycol)400; pharmaceutically acceptable oils such as fatty acid, fatty acid ester or glyceride, or an acetylated fatty acid glyceride. In some embodiments, formulations suitable for parenteral administration may additionally include one or more pharmaceutically acceptable surfactants, such as a soap or detergent; suspending agent such as pectin, carbomers, methylcellulose, hydroxypropylmethylcellulose, or carboxymethylcellulose; an emulsifying agent; pharmaceutically acceptable adjuvants or combinations thereof. Additional pharmaceutically acceptable oils which may be useful in such formulations include those of petroleum, animal, vegetable or synthetic origin including, but not limited to, peanut oil, soybean oil, sesame oil, cottonseed oil, olive oil, sunflower oil, petrolatum, and mineral oil; fatty acids such as oleic acid, stearic acid, and isostearic acid; and fatty acid esters such as ethyl oleate and isopropyl myristate. Additional suitable detergents include, for example, fatty acid alkali metal, ammonium, and triethanolamine salts; cationic detergents such as dimethyl dialkyl ammonium halides, alkyl pyridinium halides, and alkylamine acetates; and anionic detergents, such as alkyl, aryl, and olefin sulfonates, alkyl, olefin, ether and monoglyceride sulfates, and sulfosuccinates. In some embodiments, non-ionic detergents including, but not limited to, fatty amine oxides, fatty acid alkanolamides and polyoxyethylenepolypropylene copolymers or amphoteric detergents such as alkyl-β-aminopropionates and 2-alkylimidazoline quaternary salts, and mixtures thereof may be useful in parenteral formulations of the invention.

In particular embodiments, alkaline salts such as ammonium salts of compounds of the invention may be prepared by the addition of, for example, tris-chloride hydroxide, bis-tris propane, N-methylglucamine, or arginine to a free base form of the compound. Such alkaline salts may be particularly well suited for use as parenterally administered forms of the compounds of the invention. Buffers, preservatives, surfactants and so on may also be added to formulations suitable for parenteral administration. For example, suitable surfactants may include polyethylene sorbitan fatty acid esters, such as sorbitan monooleate, and the high molecular weight adducts of ethylene oxide with a hydrophobic base, formed by the condensation of propylene oxide with propylene glycol.

Pharmaceutical compositions for parenteral administration may contain from about 0.5 to about 25% by weight of one or more of the compounds of the invention and from about 0.05% to about 5% suspending agent in an isotonic medium. In various embodiments, the injectable solution should be sterile and should be fluid to the extent that it can be easily loaded into a syringe. In addition, injectable pharmaceutical compositions may be stable under the conditions of manufacture and storage and may be preserved against the contaminating action of microorganisms such as bacteria and fungi.

Topical administration includes administration to the skin or mucosa, including surfaces of the lung and eye. Compositions for topical administration, may be prepared as a dry powder which may be pressurized or non-pressurized. In non-pressurized powder compositions, the active ingredients in admixture are prepared as a finely divided powder. In such embodiments, at least 95% by weight of the particles of the admixture may have an effective particle size in the range of 0.01 to 10 micrometers. In some embodiments, the finely divided admixture powder may be additionally mixed with an inert carrier such as a sugar having a larger particle size, for example, of up to 100 micrometers in diameter. Alternatively, the composition may be pressurized using a compressed gas, such as nitrogen or a liquefied gas propellant. In embodiments, in which a liquefied propellant medium is used, the propellant may be chosen such that the compound and/or an admixture including the compound do not dissolve in the propellant to any substantial extent. In some embodiments, a pressurized form of the composition may also contain a surface-active agent. The surface-active agent may be a liquid or solid non-ionic surface-active agent or may be a solid anionic surface-active agent, which in certain embodiments, may be in the form of a sodium salt.

Compositions for rectal or vaginal administration may be prepared by mixing the compounds or compositions of the invention with suitable non-irritating excipients or carriers such as for example, cocoa butter, polyethylene glycol or a suppository wax. Such carriers may be solid at room temperature but liquid at body temperature and therefore melt in the rectum or vaginal cavity and release the drugs.

In still other embodiments, the compounds or compositions of the invention can be administered in the form of liposomes. Liposomes are generally derived from phospholipids or other lipid substances that form mono- or multi-lamellar hydrated liquid crystals when dispersed in an aqueous medium. Any non-toxic, physiologically acceptable and metabolizable lipid capable of forming liposomes can be used, and in particular embodiments, the lipids utilized may be natural and/or synthetic phospholipids and phosphatidyl cholines (lecithins). Methods to form liposomes are known in the art (see, for example, Prescott, Ed., Meth. Cell Biol. 14:33 (1976), which is hereby incorprated by reference in its entirety). Compositions including one or more compounds of the invention in liposome form can contain, for example, stabilizers, preservatives, excipients and the like.

In yet other embodiments, one or more compounds of the invention may be formulated for in vitro use in, for example, an assay for inhibition of AKT or an assay that requires inhibition of AKT. In such embodiments, the composition of the invention may include one or more compounds presented herein above in a carrier that is suitable for an assay. Such carriers may be in solid, liquid or gel form and may or may not be sterile. Examples of suitable carriers include, but are not limited to, dimethylsulfoxide, ethanol, dichloromethane, methanol and the like.

Embodiments of the invention are further directed to methods for using the compounds and compositions described herein above. For example, in some embodiments, the compounds or compositions of the invention may be used in the treatment or prevention of an AKT-mediated condition. Methods of such embodiments may generally include the step of administering to a subject in need of such treatment an effective amount of a compound or a composition selected from one or more of the embodiments described above to treat, prevent or ameliorate a AKT-mediated condition, and in particular embodiments, the condition or disease may be a proliferative disorder such as, for example, cancer. In other embodiments, methods of the invention may include the step of administering to a subject in need of such treatment an effective amount of a compound or composition selected from one or more of the embodiments described above to treat, prevent or ameliorate cancer or a cell proliferation related disease. Cancers that may be treated using compositions of the invention include but not limited to skin cancers, breast cancer, colorectal cancer, colon cancer, esophageal cancer, mesothelioma, ovarian cancer, and gastric cancer. In still other embodiments, the compound or composition of the invention may be used to treat cancer by blocking tumorigenesis, inhibiting metastasis or inducing apoptosis.

The type of proliferative disorder or cancer that can be treated using compounds of the invention is not limited in embodiments of the invention. For example, cancers that may be treated using compounds of any or formulae I-IX described above include, but are not limited to, breast cancer, lung cancer, head and neck cancer, brain cancer, abdominal cancer, colon cancer, colorectal cancer, esophageal cancer, gastrointestinal cancer, glioma, liver cancer, tongue cancer, neuroblastoma, osteosarcoma, ovarian cancer, pancreatic cancer, renal cancer, prostate cancer, retinoblastoma, Wilm's tumor, multiple myeloma, skin cancer, lymphoma and blood cancer, and various forms of skin cancer and melanoma. In certain embodiments, the cancer treated using the methods of embodiments of the invention may be prostate, lung, breast, ovarian, pancreatic, skin cancer, and melanoma, and in particular embodiments, the cancer treated may be skin cancer or melanoma.

Other embodiments of the invention include methods in which one or more of the compounds or compositions described herein may be administered to a subject to inhibit or prevent a healthy subject from developing a AKT-mediated condition. As such, the compounds and compositions of the invention may be used as a prophylactic that prevents or inhibits the development of a AKT-mediated condition or disease. In such embodiments, the compound or composition may be administered to a subject who does not have an AKT-mediated condition or is not exhibiting the symptoms of an AKT-mediated condition but may be at risk of developing one to prevent or inhibit the onset of such a disorder. For example, the individual may be genetically predisposed to an AKT-mediated condition or has increased likelihood of developing such a disorder as a result of, for instance, an injury, surgery or other medical condition.

In general, methods of embodiments of the invention may include the step of administering or providing an “effective amount” or a “therapeutically effective amount” of a compound or composition of the invention to an individual. In such embodiments, an effective amount of the compounds of the invention may be any amount that produces the desired effect. As described above, this amount may vary depending on, for example, the circumstances under which the compound or composition is administered (e.g., to incite treatment or prophylactically), the type of individual, the size, health, pregression of the disease, etc. of the individual and so on. The dosage may further vary based on the severity of the condition. For example, a higher dose may be administered to treat an individual with a well-developed inflammatory condition, compared to the amount used to prevent a subject from developing the inflammatory condition. Those skilled in the art can discern the proper dosage based on such factors. For example, in some embodiments, the dosage may be within the range of about 0.01 mg/kg body weight to about 300 mg/kg body weight or between about 0.1 mg/kg body weight and about 100 mg/kg body weight, and in particular embodiments, the dosage may be from about 0.1 mg/kg body weight to about 10 mg/kg body weight. Of course, any individual dosage encompassed within these ranges are included in various embodiments of the invention.

The administration schedule may also vary. For example, in some embodiments, the compounds or compositions of the invention may be administered in a single dose once per day or once per week. In other embodiments, the compounds or compositions of the invention may be administered in two, three, four or more doses per day or per week. For example, in one embodiment, an effective amount for a single day may be divided into separate dosages that may contain the same or a different amount of the compound or composition and may be administered several times throughout a single day. Without wishing to be bound by theory, the dosage per administration and frequency of administration may depend, for example, on the specific compound or composition used, the condition being treated, the severity of the condition being treated, and the age, weight, and general physical condition of the individual to which the compound or composition is administered and other medications which the individual may be taking. In another exemplary embodiment, treatment may be initiated with smaller dosages that are less than the optimum dose of the compound, and the dosage may be increased incrementally until a more optimum dosage is achieved.

In each of the embodiments above, the compound administered can be provided as a pharmaceutical composition including compound as described above and a pharmaceutically acceptable excipient, or a pure form of the compound may be administered.

In additional embodiments, the compound or composition of the invention may be used alone or in combination with one or more additional agents. For example, in some embodiments, a compound or composition of invention may be formulated with one or more additional anti-inflammatory agents, anti-cancer agents or combinations thereof such that the pharmaceutical composition obtained including the compound or composition of the invention and the one or more additional agents can be delivered to an individual in a single dose. In other embodiments, the compound or composition of the invention may be formulated as a separate pharmaceutical composition that is delivered in a separate dose from pharmaceutical compositions including the one or more additional agents. In such embodiments, two or more pharmaceutical compositions may be administered to deliver effective amounts of a compound or composition of the invention and the one or more additional agents. For example, in some embodiments, one or more compound of formula I-IX may be administered in combination with or co-administered with doxorubicin, paclitaxel, methotrexate, tamoxifen, cyclophosphamide, vincristine, etoposide, streptozotocin and 5-fluorouracil, and in particular embodiments, one or more of the compounds of the invention may be administered with paclitaxel.

Method of certain embodiments of the invention may include the step of selectively inhibiting AKT by, for example, contacting AKT with a compound or composition according to the invention. In such embodiments, the AKT may be contained within a living organism, living tissue or one or more living cells to provide in vivo inhibition, or the AKT may be isolated to provide in vitro inhibition. For example, compounds or compositions described herein may be useful in in vitro drug discovery assays in which the efficacy and/or potency of other AKT inhibitors. The amount of the compound or composition of the invention used to inhibit AKT not necessarily the same when used in vivo compared to in vitro. For example, factors such as pharmacokinetics and pharmacodynamics of a particular compound may require that a larger or smaller amount of the compound be used for in vivo applications. In another embodiment, a compound or composition according to the invention may be used to form a co-crystallized complex with AKT protein.

By “selectively” is meant that the compounds and compositions described herein inhibit the activity of AKT without interfering with the activity of the other proteins. For example, compounds or compositions of the invention can be administered to a cell that contains AKT, phosphorylated AKT or AKT that is otherwise activated or not activated as well as other proteins such as, for example, TORC2, PDK1, FKHR, AFX, GSK-3β, c-RAF, Flt3, JNK2α2, JNK3, Lck, Lyn, Tie2, TrkB, IGF-R, ERK1, ERK2, MEK1, PRAK, Yeo and/or ZAP-70. For instance, in some embodiments, the method of the invention can inhibit greater than about 80% of the activity of AKT while inhibiting less than about 5%, about 10%, about 20% or about 30% of the activity of other proteins such as those listed above.

One skilled in the art can evaluate the ability of a compound to inhibit or modulate the activity of a AKT and/or prevent, treat, or inhibit an conditions associated with AKT by one or more assays known in the art.

### EXAMPLES

Although the present invention has been described in considerable detail with reference to certain preferred embodiments thereof, other versions are possible. Therefore the spirit and scope of the appended claims should not be limited to the description and the preferred versions contained within this specification. Various aspects of the present invention will be illustrated with reference to the following non-limiting examples. The following examples are for illustrative purposes only and are not to be construed as limiting the invention in any manner.

### Example 1

### Synthesis

The compounds of the invention can be synthesized by any method known in the art, and embodiments of the invention further include methods for preparing or the compounds described above. All commercial reagents were used without further purification. Analytical thin-layer chromatography (TLC) was carried out on pre-coated Silica Gel F254 plates. TLC plates were visualized with UV light (254 nm). ¹H NMR spectra were recorded at 250, 300, or 500 MHz and ¹³C NMR at 62.5, 75, or 125 MHz. Chemical shifts (δ) are expressed in ppm and are internally referenced (7.26 ppm for ¹H NMR and 77.00 ppm for ¹³C NMR in CDCl₃, 2.50 ppm for ¹H NMR and 39.50 ppm for ¹³C NMR in DMSO-d₆). Mass spectra and high resolution mass spectra were obtained in the Mass Spectrometry Laboratory in the Department of Chemistry at the University of Arizona. Various properties of the synthesized compounds are provided in table I below. Melting points are uncorrected.

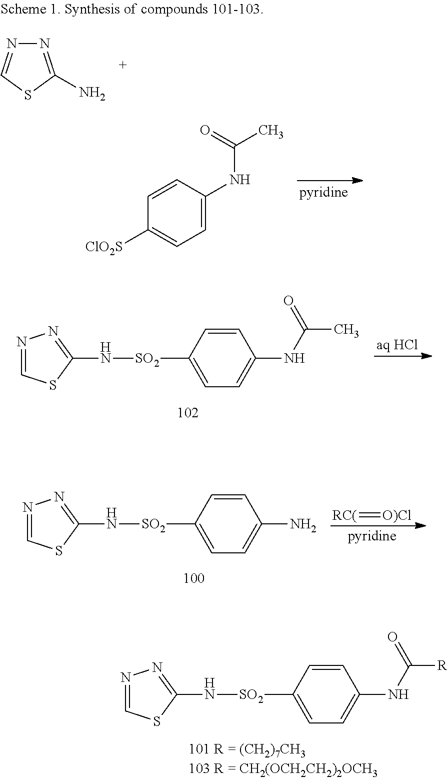

N-(4-(N-1,3,4-Thiadiazol-2-ylsulfamoyl)phenyl)acetamide (102). 2-Amino-1,3,4-thiadiazole (500 mg, 4.95 mmol) was suspended in pyridine (1.26 mL). p-Acetamidobenzenesulfonyl chloride (1.2 g, 5.15 mmol) was added and the mixture was heated to 95° C. for 1 h. The mixture was dissolved in 10% aqueous HCl and extracted with ethyl acetate. The organic extracts were washed with water and dried over anhydrous Na₂SO₄. Evaporation of the solvent yielded the crude product (1.4 g, 4.7 mmol, 95%). Recrystallization from CH₂Cl₂/MeOH gave pure product, mp 216-217° C. (lit¹ mp 214-215° C.); ¹H NMR (250 MHz, CDCl₃) δ 2.07 (3, s), 7.73 (4, s), 8.74 (1, s), 10.35 (1, s), 14.35 (1, br s); ¹³C NMR (62.5 MHz, DMSO) δ 24.2, 118.7, 127.0, 135.6, 143.0, 144.9, 167.2, 169.

4-Amino-N-(1,3,4-thiadiazol-2-yl)benzenesulfonamide (100). Compound 102 (1.0 g, 3.6 mmol) was suspended in 3N HCl (10 mL) and heated to reflux for 30 min. The acidic mixture was neutralized with Na₂CO₃ solution. The precipitated product was collected by filtration, washed with water, and dried to give the product (450 mg, 1.8 mmol, 49%), mp 226° C. (lit² mp 221-222° C.); ¹H NMR (250 MHz, CDCl₃) δ 5.95 (2, s), 6.57 (2, d, J=6.5 Hz), 7.41 (2, d, J=6.5 Hz), 8.68 (1, s), 14.03 (1, br s).

N-(4-(N-1,3,4-Thiadiazol-2-ylsulfamoyl)phenyl)decanamide (101).

Compound 100 (50 mg, 0.20 mmol) was suspended in pyridine (0.3 mL). Decanoyl chloride (39.1 mg, 0.21 mmol) was added gradually over 15 min. The reaction mixture was heated to 95° C. and stirred at this temperature for 1 h, then poured into 10% aqueous HCl solution and extracted with EtOAc (3×0.5 mL). The combined organic extracts were washed with water (3×5 mL), brine (3×5 mL), and dried over anhydrous Na₂SO₄. Evaporation of the solvent yielded the product (80 mg, 0.20 mmol, 95%). It was recrystallized from hexanes/ethyl acetate to yield an analytical sample, mp 151-152° C.; ¹H NMR (250 MHz, CD₃OD) δ 0.88 (3, t, J=7.5 Hz), 1.24-1.45 (12, m), 1.68 (2, t, J=7.5 Hz), 2.37 (2, t, J=7.5 Hz), 7.72 (2, d, J=8.5 Hz), 7.79 (2, d, J=8.5 Hz), 8.49 (1, S); ¹³C NMR (125 MHz, CD₃OD) δ 14.4, 23.7, 26.7, 30.3, 30.4, 30.5, 30.6, 33.0, 38.1, 102.4, 128.3, 137.5, 144.0, 145.0, 170.0, 174.9; MS (ESI⁺) 411.1 (M+H)⁺; HRMS (IonSpec. HiRES ESI⁺) calcd. for C₁₈H₂₇N₄O₃S₂ (M+H)⁺411.1525, obsd. 411.1524.

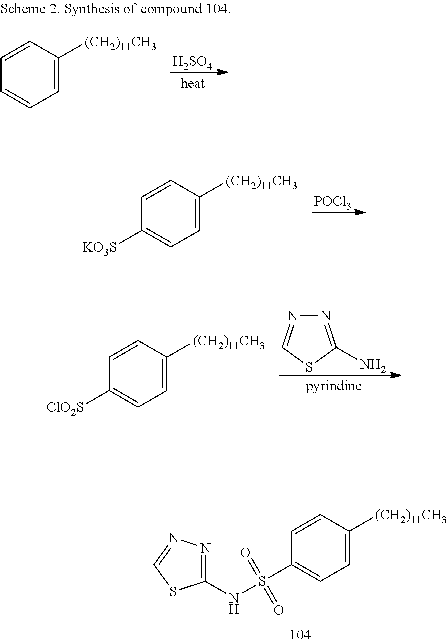

4-Dodecyl-N-(1,3,4-thiadiazol-2-yl)benzenesulfonamide (104). 2-Amino-1,3,4-thiadiazole (439 mg, 4.3 mmol) was suspended in pyridine (1.5 mL). p-Dodecylbenzenesulfonyl chloride (1.0 mg, 2.9 mmol) was added slowly at 0° C. The reaction mixture was then heated to 95° C. and was stirred at this temperature for 1 h. The reaction mixture was then added to aqueous 10% HCl (15 mL) and the resulting mixture extracted with ethyl acetate (3×30 mL). The organic extracts were washed with water (3×50 mL), brine (3×50 mL), dried over anhydrous Na₂SO₄, filtered, and volatiles evaporated to yield a solid mass. Chromatography on silica gel (70-230 mesh) eluted with 2% MeOH in CH₂Cl₂ gave the product (600 mg, 1.5 mmol, 51%). Recrystallization from hexanes:ethyl acetate (3:7) gave an analytical sample, mp 126-127° C.; NMR (500 MHz, CDCl₃) δ 0.87 (3, t, J=6.5 Hz), 1.20-1.36 (18, m), 1.54-1.63 (2, m), 2.62 (2, t, J=7.5 Hz), 7.25 (2, d, J=8.0 Hz), 7.83 (2, d, J=8.0 Hz), 8.28 (1, s), 12.81 (1, br s); ¹³C NMR (125 MHz, CDCl₃) δ 14.0, 22.6, 29.2, 29.3, 29.4, 29.5, 29.6, 31.0, 31.8, 35.8, 126.4, 128.9, 138.0, 142.8, 148.5, 167.5; MS (LCQ, ESI⁺) Calcd for C₂₀H₃₂N₃O₂S₂ 410.1936. found 410.10 (M+H)⁺; HRMS (ESI⁺, m/z) Calcd C₂₀H₃₂N₃O₂S₂ 410.1936. found 410.1932 (M+H)⁺.

p-Dodecylbenzenesulfonyl Chloride. A mixture of 1-phenyldodecane (7.5 g, 30.5 mmol) and concentrated H₂SO₄ (8.4 mL) was stirred vigorously at 90° C. for 1 h, cooled to room temperature, and then gradually poured with stirring into 10% aqueous KOH solution (175 mL). The resulting white precipitate was collected by filtration, washed with cold water (40 mL) and dried to give potassium 4-dodecylbenzene sulfonate (10.6 g, 29.1 mmol, 84%). This salt (10.0 g, 27.5 mmol) and POCl₃ (4.2 g, 27.4 mmol) were stirred at room temperature and gradually heated to 170° C. The hot reaction mixture was poured into cold water and extracted with CHCl₂. The organic layer was washed with water, dried over anhydrous Na₂SO₄, and filtered. Evaporation of the volatiles yielded p-dodecylbenzenesulfonyl chloride as a pale yellow liquid (9.2 g, 97%) which eventually became crystalline, mp 33° C.; ¹H NMR (300 MHz, CDCl₃) δ 0.88 (t, 3H, J=6.5), 1.20-1.38 (m, 18H), 1.60-1.68 (m, 2H), 2.72 (t, 2H, J=7.5 Hz), 7.40 (d, 2H, J=8.4 Hz), 7.79 (d, 2H, J=8.4 Hz); ¹³C NMR (75 MHz, CDCl₃) δ 14.1, 22.6, 29.1, 29.3, 29.3, 29.5, 29.6, 30.9, 31.9, 36.0, 127.0, 129.6, 141.7, 151.6.

4-Dodecyl-N-(5-methyl-1,3,4-thiadiazol-2-yl)benzenesulfonamide (108). 2-Amino-5-methyl-1,3,4-thiadiazole (150 mg, 1.3 mmol) was suspended in pyridine (0.5 mL). p-Dodecylbenzenesulfonyl chloride (300 mg, 0.87 mmol) was added slowly at 0° C. The reaction mixture was then heated to 95° C. and was stirred at this temperature for 1 h. The reaction mixture was then added to aqueous 10% HCl (5 mL) and the resulting mixture extracted with ethyl acetate (3×10 mL). The organic extracts were washed with water, brine, dried over anhydrous Na₂SO₄, filtered, and volatiles evaporated to yield a solid mass. Chromatography on silica gel (70-230 mesh) eluted with 2% MeOH in CH₂Cl₂ gave the product (310 mg, 0.73 mmol, 84%). Recrystallization from hexanes:ethyl acetate (3:7) gave an analytical sample, mp 149-150° C.; ¹H NMR (500 MHz, CDCl₃) δ 0.88 (3, t, J=7.0 Hz), 1.20-1.36 (18, m), 1.54-1.63 (2, m), 2.51 (3, s), 2.63 (2, t, J=7.5 Hz), 7.25 (2, d, J=7.5 Hz), 7.83 (2, d, J=7.5 Hz), 12.36 (1, br s); ¹³C NMR (125 MHz, CDCl₃) δ 14.1, 16.5, 22.7, 29.2, 29.3, 29.4, 29.5, 29.6, 31.1, 31.9, 35.9, 126.4, 128.8, 138.3, 148.3, 154.1, 168.6; MS (ESI⁺, m/z) Calcd for C₂₁H₃₄N₃O₂S₂ 424.2092 found 424.20 (M+H)⁺; HRMS (ESI⁺, m/z) Calcd for C₂₁H₃₄N₃O₂S₂ 424.2092. found 424.2085 (M+H)⁺.

4-Dodecyl-N-(5-ethyl-1,3,4-thiadiazol-2-yl)benzenesulfonamide (112). 2-Amino-5-ethyl-1,3,4-thiadiazole (169 mg, 1.3 mmol) was suspended in pyridine (0.5 mL). p-Dodecylbenzenesulfonyl chloride (300 mg, 0.87 mmol) was added slowly at 0° C. The reaction mixture was then heated to 95° C. and was stirred at this temperature for 1 h. The reaction mixture was then added to aqueous 10% HCl (5 mL) and the resulting mixture extracted with ethyl acetate (3×10 mL). The organic extracts were washed with water, brine, dried over anhydrous Na₂SO₄, filtered, and volatiles evaporated to yield a solid mass. Chromatography on silica gel (70-230 mesh) eluted with 2% MeOH in CH₂Cl₂ gave the product (225 mg, 0.51 mmol, 59%). Recrystallization from hexanes:ethyl acetate (3:7) gave an analytical sample, mp 93-94° C.; ¹H NMR (500 MHz, CDCl₃) δ 0.88 (3, t, J=6.5 Hz), 1.20-1.36 (18, m), 1.33 (3, t, J=7.5 Hz), 1.54-1.63 (2, m), 2.63 (2, t, J=7.5 Hz), 2.84 (2, q, J=7.5 Hz), 7.25 (2, d, J=8.5 Hz), 7.83 (2, d, J=8.5 Hz), 12.30 (1, br s); ¹³C NMR (125 MHz, CDCl₃) δ 12.6, 14.1, 22.7, 24.4, 29.2, 29.3, 29.4, 29.5, 29.6, 31.1, 31.9, 35.9, 126.5, 128.8, 138.4, 148.2, 160.1 168.2; MS (ESI⁺, m/z) Calcd for C₂₂H₃₆N₃O₂S₂ 438.2249. found 438.30 (M+H)⁺; HRMS (ESI⁺, m/z) Calcd for C₂₂H₃₆N₃O₂S₂ 438.2249. found 438.2247 (M+H)⁺.

N-(5-tert-Butyl-1,3,4-thiadiazol-2-yl)-4-dodecylbenzenesulfonamide (116).

2-Amino-5-tert-butyl-1,3,4-thiadiazole (204 mg, 1.3 mmol) was suspended in pyridine (0.5 mL). p-Dodecylbenzenesulfonyl chloride (300 mg, 0.87 mmol) was added slowly at 0° C. The reaction mixture was then heated to 95° C. and was stirred at this temperature for 1 h. The reaction mixture was then added to aqueous 10% HCl (5 mL) and the resulting mixture extracted with ethyl acetate (3×10 mL). The organic extracts were washed with water, brine, dried over anhydrous Na₂SO₄, filtered, and volatiles evaporated to yield a solid mass. Chromatography on silica gel (70-230 mesh) eluted with 2% MeOH in CH₂Cl₂ gave the product (350 mg, 0.75 mmol, 87%). Recrystallization from hexanes:ethyl acetate (3:7) gave an analytical sample, mp 117-118° C.; ¹H NMR (500 MHz, CDCl₃) δ 0.88 (3, t, J=6.5 Hz), 1.20-1.36 (18, m), 1.38 (9, s), 1.56-1.64 (2, m), 2.63 (2, t, J=7.5 Hz), 7.25 (2, d, J=8.0 Hz), 7.86 (2, d, J=8.0 Hz), 12.24 (1, br s); ¹³C NMR (125 MHz, CDCl₃) δ 14.1, 22.7, 29.2, 29.3, 29.4, 29.5, 29.6, 29.7, 31.1, 31.8, 35.8, 36.5, 126.5, 128.7, 138.5, 148.1, 167.8, 168.0; MS (ESI⁺, m/z) Calcd for C₂₄H₄₀N₃O₂S₂ 466.3. found 466.2 (M+H)⁺; HRMS (ESI⁺, m/z) Calcd for C₂₄H₄₀N₃O₂S₂ 466.2562. found 466.2562 (M+H)⁺.

2-(5-(4-Dodecylphenylsulfonamido)-1,3,4-thiadiazol-2-yl)acetic Acid (120). Distilled water (3.0 mL) and 10% aqueous NaOH (0.65 mL) were added to compound 37 (200 mg, 0.40 mmol) and the mixture was heated under reflux for 2 h. The pH of the solution was then adjusted to 4.0 by addition of 1.0 M HCl, the resulting precipitate was isolated by filtration, washed with cold water, and dried to give 161 mg (0.34 mmol, 86%) of the product as a solid, mp 194-195° C.; ¹H NMR (300 MHz, DMSO-d₆) δ 0.85 (t, 3H, J=6.6 Hz), 1.23 (m, 18H), 1.53 (m, 2H), 2.57 (t, 2H, J=7.5 Hz), 7.24 (d, 2H, J=8.1 Hz), 7.61 (d, 2H, J=7.8 Hz); ¹³C NMR (75 MHz, DMSO-d₆) δ 14.0, 22.1, 28.8, 28.9, 29.1, 30.7, 31.3, 34.9, 37.4, 125.8, 128.4, 141.2, 146.0, 153.3, 168.9, 170.8; MS (LCQ, ESI⁺) Calcd for C₂₂H₃₄N₃O₄S₂ 468.2. found 468.2 (M+H)⁺; HRMS (ESI⁺, m/z) Calcd for C₂₂H₃₄N₃O₄S₂ 468.1991. found 468.1977 (M+H)⁺.

Ethyl 2-(5-(4-Dodecylphenylsulfonamido)-1,3,4-thiadiazol-2-yl)acetate (120E). To a solution of p-dodecylbenzenesulfonyl chloride (1.01 g, 2.94 mmol) in pyridine (10 mL) was added ethyl 2-(5-amino-1,3,4-thiadiazol-2-yl)acetate (500 mg, 2.67 mmol). The reaction mixture was stirred at room temperature for 4.5 h, then 2 M HCl (20 mL) was added to quench the reaction. The mixture was extracted with ethyl acetate (3×50 mL). The organic extracts were washed with water (20 mL), brine (20 mL), dried over Na₂SO₄, filtered, and concentrated. The residue was purified by chromatography over silica gel (70-230 mesh) eluted with CH₂Cl₂:methanol 19:1 to give the product as a solid, mp 108-109° C., in 43% yield (570 mg, 1.15 mmol); ¹H NMR (300 MHz, CDCl₃) δ 0.87 (t, 3H, J=7.2 Hz), 1.24-1.34 (m, 21H), 1.55-1.66 (m, 2H), 2.63 (t, 2H, J=7.2 Hz), 3.88 (s, 2H), 4.25 (q, 2H, J=7.5 Hz), 7.25 (d, 2H, J=8.1 Hz), 7.81 (d, 2H, J=7.8 Hz); ¹³C NMR (75 MHz, CDCl₃) δ 14.0, 14.1, 22.7, 29.3, 29.5, 29.6, 29.7, 31.2, 31.9, 35.9, 38.1, 61.9, 126.7, 128.4, 138.8, 147.2, 152.1, 168.3, 170.3; MS (LCQ, ESI⁺) Calcd for C₂₄H₃₈N₃O₄S₂ 496.2. found 496.2 (M+H)⁺; HRMS (ESI⁺, m/z) Calcd for C₂₄H₃₈N₃O₄S₂ 496.2304. found 496.2295 (M+H)⁺.

Ethyl 2-(5-Amino-1,3,4-thiadiazol-2-yl)acetate. Thiosemicarbazide (1.0 g, 11.0 mmol) and ethyl 3-ethoxy-3-iminopropionate hydrochloride (2.0 g, 10.0 mmol) were mixed in glacial acid (2 mL) for 10 min at 55° C. and then boiled for 1.5 h. The reaction mixture was evaporated, diluted with cold water, carefully neutralized with NaHCO₃, and cooled to 5° C. The precipitate was collected and crystallized from water to yield 0.88 g (4.70 mmol, 47%) of the product, mp 149-150° C.; ¹H NMR (300 MHz, DMSO-d₆) δ 1.19 (t, 3H, J=7.2 Hz), 3.96 (s, 2H), 4.10 (q, 2H, J=6.9 Hz), 7.11 (s, 2H); ¹³C NMR (75 MHz, DMSO-d₆) δ 14.0, 35.4, 60.9, 150.4, 168.9, 169.6.

Ethyl 5-(4-dodecylphenylsulfonamido)-1,3,4-thiadiazole-2-carboxylate (124E). To a solution of p-dodecylbenzenesulfonyl chloride (260 mg, 0.75 mmol) in pyridine (3 mL) was added ethyl 5-amino-1,3,4-thiadiazole-2-carboxylate (100 mg, 0.58 mmol). The reaction mixture was stirred at room temperature for 4.5 h, then 2 M HCl was added to quench the reaction. The mixture was extracted with ethyl acetate (3×40 mL). The organic extracts were washed with water (20 mL), brine (20 mL), dried over Na₂SO₄, filtered, and concentrated. The residue was purified by chromatography over silica gel (70-230 mesh) eluted with CH₂Cl₂:methanol 49:1 to give the product as a solid, mp 96-97° C., in 34% yield (95 mg, 0.20 mmol); ¹H NMR (300 MHz, CDCl₃) δ 0.85 (t, 3H, J=6.6 Hz), 1.20-1.35 (m, 21H), 1.57 (m, 2H), 2.60 (t, 2H, J=7.0 Hz), 4.43 (q, 2H, J=7.2 Hz), 7.26 (d, 2H, J=8.0 Hz), 7.77 (d, 2H, J=7.7 Hz); ¹³C NMR (300 MHz, CDCl₃) δ 14.1, 22.7, 29.3, 29.4, 29.6, 29.6, 31.1, 31.9, 35.9, 63.4, 126.6, 128.9, 136.9, 145.8, 159.9, 163.7, 167.9; MS (LCQ, ESI⁺) Calcd for C₂₃H₃₆N₃O₄S₂ 482.2. found 482.1 (M+H)⁺; HRMS (ESI⁺, m/z) Calcd for C₂₃H₃₆N₃O₄S₂ 482.2140. found 482.2134 (M+H)⁺.

4-Dodecyl-N-(5-(hydroxymethyl)-1,4-thiadiazol-2-yl)benzenesulfonamide (128). To a solution of p-dodecylbenzenesulfonyl chloride (200 mg, 0.58 mmol) in pyridine (3 mL) was added 2-amino-5-hydroxymethyl-1,3,4-thiadiazole (70 mg, 0.53 mmol). The reaction mixture was stirred at room temperature for 4.5 h, then 2 M HCl (8 mL) was added to quench the reaction. The mixture was extracted with ethyl acetate (3×20 mL). The organic extracts were washed with water (10 mL), brine (10 mL), dried over Na₂SO₄, filtered, and concentrated. The residue was purified by chromatography on silica gel (70-230 mesh) eluted with CH₂Cl₂:methanol 19:1 to give the product as a solid, mp 138-139° C., in 65% yield (151 mg, 0.34 mmol); ¹H NMR (300 MHz, DMSO-d₆) δ 0.84 (t, 3H, J=6.6 Hz), 1.22 (m, 18H), 1.54-1.57 (m, 2H), 2.64 (t, 2H, J=7.8 Hz), 4.57 (s, 2H), 6.05 (br, 1H), 7.35 (d, 2H, J=8.1 Hz), 7.67 (d, 2H, J=7.8 Hz); ¹³C NMR (75 MHz, DMSO-d₆) δ 13.9, 22.1, 28.6, 28.7, 28.8, 29.0, 30.6, 31.3, 34.9, 58.4, 125.8, 128.9, 139.2, 147.5, 161.1, 167.5; MS (LCQ, ESI⁺) Calcd for C₂₁H₃₄N₃O₃S₂ 440.2. found 440.2 (M+H)⁺; HRMS (ESI⁺, m/z) Calcd for C₂₁H₃₄N₃O₃S₂ 440.2042. found 440.2029 (M+H)⁺.

2-Amino-5-hydroxymethyl-1,3,4-thiadiazole. Thiosemicarbazide (3.0 g, 32.9 mmol) and glyconitrile (55% in water, 3.10 g, 29.9 mmol) were added to trifluoroacetic acid (24 mL). The mixture was heated to 63° C. for 2 h and then kept at room temperature for 72 h, after which time the solvent was removed. The residue was dissolved in distilled water (10 mL) and neutralized with 1M NaOH, then stirred for 2 h at room temperature. The precipitate was collected by filtration and recrystallized from water to yield 2.5 g (19.1 mmol, 64%) of the product, mp 185-186° C.; NMR (300 MHz, DMSO-d₆) δ 4.54 (d, 2H, J=6.0 Hz), 5.75 (t, 1H, J=6.0 Hz), 7.08 (s, 2H); ¹³C NMR (75 MHz, DMSO-d₆) δ 58.5, 160.9, 169.2.

N-(4-(N-(5-Methyl-1,3,4-thiadizol-2-yl)sulfamoyl)phenyl)acetamide (106). 2-Amino-5-methyl-1,3,4-thiadiazole (250 mg, 2.19 mmol) was suspended in pyridine (0.5 mL). N-Acetylsulfanilyl chloride (410 mg, 1.75 mmol) was added slowly at 0° C. The reaction mixture was then heated to 95° C. and was stirred for 1 h. The reaction mixture was then added to aqueous 3N HCl and the mixture extracted with ethyl acetate. The organic extracts were washed with water (3×20 mL), brine (3×20 mL), dried over anhydrous Na₂SO₄, filtered, and volatiles evaporated. The residue was crystallized from MeOH to give the product (491 mg, 1.6 mmol, 97%) as a solid, mp 239-240° C.; ¹H NMR (500 MHz, DMSO) δ 2.07 (3, s), 2.44 (3, s), 7.74 (4, s), 10.82 (1, s), 13.85 (1, s); ¹³C NMR (125 MHz, DMSO) δ 16.1, 24.1, 118.6, 126.9, 135.7, 142.8, 154.3, 167.7, 168.9; MS (ESI⁺, m/z) Calculated for C₁₁H₁₃N₄O₃S₂ 313.0. found 313.0 (M+H)⁺; HRMS (FAB⁺, m/z) Calculated for C₁₁H₁₃N₄O₃S₂ 313.0429. found 313.0428 (M+H)⁺.

4-Amino-N-(5-methyl-1,3,4-thiadiazol-2-yl)benzenesulfonamide (105). Compound 106 (250 mg, 0.8 mmol) was suspended in 3 N HCl (4 mL) and the suspension heated to reflux for 30 min. Following neutralization with saturated aqueous Na₂CO₃ solution, the precipitated product was collected by filtration, washed with water (3×20 mL), and dried under vacuum. The residue was crystallized from MeOH to give the product (155 mg, 0.58 mmol, 72%) as a solid, mp 207-208° C. (lit mp 208)¹; ¹H NMR (500 MHz, DMSO) δ 2.47 (3, s), 5.89 (2, s), 6.58 (2, d, J=8.5 Hz), 7.40 (2, d, J=8.5 Hz), 10.48 (1, s); ¹³C NMR (125 MHz, DMSO) δ 16.0, 112.5, 127.2, 127.6, 152.4, 153.6, 166.8.

N-(4-(N45-Methyl-1,3,4-thiadiazol-2-yl)sulfamoyl)phenyl)decanamide (107). Compound 105 (250 mg, 0.93 mmol) was suspended in pyridine (0.5 mL). Decanoyl chloride (141 mg, 0.74 mmol) was added slowly at 0° C. The reaction mixture was then heated to 95° C. and was stirred for 1 h. The reaction mixture was then added to aqueous 3 N HCl solution (5 mL) and the mixture extracted with ethyl acetate (3×10 mL). The organic extracts were washed with water (3×20 mL), brine (3×20 mL), dried over anhydrous Na₂SO₄, and filtered. Evaporation of the solvent left a residue which was crystallized from hexanes and ethyl acetate (1:2) to give the product (297 mg, 0.70 mmol, 95%) as a solid, mp 141-142° C.; ¹H NMR (500 MHz, DMSO) δ 0.82 (3, t, J=7.0 Hz), 1.10-1.30 (12, m), 1.54-1.63 (2, m), 2.32 (2, t, J=7.0 Hz), 2.45 (3, s), 8.25 (2, d, J=8.0 Hz), 8.28 (2, d, J=8.0 Hz), 10.25 (1, s), 13.87 (1, s); ¹³C NMR (125 MHz, DMSO) δ 13.9, 16.0, 22.1, 24.9, 28.5, 28.6, 28.8, 28.9, 31.2, 36.4, 118.5, 126.8, 135.5, 142.7, 154.1, 167.6, 171.7; MS (LCQ, ESI⁺) Calculated for C₁₉H₂₉N₄O₃S₂ 425.2. found 425.1 (M+H)⁺; HRMS (FAB⁺, m/z) Calculated for C₁₉H₂₉N₄O₃S₂ 425.1681. found 425.1678 (M+H)⁺.

N-(4-(N-(5-Ethyl-1,3,4-thiadizol-2-yl)sulfamoyl)phenyl)acetamide (110). 2-Amino-5-ethyl-1,3,4-thiadiazole (250 mg, 1.93 mmol) was suspended in pyridine (0.5 mL). N-Acetylsulfanilyl chloride (361 mg, 1.54 mmol) was added slowly at 0° C. The reaction mixture was then heated to 95° C. and was stirred for 1 h. The reaction mixture was then added to aqueous 3N HCl and the mixture extracted with ethyl acetate. The organic extracts were washed with water (3×20 mL), brine (3×20 mL), dried over anhydrous Na₂SO₄, filtered, and volatiles evaporated. The residue was crystallized from MeOH to give the product (350 mg, 1.07 mmol, 70%) as a solid, mp 197-198° C.; NMR (500 MHz, DMSO) δ 1.28 (3, t, J=7.0 Hz), 2.07 (3, s), 2.82 (2, q, J=7.0 Hz), 7.72 (4, s), 10.32 (1, s), 13.91 (1, s); ¹³C NMR (125 MHz, DMSO) δ 12.2, 23.7, 24.1, 48.6, 118.5, 126.9, 135.6, 142.7, 159.8, 167.3, 168.9; MS (LCQ, ESI⁺) Calculated for C₁₂H15N₄O₃S₂ 327.1. found 327.1 (M+H)⁺; HRMS (FAB⁺, m/z) Calculated for C₁₂H₁₅N₄O₃S₂ 327.0586. found 327.0585 (M+H)⁺.

4-Amino-N-(5-ethyl-1,3,4-thiadiazol-2-yl)benzenesulfonamide (109). Compound 110 (200 mg, 0.61 mmol) was suspended in 3 N HCl (3 mL) and the suspension heated to reflux for 30 min. Following neutralization with saturated aqueous Na₂CO₃ solution, the precipitated product was collected by filtration, washed with water (3×15 mL), and dried under vacuum. The residue was crystallized from MeOH to give the product (120 mg, 0.42 mmol, 69%) as a solid, mp 190-191° C.; NMR (500 MHz, DMSO) δ 1.20 (3, t, J=7.5 Hz), 2.79 (2, q, J=7.5 Hz), 5.91 (2, S), 6.57 (2, d, J=8.5 Hz), 7.41 (2, d, J=8.5 Hz), 13.65 (1, s); ¹³C NMR (125 MHz, DMSO) δ 12.3, 23.6, 112.5, 127.1, 127.6, 152.5, 159.1, 166.8; MS (LCQ, ESI⁺) Calculated for C₁₀H₁₃N₄O₂S₂ 285.0. found 285.0 (M+H)⁺; HRMS (FAB⁺, m/z) Calculated for C₁₀H₁₃N₄O₂S₂ 285.0480. found 285.0478 (M+H)⁺.

N-(4-(N-(5-Ethyl-1,3,4-thiadiazol-2-yl)sulfamoyl)phenyl)decanamide (111). Compound 109 (250 mg, 0.88 mmol) was suspended in pyridine (1.3 mL). Decanoyl chloride (134 mg, 0.70 mmol) was added slowly at 0° C. The reaction mixture was then heated to 95° C. and was stirred for 1 h. The reaction mixture was then added to aqueous 3 N HCl solution (4.5 mL) and the mixture extracted with ethyl acetate (3×10 mL). The organic extracts were washed with water (3×20 mL), brine (3×20 mL), dried over anhydrous Na₂SO₄, and filtered. Evaporation of the solvent left a residue which was crystallized from hexanes and ethyl acetate (1:2) to give the product (372 mg, 0.85 mmol, 97%) as a solid, mp 121-122° C.; ¹H NMR (500 MHz, DMSO) δ 0.82 (3, t, J=7.0 Hz), 1.17-1.30 (14, m), 1.57 (2, t, J=7.0 Hz), 2.32 (3, t, J=7.0 Hz), 2.80 (2, q, J=7.0 Hz), 7.72 (2, d, J=8.5 Hz), 7.76 (2, d, J=8.5 Hz), 10.21 (1, s), 13.89 (1, s); ¹³C NMR (125 MHz, DMSO) δ 12.2, 13.9, 22.1, 23.6, 24.9, 28.6, 28.7, 28.8, 28.9, 31.2, 36.5, 118.5, 126.8, 135.5, 142.7, 159.7, 167.2, 171.8; MS (LCQ, ESI⁺) Calculated for C₂₀H₃₁N₄O₃S₂ 439.2. found 439.1 (M+H)⁺; HRMS (FAB⁺, m/z) Calculated for C₂₀H₃₁N₄O₃S₂ 439.1838. found 439.1843 (M+H)⁺.

N-(4-(N-(5-tert-Butyl-1,3,4-thiadiazol-2-yl)sulfamoyl)phenyl)acetamide (114). 2-Amino-5-tert-butyl-1,3,4-thiadiazole (1.0 g, 6.36 mmol) was suspended in pyridine (1.6 mL). N-Acetylsulfanilyl chloride (1.9 g, 5.1 mmol) was added slowly at 0° C. The reaction mixture was then heated to 95° C. and was stirred for 1 h. The reaction mixture was then added to aqueous 3N HCl and the mixture extracted with ethyl acetate. The organic extracts were washed with water (3×65 mL), brine (3×65 mL), dried over anhydrous Na₂SO₄, filtered, and volatiles evaporated. The residue was crystallized from MeOH to give the product (1.59 mg, 4.3 mmol, 84%) as a solid, mp 137-138° C.; ¹H NMR (500 MHz, DMSO) δ 1.28 (9, t, J=7.0 Hz), 2.08 (3, s), 7.73 (2, d, J=8.5 Hz), 7.78 (2, d, J=8.5 Hz), 10.48 (1, s), 14.00 (1, brs); ¹³C NMR (125 MHz, DMSO) δ 24.1, 29.3, 36.1, 118.6, 126.8, 135.6, 142.8, 166.9, 167.2, 169.0; MS (LCQ, ESI⁺) Calculated for C₁₄H₁₉N₄O₃S₂ 355.1. found 355.1 (M+H)⁺; HRMS (FAB⁺, m/z) Calculated for C₁₄H₁₉N₄O₃S₂ 355.0899. found 355.0900 (M+H)⁺.

4-Amino-N-(5-tert-butyl-1,3,4-thiadiazol-2-yl)benzenesulfonamide (113). Compound 114 (1.0 g, 2.82 mmol) was suspended in 3 N HCl (15 mL) and the suspension heated to reflux for 30 min. Following neutralization with saturated aqueous Na₂CO₃ solution, the precipitated product was collected by filtration, washed with water (70 mL), and dried under vacuum. The residue was crystallized from MeOH to give the product (655 mg, 2.1 mmol, 74%) as a solid, mp 220-221° C.; ¹H NMR (500 MHz, DMSO) δ 1.28 (9, s), 5.91 (2, br s), 6.60 (2, d, J=7.0 Hz), 7.45 (2, d, J=7.0 Hz), 13.95 (1, br s); ¹³C NMR (125 MHZ, DMSO) δ 29.3, 36.0, 112.6, 127.3, 127.7, 152.5, 166.1, 166.6; MS (LCQ, ESI⁺) Calculated for C₁₂H₁₇N₄O₂S₂ 313.1. found 313.0 (M+H)⁺; HRMS (FAB⁺, m/z) Calculated for C₁₂H₁₇N₄O₂S₂ 313.0793. found 313.0793 (M+H)⁺.

N-(4-(N-(5-tert-Butyl-1,3,4-thiadiazol-2-yl)sulfamoyl)phenyl)decanamide (115). Compound 113 (250 mg, 0.80 mmol) was suspended in pyridine (1.5 mL). Decanoyl chloride (122 mg, 0.64 mmol) was added slowly at 0° C. The reaction mixture was then heated to 95° C. and was stirred for 1 h. The reaction mixture was then added to aqueous 3 N HCl solution (4 mL) and the mixture extracted with ethyl acetate (3×10 mL). The organic extracts were washed with water (3×20 mL), brine (3×20 mL), dried over anhydrous Na₂SO₄, and filtered. Evaporation of the solvent left a residue which was crystallized from hexanes and ethyl acetate (1:2) to give the product (294 mg, 0.63 mmol, 98%) as a solid, mp 156-157° C.; ¹H NMR (500 MHz, DMSO) δ 0.80 (3, t, J=7.0 Hz), 1.15-1.33 (21, m), 1.56 (2, t, J=7.0 Hz), 2.32 (3, t, J=7.0 Hz), 7.74 (2, d, J=8.0 Hz), 7.77 (2, d, J=8.0 Hz), 10.21 (1, s), 13.90 (1, s); ¹³C NMR (125 MHz, DMSO): δ 13.9, 22.1, 25.0, 28.6, 28.7, 28.8, 28.9, 29.3, 31.1, 36.0, 36.5, 118.6, 126.9, 135.7, 142.9, 167.0, 167.2, 171.9; MS (LCQ, EST⁺) Calculated for C₂₂H₃₅N₄O₃S₂ 467.2. found 467.2 (M+H)⁺; HRMS (FAB⁺, m/z) Calculated for C₂₂H₃₅N₄O₃S₂ 467.2151. found 467.2131 (M+H)⁺.

Ethyl 2-(5-(4-Acetamidophenylsulfonamido)-1,3,4-thiadiazol-2-yl)acetate (118E). To a solution of p-acetamidobenzenesulfonyl chloride (275 mg, 1.18 mmol) in pyridine (5 mL) was added ethyl 2-(5-amino-1,3,4-thiadiazol-2-yl)acetate (200 mg, 1.07 mmol). The reaction mixture was stirred at room temperature for 4.5 h, then 2 M HCl (10 mL) was added to quench the reaction. The mixture was extracted with ethyl acetate (3×50 mL). The organic extracts were washed with water (20 mL), brine (20 mL), dried over Na₂SO₄, filtered, and concentrated. The residue was purified by chromatography over silica gel (70-230 mesh) eluted with CH₂Cl₂:methanol 19:1 to give the product as a solid, mp 156-157° C., in 76% yield (312 mg, 0.81 mmol); ¹H NMR (300 MHz, DMSO-d₆) δ 1.20 (t, 3H, J=7.0 Hz), 2.07 (s, 3H), 4.06 (s, 2H), 4.15 (q, 2H, J=7.0 Hz), 7.72 (m, 4H), 10.29 (s, 1H); ¹³C NMR (75 MHz, DMSO-d₆) δ 14.0, 24.1, 35.7, 61.3, 118.6, 127.0, 135.5, 142.9, 151.6, 167.8, 168.1, 169.0; MS (LCQ, ESI⁺) Calcd for C₁₄H₁₇N₄O₅S₂ 385.1. found 385.1 (M+H)⁺; HRMS (ESI⁺, m/z) Calcd for C₁₄H₁₇N₄O₅S₂ 385.0640. found 385.0638 (M+H)⁺.

2-(5-(4-Aminophenylsulfonamido)-1,3,4-thiadiazol-2-yl)acetic Acid (117). Distilled water (3.0 mL) and 10% aqueous NaOH (1.5 mL) were added to compound 118E (300 mg, 0.78 mmol) and the mixture was heated under reflux for 2 h. The pH of the solution was then adjusted to 4.0 by addition of 1.0 M HCl, the resulting precipitate was isolated by filtration, washed with cold water, and dried to give 201 mg (0.64 mmol, 82%) of the product as a solid, mp 209-210° C.; ¹H NMR (600 MHz, DMSO-d₆) δ 3.59 (s, 2H), 6.52 (d, 2H, J=8.1 Hz), 7.42 (d, 2H, J=8.9 Hz); ¹³C NMR (75 MHz, DMSO-d₆) δ 36.6, 113.3, 127.8, 128.4, 152.4, 153.3, 167.7, 170.4; MS (LCQ, ESI⁺) Calcd for C₁₀H₁₁N₄O₄S₂ 315.0. found 315.0 (M+H)⁺; HRMS (ESI⁺, m/z) Calcd for C₁₀H₁₁N₄O₄S₂ 315.0222. found 315.0220 (M+H)⁺.

2-(5-(4-Acetamidophenylsulfonamido)-1,3,4-thiadiazol-2-yl)acetic Acid (118). To a solution of compound 118E (128 mg, 0.33 mmol) in THF (15 mL) was added 0.1 M aqueous LiOH (3.75 mL) and the mixture was stirred at room temperature. After 24 h, the resultant solution was acidified to pH 4 and the mixture was extracted with ethyl acetate (3×50 mL). The combined organic extracts were washed with water (20 mL) and concentrated to give the crude product, which was further purified by chromatography on 70-230 mesh silica gel eluted with CH₂Cl₂:methanol:water 40:10:1 to afford 104 mg (0.29 mmol, 88%) of the product as a solid, mp 206-207° C.; ¹H NMR (300 MHz, DMSO-d₆) δ 2.05 (s, 3H), 3.81 (s, 2H), 7.65 (m, 4H); ¹³C NMR (75 MHz, DMSO-d₆) δ 24.8, 37.3, 119.0, 127.5, 137.9, 142.6, 153.3, 169.4, 169.5, 170.9; MS (LCQ, ESI⁺) Calcd for C₁₂H₁₃N₄O₅S₂ 357.0. found 357.0 (M+H)⁺; HRMS (ESI⁺, m/z) Calcd for C₁₂H₁₃N₄O₅S₂ 357.0327. found 357.0326 (M+H)⁺.

Ethyl 2-(5-(4-Decanamidophenylsulfonamido)-1,3,4-thiadiazol-2-yl)acetate (199E). To a solution of the 4-decanamidobenzenesulfonyl chloride (608 mg, 1.76 mmol) in pyridine (8 mL) was added ethyl 2-(5-amino-1,3,4-thiadiazol-2-yl)acetate (300 mg, 1.60 mmol). The reaction mixture was stirred at room temperature for 4.5 h, than 2 M HCl was added to quench the reaction. The mixture was extracted with ethyl acetate (3×40 mL). The organic extracts were washed with water (30 mL), brine (30 mL), dried over Na₂SO₄, filtered, and concentrated. The residue was purified by chromatography over silica gel (70-230 mesh) eluted with CH₂Cl₂:methanol 19:1 to give the product as a solid, mp 89-90° C., in 63% yield (500 mg, 1.01 mmol); ¹H NMR (300 MHz, CDCl₃) δ 0.87 (t, 3H, J=6.9 Hz), 1.25-1.34 (m, 15H), 1.65-1.76 (m, 2H), 2.39 (t, 2H, J=7.5 Hz), 3.87 (s, 2H), 4.24 (q, 2H, J=7.2 Hz), 7.58 (d, 2H, J=9.0 Hz), 7.74 (d, 2H, J=8.7 Hz); ¹³C NMR (75 MHz, DMSO-d₆) δ 14.0, 14.0, 22.0, 25.5, 29.0, 29.0, 29.2, 31.3, 36.4, 37.9, 60.1, 118.9, 127.0, 139.4, 142.3, 154.0, 168.9, 169.9, 172.3; MS (LCQ, ESI⁺) Calcd for C₂₂H₃₃N₄O₅S₂ 497.2. found 497.1 (M+H)⁺; HRMS (ESI⁺, m/z) Calcd for C₂₂H₃₃N₄O₅S₂ 497.1875. found 497.1873 (M+H)⁺.

4-Decanamidobenzenesulfonyl Chloride. Aniline (2.03 g, 25.0 mmol) was dissolved in CH₂Cl₂ (30 mL). To the solution were added pyridine (2.22 mL, 27.5 mmol) and decanoyl chloride (5.25 g, 27.5 mmol) in an ice bath. After stirring for 3 h at room temperature, the reaction mixture was poured into 1M HCl (30 mL) and the mixture extracted with CH₂Cl₂ (3×100 mL). The organic extracts were washed with water (50 mL), brine (50 mL), dried over Na₂SO₄, filtered, and concentrated to give 5.62 g (22.8 mmol, 91%) of N-phenyldecanamide as a white solid, mp 65-66° C. (lit^{s} mp 65-66° C.); ¹H NMR (300 MHz, CDCl₃) δ 0.87 (t, 3H, J=6.9 Hz), 1.26 (m, 12H), 1.72 (m, 2H), 2.35 (t, 2H, J=7.8 Hz), 7.10 (t, 2H, J=7.8 Hz), 7.31 (t, 1H, J=7.8 Hz) 7.50 (t, 2H, J=7.9 Hz); ¹³C NMR (75 MHz, CDCl₃) δ 13.9, 22.5, 25.7, 29.2, 29.2, 29.3, 29.3, 31.7, 37.5, 120.1, 124.0, 128.7, 138.1, 172.3.

2-(5-(4-Decanamidophenylsulfonamido)-1,3,4-thiadiazol-2-yl)acetic Acid (119). To a solution of compound 119E (160 mg, 0.32 mmol) in THF (15 mL) was added 0.1 M aqueous LiOH (3.2 mL) and the mixture was stirred at room temperature. After 24 h, the resultant solution was acidified to pH 4 and the mixture was extracted with ethyl acetate (4×40 mL). The combined organic extracts were washed with water (20 mL) and concentrated to give the crude product, which was further purified by chromatography on 70-230 mesh silica gel eluted with CH₂Cl₂:methanol:water 40:10:1 to afford 125 mg (0.27 mmol, 83%) of the product as a solid, mp 190-191° C.; NMR (300 MHz, DMSO-d₆) δ 0.84 (t, 3H, J=7.2 Hz), 1.24 (m, 12H), 1.56 (m, 2H), 2.29 (t, 2H, J=7.5 Hz), 3.63 (s, 2H), 7.59-7.61 (m, 4H); ¹³C NMR (75 MHz, DMSO-d₆) δ 14.0, 22.1, 25.0, 28.7, 28.8, 28.9, 31.3, 36.4, 37.6, 118.2, 126.8, 138.4, 141.4, 153.2, 169.0, 169.1, 171.7; MS (LCQ, ESI⁺) Calcd for C₂₀H₂₉N₄O₅S₂ 469.2. found 469.1 (M+H)⁺; HRMS (ESI⁺, m/z) Calcd for C₂₀H₂₉N₄O₅S₂ 469.1579. found 469.1570 (M+H)⁺.

N-(4-(N-(5-(hydroxymethyl)-1,3,4-thiadiazol-2-yl)sulfamoyl)phenyl)acetamide (126). To a solution of p-acetamidobenzenesulfonyl chloride (510 mg, 2.18 mmol) in pyridine (6 mL) was added 2-amino-5-hydroxymethyl-1,3,4-thiadiazole (260 mg, 1.98 mmol). The reaction mixture was stirred at room temperature for 4.5 h, then 2 M HCl (20 mL) was added to quench the reaction. The mixture was extracted with ethyl acetate (4×50 mL). The organic extracts were washed with water (40 mL), brine (40 mL), dried over Na₂SO₄, filtered, and concentrated. The residue was purified by chromatography on silica gel (70-230 mesh) eluted with CH₂Cl₂:methanol 9:1 to give the product as a solid, mp 101-102° C., in 82% yield (533 mg, 1.62 mmol); NMR (300 MHz, DMSO-d₆) δ 2.07 (s, 3H), 4.56 (d, 2H, J=5.1 Hz), 6.09 (t, 1H, J=4.8 Hz), 7.73 (m, 4H); ¹³C NMR (75 MHz, DMSO-d₆) δ 24.8, 59.1, 119.3, 127.7, 136.2, 143.5, 161.7, 168.1, 169.6; MS (LCQ, ESI⁺) Calcd for C₁₁H₁₃N₄O₄S₂ 329.0. found 329.1 (M+H)⁺; HRMS (ESI⁺, m/z) Calcd for C₁₁H₁₃N₄O₄S₂ 329.0378. found 329.0376 (M+H)⁺.

4-Amino-N-(5-(hydroxymethyl)-1,3,4-thiadiazol-2-yl)benzenesulfonamide (125). Distilled water (3.0 mL) and 10% NaOH (1.5 mL) were added to compound 126 (328 mg, 0.94 mmol) and the mixture was heated under reflux for 2 h. The pH of the solution was then adjusted to 4.0 by addition of 1.0 M HCl and the mixture was extracted with ethyl acetate (3×50 mL). The combined organic extracts were washed with water (20 mL) and concentrated to give a crude product which was purified by chromatography on silica gel eluted with CH₂Cl₂:methanol 4:1 to afford 182 mg (0.64 mmol, 68%) of the product as a solid, mp 89-90° C.; NMR (300 MHz, DMSO-d₆) δ 4.54 (s, 2H), 5.91 (br, 1H), 6.55 (d, 2H, J=8.7 Hz), 7.39 (d, 2H, J=9.0 Hz); ¹³C NMR (75 MHz, DMSO-d₆) δ 59.1, 113.2, 128.0, 128.4, 153.2, 161.0, 167.5; MS (LCQ, ESI⁺) Calcd for C₉H₁₁N₄O₃S₂ 287.0. found 287.0 (M+H)⁺; HRMS (ESI⁺, m/z) Calcd for C₉H₁₁N₄O₃S₂ 287.0273. found 287.0269 (M+H)⁺.

N-(4-(N-(5-Sulfamoyl-1,3,4-thiadiazol-2-yl)sulfamoyl)phenyl)acetamide (138). 5-Amino-1,3,4-thiadiazolo-2-sulfonamide (540 mg, 3.0 mmol) was dissolved in aqueous NaOH (2.5 M, 1.6 mL) and the solution was cooled to 10° C. 4-Acetamidobenzenesulfonyl chloride (140 mg, 0.6 mmol) and aqueous NaOH (5M, 0.3 mL) were added to this solution and the mixture was stirred at 10° C. until all the sulfonyl chloride had reacted. This procedure was repeated four times (a total of 3.0 mmol of the sulfonyl chloride and 1.5 mL of 5M NaOH). The solution was stirred for 5 h at room temperature, then brought to pH 2 with aqueous 5% HCl. The precipitated product was collected by filtration, washed with cold water, and air-dried. Recrystallization from 95% aqueous ethanol afforded the product (710 mg, 1.88 mmol, 63%), mp 280-281° C. (lit¹⁶ mp 285-290° C.); ¹H NMR (300 MHz, DMSO-d₆) δ 2.06 (s, 3H), 7.74 (s, 4H), 8.45 (s, 2H), 10.32 (s, 1H); ¹³C NMR (75 MHz, DMSO-d₆) δ 24.2, 118.7, 127.2, 134.7, 143.3, 157.9, 167.2, 169.1; LRMS (LCQ, ESI⁻) calcd for C₁₀H₁₀N₅O₅S₃ 376.0. found 376.0 (M−H)⁻; HRMS (ESI⁻, m/z) calcd for C₁₀H₁₀N₅O₅S₃ 375.9850. found 375.9850 (M−H)⁻.

5-Amino-1,3,4-thiadiazolo-2-sulfonamide. A solution of acetazolamide (15 g, 67.5 mmol, from Aldrich) in a mixture of ethanol (100 mL) and concentrated hydrochloride acid (30 mL) was heated at reflux for 4.5 h, during which time a solid slowly deposited. Upon cooling the solution, the solvents were removed in vacuo and the solid residue was redissolved in H₂O (75 mL). The solution was basified to pH 7 with 5 M sodium hydroxide, the precipitated product was collected by filtration, and then recrystallized from water to give the product (10.6 g, 58.9 mmol, 87%), mp 228-229° C. (lit¹⁵ mp 230-232° C.); ¹H NMR (300 MHz, DMSO-d₆) δ 8.06 (s, 2H), 7.81 (s, 2H); ¹³C NMR (75 MHz, DMSO-d₆) 171.9, 158.1.

5-(4-Aminophenylsulfonamido)-1,3,4-thiadiazole-2-sulfonamide (131). Compound 138 (1.0 g, 2.6 mmol) was heated at reflux with aqueous HCl (6 M, 10 mL) for 50 min. The homogeneous solution was evaporated to dryness and the residue was taken up in distilled water (10 mL). The pH was adjusted to 9 with 25% aqueous ammonia, the resulting solution was filtered to remove insoluble matter, and the solution acidified to pH 4 with glacial acetic acid. Cooling the solution overnight gave a solid, which was collected by filtration, washed with cold water, and air-dried. Recrystallization from 20% ethanol/H₂O gave the pure product (500 mg, 1.5 mmol, 57%), mp 241-242° C. (lit¹⁷ mp 247-248° C.); ¹H NMR (300 MHz, DMSO-d₆) δ 6.58 (d, 2H, J=7.8 Hz), 7.43 (d, 2H, J=8.1 Hz), 8.43 (s, 2H); ¹³C NMR (75 MHz, DMSO-d₆) δ 112.8, 125.9, 128.0, 153.1, 157.6, 166.0; LRMS (LCQ, ESI⁺) calcd for C₈H₁₀N₅O₄S₃ 336.0. found 335.8 (M+H)⁺; HRMS (ESI⁺, m/z) calcd for C₈H₁₀N₅O₄S₃ 335.9889. found 335.9883 (M+H)⁺.

Ethyl 5-(4-Acetamidophenylsulfonamido)-1,3,4-thiadiazole-2-carboxylate (122E). To a solution of p-acetamidobenzenesulfonyl chloride (1.98 g, 8.47 mmol) in pyridine (20 mL) was added ethyl 5-amino-1,3,4-thiadiazole-2-carboxylate (1.2 g, 7.06 mmol). The reaction mixture was stirred at room temperature for 4.5 h, than 2 M HCl (50 mL) was added to quench the reaction. The mixture was extracted with ethyl acetate (3×60 mL). The organic extracts were washed with water (50 mL), brine (50 mL), dried over Na₂SO₄, filtered, and concentrated. The residue was purified by chromatography on silica gel (70-230 mesh) eluted with CH₂Cl₂:methanol 19:1 to give the product as a solid, mp 201-202° C., in 73% yield (1.91 g, 5.15 mmol); ¹H NMR (300 MHz, DMSO-d₆) δ 1.29 (t, 3H, J=6.9 Hz), 2.08 (s, 3H), 4.37 (q, 2H, J=7.8 Hz), 7.74 (m, 4H), 10.32 (s, 1H); ¹³C NMR (75 MHz, DMSO-d₆) δ 13.9, 14.0, 24.1, 62.9, 118.6, 127.1, 134.9, 143.2, 147.2, 157.5, 167.6, 169.0; MS (LCQ, ESI⁺) Calcd for C₁₃H₁₅N₄O₅S₂ 371.0. found 371.0 (M+H)⁺; HRMS (ESI⁺, m/z) Calcd for C₁₃H₁₅N₄O₅S₂ 371.0484. found 371.0472 (M+H)⁺.

Ethyl 5-(4-Decanamidophenylsulfonamido)-1,3,4-thiadiazole-2-carboxylate (123E). To a solution of 4-decanamidobenzenesulfonyl chloride (220 mg, 0.64 mmol) in pyridine (4 mL) was added ethyl 5-amino-1,3,4-thiadiazole-2-carboxylate (100 mg, 0.58 mmol). The reaction mixture was stirred at room temperature for 4.5 h, then 2 M HCl (10 mL) was added to quench the reaction. The mixture was extracted with ethyl acetate (3×30 mL). The organic extracts were washed with water (20 mL), brine (20 mL), dried over Na₂SO₄, filtered, and concentrated. The residue was purified by chromatography on silica gel (70-230 mesh) eluted with CH₂Cl₂:methanol 9:1 to give the product as a solid, mp 101-102° C., in 65% yield (183 mg, 0.38 mmol); ¹H NMR (300 MHz, DMSO-d₆) δ 0.83 (t, 3H, J=6.6 Hz), 1.22-1.32 (m, 15H), 1.56 (m, 2H), 2.31 (t, 2H, J=6.0 Hz), 4.33 (q, 2H, J=7.6 Hz), 7.71 (m, 4H), 10.19 (s, 1H); ¹³C NMR (75 MHz, DMSO-d₆) δ 14.6, 14.6, 22.8, 25.6, 29.3, 29.4, 29.5, 29.6, 31.3, 31.9, 37.1, 62.8, 119.1, 127.6, 136.8, 143.2, 147.7, 159.2, 170.3, 172.5; MS (LCQ, ESI⁺) Calcd for C₂₁H₃₁N₄O₅S₂ 483.2. found 483.1 (M+H)⁺; HRMS (ESI⁺, m/z) Calcd for C₂₁H₃₁N₄O₅S₂ 483.1736. found 483.1728 (M+H)⁺.

N-(4-(N-(5-(hydroxymethyl)-1,3,4-thiadiazol-2-yl)sulfamoyl)phenyl)decanamide (127). To a solution of 4-decanamidobenzenesulfonyl chloride (435 mg, 1.26 mmol) in pyridine (5 mL) was added 2-amino-5-hydroxymethyl-1,3,4-thiadiazole (150 mg, 1.15 mmol). The reaction mixture was stirred at room temperature for 4.5 h, then 2 M HCl (15 mL) was added to quench the reaction. The mixture was extracted with ethyl acetate (3×30 mL). The organic extracts were washed with water (20 mL), brine (20 mL), dried over Na₂SO₄, filtered, and concentrated. The residue was purified by chromatography on silica gel (70-230 mesh) eluted with CH₂Cl₂:methanol 9:1 to give the product as a solid, mp 69-70° C., in 73% yield (370 mg, 0.84 mmol); ¹H NMR (300 MHz, DMSO-d₆) δ 0.84 (t, 3H, J=7.2 Hz), 1.23 (m, 12H), 1.54-1.57 (m, 2H), 2.29 (t, 2H, J=7.5 Hz), 4.57 (d, 2H, J=4.8 Hz), 6.08 (t, 1H, J=5.0 Hz), 7.73 (m, 4H), 10.22 (s, 1H), 14.01 (s, 1H); ¹³C NMR (75 MHz, DMSO-d₆) δ 14.6, 22.8, 25.6, 30.0, 29.4, 29.5, 29.6, 31.9, 37.1, 59.1, 119.3, 127.6, 136.1, 143.5, 161.7, 168.1, 172.6; MS (LCQ, ESI⁺) Calcd for C₁₉H₂₉N₄O₄S₂ 441.2. found 441.1 (M+H)⁺; HRMS (ESI⁺, m/z) Calcd for C₁₉H₂₉N₄O₄S₂ 441.1630. found 441.1624 (M+H)⁺.

N-(4-(N-(5-Sulfamoyl-1,3,4-thiadiazol-2-yl)sulfamoyl)phenyl)decanamide (139). 5-(4-Aminophenylsulfonamido)-1,3,4-thiadiazole-2-sulfonamide (7, 50 mg, 0.15 mmol) was suspended in anhydrous acetonitrile (5 mL). Triethylamine (17.1 mg, 0.17 mmol) was added with stirring at 0° C. A solution of decanoyl chloride (32.4 mg, 0.17 mmol) dissolved in anhydrous acetonitrile (1 mL) was added dropwise, and the reaction mixture was stirred at 0° C. for 2 h and overnight at room temperature. Volatiles were removed in vacuo and the residue was washed with water (5 mL). The residue was subjected to chromatography on silica gel (70-230 mesh) eluted with CH₂Cl₂:methanol 9:1, giving the pure product as a solid (42 mg, 0.09 mmol, 60% yield), mp 242-243° C.; ¹H NMR (300 MHz, DMSO-d₆) δ 0.84 (t, 3H, J=6.9 Hz), 1.24 (m, 12H), 1.56 (m, 2H), 2.32 (t, 2H, J=7.5 Hz), 7.66 (s, 4H), 7.91 (s, 2H), 10.13 (s, 1H); ¹³C NMR (75 MHz, DMSO-d₆) δ 14.1, 22.3, 25.2, 28.8, 29.0, 31.5, 36.6, 118.6, 127.0, 137.4, 142.2, 157.8, 170.8, 172.1; LRMS (LCQ, ESI) calcd for C₁₈H₂₆N₅O₅S₃ 488.1. found 488.1 (M−H)⁻; HRMS (ESI⁻, m/z) calcd for C₁₈H₂₆N₅O₅S₃ 488.1102. found 487.1101 (M−H)⁻.

5-(4-Dodecylphenylsulfonamido)-1,3,4-thiadiazole-2-sulfonamide (140). 5-Amino-1,3,4-thiadiazolo-2-sulfonamide (200 mg, 1.1 mmol) was suspended in anhydrous acetonitrile (5 mL). Triethylamine (123 mg, 1.2 mmol) was added with stirring at 0° C. followed by a solution of 4-dodecylbenzenesulfonyl chloride (383 mg, 1.1 mmol) in anhydrous acetonitrile (3 mL). The reaction mixture was stirred overnight at room temperature. Volatiles were then removed in vacuo and the residue was washed with water (5 mL) in order to eliminate the ammonium salt. The crude solid was subjected to chromatography on silica gel (70-230 mesh) eluted with CH₂Cl₂:methanol 19:1 to give the product in 39% yield. Recrystallization from absolute ethanol and a second round of chromatography gave an analytic sample, mp 249-250° C.; ¹H NMR (300 MHz, DMSO-d₆) δ 0.85 (t, 3H, J=6.6 Hz), 1.23 (m, 18H), 1.55 (m, 2H), 2.58 (t, 2H, J=7.2 Hz), 7.23 (d, 2H, J=7.8 Hz), 7.34 (s, 2H), 7.59 (d, 2H, J=8.1 Hz); ¹³C NMR (75 MHz, DMSO-d₆) δ 13.9, 22.1, 28.7, 28.9, 29.0, 29.1, 30.8, 31.3, 34.9, 126.2, 127.8, 143.3, 145.1, 161.2, 170.9; LRMS (LCQ, ESI⁻) calcd for C₂₀H₃₁N₄O₄S₃ 487.2. found 487.1 (M−H)⁻; HRMS (ESI⁻, m/z) calcd for C₂₀H₃₁N₄O₄S₃ 487.1513. found 487.1514 (M−H)⁻.

4-Butyl-N-(1,3,4-thiadiazol-2-yl)benzenesulfonamide (155). To a stirred solution of 2-amino-1,3,4-thiadiazole (2.0 g, 19.7 mmol) in pyridine (30 mL) under argon at −20° C. was added p-butylbenzenesulfonyl chloride (4.89 g, 21 mmol) over 10 min. The reaction mixture was stirred at room temperature for 16 hours. Water (300 mL) was added to quench the reaction. The mixture was extracted with CH₂Cl₂ and the organic extracts washed with 2N HCl (2×150 mL), brine, dried over anhydrous Na₂SO₄, filtered, and concentrated. The residue was purified by flash chromatography on silica gel eluted with methanol:DCM 1:33 to give the product (3.46 g, 11.6 mmol, 59% yield) as a solid, mp 120-121° C.; ¹H NMR (300 MHz, CDCl₃) δ 0.91 (t, 3H, J=7 Hz), 1.29-1.37 (m, 2H), 1.56-1.61 (m, 2H), 2.65 (t, 2H, J=7 Hz), 7.27 (d, 2H, J=8 Hz), 7.84 (d, 2H, J=8 Hz), 8.25 (s, 1H); ¹³C NMR (75 MHz, CDCl₃) 13.9, 22.3, 33.2, 33.6, 126.5, 129.1, 138.1, 142.7, 148.6, 167.4; MS (Q-TOF) Calcd for C₁₂H₁₆N₃O₂S₂ 298.0684. found 298.0695 (M+H)⁺; Calcd for C₁₂H₁₅N₃NaO₂S₂ 320.0503. found 320.0361 (M+Na)⁺.

p-Butylbenzenesulfonyl Chloride. To a solution of butylbenzene (4.13 g, 30.8 mmol) in CHCl₃ (50 mL) was added chlorosulfonic acid (17 mL, 29.8 g, 256 mmol) and the mixture was stirred at rt for 20 h. The mixture was poured on ice (200 mL) and extracted with EtOAc (3×100 mL). The combined extracts were washed with water, a solution of NaHCO₃, and water, dried (Na₂SO₄), and concentrated in vacuo. The yellow oily residue (ca 88% yield) was used without further purification in the next reaction; ¹H NMR (300 MHz, CDCl₃) δ 0.94 (t, 3H, J=7 Hz), 1.34-1.41 (m, 2H), 1.62-1.67 (m, 2H), 2.73 (t, 2H, J=8 Hz), 7.41 (d, 2H, J=8 Hz), 7.94 (d, 2H, J=8 Hz).

4-Octyl-N-(1,3,4-thiadiazol-2-yl)benzenesulfonamide (153). To a stirred solution of 2-amino-1,3,4-thiadiazole (2.0 g, 19.7 mmol) in pyridine (30 mL) under argon at −20° C. was added p-octylbenzenesulfonyl chloride (6.06 g, 21 mmol) over 10 min. The reaction mixture was stirred at room temperature for 16 hours. Water (300 mL) was added to quench the reaction. The mixture was extracted with CH₂Cl₂ and the organic extracts washed with 2N HCl (2×150 mL), brine, dried over anhydrous Na₂SO₄, filtered, and concentrated. The residue was purified by flash chromatography on silica gel eluted with methanol:DCM 1:33 to give the product (3.83 g, 10.8 mmol, 55% yield) as a solid, mp 123-124° C.; ¹H NMR (300 MHz, CDCl₃) δ 0.87 (t, 3H, J=7 Hz), 1.36 (m, 10H), 1.59 (m, 2H), 2.63 (t, 2H, J=7 Hz), 7.27 (d, 2H, J=8 Hz), 7.82 (d, 2H, J=8 Hz), 8.23 (s, 1H); ¹³C NMR (75 MHz, CDCl₃) 14.1, 22.6, 29.2, 29.3, 29.4, 31.1, 31.8, 35.9, 126.5, 129.0, 138.1, 142.6, 148.7, 167.3; MS (Q-TOF) Calcd for C₁₆H₂₄N₃O₂S₂ 354.1310. found 354.1211 (M+H)⁺; Calcd for C₁₆H₂₃N₃NaO₂S₂ 376.1129. found 376.1154 (M+Na)⁺.

p-Octylbenzesulfonyl Chloride. To a solution of 1-phenyloctane (5.86 g, 30.8 mmol) in CHCl₃ (50 mL) was added chlorosulfonic acid (17 mL, 29.8 g, 256 mmol) and the mixture was stirred at rt for 20 h. The mixture was poured on ice (200 mL) and extracted with EtOAc (3×100 mL). The combined extracts were washed with water, a solution of NaHCO₃, and water, dried (Na₂SO₄), and concentrated in vacuo. The yellow oily residue (ca 80% yield) was used without further purification in the next reaction; ¹H NMR (300 MHz, CDCl₃) δ 0.87 (t, 3H, J=7 Hz), 1.27-1.32 (m, 10H), 1.64-1.66 (m, 2H), 2.72 (t, 2H, J=8 Hz), 7.42 (d, 2H, J=8 Hz), 7.93 (d, 2H, J=8 Hz).

4-Hexyl-N-(1,3,4-thiadiazol-2-yl)benzenesulfonamide (154). To a stirred solution of 2-amino-1,3,4-thiadiazole (2.0 g, 19.7 mmol) in pyridine (30 mL) under argon at −20° C. was added p-hexylbenzenesulfonyl chloride (5.48 g, 21 mmol) over 10 min. The reaction mixture was stirred at room temperature for 16 hours. Water (300 mL) was added to quench the reaction. The mixture was extracted with CH₂Cl₂ and the organic extracts washed with 2N HCl (2×150 mL), brine, dried over anhydrous Na₂SO₄, filtered, and concentrated. The residue was purified by flash chromatography on silica gel eluted with methanol:DCM 1:33 to give the product (3.72 g, 11.4 mmol, 58% yield) as a solid, mp 125-126° C.; NMR (300 MHz, CDCl₃) δ 0.88 (t, 3H, J=7 Hz), 1.28 (m, 6H), 1.58 (m, 2H), 2.63 (t, 2H, J=7 Hz), 7.27 (d, 2H, J=8 Hz), 7.83 (d, 2H, J=8 Hz), 8.24 (s, 1H); ¹³C NMR (75 MHz, CDCl₃) 14.1, 22.6, 28.9, 31.1, 31.6, 35.9, 126.5, 129.0, 138.1, 142.6, 148.6, 167.4; MS (Q-TOF) Calcd for C₁₄H₂₀N₃O₂S₂ 326.0997. found 326.0931 (M+H)⁺; Calcd for C₁₄H₁₉N₃NaO₂S₂ 348.0816. found 348.0816 (M+Na)⁺.

p-Hexylbenzenesulfonyl Chloride. To a solution of 1-hexylbenzene (5.00 g, 30.8 mmol) in CHCl₃ (50 mL) was added chlorosulfonic acid (17 mL, 29.8 g, 256 mmol) and the mixture was stirred at rt for 20 h. The mixture was poured on ice (200 mL) and extracted with EtOAc (3×100 mL). The combined extracts were washed with water, a solution of NaHCO₃, and water, dried (Na₂SO₄), and concentrated in vacuo. The yellow oily residue (ca 81% yield) was used without further purification in the next reaction; ¹H NMR (300 MHz, CDCl₃) δ 0.88 (t, 3H, J=7 Hz), 1.30-1.35 (m, 6H), 1.55-1.63 (m, 2H), 2.59 (t, 2H, J=8 Hz), 7.38 (d, 2H, J=8 Hz), 7.89 (d, 2H, J=8 Hz).

4-Tetradecyl-N-(1,3,4-thiadiazol-2-yl)benzenesulfonamide (156). To a solution of p-tetradecylbenzenesulfonyl chloride (440 mg, 1.18 mmol) in pyridine (8 mL) was added 1,3,4-thiadiazol-2-amine (179 mg, 1.77 mmol). The reaction mixture was stirred at room temperature for 6 hours, then 2 M HCl (40 mL) was added to quench the reaction. The mixture was extracted with ethyl acetate (3×50 mL), the organic layer was washed with water (40 mL) and brine (40 mL), dried over anhydrous Na₂SO₄ and concentrated. The residue was purified by chromatography over silica gel (70-230 mesh) eluted with methanol:DCM 1:19 to give the product as a solid (240 mg, 0.55 mmol, 47% yield), mp 116-117° C.; NMR (300 MHz, CDCl₃) δ 0.88 (t, 3H, J=6.9 Hz), 1.25 (m, 22H), 1.60 (m, 2H), 2.64 (t, 2H, J=7.2 Hz), 7.29 (d, 2H, J=8.4 Hz), 7.84 (d, 2H, J=8.4 Hz), 8.23 (s, 1H); ¹³C NMR (75 MHz, CDCl₃) 14.1, 22.6, 29.2, 29.3, 29.4, 29.5, 29.6, 31.1, 31.9, 35.9, 126.5, 128.9, 138.1, 142.6, 148.6, 167.4; MS (LCQ, ESI⁺) Calcd for C₂₂H₃₆N₃O₂S₂ 438.2. found 438.3 (M+H)⁺; HRMS (ESI⁺, m/z) Calcd for C₂₂H₃₆N₃O₂S₂ 438.2243. found 438.2243 (M+H)⁺.

p-Tetradecylbenzenesulfonyl Chloride. To a solution of 1-phenyloctadecane (0.69 g, 2.5 mmol) in CHCl₃ (5 mL) was added chlorosulfonic acid (0.5 mL, 7.5 mmol) and the mixture was stirred at rt for 22 h. The mixture was poured on ice and extracted with CH₂Cl₂. The combined extracts were washed with water, a solution of NaHCO₃, and water, dried (Na₂SO₄), and concentrated in vacuo. The residue was chromatographed on silica gel (70-230 mesh) with hexane/ethyl acetate (49:1) to give the product as a white solid (0.63 g, 1.7 mmol, 68%), mp 32-33° C.; ¹H NMR (300 MHz, CDCl₃) δ 0.88 (t, 3H, J=7.2 Hz), 1.25 (m, 22H), 1.65 (m, 2H), 2.72 (t, 2H, J=7.8 Hz), 7.42 (d, 2H, J=8.4 Hz), 7.93 (d, 2H, J=8.4 Hz); ¹³C NMR (75 MHz, CDCl₃) 14.1, 22.6, 29.1, 29.3, 29.5, 29.6, 29.7, 30.9, 31.9, 36.0, 126.9, 129.5, 141.7, 151.6.

4-Hexadecyl-N-(1,3,4-thiadiazol-2-yl)benzenesulfonamide (157). To a solution of p-hexadecylbenzenesulfonyl chloride (600 mg, 1.50 mmol) in pyridine (8 mL) was added 1,3,4-thiadiazol-2-amine (228 mg, 2.25 mmol). The reaction mixture was stirred at room temperature for 6 hours, then 2 M HCl (40 mL) was added to quench the reaction. The mixture was extracted with ethyl acetate (3×50 mL), the organic layer was washed with water (40 mL) and brine (40 mL), dried over anhydrous Na₂SO₄ and concentrated. The residue was purified by chromatography over silica gel (70-230 mesh) eluted with methanol:DCM 1:19 to give the product as a solid (320 mg, 0.69 mmol, 46% yield), mp 118-119° C.; ¹H NMR (300 MHz, CDCl₃) δ 0.88 (t, 3H, J=6.9 Hz), 1.25 (m, 26H), 1.59 (m, 2H), 2.64 (t, 2H, J=8.1 Hz), 7.29 (d, 2H, J=7.8 Hz), 7.84 (d, 2H, J=7.8 Hz), 8.23 (s, 1H); ¹³C NMR (75 MHz, CDCl₃) 14.1, 22.7, 29.2, 29.3, 29.4, 29.6, 29.7, 31.1, 31.9, 35.9, 126.5, 128.9, 138.1, 142.5, 148.7, 167.5; MS (LCQ, ESI⁺) Calcd for C₂₄H₄₀N₃O₂S₂ 466.3. found 466.3 (M+H)⁺; HRMS (ESI⁺, m/z) Calcd for C₂₄H₄₀N₃O₂S₂ 466.2556. found 466.2558 (M+H)⁺.

p-Hexadecylbenzenesulfonyl Chloride. To a solution of 1-phenyloctadecane (0.76 g, 2.5 mmol) in CHCl₃ (5 mL) was added chlorosulfonic acid (0.5 mL, 7.5 mmol) and the mixture was stirred at rt for 22 h. The mixture was poured on ice and extracted with CH₂Cl₂. The combined extracts were washed with water, a solution of NaHCO₃, and water, dried (Na₂SO₄), and concentrated in vacuo. The residue was chromatographed on silica gel (70-230 mesh) with hexane/ethyl acetate (49:1) to give the product as a white solid (0.71 g, 1.8 mmol, 72%), mp 35-36° C.; ¹H NMR (300 MHz, CDCl₃) δ 0.88 (t, 3H, J=7.2 Hz), 1.25 (m, 26H), 1.62 (m, 2H), 2.72 (t, 2H, J=7.8 Hz), 7.42 (d, 2H, J=8.4 Hz), 7.95 (d, 2H, J=8.4 Hz); ¹³C NMR (75 MHz, CDCl₃) 14.4, 22.9, 29.4, 29.64, 29.8, 29.9, 31.2, 32.2, 36.3, 127.3, 129.8, 142.0, 151.9.

4-Octadecyl-N-(1,3,4-thiadiazol-2-yl)benzenesulfonamide (158). To a solution of p-octadecylbenzenesulfonyl chloride (500 mg, 1.17 mmol) in pyridine (8 mL) was added 1,3,4-thiadiazol-2-amine (177 mg, 1.75 mmol). The reaction mixture was stirred at room temperature for 6 hours, then 2 M HCl (40 mL) was added to quench the reaction. The mixture was extracted with ethyl acetate (3×50 mL), the organic layer was washed with water (40 mL) and brine (40 mL), dried over anhydrous Na₂SO₄ and concentrated. The residue was purified by chromatography over silica gel (70-230 mesh) eluted with methanol:DCM 1:19 to give the product as a solid (296 mg, 0.60 mmol, 51% yield), mp 116-117° C.; ¹H NMR (300 MHz, CDCl₃) δ 0.86 (t, 3H, J=6.9 Hz), 1.25 (m, 30H), 1.60 (m, 2H), 2.64 (t, 21-1, J=7.8 Hz), 7.29 (d, 2H, J=7.8 Hz), 7.82 (d, 2H, J=7.8 Hz), 8.21 (s, 1H); ¹³C NMR (75 MHz, CDCl₃) 14.0, 22.7, 29.2, 29.3, 29.4, 29.5, 29.6, 29.7, 31.1, 31.9, 35.9, 126.5, 128.9, 138.1, 142.6, 148.6, 167.4; MS (LCQ, ESI⁺) Calcd for C₂₆H₄₄N₃O₂S₂ 494.3. found 494.2 (M+H)⁺; HRMS (ESI⁺, m/z) Calcd for C₂₆H₄₄N₃O₂S₂ 494.2869. found 494.2869 (M+H)⁺.

p-Octadecylbenzenesulfonyl Chloride. To a solution of 1-phenyloctadecane (0.84 g, 2.5 mmol) in CHCl₃ (5 mL) was added chlorosulfonic acid (0.5 mL, 7.5 mmol) and the mixture was stirred at rt for 22 h. The mixture was poured on ice and extracted with CH₂Cl₂. The combined extracts were washed with water, a solution of NaHCO₃, and water, dried (Na₂SO₄), and concentrated in vacuo. The residue was chromatographed on silica gel (70-230 mesh) with hexane/ethyl acetate (49:1) to give the product as a white solid (0.60 g, 1.4 mmol, 56%), mp 43-44° C.; NMR (300 MHz, CDCl₃) δ 0.86 (t, 3H, J=6.9 Hz), 1.25 (m, 30H), 1.65 (m, 2H), 2.72 (t, 2H, J=7.8 Hz), 7.42 (d, 2H, J=8.4 Hz), 7.93 (d, 2H, J=8.4 Hz); ¹³C NMR (75 MHz, CDCl₃) 14.1, 22.7, 29.2, 29.4, 29.5, 29.7, 30.9, 31.9, 36.0, 127.1, 129.6, 141.8, 151.7.

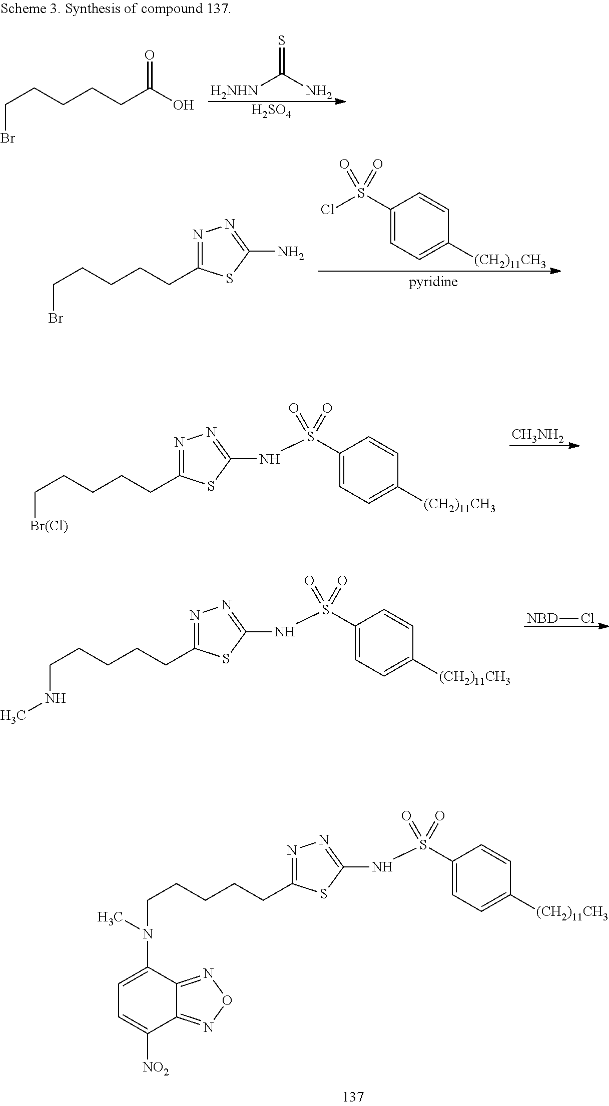

4-Dodecyl-N-(5-(5-(methyl(7-nitrobenzo[c][1,2,5]oxadiazol-4-yl)amino)pentyl)-1,3,4-thiadiazol-2-yl)benzenesulfonamide (137). 4-Chloro-7-nitro-2,1,3-benzoxadiazole (NBD-Cl) (18 mg, 0.085 mmol) was dissolved in methanol (1 mL). After the addition of 4-dodecyl-N-(5-(5-(methylamino)pentyl)-1,3,4-thiadiazol-2-yl)benzenesulfonamide (43 mg, 0.085 mmol) and NaHCO₃ (7 mg, 0.085 mmol) in methanol (2 mL), the solution was stirred for 2 h at 40° C. The reaction mixture was evaporated to dryness under reduced pressure and the residue was chromatographed on silica gel 60 (70-230 mesh) eluted with CH₂Cl₂:MeOH 49:1. Compound 137 was obtained in 53% yield (30 mg, 0.045 mmol), mp 102-104° C. ¹H NMR (300 MHz, CDCl₃) δ 0.87 (t, 3, J=7.2 Hz), 1.25 (m, 18), 1.51-1.57 (m, 4), 1.79-1.85 (m, 4), 2.63 (t, 2, J=6.6 Hz), 2.84 (t, 2, J=7.5 Hz), 3.45 (s, 3), 4.14 (s, 2), 6.11 (d, 1, J=9.3 Hz), 7.27 (d, 2, J=8.1 Hz), 7.79 (d, 2, J=8.4 Hz), 8.44 (d, 1, J=9.0 Hz); ¹³C NMR (75 MHz, CDCl₃) δ 14.1, 22.6, 25.7, 27.7, 29.2, 29.3, 29.4, 29.5, 29.6, 30.4, 31.1, 31.9, 35.9, 55.6, 101.2, 126.5, 128.9, 135.4, 138.3, 145.3, 148.5, 154.7, 158.3, 163.8, 167.9; HRMS (ESI⁺, m/z) calculated for C₃₂H₄₆N₇O₅S₂ 672.3002, observed 672.2996 (M+H)⁺.

5-(5-Bromopentyl)-1,3,4-thiadiazol-2-amine. 6-Bromohexanoic acid (5.35 g, 27.4 mmol), concentrated sulphuric acid (15 mL), and thiosemicarbazide (3.0 g, 32.9 mmol) were slowly heated to 80-90° C. for 12 h. After cooling, the content was poured onto crushed ice. The mixture was neutralized with 10% aqueous ammonia and extracted with ethyl acetate (3×100 mL). The organic extracts were washed with 10% Na₂CO₃ (2×50 mL), water (100 mL), and brine (100 mL), dried over Na₂SO₄, filtered, and concentrated. The residue was purified by chromatography over silica gel 60 (70-230 mesh) eluted with CH₂Cl₂:MeOH 19:1 to give the product as a solid, mp 128-130° C., in 59% yield (4.03 g, 16.2 mmol). ¹H NMR (300 MHz, CDCl₃) δ 1.51-1.60 (m, 2), 1.71-1.79 (m, 2), 1.81-1.92 (m, 2), 2.92 (t, 2, J=7.5 Hz), 3.40 (t, 2, J=6.9 Hz), 5.33 (s, 2); ¹³C NMR (75 MHz, CDCl₃) δ 26.9, 28.1, 29.3, 31.8, 35.0, 158.1, 168.2; HRMS (ESI⁺, m/z) calculated for C₇H₁₃BrN₃S 250.0014, observed 250.0005 (M+H)⁺.

N-(5-(5-Bromopentyl)-1,3,4-thiadiazol-2-yl)-4-dodecylbenzene sulfonamide. To a solution of 4-dodecylbenzenesulfonyl chloride (1.53 g, 4.42 mmol) in pyridine (15 mL) was added 5-(5-bromopentyl)-1,3,4-thiadiazol-2-amine. (1.00 g, 4.02 mmol). The reaction mixture was stirred at room temperature for 5 h, then 2 mol/L HCl (25 mL) was added to quench the reaction. The mixture was extracted with ethyl acetate (3×50 mL). The organic extracts were washed with water (50 mL) and brine (50 mL), dried over Na₂SO₄, filtered, and concentrated. The residue was purified by chromatography over silica gel 60 (70-230 mesh) eluted with CH₂Cl₂:MeOH 49:1 to give 1.39 g of product as a solid contaminated with N-(5-(5-chloropentyl)-1,3,4-thiadiazol-2-yl)-4-dodecyl benzenesulfonamide in about 60% yield. ¹H NMR (300 MHz, CDCl₃) δ 0.87 (t, 3, J=6.9 Hz), 1.25-1.30 (m, 18), 1.55-1.58 (m, 4), 1.73-1.88 (m, 4), 2.64 (t, 2, J=7.8 Hz), 2.83 (t, 2, J=7.8 Hz), 3.42 (t, 1, J=6.6), 3.55 (t, 1, J=6.3 Hz), 7.27 (d, 2, J=8.1 Hz), 7.84 (d, 2, J=8.1 Hz); ¹³C NMR (75 MHz, CDCl₃) 14.1, 22.6, 26.0, 27.3, 27.4, 27.5, 29.3, 29.4, 29.5, 29.6, 29.7, 30.5, 31.1, 31.8, 31.9, 32.0, 33.2, 35.8, 44.5, 126.5, 128.9, 138.3, 148.3, 158.5, 168.2; HRMS (ESI⁺, m/z) calculated for C₂₅H₄₁BrN₃O₂S₂ 558.1824, observed 558.1819 (M+H)⁺; HRMS (ESI⁺, m/z) calculated for C₂₅H₄₁ClN₃O₂S₂ 514.2329, observed 514.2330 (M+H)⁺.

4-Dodecyl-N-(5-(5-(methylamino)pentyl)-1,3,4-thiadiazol-2-yl)benzenesulfonamide. A mixture of N-(5-(5-bromopentyl)-1,3,4-thiadiazol-2-yl)-4-dodecylbenzenesulfonamide (100 mg, 0.18 mmol), CH₃NH₂ (0.42 mL, 40% solution in water, 5.4 mmol), K₂CO₃ (25 mg, 0.18 mmol), and KI (30 mg, 0.18 mmol) was heated at reflux for 2 d. The reaction mixture was diluted with ether (50 mL), washed with brine (20 mL), dried over Na₂SO₄, filtered, and concentrated. The crude product was purified by chromatography over silica gel 60 (70-230 mesh) eluted with CH₂Cl₂:methanol 2:3 to give the product as a solid, mp 158-160° C., in 61% yield (56 mg, 0.11 mmol). ¹H NMR (300 MHz, CDCl₃) δ 0.87 (t, 3, J=7.2 Hz), 1.25-1.36 (m, 18), 1.53-1.67 (m, 8), 2.57-2.61 (m, 5), 2.68 (t, 2, J=7.2 Hz), 2.96 (t, 2, J=6.9 Hz), 7.20 (d, 2, J=8.4 Hz), 7.75 (d, 2, J=8.1 Hz); ¹³C NMR (75 MHz, CDCl₃) δ 14.1, 22.7, 25.5, 28.3, 29.3, 29.4, 29.5, 29.7, 30.5, 31.2, 31.9, 33.1, 35.8, 60.0, 126.1, 128.5, 140.7, 146.7, 163.7, 170.7; HRMS (ESI⁺, m/z) calculated for C₂₆H₄₅N₄O₂S₂ 509.2984, observed 509.2972 (M+H)⁺.

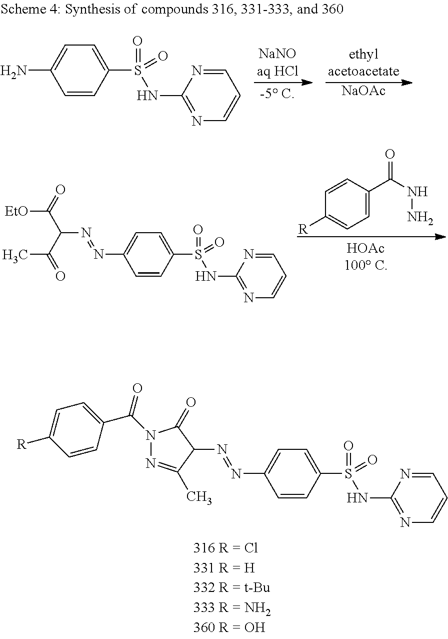

(E)-4-((1-(4-chlorobenzoyl)-3-methyl-5-oxo-4,5-dihydro-1H-pyrazol-4-yl)diazenyl)-N-(pyrimidin-2-yl)benzenesulfonamide (316). Sulfadiazine is diazotizated with sodium nitrite under acidic conditions, followed by treatment of the diazonium salt with ethyl acetoacetate and sodium acetate to give p-ketoester in 95% yield. Condensation of p-ketoester with different benzoylhydrazides (4-chlorobenzohydrazide) in glacial acetic acid at 100° C. produced compound 316 and other similar compounds in yields ranging from about 19%-71%. Compound 335 was prepared by treatment of compound I with sodium hydride and methyl iodide in THF.

TABLE 1

| Compound | Mp | Yield |
|---|---|---|
| Number | (° C.) | (%) |
| 100 | 226 | 49 |
| 101 | 151-152 | 95 |
| 102 | 216-217 | 95 |
| 104 | 126-127 | 51 |
| 105 | 207-208 | 72 |
| 106 | 239-240 | 97 |
| 107 | 141-142 | 95 |
| 108 | 149-150 | 84 |
| 109 | 190-191 | 69 |
| 110 | 197-198 | 70 |
| 111 | 121-122 | 97 |
| 112 | 93-94 | 59 |
| 113 | 220-221 | 74 |
| 114 | 137-138 | 84 |
| 115 | 156-157 | 98 |
| 116 | 117-118 | 87 |
| 117 | 209-210 | 82 |
| 118 | 206-207 | 88 |
| 118E | 156-157 | 76 |
| 119 | 190-191 | 83 |
| 119E | 89-90 | 63 |
| 120 | 194-195 | 86 |
| 120E | 108-109 | 43 |
| 122E | 201-202 | 73 |
| 123E | 101-102 | 65 |
| 124E | 96-97 | 34 |
| 125 | 89-90 | 68 |
| 126 | 101-102 | 82 |
| 127 | 69-70 | 73 |
| 128 | 138-139 | 65 |

### Example 2

### In Silico Screening

Computational docking was employed to study the interactions between the AKT1 PH domain and its inhibitors. One of the high resolution (0.98 \acute{Å}) complex AKT PH domain crystal structures (1UNQ) was retrieved from Protein Data Bank (PDB) for docking simulations. Based on structural analysis and literature (28-30), residues Lys¹⁴, Glu¹⁷, Arg²³ and Arg⁸⁶ around the inositol-(1,3,4,5)-tetrakisphosphate (Ins(1,3,4,5)P₄) ligand were found to be essential for the protein-ligand interactions because they are involved in hydrogen bonds and responsible for the protein conformational change induced by the ligand binding. The binding pocket was, therefore, defined to include all residues within 6.5 \acute{Å} around these four residues. Before docking, the ligand and crystal waters were removed from the complex structure, and then hydrogen atoms were added to the protein. The PDB 2PQR (30) was utilized to prepare the protein structures such as placing missing hydrogens, calculating the pKa values of protein residues, and so on. Default parameters were applied unless stated otherwise.

Commercially available docking packages, FlexX (FlexX [1.20.1], BioSolveIT GmbH: Sankt Augustin, Germany, 2007), GOLD (GOLD [3.2], CCDC: Cambridge, UK, 2007) and Glide (Glide [4.5], Schrodinger: Portland, Oreg., 2007), were used to dock the original ligand Ins(1,3,4,5)P₄ into the binding pocket to evaluate the applicability of each docking package to this target. FlexX produced 100 different docking poses for each ligand within the active site. No early determination was allowed in GOLD to terminate docking on a given ligand. The flexibility of ligand was taken into account by GOLD via flipping the ring corners and hydrogen atoms of the protonated carboxylic acids. Internal hydrogen bonds were included to restrict the flexibility. Glide was set to permit the conformational modification of amide bonds in order to consider the docking flexibility while the protein was treated as a rigid body. The best poses (poses with best scores) from these docking algorithms were re-evaluated using X-score to calculate their potential binding affinities. Because all showed reasonable predictions (small RMSD) of the binding mode compared with the crystal structure, all three programs were employed for all docking studies using default parameters unless otherwise noted. Among them GOLD could reproduce the crystal structure with the best predictions, and thus its docking results were used if there were any inconsistencies from the three packages.

GOLD, FlexX and Glide algorithms were employed to dock the compounds into the binding pocket of the AKT PH domain, see e.g. Table 3. The GOLD algorithm showed consistently better predictability for compound 100 and related compounds than either the FlexX or the Glide algorithms and thus was used to calculate the predicted binding affinities (K_{D} values) by X-score. Docking programs and their related scoring functions cannot successfully rank putative ligands by binding affinity. Instead, these same functions were used to classify active and inactive ligands for the analog series in this system. The docking values were directly compared to the measured binding affinities obtained using surface plasmon resonance spectroscopy, see e.g., Table 2 and FIG. 6A. SPR was carried out by injecting the compounds over the surface of expressed and isolated AKT at the indicated concentrations and measuring binding of the compounds to the protein target.

A 3D pharmacophore search was carried out as described above based on the hydrogen-bonding pattern between the inositol(1,3,4,5)-tetrakisphosphate ligand and the PH domain of AKT (1H10) using UNITY (Tripos, L.P.). A virtual library of approximately 300,000 compounds generated from databases (the NCI Chemical and Natural Products Library, the Maybridge Available Chemicals Directory, and the LeadQuest Chemical Library) was searched. Twenty compounds from each database were selected, the compounds were pooled and duplicates removed. This process lead to the identification of the initial four compounds shown in Table 2, each of these compounds was examined in the active site using hand modeling and structure-based design. The four compounds identified using a pharmacophore screen (7% hit rate) each contain a series of ring structures connected by short flexible linker regions. The IC₅₀ of these compounds ranged from 1 μmol/L to 50 μmol/L in a cellular AKT inhibition assay. Although compound 316 contains the undesirable alkyl, aryl-azo moiety, and compound 389 has a fairly high calculated LogP (4.4). Each of these compounds is a weak acid and will be an anion in typical intracellular compartments, which may allow binding to the strongly basic binding site of the PH domain.

TABLE 2
Structures, predicted in silico properties, ADME properties and biological activities of
four novel hits

| | | | | | | | AKT | Cell |
|---|---|---|---|---|---|---|---|---|
| | | | | | | | inhibition^{‡} | survival^{§} |
| Compound | FlexX | Gold | Glide | | Caco-2 Pe* | K_{D}^{†} | (IC₅₀, | (IC₅₀, |
| Number | score | fitness | score | LogP | (10⁻⁶ cm/s) | (μmol/L) | μmol/L) | μmol/L) |
| 316 | −34.84 | 60.94 | −2.75 | 3.7 | 163.9 | 0.39 ± 0.04 | 24.0 | 25.0 |
| 345 | −43.63 | 63.78 | −3.80 | 0.7 | 0.1 | 1.79 ± 0.26 | 50.0 | >100 |
| 389 | −35.44 | 54.25 | −3.80 | 2.6 | 124.2 | 4.58 ± 1.72 | 5.0 | >100 |
| 415 | −27.02 | 64.36 | −3.62 | 1.4 | 0.8 | 6.27 ± 1.16 | 1.0 | 3.1 |

*Caco-2 permeability (Pe) is calculated for pH = 7.4 and rpm = 500.
^{†}The K_{D} was obtained using SPR spectroscopy.
^{‡}Inhibition of AKT was measured by Western blots using specific antibodies against phospho-Ser⁴⁷³-AKT in HT-29 lung cancer cells.
^{§}Cell survival was measured using an MTT assay in HT-29 lung cancer cells.

To obtain additional SAR data and develop reliable binding models in the AKT system, a database of approximately 2.3 million unique compounds was assembled from vendor databases. After an initial collection of several hundred compounds was identified, a subset of 46 compounds was selected manually based on the following criteria: conservative analogs of the known hits, explore a range of new SAR data, challenge the need for an anion in the hits, and avoid non-medicinal, toxic, reactive and unstable functional groups.

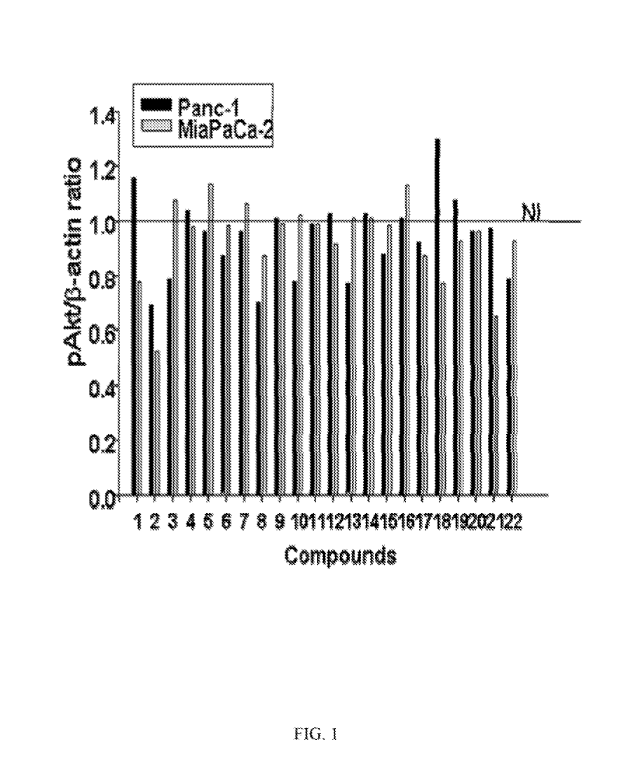

An in silico screen of the subset of 46 compounds was conducted to identify small molecules that would be expected to bind to the PH domain of AKT, and twenty-two of these compounds were identified and tested for their ability to inhibit phospho-Ser⁴⁷³-AKT in Panc-1 (FIG. 1, black bars) and MiaPaCa-2 (FIG. 1, grey bars) pancreatic cancer cells. Human MiaPaca-2, BxPC-3 and Panc-1 pancreatic cancer cells were obtained from the American Type Culture Collection. Cells were maintained and drug treated as described in Mahadevan D, Powis G, Mash E A, et al. Discovery of a novel class of AKT pleckstrin homology domain inhibitors. Mol Cancer Ther 7:2621 (2008) which is hereby incorporated by reference in its entirety. Two compounds, 100 and 455 (9% hit rate), were found to be active against AKT in MiaPaCa-2 cells with IC₅₀ values of 20 μmol/L and 25 μmol/L, respectively. Furthermore they did not exhibit cytotoxicity in either cell line tested as indicated from Table 2.

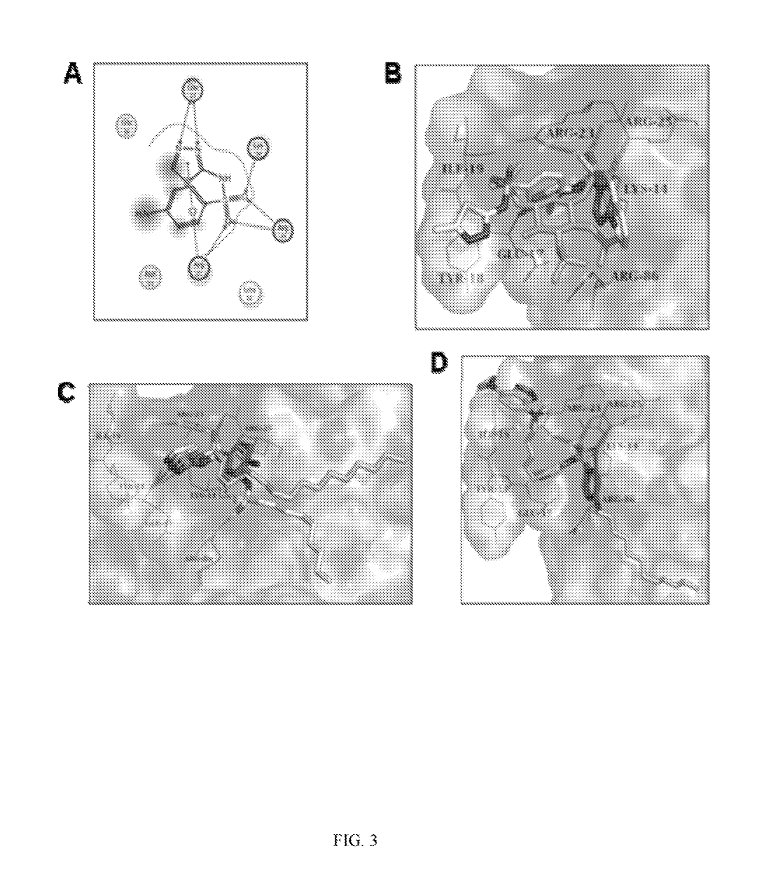

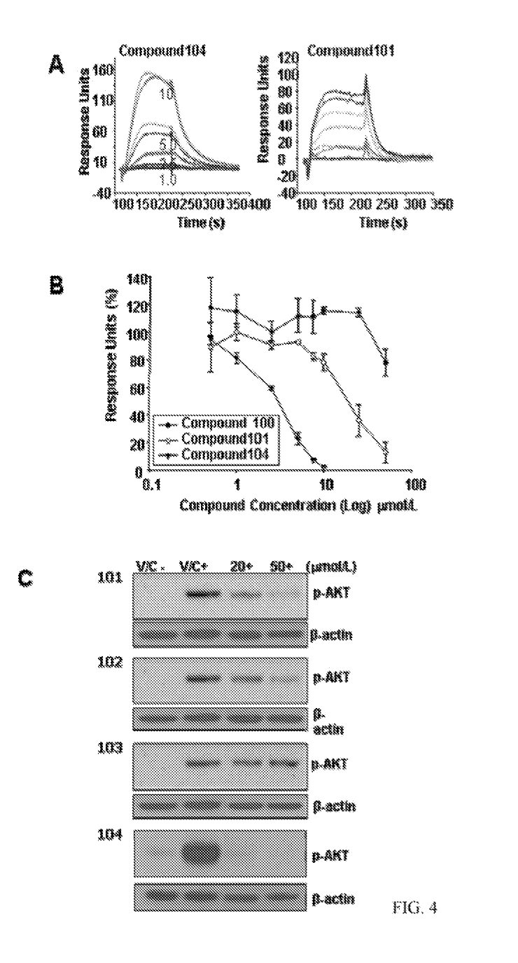

To further improve the potency of these two compounds, several computational approaches were employed to study their binding to the PH domain of AKT as well as their ADMET properties. According to the docking studies using the GOLD algorithm, the sulfonyl moiety of compound 100 acts as a hydrogen bond acceptor interacting with residues Arg²³, Arg²⁵ and Lys¹⁴ while hydrogen bonding interactions were observed between the nitrogen atoms in the thiadiazolyl group and residue Glu¹⁷ as shown in FIG. 3A. The hydrogen bonding interactions between compound 100 and the protein are similar to those in the original 1UNQ complex as shown in FIG. 3B. In particular, the sulfonyl group interacts with the protein by mimicking the 3-position phosphate of the Ins(1,3,4,5)P₄ ligand. In contrast to compound 100, compound 455 possesses two sulfonyl fragments, which may mimic the 1- and 3-position phosphate groups on the inositol ring and interact with Arg²³, Arg²⁵ and Lys¹⁴. The positively charged guanidinium cation of Arg²³ interacts with one of the benzyl rings of compound 100 via charge-charge interaction. Stacking interactions were observed between the thiadiazole ring of compound 455 and the phenyl ring of Tyr¹⁸.

TABLE 3
Compound structures, modeling properties and biological activities

| | | | | pAKT | |
|---|---|---|---|---|---|
| | | | | inhibition^{†} | |
| Compound | FlexX | | X-score* | (IC₅₀, | Cell viability^{‡} |
| Number | score | G-score | (pK_{d}) | μmol/L) | (IC₅₀, μmol/L) |
| 436 | −29.2 | −136 | 5.86 | N/I | N/I |
| 100 | −27.4 | −61.5 | 4.59 | 20 | N/I |
| 437 | −23.5 | −71.4 | 5.16 | N/I | N/I |
| 438 | −26.5 | −65.3 | 5.79 | N/I | N/I |
| 439 | −36.0 | −73.6 | 6.42 | 50 | N/I |
| 440 | −35.8 | −32.0 | 4.99 | N/I | N/I |
| 441 | −33.7 | −47.2 | 5.77 | 25 | N/I |
| 442 | −37.8 | −83.4 | 6.18 | N/I | N/I |
| 443 | −31.5 | −31.7 | 5.79 | N/I | N/I |
| 444 | −24.8 | −40.8 | 5.1 | 50 | N/I |
| 445 | −33.1 | −116.0 | 5.7 | 50 | N/I |
| 446 | −26.0 | −89.7 | 5.29 | N/I | N/I |
| 447 | −26.5 | −116.0 | 5.58 | N/I | N/I |
| 448 | −29.1 | −166.0 | 5.76 | N/I | 80 |
| 449 | −30.0 | −113.0 | 5.64 | N/I | 190 |
| 450 | −25.3 | −75.0 | 4.92 | 50 | N/I |
| 451 | −25.4 | −96.0 | 5.38 | N/I | N/I |
| 452 | −29.9 | −133.0 | 5.81 | N/I | N/I |
| 453 | −30.0 | −119.0 | 5.58 | N/I | N/I |
| 454 | −28.6 | −122.0 | 5.53 | N/I | N/I |
| 455 | −33.4 | −91.5 | 5.76 | 25 | N/I |
| 456 | −39.7 | −94.4 | 5.44 | 50 | N/I |

*Calculated pK_{d} was obtained from the X-score.
^{†}Inhibition of AKT was measured by Western blotting using specific antibodies against phospho-Ser⁴⁷³-AKT in MiaPaCa-2 cells; N/I, for no inhibition at the highest concentration tested.
^{‡}Inhibition of cell proliferation was estimated by viability assay as described in the Materials and Methods; N/I, for no inhibition at the highest concentration tested.

### Example 3

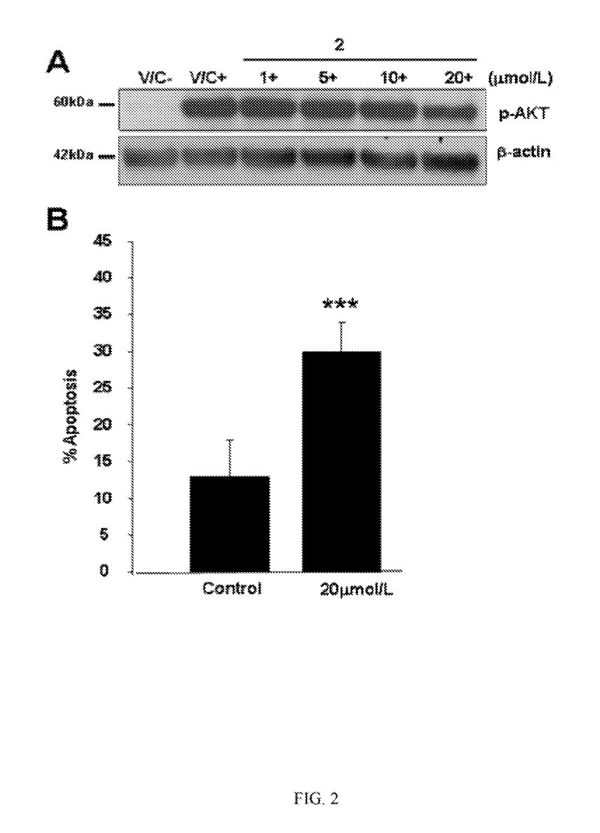

Experimental cellular AKT inhibition analysis demonstrated that compounds 100, 441 and 455 had approximately the same affinity, yet compound 100 had significantly better ligand efficiency (FIG. 1, FIG. 2, and Table 2). The smaller size of compound 100 may afford greater freedom for structural modification and optimization and therefore was selected for hit-to-lead optimization. Analysis of docking poses showed that the phenyl ring of compound 100 points away from the binding site, and so modifications of the para-amino group were not predicted to affect the binding (FIG. 3C). Our docking results indicated that compound 455 might be stronger binder than compound 100. Therefore, the Caco-2 cell permeability of the molecule based on the Absorption, distribution, metabolism, and toxicological (ADMET) modeling predictions may be enhanced by modificating by, for example, attaching a flexible hydrophobic group. The ADMET properties, such as Caco-2 permeability and LogP values, were calculated using ADMET predictors and ADME Boxes (ADME Boxes [4.0], Pharma Algorithms: Toronto, Ontario, Canada, 2007).

Three compounds have a hydrophobic group attached to the phenyl of compound 100 were derived, compounds 101-104 and computationally docked into the PH domain of AKT, synthesized, and experimentally tested for AKT binding and inhibitory activity. The docking results and calculated ADMET properties for compounds 101-104 are summarized in Table 4. The docking studies suggested that compound 101 might be a better inhibitor than compound 100 with a higher LogP and Caco-2 permeability.

TABLE 4
Predicted in silico properties and ADMET properties

| Com- | | | | | | Caco-2 | |
|---|---|---|---|---|---|---|---|
| pound | | | | | | permea- | |
| num- | FlexX | Glide | Gold | X-score | K_{D}^{†} | bility^{‡} | |
| ber* | score | score | fitness | (pK_{D}) | (μmol/L) | (10⁻⁶ cm/s) | LogP |
| 100 | −26.43 | −2.97 | 50.97 | 4.82 | 15.13 | 0.3 | 0.13 |
| 101 | −21.38 | −2.52 | 57.37 | 4.99 | 10.23 | 10.1 | 4.93 |
| 102 | −27.12 | −3.79 | 49.16 | 4.99 | 10.23 | 0.8 | 0.34 |
| 103 | −30.36 | −3.31 | 57.30 | 4.69 | 20.41 | 1.0 | 0.59 |
| 104 | −14.05 | −1.55 | 60.70 | 4.87 | 13.49 | 0.1 | 7.54 |

^{†}The K_{D} was obtained from the X-Score (pK_{D}) in mol/L.
^{‡}Caco-2 permeability is calculated for pH = 7.4 and rpm = 500.

Examining Table 4, if compounds 100, 101, and 104 considered active, then Glide and FlexX categorize the five compounds incorrectly. While GOLD and X-score correctly place compound 102 as the least active, Glide and FlexX place compound 103 as either among the most active. Likewise, the 95% confidence interval of the mean FlexX, G-score or X-score for the inactive and active ligands, compounds 100, 439, 441, 444, 445, 450, 455, and 456 using pAKT IC₅₀, may have significant overlap. Therefore, docking scores may not successfully differentiate active from inactive ligands among the series represented. Despite this negative affinity categorization, the binding modes predicted by the docking experiments were helpful in the design of the most potent compounds.

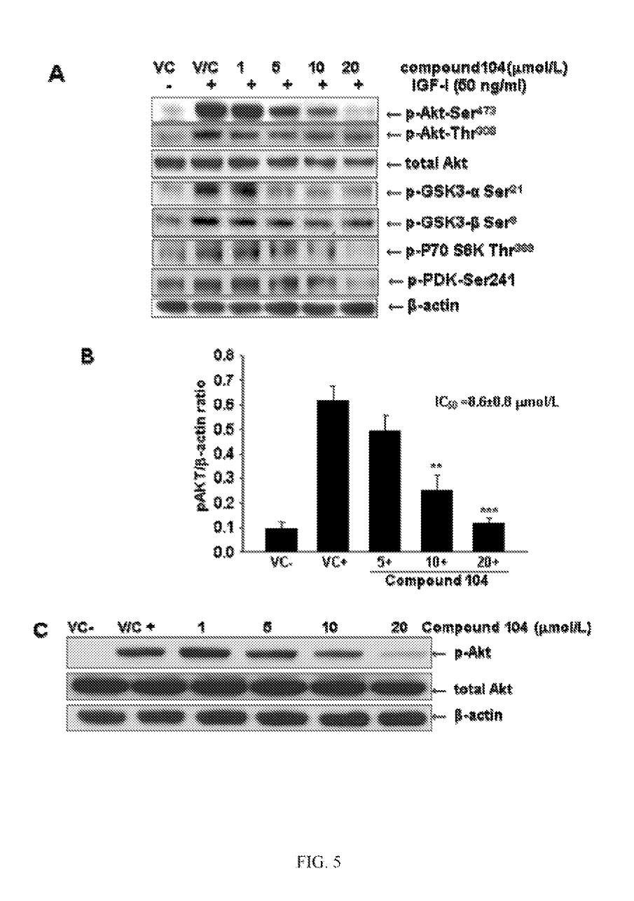

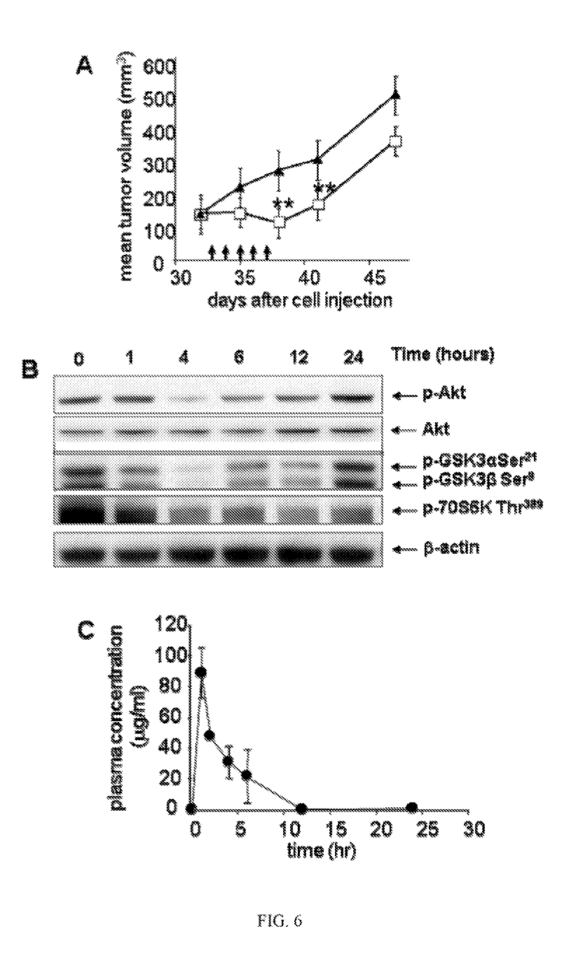

The predicted in silico were verified in cellular assays of AKT inhibition (Table 5). The K_{D} measured using SPR spectroscopy binding assays for compound 100 and compound 101 was 0.45 μmol/L and 19.6 μmol/L, respectively. SPR interaction analyses were performed with a Biacore 2000, using Biacore 2000 Control Software v3.2 and BIAevaluation v4.1 analysis software (Biacore) as described in Mol Cancer Ther 7:2621 (2008). For the competitive binding assays and the Kᵢ determination, PtdIns(3,4,5)phosphate-biotin labeled liposomes (Echelon Biosciences) and SA chips were used with increasing concentrations of the compound tested. Data generated using these techniques indicate that compound 101 appears to inhibit AKT at lower concentration than compound 100. By comparison, PtdIns(3,4,5)P₃, a native substrate of AKT, appear to bind the PH domain of AKT with a K_{D} of 3.08±0.49 mol/L. Compound 101 was further predicted to have better Caco-2 permeability than compound 100, which could explain its low IC₅₀ exhibited in the cellular AKT inhibition assay. Interestingly, calculation of a Kᵢ using liposome displacement and SPR spectroscopy indicate that compound 101 can displace PtdIns-3,4,5-phosphates liposomes at lower concentrations than compound 100 (FIG. 4B and Table 5).

In order to determine whether or not compound 101 is a prodrug of compound 100, a non-amide analog, compound 104, was synthesized and experimentally evaluated. As shown in FIG. 3C, docking studies indicate that the modification did not change the binding mode, and compound 104 showed a higher GOLD fitness of the binding to the PH domain. A lower IC₅₀ of 6.3±0.9 μmol/L for AKT inhibition was observed for this compound in Panc-1 cells (Table 5). However, low Caco-2 cell permeability was predicted for compound 104 with a high LogP value compared to compound 101. Consistent with the prediction, the Kᵢ for compound 104 was significantly lower than those of compounds 100 and 104. For comparison, the displacement of diC8-PtdIns(3,4,5)P₃ exhibited a Kᵢ around 0.3 μmol/L.

TABLE 5
Biochemical and biological activities*^{†}

| | | pAKT | | Cell |
|---|---|---|---|---|
| | | inhibition^{§} | Apoptosis^{\|\|} | survival** |
| Compound | K_{D}^{‡} and Kᵢ^{‡} | (IC₅₀, | at 20 μmol/L | IC₅₀, |
| number | (μmol/L) | μmol/L) | (%) | μmol/L) |
| 100 | K_{D} = 0.45 ± 0.1 | 20/25 | 24.3 ± 3.2/ | NI/NI |
| | Kᵢ > 50.0 | | 25.7 ± 2.6 | |
| 101 | K_{D} = 19.6 ± 4.9 | 10/15 | 28.7 ± 0.3/ | 127/90 |
| | Kᵢ = 21.8 ± 1.8 | | 20.0 ± 1.5 | |
| 102 | K_{D} = NB | >50/>50 | 6.8 ± 0.9/ | NI/NI |
| | Kᵢ > 50 | | 10.3 ± 2.1 | |
| 103 | K_{D} = NB | >50/>50 | 11.4 ± 0.5/ | NI/NI |
| | Kᵢ > 50 | | 18.7 ± 3.1 | |
| 104 | K_{D} = 40.8 ± 2.5 | 6.3 ± 0.9/10 | 40.0 ± 2.9/ | 65/30 |
| | Kᵢ = 2.4 ± 0.6 | | 31.3 ± 1.6 | |

*All biological tests were made in Panc-1 (numbers on the left) and MiaPaca-2 (number on the right) pancreatic cell lines.
^{†}NI, for not inhibitory and NB for not binding.
^{‡}K_{D} and Kᵢ (μM) were determined using purified AKT PH domain and SPR spectroscopy (Biacore 2000). The Kᵢ for PtdIns(3,4,5)trisphosphate was 0.26 μmol/L.
^{§}Inhibition of AKT was measured by Western blots using specific antibodies against phospho-Ser⁴⁷³-AKT.
^{||}Percentage of apoptosis was obtained by a morphological assay at 20 μmol/L.
**Cell survival was measured using an MTT assay.

Further compounds were prepared as described in Example 1 and characterized using the protocols described above. Such compounds are provided in Table 6 and Table 7 below. Compound 104 data are provided in each table for reference.

TABLE 6
Predicted in Silico Properties and ADME properties

| Com- | | | | | | Caco-2 | |
|---|---|---|---|---|---|---|---|
| pound | | | | X- | | Perme- | |
| Num- | FlexX | Glide | Gold | score | K_{D}² | ability³ | |
| ber¹ | score | score | fitness | (pK_{D}) | (μM) | (10⁻⁶cm/s) | LogP |
| 104 | −14.05 | −1.55 | 60.70 | 4.87 | 13.49 | 0.1 | 7.54 |
| 108 | −14.22 | −2.35 | 58.50 | 5.08 | 8.32 | 0.0 | 8.01 |
| 112 | −12.95 | −1.39 | 63.62 | 5.12 | 7.59 | 0.0 | 8.35 |
| 116 | −15.41 | −1.19 | 64.73 | 5.39 | 4.07 | 0.0 | 8.94 |
| 120 | −37.34 | −4.93 | 68.93 | | | 4.6 | 3.95 |
| 120E | −16.78 | −1.79 | 73.72 | | | 0.0 | 7.97 |
| 124E | −21.97 | −1.85 | 59.31 | | | 0.0 | 7.91 |
| 128 | −27.89 | −1.64 | 59.60 | | | 0.2 | 6.73 |
| 140 | −28.51 | −1.96 | 51.27 | | | 0.0 | 6.50 |
| 106 | −27.13 | −3.35 | 50.38 | | | 1.4 | 0.75 |
| 110 | −25.56 | −3.30 | 52.40 | 5.22 | 6.03 | 2.1 | 1.09 |
| 114 | −26.11 | −3.34 | 53.56 | | | 6.2 | 1.91 |
| 118 | −38.16 | −5.64 | 61.94 | | | 0.1 | 0.01 |
| 118E | −24.76 | −2.79 | 61.28 | | | 1.6 | 1.08 |
| 122E | −31.83 | −2.53 | 49.47 | | | 1.0 | 0.72 |
| 126 | −27.34 | −2.45 | 50.97 | | | 0.1 | −0.28 |
| 138 | −38.27 | −3.08 | 51.03 | | | 0.0 | 0.33 |
| 105 | −26.684 | −2.25 | 51.85 | | | 0.5 | 0.56 |
| 109 | −22.14 | −2.67 | 53.30 | 5.11 | 7.76 | 0.8 | 0.90 |
| 113 | −22.71 | −2.76 | 54.57 | | | 2.3 | 1.73 |
| 117 | −34.77 | −6.28 | 70.27 | | | 0.0 | −0.15 |
| 125 | −27.89 | −2.66 | 52.34 | | | 0.1 | −0.46 |
| 131 | −37.12 | −3.40 | 53.50 | | | 0.0 | −0.05 |
| 107 | −18.95 | −2.37 | 60.19 | | | 3.2 | 5.49 |
| 111 | −19.420 | −1.58 | 59.61 | 5.28 | 5.25 | 1.5 | 5.83 |
| 115 | −21.01 | −1.87 | 59.62 | | | 0.2 | 6.69 |
| 119 | −31.10 | −4.93 | 68.93 | | | 4.6 | 3.95 |
| 119E | −20.18 | −2.16 | 72.43 | | | 3.0 | 5.41 |
| 123E | −24.46 | −2.66 | 55.90 | | | 3.9 | 5.28 |
| 127 | −21.22 | −2.73 | 60.30 | | | 9.0 | 4.28 |
| 129 | −26.61 | −3.50 | 50.95 | | | 0.2 | 0.23 |
| 155 | −38.45 | −2.88 | 61.08 | | | 0.7 | 6.91 |
| 154 | −33.99 | −2.10 | 53.78 | | | 77.3 | 3.91 |
| 153 | −33.00 | −2.06 | 55.99 | | | 13.8 | 5.30 |

^{†}The K_{D} was obtained from the X-Score (pK_{D}) in mol/L.
^{‡}Caco-2 permeability is calculated for pH = 7.4 and rpm = 500.

TABLE 7
Biochemical and biological activities^{1,2}

| | | % pAKT | Apoptosis⁵ | Cell |
|---|---|---|---|---|
| | K_{D}³ and Kᵢ³ | Inhibition⁴, | at 20 μM | Survival⁶, |
| Compounds | (μM) | At 10 μM | (%) | IC₅₀ (μM) |
| 104 | K_{D} = 40.8 ± 2.5 | 6.3 ± 0.9/10 | 40.0 ± 2.9/ | 65/30 |
| | Kᵢ = 2.4 ± 0.6 | 64 | 31.3 ± 1.6 | |
| 108 | K_{D} = 48.7/36.3 | 64 | | BxPC3 61 |
| 112 | K_{D} = 73.0/7.9 | 88 | | 32 |
| 116 | K_{D} = 587 | 36 | | 45 |
| 120 | K_{D} = 110/114 | 67 | | >100 |
| 120E | K_{D} = 20.7 | 62 | | 70 |
| 124E | K_{D} = 736/616 | 72 | | 36 |
| 128 | K_{D} = 429 | 68 | | 37 |
| 140 | K_{D} = 4.6 | 29 | | 85 |
| 106 | NB | NI | | |
| 110 | NB | 45 | | |
| 114 | NB | 20 | | |
| 118 | NB | 19 | | |
| 118E | NB | 55 | | |
| 122E | NB | NI | | |
| 126 | NB | NI | | |
| 138 | NB | NI | | |
| 105 | NB | 11 | | |
| 109 | | 16 | | |
| 113 | NB | 13 | | |
| 117 | NB | 23 | | |
| 125 | NB | 10 | | |
| 131 | NB | 18 | | |
| 107 | K_{D} = 46.7 | 11 | | |
| 111 | K_{D} = 178.0 | 22 | | |
| 115 | K_{D} = 284.0 | 16 | | |
| 119 | K_{D} = 281.0 | 42 | | |
| 119E | K_{D} = 105.0 | NI | | |
| 123E | K_{D} = 109.0 | 10 | | |
| 127 | K_{D} = 24.5 | 11 | | |
| 129 | | | | |
| 155 | K_{D} = 23.7 | NI | | |
| | Kᵢ > 50.0 | | | |
| 154 | K_{D} = 19.1 | NI | | |
| | Kᵢ > 50 | | | |
| 153 | K_{D} = 25.8 | 10 | | |
| | Kᵢ = 8.4 | | | |
| 156 | K_{D} = 58.9 | 70 | | |
| | Kᵢ = 6.7 | | | |
| 157 | K_{D} = 987.0 | 50 | | |
| | Kᵢ = 6.9 | | | |
| 158 | NB | NI | | |
| | Kᵢ = 11.4 | | | |

¹All biological tests were made in BxPC-3 pancreatic cell lines.
²NI, for not inhibitory and NB for not binding.
³K_{D} and Kᵢ (μM) were determined using purified AKT PH domain and SPR spectroscopy (Biacore 2000). The Kᵢ for PtdIns(3,4,5)trisphosphate was 0.26 μM.
⁴Inhibition of AKT was measured by Western blots using specific antibodies against phospho-Ser⁴⁷³-AKT in BxPC-3.
⁵Percentage of apoptosis was obtained by a morphological assay at 20 μM.
⁶Cell survival was measured using an MTT assay.

### Example 4

### Biological Activity

AKT inhibition leads to cellular apoptosis. Therefore, the ability of compounds 100 and 101 to 104 to induce cellular apoptosis was measured and correlated with the inhibition of AKT phosphorylation measured by Western blot analysis of phospho-Ser⁴⁷³-AKT, see FIGS. 4 and 2. Inhibition of the phosphorylation of AKT and its downstream targets was measured by Western blotting using rabbit polyclonal antibodies to phospho-Ser⁴⁷³-AKT, phospho-Thr³⁰⁸-AKT, total-AKT, phospho-Ser⁹-GSK3β, .phospho-Ser²¹-GSK3β, phospho-Ser²⁴¹-PDK1 and phospho-Thr³⁸⁹p70S6-kinase (New England Biolabs/Cell Signaling Technology Inc.) using β-Actin as a loading control as described in Mol Cancer Ther 7:2621 (2008). Bands corresponding to phospho-Ser⁴⁷³-AKT and total AKT were quantified using Eagle Eye software (BioRad) and Kodak X-Omat™ Blue XB (NEN™, Life Science Products). Cell growth inhibition was determined using a microcytotoxicity assay and apoptosis was measured as described in Mol Cancer Ther 7:2621 (2008). These protocols were performed with compounds 100 and 455 as shown in FIG. 2. Apoptosis was directly correlated with the inhibition of AKT observed at 20 μmol/L by Western blot for both initial hits, compounds 100 and 455, see FIG. 2. Compounds 100 and 101 to 104 were also tested for their ability to inhibit cellular AKT activity as shown in FIG. 4C and to induce apoptosis as indicated in Table 5. These compounds induced apoptosis and inhibited AKT phosphorylation.

Additionally, in vitro binding assays using SPR spectroscopy were performed to directly determine the affinities of the lead compounds for the target PH domain. FIG. 5A shows representative sensorgrams obtained for the direct binding of compounds 101 and 104 and K_{D} was calculated (Table 5). Compounds 102 and 103 did not appear to bind directly to the PH domain of AKT. These results correlate with a very weak inhibition of cellular AKT and weak induction of apoptosis. On the contrary, compound 104 exhibited all the characteristics of an AKT inhibitor with an IC₅₀ of 6.3±0.9 μmol/L in Panc-1 cells, a strong induction of apoptosis at 20 μmol/L and some cellular cytotoxity. These data correlate with a low K_{D} for the compound to the PH domain as measured by SPR spectroscopy. Interestingly again, the measurement of the Kᵢ appears to be the most reliable and predictive assay for compound cellular efficacy.

Moreover, for selectivity purposes, the binding of compound 104 to the PH domain of PDK1 was tested and a K_{D} of 90.1 μmol/L, a Kᵢ of 5.5 μmol/L was obtained. These values correlated well with the Gold score obtained for the compound to the PH domain of PDK (53.5) as compared to 60.7 for the PH domain of AKT. These data suggest that compound 104 may represent an AKT selective compound with some activity on PDK1 at higher concentrations.

The biochemical properties of compound 104 on AKT function in BxPC-3 cells is summarizes in Table 5 (IC₅₀=8.6±0.8 μmol/L), and its effects on downstream targets are shown in FIGS. 4A and B and. In brief, compound 104 was able to reduce the phosphorylation of AKT on Ser⁴⁷³ and less strongly on Thr³⁰⁸ without affecting AKT expression. Furthermore, GSK3β and p70S6K phosphorylation were inhibited in a dose-dependent manner by compound 104. Phosphorylation of PDK1 Ser²⁴¹ was only slightly affected by compound 104 and was only affected at high concentrations of compound 104. These data appear to be in agreement with the SPR results and confirm the selectivity of compound 104 for AKT at low concentrations.

To further describe the action of compound 104, the fluorescent analog compound 137 was used (Scheme 3 and synthesis above). The addition of the fluorescent NBD moiety does not appear to alter the binding of compound 137 to the protein as indicated in FIG. 3D and compound 137 inhibited AKT phosphorylation in a fashion similar to 104 based on AKT inhibition in BxPC-3 cells as shown in FIG. 5C. Finally, confocal microscopy was used to determining the intracellular location of compound 137, which was found to be mainly located in the cytosol and/or lipid vesicles. BxPC-3 cells were grown on coverslips in DMEM plus 10% FBS media. Following 4 h of incubation with 10 Ξmol/L of compound 137 or with a DMSO control, cells were washed twice in PBS and fixed using 4% par formaldehyde. Coverslips were washed four times in PBS and mounted using mounting media containing DAPI obtained from Molecular Probes Invitrogen. Slides were then visualized using a Nikon PCM2000 confocal microscope (Nikon Instruments Inc.). Without wishing to be bound by theory, the accumulation of compound 137 in the cytosol suggests that AKT may trapped in the cytosol as a result of compound 104 administration as indicated in FIG. 5C.

The anti-tumor activity of compound 104 measured against BxPC-3 pancreatic cancer xenografts in scid mice a dose of 125 mg/kg of compound 104 was administered i.p., twice a day for 5 d is shown in FIG. 6A. For these experiments, approximately 1×10⁷ BxPC-3 pancreatic cancer cells in log cell growth suspended in 0.1 mL PBS were injected subcutaneously (s.c.) into the flanks of female severe combined immunodeficient (scid) mice. When the tumors reached volumes of approximately 150 mm³, the mice were stratified into groups of eight animals having approximately equal mean tumor volumes. Compound 104 was suspended in 0.2 mL of an aqueous solution containing 2.5% ethanol and 20% Trappsol® (Cyclodextrin Technologies Development Inc.) by intraperitoneal (i.p.) injection at a dose of 125 mg/kg twice a day for 5 d. The animals were weighed weekly. Tumor diameters, measured twice weekly at right angles (dₛₕₒᵣₜ and d_{long}) using electronic calipers, were converted to volume by the formula, volume=(dₛₕₒᵣₜ)²×(d_{long})/2 (32). Significant anti-tumor activity with cessation of tumor growth and even regression during the course of treatment can be observed by such treatment. Notably, tumor growth appears to have resumed at its original rate when the drug was removed (FIG. 6A).

This observation was tested using pharmacodynamic and pharmacokinetic studies. Pancreatic cancer cells (1×10⁷ BxPC-3) were injected s.c. into the flanks of female scid mice and allowed to grow to approximately 300 mm³. Mice received a single i.p. dose of compound 104 of 125 mg/kg suspended in 0.2 mL of 0.25% ethanol/20% Trappsol® in water. Mice were killed after 1, 4, 6, 12 or 24 h, blood was collected into heparinized tubes, and plasma was stored frozen. The frozen tumors were removed and immediately frozen in liquid N₂. The tumors were then homogenized in 50 mmol/L HEPES buffer, pH 7.5, 50 mM NaCl, 1% Nonidet® P40 and 0.25% sodium deoxycholate. Western blotting was performed as described above. Plasma levels of compound 104 were measured by reverse phase high pressure liquid chromatography as described in Mol Cancer Ther 7:2621 (2008). Preliminary studies indicate that compound 104 is not toxic in single doses up to 250 mg/kg, which may be the maximum dose administered. As shown in FIG. 6B, a single 125 mg/kg i.p dose of compound 104 resulted in up to 70% inhibition at 6 hours, which is reduced to 50% inhibition after 12 hours and has returned to about untreated levels after 24 hours as measured by phospho-Ser⁴⁷³-AKT concentration. These results correlate well with the plasma concentrations of compound 104 following the single dose as shown in FIG. 4C. Indeed, between 1 and 6 h, a peak corresponding to compound 104 was detected in the plasma.

### Example 5

### AKT Binding Alkylene R¹

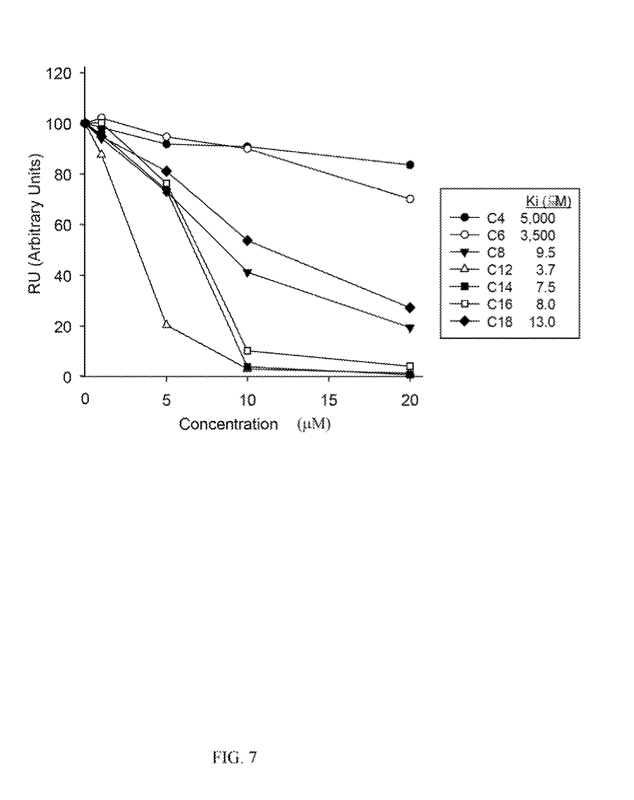

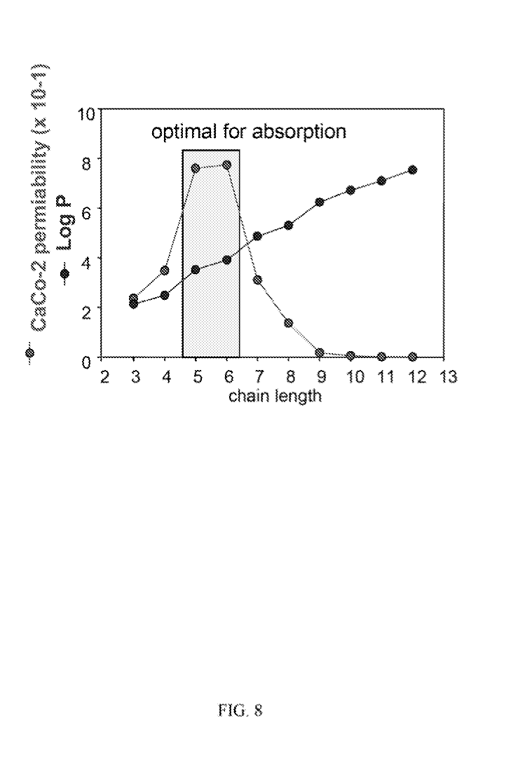

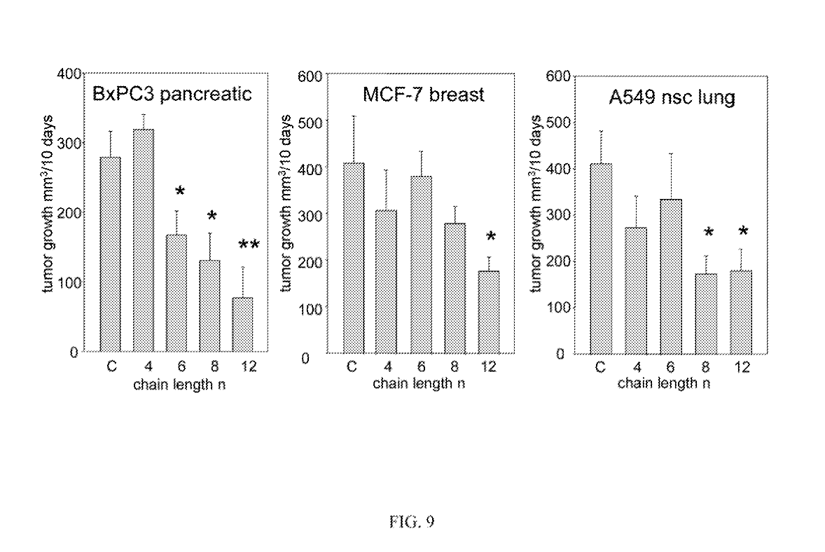

Analogs of compound 104 having different alkyl chain lengths were synthesized and tested to determine whether reducing the lipophilicity through a reduction in the carbon chain length and increasing the CaCO-2 permeability could improve antitumor activity. A series of compounds having an R¹ of a C4 (compound 155), C6 (compound 154), C8 (compound 153), C14 (compound 156), C16 (compound 157), and C18 (compound 158) alkyl chains ¹ was synthesized, characterized and compared to compound 104 (C12). Initially, surface plasmon resonance spectroscopy (SPR) was used to measure the binding affinity (Kᵢ) of compound 104, and 153 to 158 to the PH domain of AKT by competitive binding of each compound with the natural ligand, PI(3,4,5)-triphosphate. FIG. 7 shows binding curves for each compound. These data suggest that the binding affinity of compound 104 was at a maximum when the alkyl chain length was 12 (compound 104). The calculated CaCo-2 permeability of compounds 104 and 153 to 158 was is provided in FIG. 8 and appear to indicate optimal absorption occurs with compounds having a alkyl chain of 5 or 6 carbons. Therefore, the efficacy of compound 155 (C4), compound 154 (C6), and compound 153 (C8) were tested by administering 200 mg/kg of each of compounds 104, and 153 to 154 twice a day for 10 days to treat subcutaneous xenografts of BXPC3 pancreatic tumor cells, MCF-7 breast tumor cells, and A549 nscl lung cancer cells and determining the tumor growth rate. The results are provide in FIG. 9 and suggest that compound (C 12) had the best antitumor activity in each of the tumors tested, followed by compound 153 (C8) and compound 154 (C6). Compound 155 (C4) appears to be inactive. Thus, carbon chain length may be a determinant of antitumor activity.

### Example 6

### Antitumor Activity of Compound 104

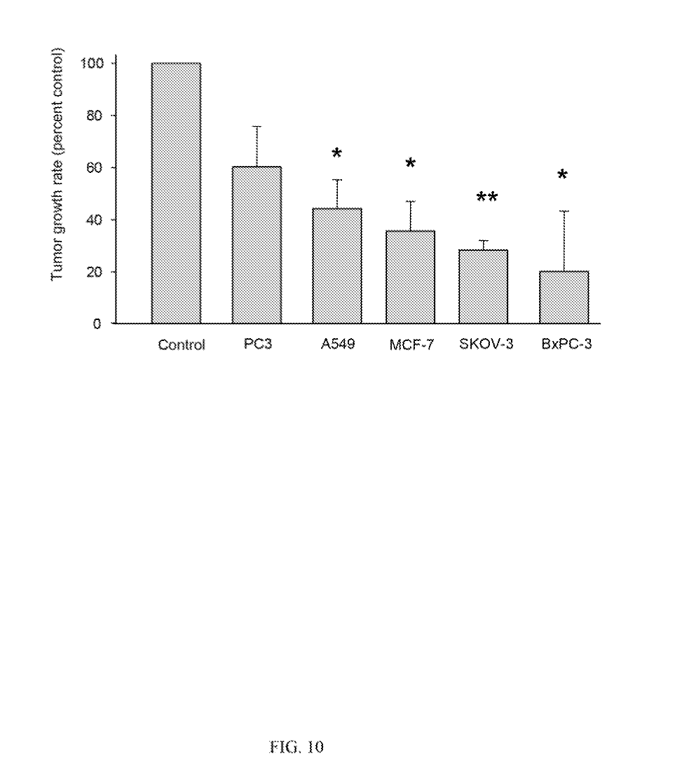

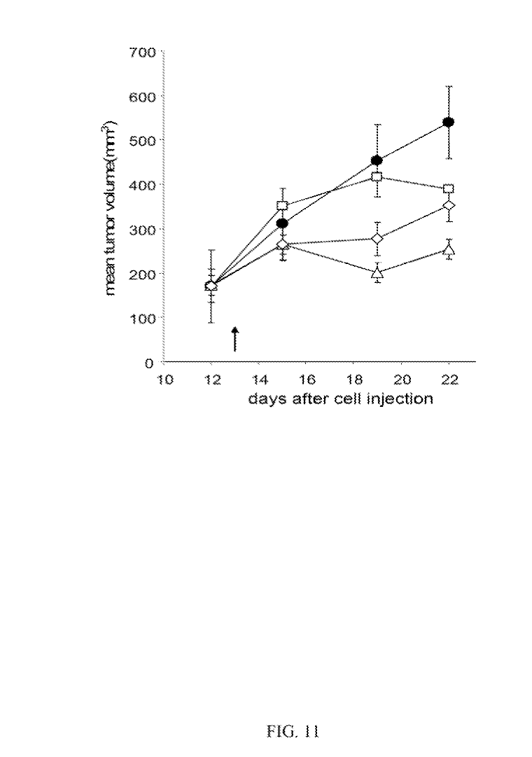

Female scid Mice were administered 0.1 ml of compound 104 or its analogs formulated at a concentration up to 50 mg/ml in a 8:2 mixture of Labrafil® (oleoyl macrogolglycerides): Labrasol® (caprylocaproyl macrogolglycerides) which was administered orally by gavage twice a day for 5 or 10 days as follows: PC3 prostate cancer 125 mg/kg twice a day (BID)×5 days; A549 nsc lung cancer 200 mg/kg BID×10 days; MCF-7 breast cancer 200 mg/kg BID×10 days; SKOV-3 ovarian cancer 250 mg/kg BID×10 days; BxPC-3 pancreatic cancer 250 mg/kg BID×5 days. Table 6 shows the antitumor activity of compound 104 at doses of 125 to 250 mg/kg in xenografts of different tumor types. Results are expressed as the growth rate of the compound 104 in treated tumors relative to the control tumors, and are illustrated graphically in FIG. 10. These data suggest that compound 104 provided up to about 80% inhibition of tumor growth in the most sensitive tumors. The pattern of inhibition in different tumors is similar to that of PI-3-kinase inhibitor suggesting that compound 104 may inhibit the PI-3-Kinase/PDK1/AKT signaling pathway.

TABLE 8
Antitumor activity of compound 104

| | vol- | | | Growth | | |
|---|---|---|---|---|---|---|
| | ume | | | rate | | |
| | at start | Dose | | mm³/ | T/C | |
| Tumor ¹ | mm³ | mg/kg | Schedule | 10 days | % | p value ³ |
| BxPC-3 | 156 | control ² | BID × 5 D | 228 ± 46 | | |
| | | 125 | BID × 5 D | 67 ± 35 | 29.4 | 0.030 |
| | | 250 | BID × 5 D | 46 ± 53 | 20.1 | 0.027 |
| | 97 | control ² | BID × 10 D | 279 ± 37 | | |
| | | 100 | BID × 10 D | 181 ± 52 | 64.8 | NS ⁴ |
| | | 200 | BID × 10 D | 77 ± 44 | 27.6 | 0.004 |
| PC3 | 229 | control ² | BID × 5 D | 780 ± 161 | | |
| | | 125 | BID × 5 D | 470 ± 121 | 60.3 | NS ⁴ |
| SKOV-3 | 192 | control ² | BID × 10 D | 432 ± 59 | | |
| | | 250 | BID × 10 D | 122 ± 16 | 28.3 | 0.001 ⁴ |
| A549 | 157 | control ² | BID × 10 D | 413 ± 37 | | |
| | | 200 | BID × 10 D | 182 ± 47 | 44.1 | 0.016 ⁴ |
| MCF-7 | 142 | control ² | BID × 10 D | 410 ± 101 | | |
| | | 100 | BID × 10 D | 383 ± 139 | 93.4 | NS ⁴ |
| | | 200 | BID × 10 D | 156 ± 30 | 38.0 | 0.042 |

¹ 8 mice per group;
² control received vehicle only ( );
³ compared to vehicle control;
⁴ not significantly different p > 0.05

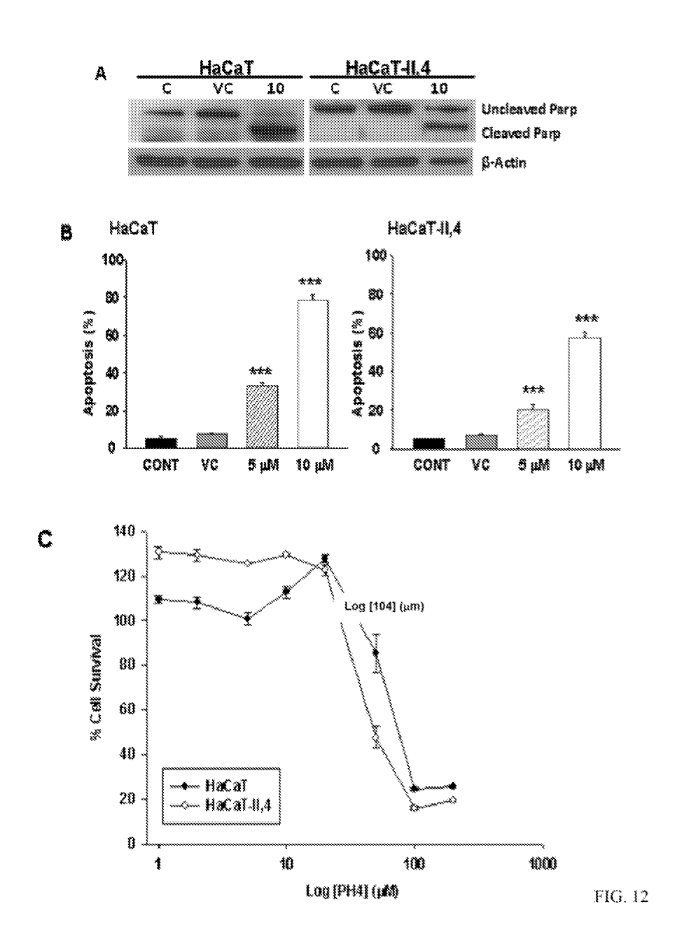

To determine the efficacy of compound 104 as a sensitizer for tumor cells, compound 104 was administered alone or in combination with paclitaxel to scid mice with subcutaneous MCF-7 human breast cancer xenografts. Female scid mice with a s.c. implanted 60 day estradiol release pellets were injected s.c. with 10⁷ MCF-7 human breast cancer cells. When the tumors reached about 10 mm³ the mice were statified into groups of 8 mice and dosing was started on day 13 as indicated by the arrow (↑) in FIG. 11. Vehicle control mice () were administered 0.1 ml of 2:8 Labraso®l:Labrafil® orally twice per day for 10 days; compound 104 only mice (⋄) were administered 200 mg/kg of compound 104 formulated as described above orally twice per day for 10 days; paclitaxel only mice (□) were administed 10 mg/kg of paclitaxel i.p. injection every other day for 5 doses; and combination mice (Δ) were administed 200 mg/kg of compound 104 orally twice a day for 10 days and 10 mg/kg of paclitaxel by i.p. injection every other day for 5 doses. As indicated in FIG. 11, compound 104 appears to have inhibited tumor growth, and the combination of compound 104 and paclitaxel showed improved antitumor activity over either compound 104 or paclitaxel alone.

### Example 7

### Compound 104 in HaCaT Cells

Human HaCaT, an immortalized cell line derived from adult human skin keratinocytes, and HaCat-II,4, HaCaT cells that were transfected with H-ras, were maintained in bulk culture in Dulbecco's modified Eagle medium (DMEM) supplemented with 10% heat-inactivated fetal bovine serum (FBS), 100 U/ml penicillin and 100 □g/ml streptomycin in a 5% CO₂ atmosphere. Cells were passaged using 0.25% trypsin and 0.02% EDTA and confirmed to be mycoplasma free by testing them with an ELISA kit. Normal morphogenesis and differentiation features of skin keratinocytes are retained in the HaCaT cultures. Compound 104 was prepared in DMSO at a stock concentration of 10 mM and then added at different concentrations directly into the culture media of the cells. HaCaT cells and ras-transformed HaCaT cells were incubated with DMSO vehicle control or 10 μM compound 104 for 3 hours.

FIG. 12 summarizes the effects of compound 104 in HaCaT and ras-transformed HaCaT and HaCat-II,4 cells. Apoptosis of treated HaCaT cells was measured by PARP cleavage observed through Western blotting. Cells were treated with increasing concentrations of compound 104 for three days and cell proliferation was evaluated using a MTT assay. FIG. 12A shows representative results from Western blot experiments. Both PARP and cleaved PARP are observed as independent species on the blot, and β-actin was used as an internal control. Statistically significant increases in apoptosis were noted in both cell lines in presence of 5 or 10 μM of compound 104 (p<0.001). As indicated in FIG. 12B, compound 104 induces PARP cleavage at 10 μM and induced 80% of apoptosis in HaCaT cells and 60% in HaCat-H,4 cells, while it did not affect cell survival at this concentration as shown in FIG. 12C. Survival was not affected until much higher concentrations were used as these data indicate that compound 104 exhibits an IC₅₀ of about 40 μM for HaCaT and 60 μM for HaCat-II,4, 4 and 6 times that of concentrations required for PARP cleavage.

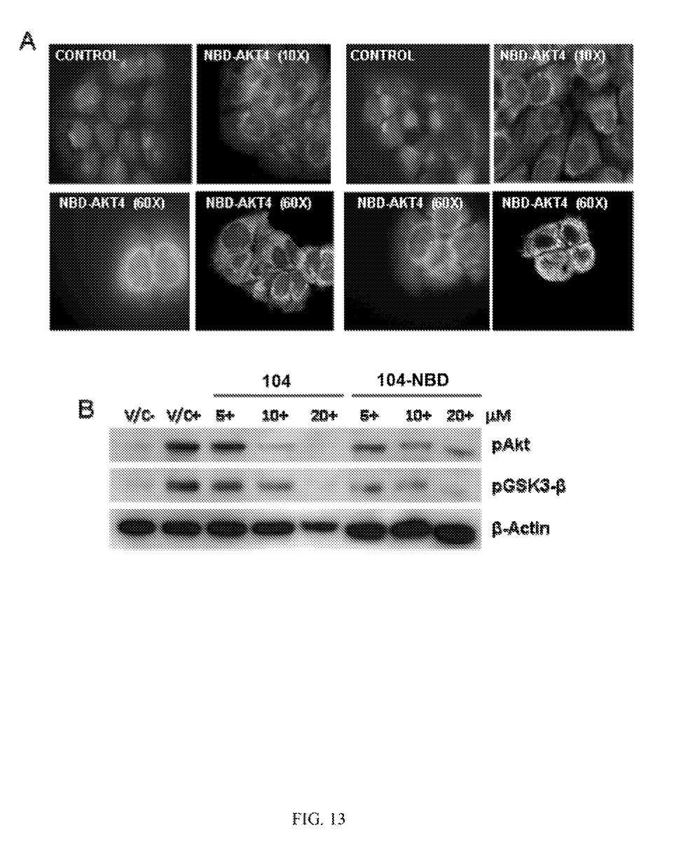

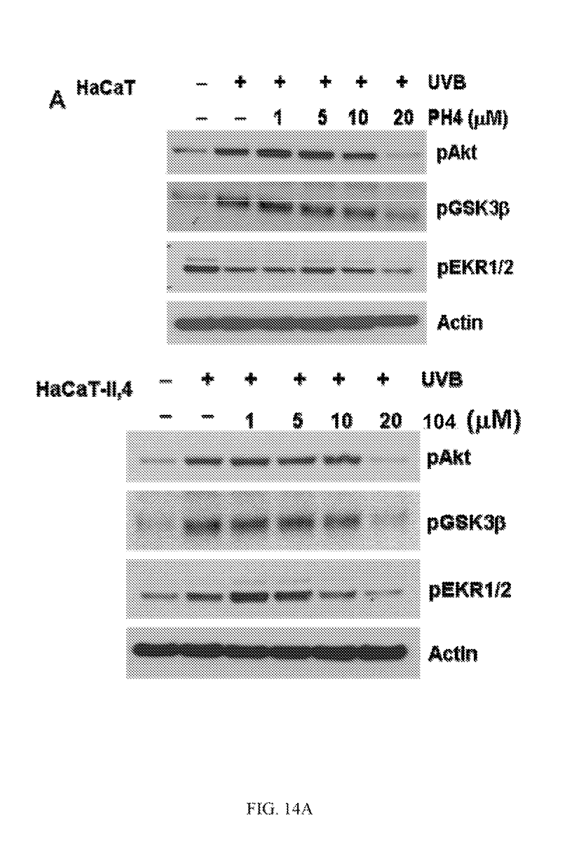

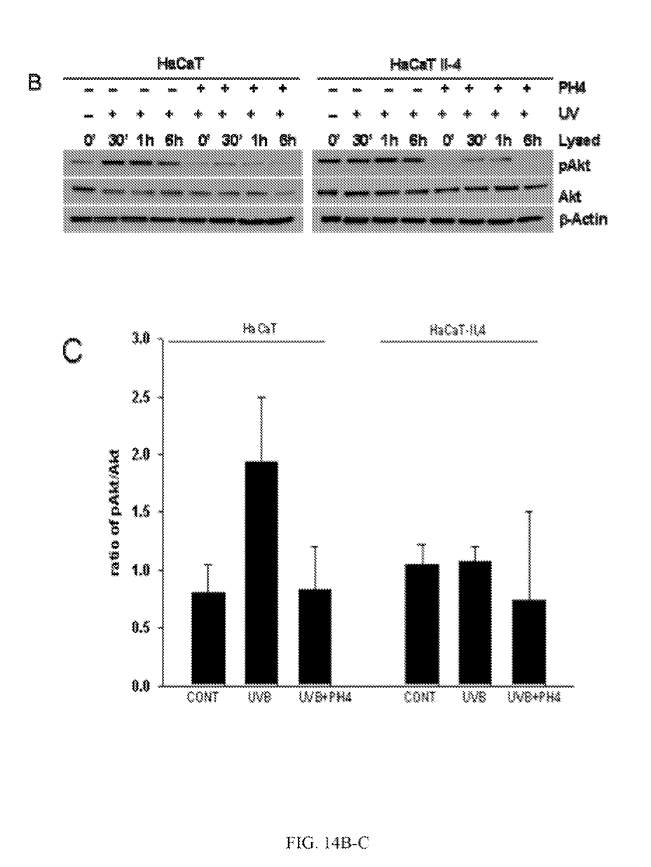

To better characterize the mechanism of action for compound 104, a compound 104 analog having a fluorescent marker, 7-nitroben-2-oxa-1,3-diazole, was prepared, compound 137, and HaCaT cells were treated for 3 hours with compound 137, the cells were fixed, and then visualized them under a fluorescent microscope using FITC filters. DAPI nuclear stain was used as an internal control. As illustrated in FIG. 13A, HaCaT or HaCaT-II,4 cells contacted with compound 137 and visualization under a fluorescent microscope show that the compound 137, and thus, compound 104, may enter the cells and locate both the plasma membrane and the cytosol.

To ensure that compound 137 effects AKT phosphorylation similar to compound 104, either compound 104 or compound 137 was administered to HaCaT at various concentrations as indicated in FIG. 13B for 3 hours, the cells were then stimulated with 100 ng/ml EGF for 20 minutes and then lysed. Cells lysates were probed for phospho-Ser⁴⁷³AKT and phosphor-Ser⁹GSK3-β by Western blot analysis using rabbit polyclonal antibodies to phospho-Ser⁴⁷³-AKT, phospho-Ser9-GSK3-β or phospho-Thr²⁰²/Tyr²⁰⁴-ERK1/2 and anti-β-actin used as a loading control. These data suggest that although compound 137 possesses a fluorescent tag, it effects on Akt activity in cells HaCaT cells in the same way as compound 104.

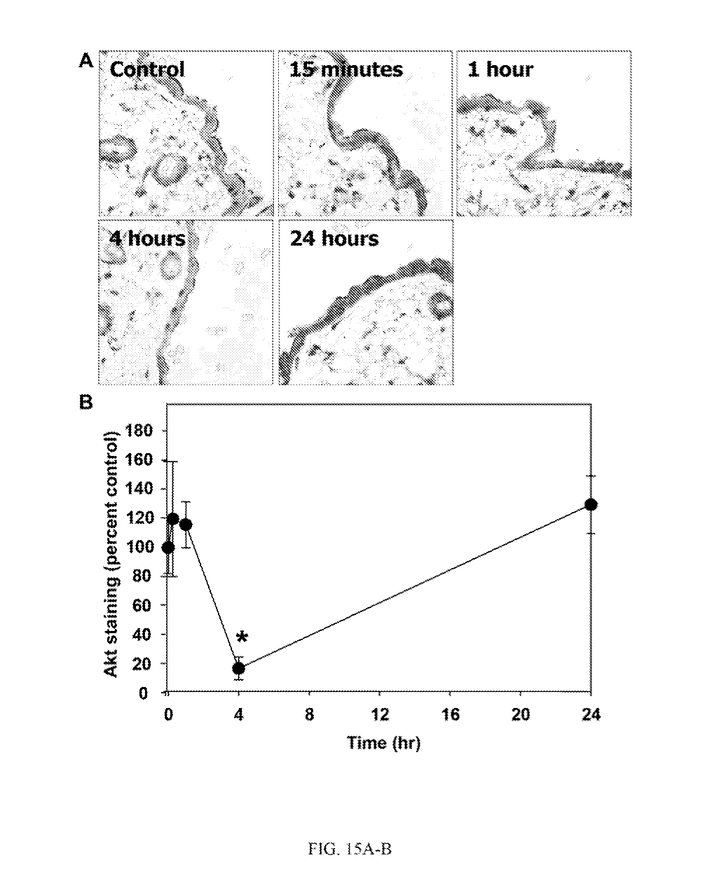

UV-B light is a major cause of non-melanoma skin cancer and induces PI3K/AKT activity in cultured human keratinocytes. Thus, the ability of compound 104 to mitigate or prevent UVB-induced AKT activation was tested. FIG. 14 A shows the effect of increasing concentrations of compound 104 on HaCaT cells (top) and HaCaT-II,4 cells (bottom) that were irradiated with a single acute dose of UV-B light (250 J/m2). Western blot analysis, as described above, was used to determine the extent of AKT phosphorylation in irradiated and control cells. As indicated, UV-B irradiation induced AKT phosphorylation in both cell lines. However, administration of 10 μM and 20 μM compound 104 appear to have reduced UVB-induced AKT phosphorylation as well as one downstream target, GSK3-β in both cell lines. Total AKT and pERK1/2 also appear to be down regulated in both cell lines. Notably, administration of 1 μM and 5 μM, or did not appear to affect UV-B indicuced AKT phosphorylation.

Data suggests that AKT activation may occur about one hour after UV-B exposure. Therefore, compound 104 activity overtime in UV-B stimulated cells was tested. Briefly, 10 μM compound 104 or DMSO vehicle control was administered to HaCaT cells and HaCaT-II,4, and a portion of the treated cells were with UV-B irradiation, and lysed, as described above, at the indicated time. Western blots prepared as described above with representative data provided in FIG. 14B suggest that UV-B irradiation induces rapid induction of AKT phosphorylation that appears to peak after about one hour, and pretreatment of irradiated cells with compound 104 may reduce phosphoylation of AKT in both cell lines. These data are represented graphically in FIG. 14C

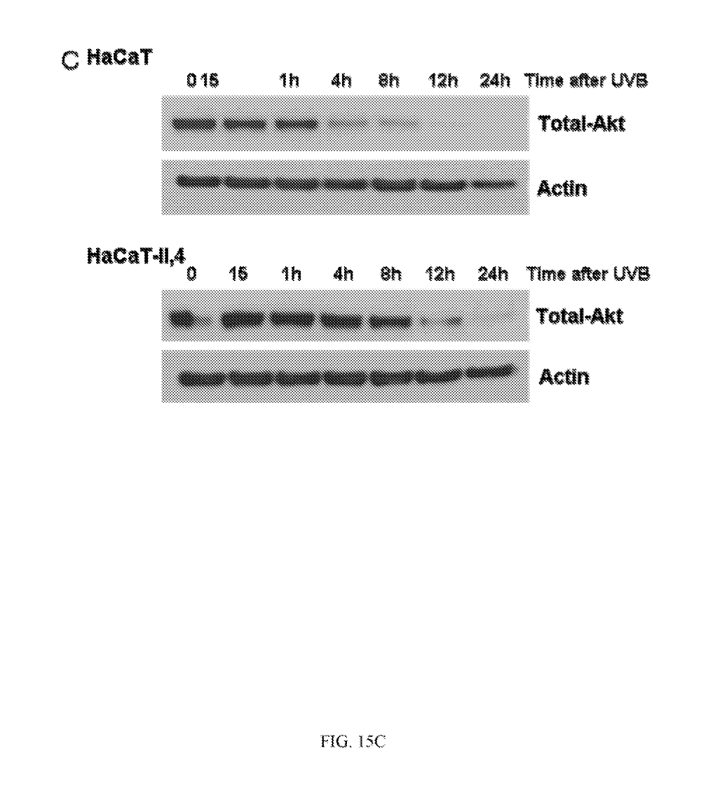

In vivo activity of compound 104 was tested by administering 20 mg/ml in 0.1 ml acetone topically to scid mice. Skin biopsies were taken and immunohistochemistry for AKT was performed on the sections. Total Akt staining was observed at the beginning of the experiment and decreased significantly overtime as indicated in FIG. 15A by the disappearance of the brown staining (AKT) between 1 and 4 hours. Notably, AKT staining reappears after 24 hours indicating that the effect of compound 104 may have dissipated. FIG. 15B shows a graphical representation quantifying AKT staining in the sections provided in FIG. 15A. Staining was measured by quantitative immunohistochemistry with correction for non-specific background staining (p<0.05). Phospho-Ser⁴⁷³-AKT was not detectable in dermal layer. FIG. 15C summarizes the effects of compound over a 24 hour period as determined by Western blot analysis performed as described above. HaCaT cells (top) and HaCaT-II,4 (bottom) were incubated after administration of 10 μM compound 104 for the indicated period of time and then lysed. These data show a decrease in total AKT was after 4 hours in HaCaT cells and after 8 hours in HaCaT-II,4 cells and are in agreement with the immunohistochemistry data above.

### Example 8

### In Silico Screening

AKT1 PH domain small molecule inhibitors were identified using the crystal structure of the AKT1 PH domain bound by Ptdlns(1,3,4,5)P4 as descried in Thomas C C, Deak M, Alessi D R, van Aalten D M, High-resolution structure of the pleckstrin homology domain of protein kinase b/AKT bound to phosphatidylinositol (3,4,5)-trisphosphate, Curr Biol 12:1256 (2002), which is hereby incorporated by reference in its entirety, using a pharmacophore query search of the National Cancer Institute database. The high-resolution crystal structure of the isolated PH domain of human AKT1 in complex lns(1,3,4,5)P₄ was utilized to define a pharmacophore pocket for screening using Unity in Sybyl (version 7.2; Tripos Inc., St Louis, Mo.). The pharmacophore pocket included all the residues of the AKT1 crystal structure within 5 Å of the lns(1,3,4,5)P₄ binding site, i.e., Lys14, Arg15, Gly16, Gtu17, Tyr18, Ile19, Lys20, Thr21, Arg23, Pro24, Arg25, Lys39, Pro51, Leu52, Asn53, Asn54, Phe55, Gln79, Ile84, Glu85, Arg86 and Phe88, and attributes to various atoms on the ligand and/or protein binding site were assigned. The defined pharmacophore pocket was then used to search virtual chemical databases and candidate compounds were identified. Various docking orientations were analyzed on the basis of FlexX scores, G-score, and X-score. Generally, the resulting scores are similar to interaction energy, and better/improved interactions are indicated by more negative values. The predicted K_{D} is calculated by pK_{D}=10 exp(-Xscore). Using the FlexX docking algorithm in Sybyl for simulated docking of these compounds into the AKT1 PH domain active site resulted in 30 different docking orientations (poses) of the ligand within the active site. In order to investigate the possibility of specific binding of the identified small molecules at the AKT1 PH domain using in silico methods, known crystal structures of the IRS1 PH domain (IRS1, PDB:1QQG) and of the PDK1 PH domain (PDK1, PDB.iWID, 1W1G) were also used for docking studies similar to those described above.

A 2,000 molecule database (National Cancer Institute) was screened using Unity in Sybyl as described above. These compounds were docked and then ranked based on their docking scores. One of these molecules compound 316 exhibited good FlexX score and G-score values as summarized in Table 7 and was selected as a lead for future studies. The predicted binding affinity (K_{D}) of compound 316 to the AKT1 PH domain was 1.2 μM, which was three times better than the lipid-based compound, DPIEL with a predicted K_{D} of 4.0 μM.

TABLE 9
Calculated docking scores

| | AKT1 | | | PDK1 | | | RS1 | | |
|---|---|---|---|---|---|---|---|---|---|
| | FlexX | G | cK_{D} | FlexX | G | cK_{D} | FlexX | G | cK_{D} |
| Compound | Score | Score | (μM) | Score | Score | (μM) | Score | Score | (μM) |
| DPIEL | NS | NS | 4.0 | NS | NS | NS | NS | NS | NS |
| 316 | −31.0 | −96.9 | 1.2 | −17.4 | −109.0 | 1.74 | −16.0 | −128.0 | 1.99 |
| 331 | −29.6 | −31.9 | 2.4 | −17.0 | −40.0 | 2.60 | −17.1 | −96.2 | 2.40 |
| 332 | −28.2 | −99.5 | 1.2 | −17.1 | −103.4 | 1.70 | −14.8 | −79.7 | 10.70 |
| 333 | −29.1 | −71.9 | 3.0 | −17.5 | −88.6 | 2.20 | −17.9 | −145.5 | 1.80 |
| 360 | −33.0 | −120.6 | 1.3 | −20.1 | −137.1 | 2.40 | −14.6 | −90.1 | 10.70 |
| 335 | −24.3 | −132.0 | 0.85 | −21.0 | −109.1 | 1.45 | −14.5 | −140.6 | 0.52 |

NS = not shown

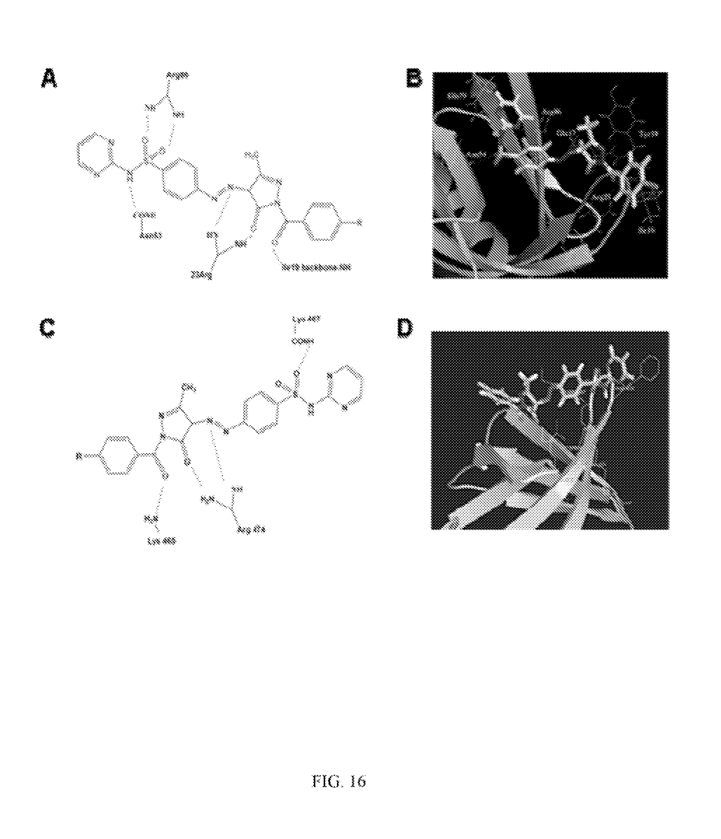

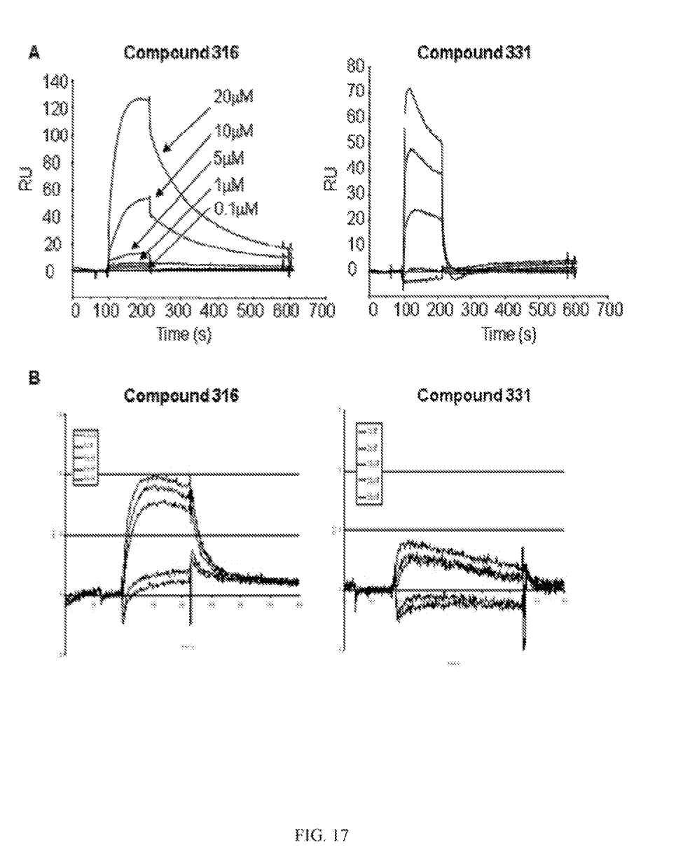

FIG. 16A shows the predicted binding of compound 316 to amino acid residues (Arg86, Asn53, Arg23 and 11e19) of the PH domain binding pocket of AKT1. Hydrogen bonding interactions are displayed as dotted lines. FIG. 16B represents hydrogen bonding interactions that occur between compound 316 and the amino acid side chains, as well as the backbone of the AKT1 PH domain binding pocket. The AKT1 PH domain is colored red and residues Arg23, Arg25 and Arg86 colored by atom type, and compound 316 is represented as capped stick and colored by atom type. The sulfonamide group appears to interact with Arg 86 through a hydrogen bond while a similar hydrogen bonding interaction is involved with the diazopyrazotyl group with Arg 23. These two arginine residues are involved in the strong interaction with the phosphate head groups of the substrate Ptdlns(1,3,4,5)P₄. Other hydrogen bonds are also established between the backbones of Ile 19 and Asn 53 with the sulfonamide function of the compound. FIG. 16C and FIG. 16D represent binding of compound 316 in the binding pocket of the PH domain of PDK1 and the interations with amino acids in the binding pocket. Notably, compound 316 is predicted to exhibit the reverse binding pose in the PH domain of PDK as compared to the PH domain of AKT 1.

Based on the data for compound 316, five structurally similar compounds, 331, 332, 333, 360 and 335 with varying side chains were synthesized as described above. The structures and docking scores for these compounds are summarized in Table 7. Analyses of the docking poses of these compounds in the PH domain of AKT1 revealed different docking orientations between compounds 316, 332 and 360 as compared to compounds 331, 333 and 335. However, these differences in docking orientations may be due to limitations of the FlexX docking simulation since there are only small changes in the structures of these compounds. Therefore, compounds 331, 332, 333, 360 and 335 are expected exhibit similar binding to the AKT1 PH domain despite their FlexX score.

Binding affinities (K_{D}) were also calculated for compounds 331, 332, 333, 360 and 335 to the PH domain of PDK1 and were found to be very similar to those for AKT1 as shown in Table 6. FIG. 16C and FIG. 16D represent binding of compound 316 in the binding pocket of the PH domain of PDK1. There appears to be greater variability between 331, 332, 333, 360 and 335 based on calculated K_{D}s for the PH domain of IRS1 with compound 335 having the greatest affinity and compounds 332 and 360 having lower affinity.

### Example 9

### Measured Binding Affinity

Binding assays using SPR and an ELISA competitive binding assay were used to measure the binding affinity (K_{D}) of the compounds to all three PH domains. SPR was carried out as described above. For ELISA competitive binding assays, a 96-well Maxisorb plate was coated with 1 pG/100 ul L-a-phosphatidylinositol(3,4,5)P₃. Purified GST-PH domains were incubated with increasing concentrations of the compounds under analysis for about 4 hours in 0.2 M carbonate buffer pH 9.4 and were added to the 96-well plate and incubated overnight at 4° C. Following incubation, the plate was washed 4 times with phosphate buffered 0.9% NaCl (PBS), blocked with 3% bovine serum albumin (BSA) in PBS and 0.01% Tween for 1 hour, washed again 4 times with PBS and mouse monoclonal anti-glutathione-S-transferase antibody in 3% BSA (1:2000) was added for 1 hr at room temperature with shaking. The plate was washed 4 times with PBS and an anti-mouse IgG horseradish peroxidase coupled antibody (dilution 1:2000 in 3% BSA) was added for 1 hr. After 4 washes with PBS, 2,2′-azinobis[3-ethylbenzothiazoline-6-sulfonic acid]-diammonium salt (ABST) was added and the reaction was allowed to develop for 30 min. A stop solution of 1% sodium dodecyl sulfate was then added and the plate was read at 405 nm in a plate reader.

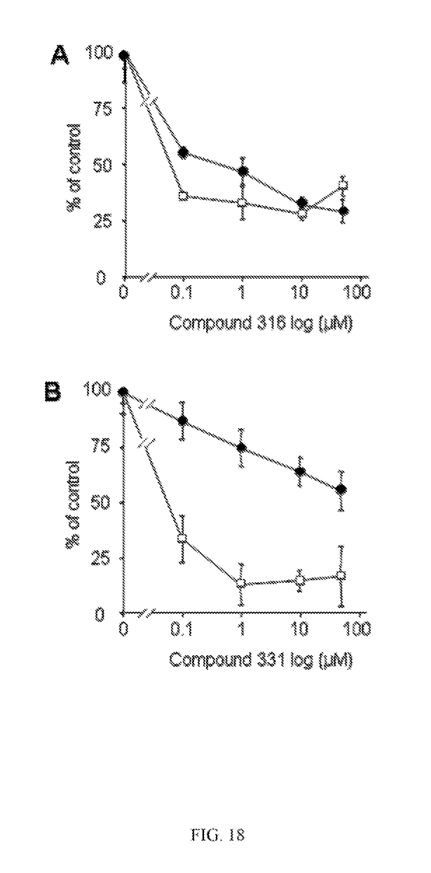

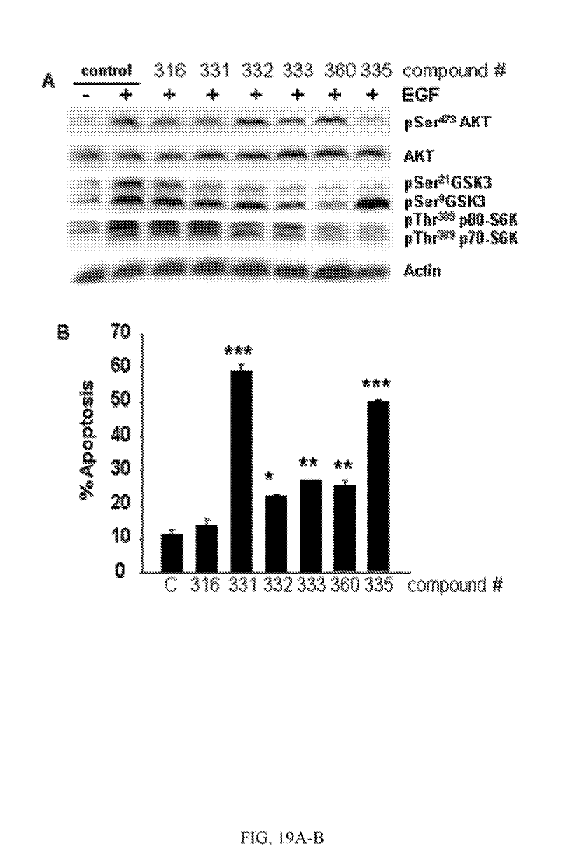

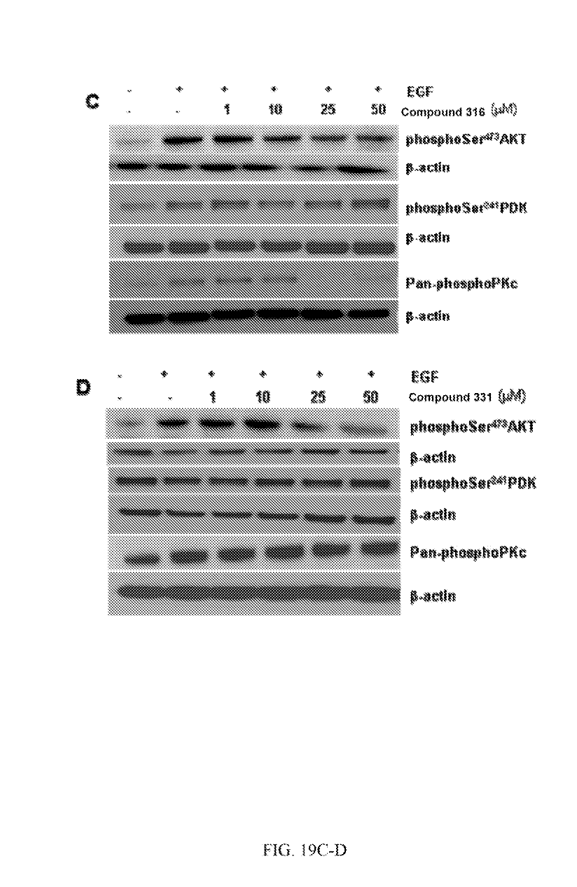

Table 8 summarizes the results obtained from the SPR measurements, and representative saturation curves as well dose response curves are shown in FIG. 17 for compounds 316 and 331 to the PH domain of AKT1 (FIG. 17A) and to the PH domain of IRS-1 (FIG. 17B). These results show an overlay plot of typical sensorgrams obtained with increasing concentrations of compound 316 or 331 as indicated by the arrows. These data correlated well with the predicted K_{D} values for the compounds for each PH domain. Interestingly, modeling suggest that compounds 316 and 331 bind in a reverse binding pose in the PH domain binding pockets of the three different PH domains, which may explain differences in the SPR binding curves. ELISA competitive binding assay conducted using the PH domains of AKT1 and IRS1 with compounds 316 (FIG. 18A) and 331 as shown in FIG. 18A and FIG. 18B, respectively, and resulted in an IC₅₀ for compounds 316 and 331 with AKT1 of 0.08 μM, and an IC50 with IRS1 for compound 316 of 1.0 μM and compound 331 of >100 μM. These data also compare well with the SPR data.

TABLE 10
Selectivity for PH Domains

| | AKT1 PH | 1RS1 PH | PDK1 PH |
|---|---|---|---|
| Compounds | mK_{D} (μM) | mK_{D} (μM) | mK_{D} (μM) |
| Ptdlns(3,4,5)P₃ | 3.08 ± 0.49 | ND | ND |
| DPIEL | 5.04 ± 0.48 | 31.56 ± 8.49 | NB |
| 316 | 0.37 ± 0.04 | 0.39 ± 0.01 | 31.28 + 9.54 |
| 331 | 3.66 ± 0.03 | NB | 0.17 ± 0.10 |
| 332 | 1.37 ± 0.25 | NB | 3.5710.96 |
| 333 | 0.51 ± 0.06 | 0.14 ± 0.02 | NB |
| 360 | 1.35 ± 0.02 | 1.74 ± 0.41 | 0.42 + 0.17 |
| 335 | 1.62 ± 0.02 | NB | 0.9810.48 |

NB = no measurable binding
ND = not determined.

Consistent with these docking studies, compounds 316, 333 and 360 exhibited low K_{D} for the PH domain of IRS1 while compounds 331, 332 and 335 do not show any binding to IRS1 PH as measured by SPR. However, compounds 333 and 335 did not bind the PH domain of PDK1 with a predicted K_{D} of 2.2 and 1.4, respectively. Taken together, these data suggest that the structural modifications in compounds 331, 332, 333, 360, and 335 as compared to compound 316 may have altered the binding positions of the compounds in the AKT1 PH domain as well as their specificity against IRS1 or PDK1 PH domains.

### Example 10

### Biological Activity

Table 9 shows inhibition of phospho-Ser⁴⁷³ AKT by compounds 316, 331, 332, 333, 360 and 335 as measured in either mouse NIH3T3 or human HT-29 colon cancer cells. All of these compounds except compound 332, the most apparently lipophilic of the compounds, inhibited phospho-Ser⁴⁷³AKT with as IC₅₀ from about 2 to about 10 fold higher than the IC₅₀ for AKT1 PH domain (see above). FIG. 19A shows typical Western blots obtained for the compounds in HT-29 colon cancer cells in which HT-29 colon cancer cells were treated with compounds I-6, at 20 μM for 2 hr and stimulated with 50 ng/nl EGF for 30 min. AKT activity was measured by Western blotting using anti-phosphoSer437 AKT antibody. Downstream targets of AKT were detected also by Western blotting using specific anti-phospho antibodies and anti-actin was used as a loading control. Compounds 331 and 335 appear to inhibit both AKT phosphorylation and GSK3 phosphorylation downstream.

FIG. 19B shows percentage of the HT-29 that undergo apoptosis as a result of administration of 20 μM of each of compounds 316, 331, 332, 333, 360 and 335. Apotposis was measured as described previously in reference Powell A A, LaRue J M, Batta A K, and Martinez J D, Bile acid hydrophobicity is correlated with induction of apoptosis and/or growth arrest in HCT116 cells, Biochem J 356:481-486 (2001), which is hereby incorporated by reference in its entirety. Briefly, HT-29 cells were grown to 70-75% confluency in 6-well tissue culture plates, and these cells were treated with the compounds for 24 hours. To measure apoptosis, 10 μl of cells were mixed with ethidium bromide and acridine orange solution (100 [μg/ml each in DMEM) and visualized by immunofluorescence for morphological changes. A minimum of 200 cells was counted and the percentage of apoptotic cells was determined. Both compound 331 and 335 induce significant apoptosis at 20 μM as compared to controls. These data suggest that compounds 331 and 335 induced apoptosis in about 50% to about 60% of cells contacted with this amount of the compounds and suggest that these compounds inhibited AKT as well as downstream targets such as GSK3 phosphorylation (FIG. 19A).

TABLE 11
Biological Properties of compounds 316, 331, 332, 333, 360 and 335

| | AKT inhibition | | | | Metabolic | | Permeability | |
|---|---|---|---|---|---|---|---|---|
| | (IC₅₀ μM) | | Cytotoxicity | | half life | Solubility | (nm/sec) | |
| Compound | NIH3T3 | HT-29 | (IC₅₀ μM) | LogP | (min) | (μM) | Caco2 | MDCK |
| 316 | 4 | 13 | 24 | 2.1 | 62 | 17.9 | 90 | 91 |
| 331 | 11 | 20 | 14 | 1.9 | 62 | 28.3 | 83 | 34 |
| 332 | >20 | >20 | 25 | 3.2 | 91 | 28.6 | 95 | 39 |
| 333 | ND | >20 | NI | 1.2 | >480 | 12.9 | 23 | 8 |
| 360 | 5 | >20 | NI | 1.5 | 138 | 13.1 | 185 | 200 |
| 335 | 3 | 5 | ND | 1.9 | ND | <0.1 | 14 | 5 |

NI = not inhibitory (IC₅₀ > 100 nM);
ND = not determined
Metabolic stability measured by incubating with HT-29 cells at maximum DMEM concentration at 37° C.
Apparent permeability (nm/sec obtained using the QikProp software (Schrodinger Inc., San Diego, CA).
<25 nm/sec = poor permeability
>500 nm/sec = excellent permeability

FIG. 19 also shows response of HT-29 cells to various concentrations of compound 316 (FIG. 19C) and compound 331 (FIG. 19D). Compound 316 (FIG. 19C) and compound 331 (FIG. 19D) were tested at the concentrations shown for 2 hr, and in HT-29 cells stimulated with 50 ng/nl EGF for 30 min. AKT activity was measured by Western blotting using anti-phospho-Ser*37 AKT antibody, PDK activity by anti-phospho-Ser241 PDK antibody as well as downstream target PKC using pan-phospho PKC antibodies. Anti-actin was used as a loading control. AKT phosphorylation appears to decrease in a concentration dependent manner as the concentrations of compounds 316 and 331 increase (FIG. 19C and FIG. 19D, respectively). Compound 316 may also inhibit phosphorylation of PDK and a downstream target of PDK, PKC (FIG. 19C). IRS1 phosphorylation could not be detected in these cells. Compound 331 appears to have inhibited AKT phosphorylation and appears to have had no effect on the phosphorylation of either PDK or PKC.

Table 8 also provides cytotoxicity was measured in HT-29 cells and appears to indicate that a cytotoxic concentration of compounds 316, 331, and 332 in about the same range as that required for inhibition of cell phospho-Ser⁴⁷³AKT while compounds 333 and 360 appear to exhibit no cytotoxicity. Additionally, Table 9 shows the stabilities of compounds 316, 331, 332, 333, 360 and 335 under cell culture conditions. These data suggest that compounds 316, 331, 332 and 360 may breakdown relatively rapid with half lives of about 1 hour to about 2 hours. However, compound 4 was much more stable and did not appear to breakdown over the time period studied. Compound 6 was too insoluble to obtain data.

### Example 11

### In Vivo Effects of the AKT1 PH Domain Inhibitors

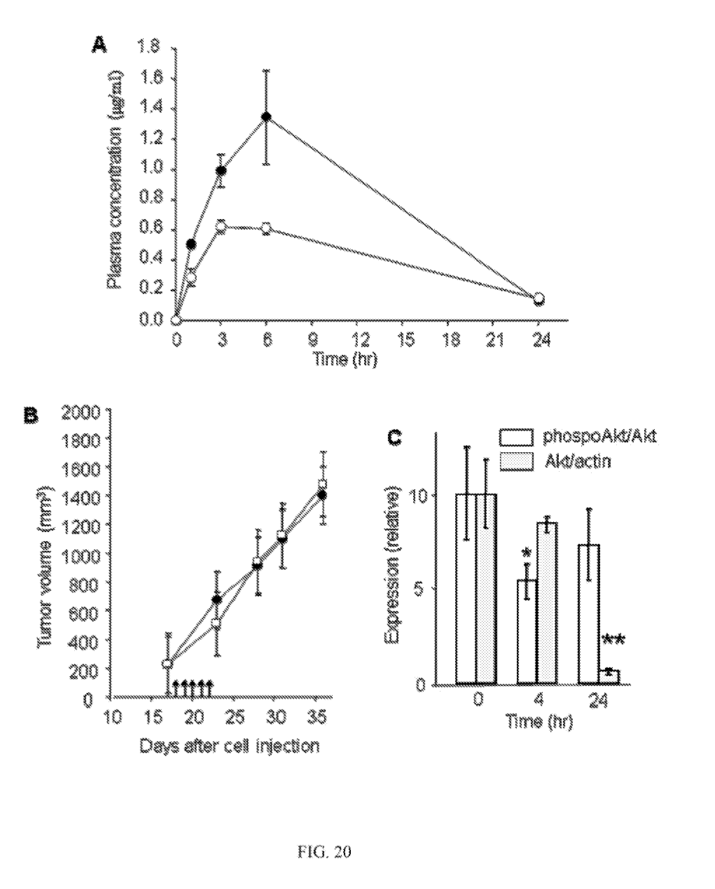

In vivo evaluation of compound 316 was carried out in female scid mice who were administered compound 316 at a dose 250 mg/kg either intraperitonealy (i.p.) or orally (p.o) by oral gavage and plasma concentrations measured. Because compound 316 is insoluble, a slurry in 25% DMSO 20% Trappsol® was prepared and administered. Preliminary studies indicate no toxicity of a single dose of up to 250 mg/kg, which was the maximum dose that could practically be administered i.p. FIG. 20A shows pharmacokinetic studies of a single dose of compound 316 of 250 mg/kg showed a peak concentration of 1.4 nM for i.p. administration () and 0.6 μM for oral administration (∘) with a relative area under the plasma concentration time curve for oral compared to i.p. administration of about 53%. Plasma concentration values are the mean of 3 mice and bars are standard error (S.E.). Five daily doses of 250 mg/kg of compound 316 by i.p. gave moderate neutropenia but no other sign of toxicity, no change in body weight, blood lymphocyte, red blood cell and platelet count, or reduction of aspartate amino transferase (AST) or amino alanine transferase (ALT). However, despite the very large doses administered, high plasma concentrations could not be achieved, and the compound was eliminated relatively rapidly over about a 24 hr period suggesting rapid metabolism or elimination. Thus, concentrations of compound 316 required to inhibit AKT based on the cell culture studies described above, about 4 nM to about 13 nM, could not be achieved.

FIG. 20B shows the antitumor activity in female scid mice with HT-29 colon cancer xenografts treated orally daily for 5 days (arrows) with vehicle alone () or a 250 mg/kg daily dose of compound 316 (□). Tumor volume values are the mean of 10 mice and bars are S.E. These anti-tumor studies indicate that compound 316 may exhibit no activity against HT-29 colon cancer when administered orally for 5 days with a daily dose of 250 mg/kg. However, as indicated in FIG. 16C, inhibition of tumor phospho-Ser-AKT was observed when the HT-29 xenograft tumors were removed and blotted for phospho-Ser-AKT 4 hours after a single 250 mg/kg dose of compound 316 (open bar) as compared to vehicle alone (filled bars), but this inhibition appears to lost after at 24 hours. AKT and phospho-Ser-AKT values are the mean of 4 mice and bars are S.E., *p<0.05,**p<0.01. Additionally, 24 hours after administration there was an unexpected significant decrease in the apparent total AKT concentration compared to an actin loading control. Taken together, the results suggest that the limited solubility of compound 316 and metabolism or elimination of compound 316 may limit the plasma concentrations that can be achieved, and this may prevent effective inhibition of AKT activity. However, compound 316 may inhibit AKT phosphorylation and may be useful to sensitize tumor cells making them more susceptible to chemotherapy and/or radiation treatment.

### Example 12

### AKT and PDK1 Inhibition in Cultured Cells

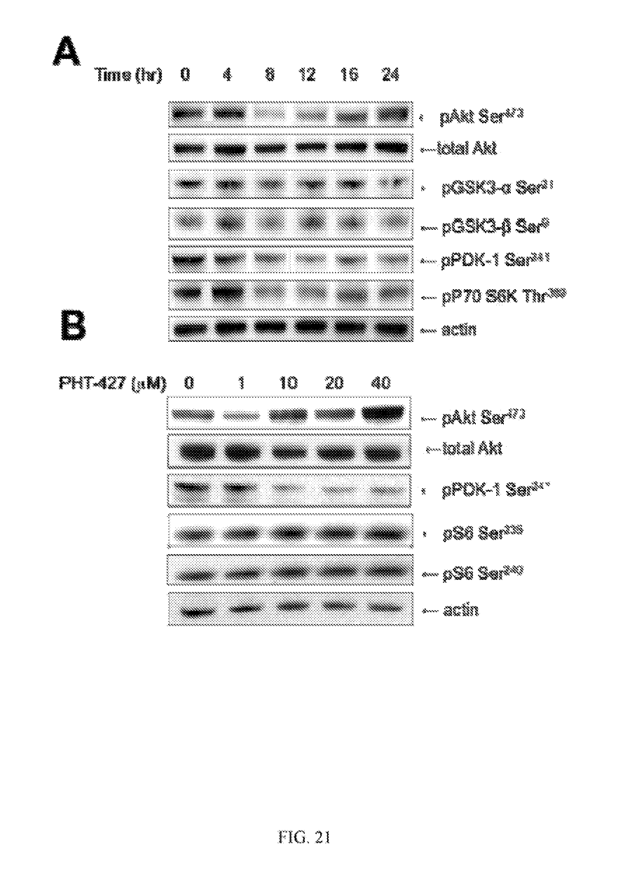

Inhibition of the phosphorylation of AKT, PDK1, and several downstream targets was measured by Western blotting using rabbit polyclonal antibodies to phospho-Ser⁴⁷³-AKT, total-AKT, phospho-Ser²¹-GSK3β phospho-Ser⁹-GSK3β, phospho-Ser²⁴¹-PDK1 and phospho-Thr³⁸⁹p70 S6-kinase (New England Biolabs/Cell Signaling Technology Inc.) using β-Actin as a loading control as described in Mol Cancer Ther 7:2621 (2008). In FIG. 21A, BxPC-3 pancreatic cancer cells were exposed to 10 μM compound 104 in media with 10% fetal bovine serum (FBS) for various times from 0 to 24 hours. As indicated, compound 104 caused an inhibition of phospho-Ser⁴⁷³-AKT levels in cells after 8 to 12 hours exposure, however an increase in phospho-Ser⁴⁷³-AKT was observed after 16 hours to 24 hours. In contrast, PDK1 activity measured by phospho-Ser²⁴¹-PDK1 was maximally inhibited by 104 at 8 to 12 hour, and this inhibition was maintained at least for 24 hour (FIG. 21A).

In FIG. 5B, Panc-1 pancreatic cancer cells were exposed to various concentrations of compound 104 in 10% FBS for 20 hours. As illustrated, inhibition of phospho-Ser⁴⁷³-AKT by 104 was also more pronounced at low concentrations, about 1 μM, and higher concentrations led to increased phosphorylation suggesting a possible feedback activation of AKT activity as has been previously reported. In contrast, PDK1 in inhibition was observed at from 10 μM to 40 μM.

### Example 13

### In Vivo AKT and PDK1 Inhibition

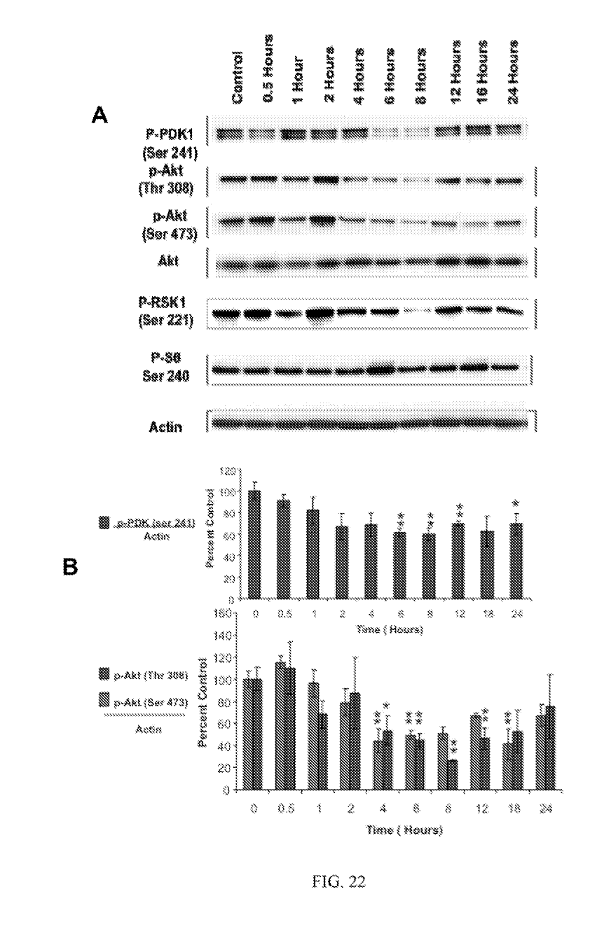

In vivo compound 104 activity was observed in scid mice. Thirty-two (32) stratified mice having about 150 mm³ tumors were selected. A control group of five (5) mice were selected and the remaining mice received 200 mg/kg p.o. QD of compound 104 group1. At the times indicated, 30 minutes, 1 hour, 2 hours, 4 hours, 6 hours, 8 hours, 12 hours, 18 hours, and 24 hours, inhibition of tumor phospho-Ser-AKT for three (3) mice was observed when the by removing the tumor and blotting for phospho-Ser-AKT, phospho-Thr-AKT, and phospho-Ser-PDK along with several downstream targets and β-actin as a control. As indicated in FIG. 22A, phospho-Ser-AKT and phospho-Thr-AKT inhibition are observed at about 4 hours, and phospho-Ser-PDK inhibition is observed at about 6 hours to about 8 hours. However, as illustrated in FIG. 22B, PDK1 phosphorylation inhibition is substantially maintained for 24 hours, while phosphorylation inhibition of AKT appears to be reduced after 18 to 24 hours.

### Example 14

### Fluorescence Measurement of Compound 104 in Skin Layers

Nude mice (three/group) will be treated with 6 to 8 novel formulations containing a compound 104 which has been modified to include a fluorescent label. Following 1, 4 and 6 hrs, the mice will be anesthetized and a full depth skin biopsy will be taken using a 2 mm core skin biopsy needle. The skin biopsy will be frozen, sectioned (5 μm thick), and taken for quantitative assessment of fluorescent drug skin penetration. The sections will be fixed for 10 minutes in acetone, rehydrated for 10 minutes in PBS, then mounted with Fluoromount-G or Vectashield mounting medium containing DAPI and fluorescence through the layers of the mouse skin will be evaluated using a fluorescent microscope.

Digitized photomicrographs of 5 fields each from 5 representative sections will be taken using a Nikon Eclipse TE300 fluorescence microscope equipped with an RS Photoelectric camera. Fluorescent penetration will be automatically circumscribed on the digitized photomicrograph at 1 tenth the maximal fluorescence at the skin surface using the Amira 3 image analysis software package. Control experiments and standard curves are run to determine the optimum cutoff for maximal dynamic range. Data is expressed as pixel count/area and the mean intensity of all 25 photomicrographs to obtain the depth of penetration. Depth of skin penetration will be used to rank the formulations based on the distance/intensity of fluorescence. Akt inhibition in the skin will be evaluated using immunohistochemistry (below).

Mouse skin will be fixed in buffered 4% formaldehyde for 24 hr followed by 70% ethanol before being embedded in paraffin and 5μ sections cut. Sections of mouse skin will be baked and dewaxed automatically, and then stained for pAkt, Akt, COX-2, pGSK3β and pTuberin using a BOND-maX autostainer and Intense Polymer Detection System. Slides will be lightly counter-stained with the onboard hematoxylin to visualize nuclei. Pictures of individual follicle sections will be taken on a Nikon e90i fully motorized upright microscope with an RS Photometrics K4 digital camera using a 10× plan-apo objective lens. Image analysis and reconstruction will be performed using SimplePCI image analysis software to quantitate inhibition.

Selection of preparations will be made by comparing the penetration profile and the Akt inhibition profile of each lotion preparation. At least two preparations will be chosen to examine in the efficacy portion of the project. The preparation that produced the greatest degree of penetration and the greatest amount of Akt inhibition along with a second preparation that produces good Akt inhibition with the least skin penetration will be compared. If all preparations produce good skin penetration then the two preparations that have the highest Akt inhibition will be selected. If there is a similar outcome for a lotion with an aqueous base as well as an alcoholic base, these two preparations will move to efficacy studies.

### Example 15

### Excipient Solubility

The solubility of compound 104 was tested in a variety of pharmaceutically acceptable excipients for oral or topical administration. Compound 104 was added in excess to 1 mL aliquots of each excipient, and for approximately 30 minutes, the mixtures were alternately vortexed, heated to 40° C., and sonicated to facilitate dissolution. The solutions were rotated at room temperature for 24 hours. After rotation, if there was no evidence of precipitated compound, additional compound 104 was added and dissolution processes were repeated. Once 24 hours had passed and compound 104 precipitation remained, samples were filtered through a 0.45 um PVDF syringe filter, the filtrate was diluted if necessary and analyzed by HPLC for compound 104 concentration. The results are reported in Table 12. The low solubilities found in the various plant based oils tested (avocado, vitamin E, soybean, sesame and safflower) were unexpected. the long hydrocarbon chain present in compound 104 would be expected to render it similar in polarity to the variety of nonpolar hydrocarbons present in plant based oils. Hence it was determined that a careful selection of excipients be made for both topical and oral preparations.

TABLE 12
Excipient Solubility Determinations for compound 104.

| | Customary | Compound 104 |
|---|---|---|
| Excipient | Application | Solubility (mg/mL) |
| N-methylpyrrolidone | Topical | 161 |
| (NMP) | | |
| Dimethylacetamide | Topical, Oral | 87.4 |
| (DMA) | | |
| Acconon MC-8 EP/NF | Topical, Oral | 83.7 |
| Benzyl Alcohol | Topical, Oral | 78.1 |
| Labrasol | Topical, Oral | 53.2 |
| Gelucire 44/14 | Oral | 35.2 |
| Labrafil | Oral | 10.4 |
| Acetone | Topical | 74 |
| Captex 200P | Oral | 6.8 |
| Capmul MCM L | Oral | 5.3 |
| Captex 355 EP/NP | Oral | 4.7 |
| Captex 810 D | Oral | 2.4 |
| Avocado Oil/Vitamin E | Topical, Oral | 1.4 |
| Oil | | |
| (50/50) by % volume | | |
| Sesame Oil | Topical, Oral | 1.2 |
| Soybean Oil | Topical, Oral | 1.1 |
| Safflower Oil | Topical, Oral | 1.1 |
| Glycerol | Topical | 0.01 |
| Aquaphor | Topical | <0.01 |
| Mineral Oil | Topical | <0.01 |
| Water | No Limitations | <0.001 |

### Example 16

### pH Solubility Profile

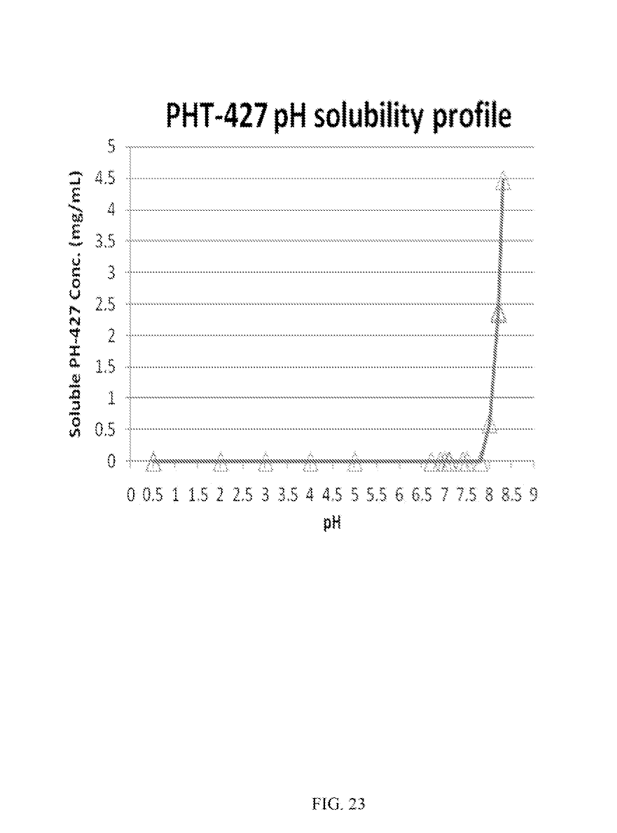

The aqueous solubility of compound 104 was determined at various pH values. compound 104 was added in excess to 1 mL aliquots of buffer solutions ranging from pH 0.5 to pH 10.5, and for approximately 30 minutes, the mixtures were alternately vortexed and sonicated to facilitate dissolution. These samples were then rotated at room temperature for 24 hours. After rotation, if there was no evidence of precipitated compound, additional compound 104 was added and the dissolution processes were repeated. Once 24 hours had passed and compound 104 precipitate remained, samples were filtered through a 0.45 um PVDF syringe filter, the filtrate was analyzed by HPLC for compound 104 concentration. The resulting pH-solubility profile is provided in FIG. 23. Compound was found to have an acidic pKa between 7.9 and 8.3 which can likely be attributed to the nitrogen of the sulfonamide group. Perhaps, more surprisingly, the compound appears to have an additional pKa above 9. this is unexpected as there do not appear to be any other acidic groups resent in the molecule. Furthermore, the dip observed in the profile at about pH 9 is generally characteristic of multiple pKas with opposing charges. such would imply compound 104 that the thiadiazole group can pick up a proton above pH 9.

HPLC Assay. The HPLC assay for analysis of sample concentrations of compound 104 used a Phenomenex, Gemini-NX; C-18; 50×2.0 mm; 3 μm; 110 Å column on a Waters HPLC. The mobile phase was isocratic Acetonitrile/H2O (80/20%) with 0.1% TFA with a flow rate of 0.5 mL/min and a column temperature of 35° C. The run time was 5 minutes and the injection volume was 2 μL. Compound 104 was monitored at a wavelengths of 270 nm (λₘₐₓ) and eluted at 1.3 minutes.

### Example 17

### Preparation of Topical Formulation

30 mg/mL compound 104 in Vehicle #1: A topical formulation containing 30 mg/mL compound 104 was prepared using a lipophilic base (Aquaphor, 50%), a cosolvent (Labrasol, 42%), and a penetration enhancer (N-methylpyrrolodone, NMP, 8%). Specifically, 50 mg of compound 104 was dissolved in 0.4 mL NMP, resulting in a 125 mg/mL compound 104 solution. Additionally, 100 mg of compound 104 was dissolved in 2.1 mL of Labrasol. The 2 solutions were mixed by vortexing to avoid exceeding the limiting solubility of compound 104 in each of the 2 individual excipients. 2.5 mL of Aquaphor was added and the entire mixture was heated and sonicated to melt the Aquaphor and mix all ingredients. The resulting formulation was slightly runny and underwent phase separation once re-established at room temperature.

49 mg/mL compound 137 in Vehicle #1: A topical formulation containing 49 mg/mL compound 104 was prepared using a lipophilic base Aquaphor (50%), a cosolvent Labrasol (42%) and a penetration enhancer (NMP, 8%). Specifically, 70 mg of compound 104 was dissolved in 0.4 mL NMP, resulting in a 175 mg/mL compound 104 solution. Additionally, 175 mg of compound 104 was dissolved in 2.1 mL of Labrasol. The 2 solutions were mixed by vortexing. 2.5 mL of Aquaphor was added and the entire mixture was heated and sonicated to melt the Aquaphor and mix all ingredients. The resulting formulation was slightly runny and underwent phase separation once re-established at room temperature.

40 mg/mL compound 104 in Vehicle #2: A topical formulation containing 40 mg/mL compound 104 was prepared using a commercial conditioning product known as Nourishing Conditioner™ brand conditioner product by ABBA (having listed ingredients of: Water, Arnica Montana Flower Extract, Calendula Officinalis Flower Extract, Chamomilla Recutita (Matricaria) Flower Extract, Prunus Serotina (Wild Chemy) Bark Extract, Lavandula Angustifolia (Lavender) Flower Extract, Cymbopogon Schoenanthus Extract, Rosmarinus Officinalis (Rosemary) Flower Extract, Passiflora Incarnata Extract, Passiflora Incarnata Fruit Extract (*Passion Flower), Cetyl Alcohol, Stearyl Alcohol, Cetrimonium Chloride, Glycerin, Lupin Amino Acids (150 mw), Hydrolyzed Soy Protein (1000 mw), Hydrolyzed Wheat Protein (2000 mw), Hydrolyzed Wheat Starch, Tocopherol Acetate, Aloe Barbadensis Leaf Juice, Algin, Citric Acid, Limonene, Methylparaben, Propylparaben, Diazolidinyl Urea, Fragrance (Parfum)). The Nourishing Conditioner™ was provided as about 50% by volume of a formulation, with a penetration enhancer (NMP) as about 50% by volume of the formulation). Specifically, 200 mg of compound 104 was dissolved in 2.5 mL of NMP. This 80 mg/mL compound 104 solution was mixed with 2.5 mL of Nourishing Conditioner™ with keratin proteins. Full mixing was achieved by vortexing. The resulting formulation was smooth, creamy and of homogenous blend.

20 mg/mL compound 104 in Vehicle #3: A topical formulation containing 20 mg/mL compound 104 was prepared using commercial conditioning product Normalizing Conditioner (NC, Graham Webb, 50%) [Ingredients: Water, Cetearyl Alcohol, Behentrimonium Methylsulfate, Propylene Glycol, Dimethicone, Hydroxyethylcellulose, Stearalkonium Chloride, Fragrance, Methylparaben, Amodimethicone, Panthenol, Alcohol Denatured, Propylparaben, Hexylcinnamal, Linalool, Cetrimonium Chloride, Butyrospermum Parkii (Shea Butter), Cyclotetrasiloxane, Trideceth 12, Citric Acid, Sodium Acetate, Sodium Benzoate, Camellia Sinensis Leaf Extract, Echinacea Purpurea (Coneflower) Root Extract] and penetration enhancer (NMP, 50%). Specifically, 160 mg of compound 104 was dissolved in 2 mL NMP to create a solution of 80 mg/mL compound 104. One mL of the 80 mg/ml compound 104 solution was mixed with 3 mL of NC by vortexing. The resulting formulation was smooth, creamy and of homogenous blend.

32 mg/mL compound 137 in Vehicle #3: A topical formulation containing 32 mg/mL compound 137 was prepared using commercial conditioning product Normalizing Conditioner (NC, Graham Webb, 50%) and penetration enhancer (NMP, 50%). Specifically, 260 mg of PHT-37 was dissolved in 2 mL NMP to create a solution of 130 mg/mL PHT-37. One mL of the 130 mg/ml PHT-37 solution was mixed with 3 mL of NC by vortexing. The resulting formulation was smooth, creamy and of homogenous blend. The formulation was filled into 3 mL syringes to deliver PHT-37 in 100 and 200 μL volume to mice skin in a penetration study. PHT-37, a fluorescent analogue of PHT-427 was used as a surrogate to determined depth of penetration following administration.

50 mg/mL Compound 104 in Vehicle #3: A topical formulation containing 50 mg/mL compound 104 was prepared using the Vehicle #3 by dissolving compound 104 (500 mg) in 5 mL NMP to create a solution of 100 mg/mL compound 104. Five mL of NC was added to the compound 104 solution and mixed by vortexing. The resulting formulation was smooth, creamy and of homogenous blend. The formulation was filled into 3 mL syringes to deliver 100 uL topically to mice skin.

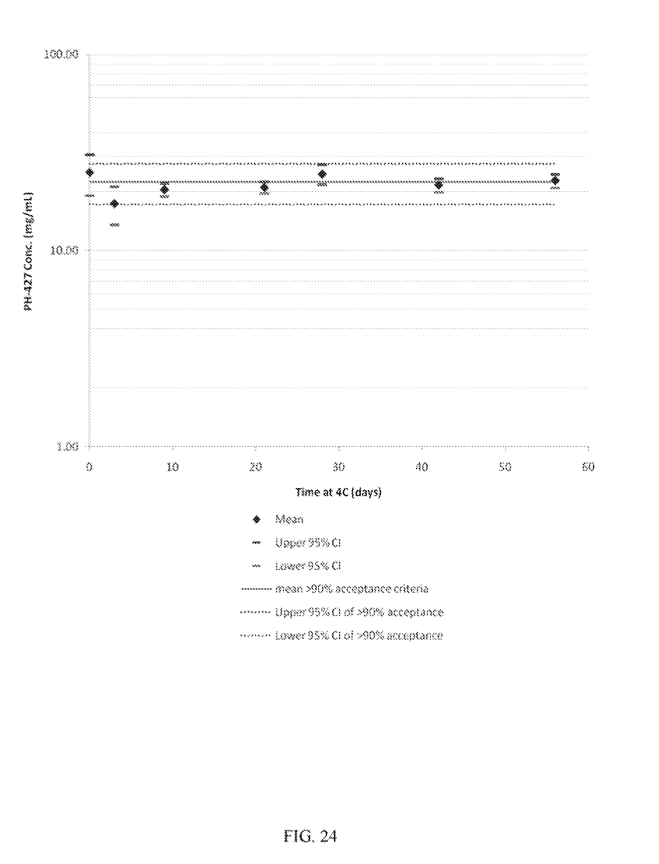

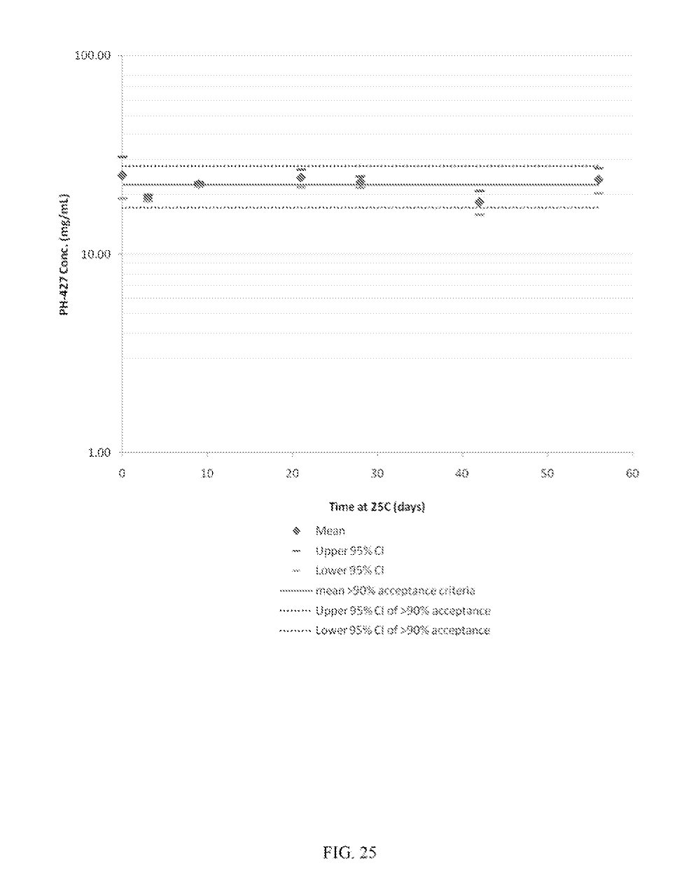

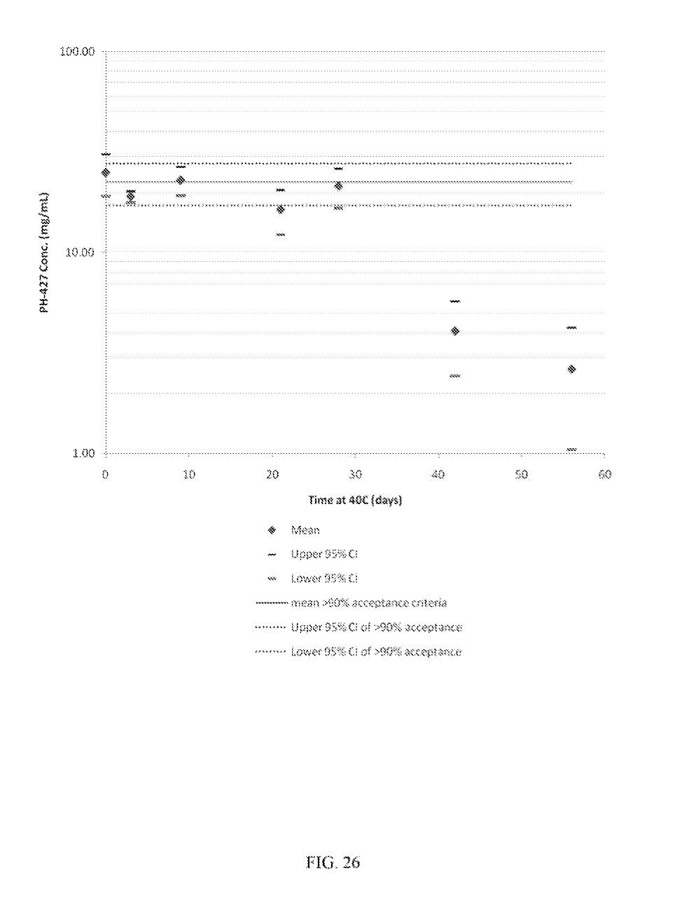

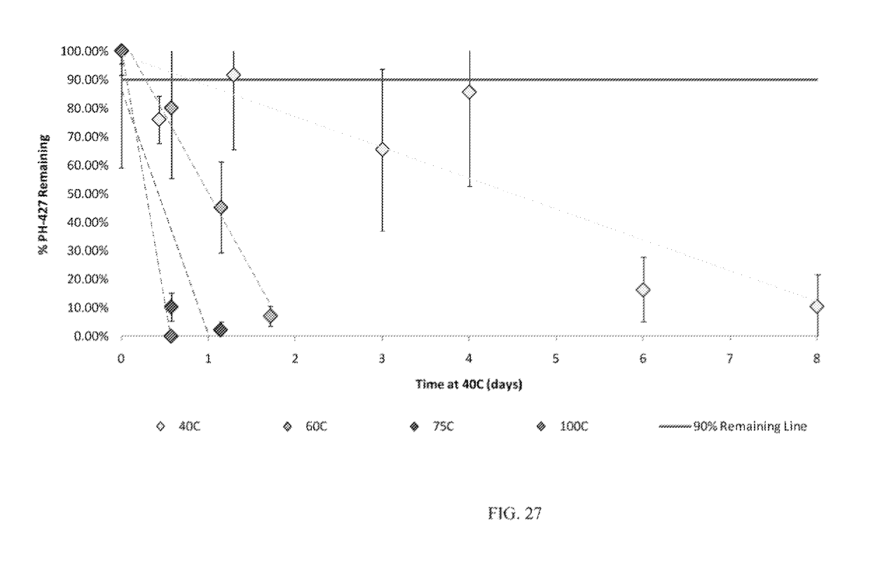

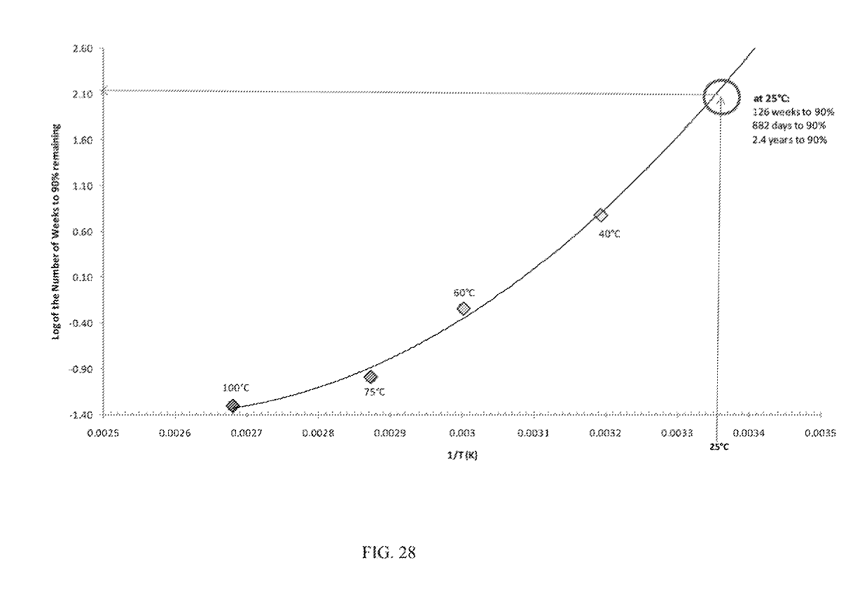

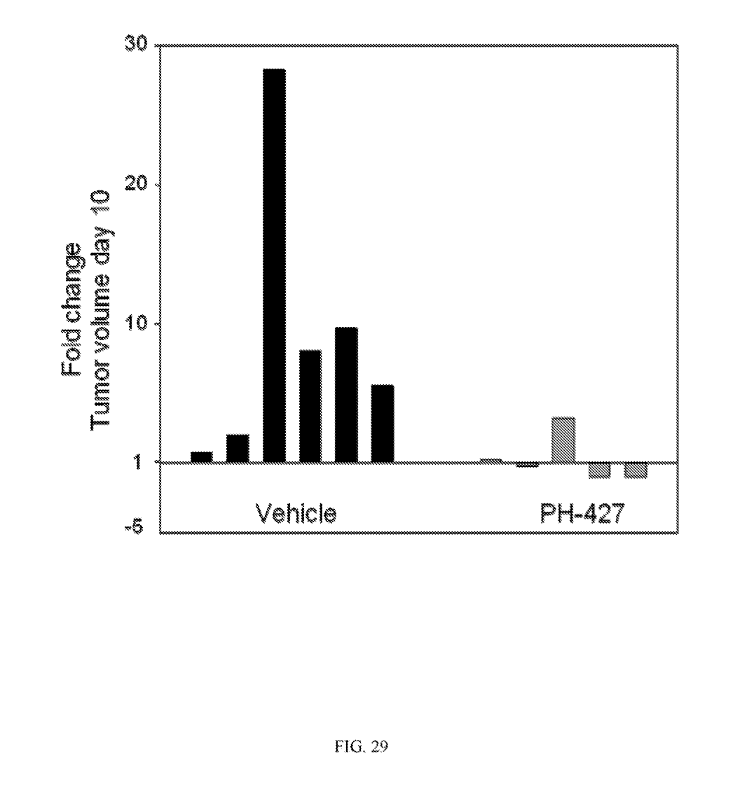

Stability of Topical Formulation in Vehicle #3: The Vehicle #3 formulation was homogenous. A formulation of 40 mg/ml compound 104 in Vehicle #3 was diluted with NC/NMP (50/50 by volume) such that the final compound 104 concentration was 20 mg/mL. This mixture was filled into multiple 3 mL syringes and stored at 3 different temperatures 4° C. (FIG. 24), 25° C. (FIG. 25), and 40° C. (FIG. 26). Samples were taken in triplicate and analyzed for compound 104 concentration by HPLC on days 0, 3, 9, 21, 28, 42 and 56. No significant degradation (at a p<0.05 level of confidence) was measured at either 4° C. (FIG. 24) or 25° C. (FIG. 25) during the 56 day period. However, significant degradation of product (p=0.01) was observed between 28 and 42 days at 40° C. (FIG. 26). This degradation may be due to the breakdown of the vehicle composition in addition to instability of compound 104.

Estimated Shelf Life of Topical Formulation in Vehicle #3: According to the FDA Guidance, the shelf life of a product is the time at which 90% of initial concentration of active agent remains under recommended storage conditions. An estimate of the shelf life of compound 103 in vehicle #3 at room temperature (25° C.) was obtained by forced degradation at 4 temperatures: 40° C., 60° C., 75° C. and 100° C. The concentration of compound 104 was plotted vs, time for each storage temperature until each concentration (including the 95% CI) passed below the 90% threshold (FIG. 27). The log of the time for each test sample to reach <90% was then plotted against the reciprocal temperature and the extrapolated equation of the line was used to determine the predicted shelf life at room temperature (FIG. 28).

In FIG. 27, the stability of compound in Vehicle #3 stored at 40° C. (yellow), 60° C. (orange), 75° C. (red) and 100° C. (pink) is shown. Error bars represent the 95% CI about the mean. The solid red line represents the 90% threshold which must be significantly passed (including the 95% CI around the mean time point value) to indicate limit of shelf life. The time to reach the 90% threshold are 6 weeks, 1.1 weeks, 0.57 weeks and 0.3 weeks for the 40° C., 60° C., 75° C. and 100° C. storage conditions, respectively.

In FIG. 28, a modified Arrhenius plot showing the log of the number of weeks to 90% on the y-axis and the reciprocal temperature on the x-axis. The colored diamonds represent the storage conditions: 40° C. (yellow), 60° C. (orange), 75° C. (red) and 100° C. (pink). The trendline (R²=0.99) is extrapolated to the 25° C. temperature point such that an estimate of shelf life at room temperature is obtained. The shelf life of compound 104 in Vehicle #3 at room temperature (25° C.) is predicted to be 2.4 years.

Compound 104 in Vehicle 5: Compound 104 (200 mg) was combined with N-methylpyrrolidone (NMP, 1.6 mL), and alternating sonicating and vortexing was carried out until solution is clear as evidenced by the absence of the Tyndall effect by laser pointer. Compound 104 (400 mg) and Labrasol™ (Gattefosse, 8.4 mL) were combined in a 50 mL round bottom flask, and alternate sonicating and vortexing was carried out until solution was clear as evidenced by the absence of the Tyndall effect by laser pointer. The first mixture was transferred into the 50 mL round bottom flask and was vortexed to mix the first and second solutions. Aquaphor™ (Eucerin, 10 g) was added to the Compound 104/NMP/Labrasol solution and this mixture was vortexed thoroughly until homogenously creamy with no visible sign of liquid remaining. The composition was filled into syringes and stored at 4° C. until used.

Compound 137 in Vehicle 5: Compound 137 (280 mg) was combined with N-methylpyrrolidone (NMP, 1.6 mL), and alternate sonicating and vortexing was carried out until solution was clear as evidenced by the absence of the Tyndall effect by laser pointer. In a separate 50 mL round bottom flask, compound 137 (560 mg) was combined with Labrasol™ (Gattefosse, 8.4 mL), and alternate sonicating and vortexing was carried out until solution was clear as evidenced by the absence of the Tyndall effect by laser pointer. The contents of the first mixture were combined with the second mixture in the 50 mL round bottom flask and were vortexed to mix the two solutions. Aquaphor™ (Eucerin, 10 g) was added to the round bottom flask containing compound 137/NMP/Labrasol solution. This solution was vortexed thoroughly until homogenously creamy and there was no visible sign of liquid remaining. The resulting cream was filled into syringes and stored at 4° C. until used.

Compound 104 in Vehicle 6: Compound 104 (1600 mg) was combined with N-methylpyrrolidone (NMP, 10 mL), and this mixture was alternately sonicated and vortexed until solution is clear as evidenced by the absence of the Tyndall effect by laser pointer. Nourishing Conditioner (NourC, ABBA; 10 mL (10 g), Ingredients: Water, Arnica Montana Flower Extract, Calendula Officinalis Flower Extract, Chamomilla Recutita (Matricaria) Flower Extract, Prunus Serotina (Wild Chemy) Bark Extract, Lavandula Angustifolia (Lavender) Flower Extract, Cymbopogon Schoenanthus Extract, Rosmarinus Officinalis (Rosemary) Flower Extract, Passiflora Incarnata Extract, Passiflora Incarnata Fruit Extract (*Passion Flower)], Cetyl Alcohol, Stearyl Alcohol, Cetrimonium Chloride, Glycerin, Lupin Amino Acids (150 mw), Hydrolyzed Soy Protein (1000 mw), Hydrolyzed Wheat Protein (2000 mw), Hydrolyzed Wheat Starch, Tocopherol Acetate, Aloe Barbadensis Leaf Juice, Algin, Citric Acid, Limonene, Methylparaben, Propylparaben, Diazolidinyl Urea, Fragrance (Parfum)) was added to the mixture. The mixture was vortexed thoroughly until homogenously creamy with no visible sign of liquid remaining. The resulting cream was filled into syringes and stored at 4° C. until used.

Compound 104 in Vehicle 7: Compound 104 (1600 mg) was combined with N-methylpyrrolidone (NMP, 10 mL), and this mixture was alternately sonicated and vortexed until solution is clear as evidenced by the absence of the Tyndall effect by laser pointer. Normalizing Conditioner (NC, Graham Webb, 10 mL (10 g), Ingredients: Water, Cetearyl Alcohol, Behentrimonium Methylsulfate, Propylene Glycol, Dimethicone, Hydroxyethylcellulose, Stearalkonium Chloride, Fragrance, Methylparaben, Amodimethicone, Panthenol, Alcohol Denatured, Propylparaben, Hexylcinnamal, Linalool, Cetrimonium Chloride, Butyrospermum Parkii (Shea Butter), Cyclotetrasiloxane, Trideceth 12, Citric Acid, Sodium Acetate, Sodium Benzoate, Camellia Sinensis Leaf Extract, Echinacea Purpurea (Coneflower) Root Extract.)) was added to the mixture. The mixture was vortexed thoroughly until homogenously creamy with no visible sign of liquid remaining. The resulting cream was filled into syringes and stored at 4° C. until used.

Compound 137 in Vehicle 7: Compound 137 (640 mg) was combined with N-methylpyrrolidone (NMP, 10 mL), and this mixture was alternately sonicated and vortexed until solution is clear as evidenced by the absence of the Tyndall effect by laser pointer. Normalizing Conditioner (NC, Graham Webb, 10 mL (10 g)) was added to the mixture. The mixture was vortexed thoroughly until homogenously creamy with no visible sign of liquid remaining. The resulting cream was filled into syringes and stored at 4° C. until used.

### Example 18

Biological Activity of Topical Formulation

To evaluate the biological activity of the topical preparation, compound 104 in vehicle #3 was used to treat mice with intradermal tumors. The female nu/nu mice received a 60 day 17-beta-estradiol pellet one day before injection with 10⁷ MCF-7 breast cancer cells intradermally using a 27 gauge needle cells into the flank. When tumors reached 40 to 200 mm³ they were treated twice a day with 0.1 ml of vehicle #3 or with 50 mg/ml compound 104 applied over the tumor. The mice were housed individually with Elizabethan collars to prevent them grooming the area with the tumor. Tumor volumes were measured 3 times a week. After 10 days mice were euthanized 4 hr after the last application of compound 104 and blood, tumor, and overlying skin removed for evaluation of compound 104 concentration and biomarker levels. Tumor volume at 10 days versus day 0 were compared and the fold change was plotted (FIG. 19). Animals treated with compound 104 showed tumor regressions or less increase in volume as compared to those treated with vehicle alone. Additionally, compound 104 absorbed through the dermal layers into plasma producing plasma levels of 5.49±1.56 μg/ml.

### Example 19

### Preparation of Oral Formulations

Oral Formulation 1: Compound 104 (799.6 mg, 99.95% w/w) was Magnesium Stearate (MgSt, 0.4 mg, 0.05% w/w) were combined into a mortar and triturated (3 cycles) with a pestel to encourage homogeneity. The contents of the mortar were transferred to a clean vial and stored at 4° C. until filling into capsules.

Oral Formulation 2: Compound 104 (200 mg, 25% w/w) was combined with Starch 1500 (Colorcon, 200 mg, 25% w/w), microcrystalline cellulose (MCC, 400 mg, 50% w/w), and stearic acid (StAc); 0.4 mg (0.05% w/w). This combination of ingredients was transferred into a mortar and triturate (3 cycles) with pestle to encourage homogeneity. The contents were transeferred to clean vial for storage at 4° C. until pressing into tablets.

Oral Formulation 3: Compound 104 (200 mg, 25% w/w) was combined with Starch 1500 (Colorcon; 200 mg, 25% w/w), microcrystalline cellulose (MCC, 400 mg, 50% w/w), magnesium stearate (MgSt, 0.4 mg, 0.05% w/w). This mixture was transeferred to a mortar and was triturate (3 cycles) with pestle to encourage homogeneity. The contents were transferred to a clean vial for storage at 4° C. until pressing into tablets.

Oral Formulation 4: Compound 104 (1000 mg, 19% w/w) was combined with Cremophor RH 40 (BASF) or polyoxyl 40 hydrogenated castor oil (800 mg, 15%) and this mixture was heated to 65° C. while alternately sonicating and vortexing to incorporate the Compound 104 into surfactant. In a separate container, water (3200 mg, 3.2 mL, 60% w/w) was heated to 65° C. and the heated water was slowly added to the Cremophor/Compound 104 mixture while stirring constantly. The mixture thickened until 1.6 mL of the water had been added. The second 1.6 mL began to thin the mixture again. Once all the water had been added, the mixture was slowly cooled to room temperature while slowly adding benzyl alcohol (200 mg, 4% w/w) and ethanol (200 mg, 4% w/w). This mixture was stored in a closed vial at 4° C. until filling into liquid gel capsules.

### Example 20

Oral Excipient Compatibility/Stability

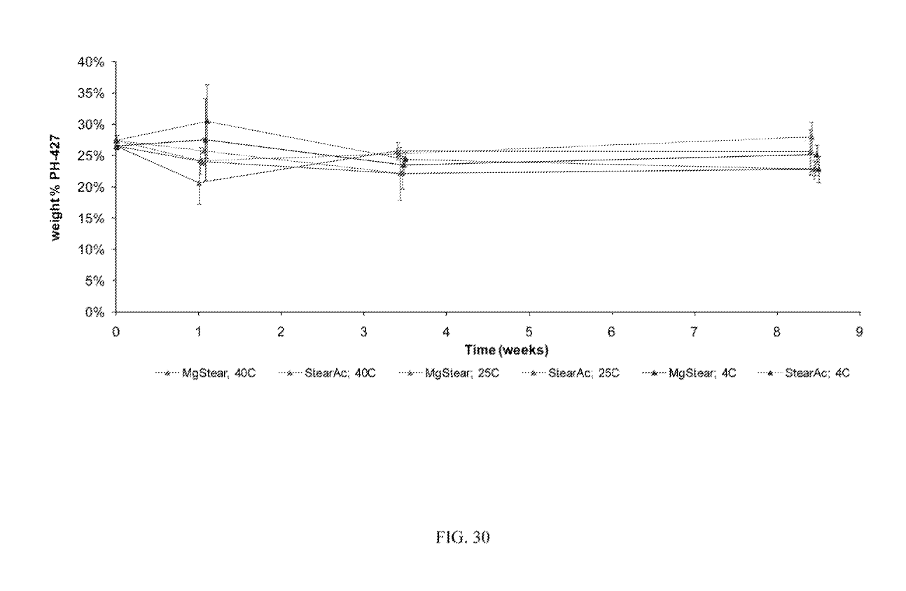

The compatibility of compound 104 was examined at 3 storage temperatures with a variety of various pharmaceutically acceptable oral excipients used in tablet and capsule products. Compound 104 was prepared in 2 product mixtures. The first conatined 200 mg of compound 104, 200 mg of starch, 400 mg of microcrystalline cellulose (MCC), and 4 mg of magnesium stearate. The second mixture contained 200 mg of compound 104, 200 mg of starch, 400 mg of microcrystalline cellulose (MCC), and 4 mg of stearic acid. Thus, both mixtures contained ratios of 24.9/24.9/49.7/0.5% w/w/w/w of PH-427/starch/MCC/magnesium stearate or stearic acid. The mixtures were triturated well using morter and pestle, filled into 3 separate vials, and stored at 3 different temperatures: 4° C., 25° C. and 40° C. Samples were taken in triplicate and analyzed for compound 104 concentration by HPLC at 0, 1 week, 3.5 weeks, and 8.5 weeks. There was no significant change in the compound 104 concentration with either of the excipients mixtures at 8.5 weeks FIG. 30.

Oral Capsule Filling. Based on known stability of compound 104 with 0.5% Magnesium Stearate (MgStear) (shown in Figure G), sizes 1 and 3 gelatin capsules were filled with 104/MgStear (95.5/0.5% by weight). Note that additional ingredients such as starch and MCC are not necessary in capsule dosage form as swelling and disintegration are not required for drug release. The fill weights were 85±5 mg and 136±5 mg for the sizes 3 and 1 capsules, respectively.

Oral Capsule Dissolution. A dissolution system similar to Apparatus #2 as described in the USP 28 (Physical tests, Chapter <711> Dissolution) was created. Specifically, a 900 mL jacketed cylindrical glass vessel was equipped with a thermometer, a basket and overhead paddle stirring mechanism. The jacketing permitted water to be circulated around the vessel at a constant temperature of 37±0.5° C. and the thermometer allowed constant monitoring of the vessel's internal temperature. Stirring speed was held consistent at 100 rpm for 30 minutes during the dissolution testing and the solution was consistently circulated through a 0.45 vim filter, into a flow cell in a spectrophotometer. The apparatus was first calibrated against 300 mg salicylic acid tablets according to the method detailed in the USP 28 (Physical tests, Chapter <711> Dissolution). Prior to introduction of the compound 104 filled capsule into the basket, the dissolution media was used as the blank at 270 nm. Once the capsule was placed in the basket and submerged into the dissolution media, the dissolved compound 104 concentration was constantly monitored for a 30 minute duration. At 30 minutes, the total amount of dissolved drug was calculated and expressed as a percentage of the total dose in the capsule. This procedure was carried out in triplicate using both USP simulated gastric fluid and USP simulated intestinal fluid. Due to insolubility of compound 140 the material in size 1 capsules was spureou because the material clogged the filter and delayed the flow of material through the apparatus. Only data from size 3 capsules is reported.

What is claimed is:

1. A pharmaceutical composition comprising:
a pharmaceutically effective amount of a small molecule that binds a Pleckstrin Homology domain (PH) of AKT protein kinases and inhibits AKT protein kinase activity;
one or more pharmaceutically acceptable carriers, excipients, or combinations thereof; and
an enteric coating formulated to release the small molecule at a pH of from about 7.0 to about 11.

2. The pharmaceutical composition of claim 1, wherein the small molecule is a compound of formula IV: 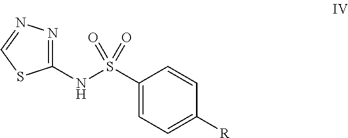
or pharmaceutically acceptable salt thereof, wherein R is an amine, methyl, alkyl, alkene, alkyne, aminoalkyl, alkyl carbamate, alkyl acetamide, alkyl sulfonyl, alkyl sulfonic acid ester, or alkyl sulfonamide.

3. The pharmaceutical composition of claim 2 or pharmaceutically acceptable salt thereof, wherein R is a linear or branched C₂-C₂₀ alkyl, linear or branched C₂-C₂₀ alkene, linear or branched C₂-C₂₀ alkyne, linear or branched C₂-C₂₀ aminoalkyl, linear or branched C₂-C₂₀ alkyl carbamate branched C₂-C₂₀ alkyl acetamide, linear or branched C₂-C₂₀ sulfonyl, linear or branched C₂-C₂₀ sulfonic acid ester, or linear or branched C₂-C₂₀ sulfonamide.

4. The pharmaceutical composition of claim 2 or pharmaceutically acceptable salt thereof, wherein R is a linear C₂-C₂₀ alkyl.

5. The pharmaceutical composition of claim 2 or pharmaceutically acceptable salt thereof, wherein R is alkyl acetamide of formula —NHC(O)CHₙCH₃ wherein n is 0 to 20.

6. The pharmaceutical composition of claim 2 or pharmaceutically acceptable salt thereof, wherein R is selected from —CH₁₁CH₃ and —NHC(O)CH₁₁CH₃.

7. The pharmaceutical composition of claim 1, wherein the compound of formula IV is:

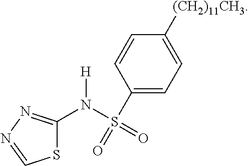

8. The pharmaceutical composition of claim 1, wherein the one or more pharmaceutically acceptable carriers, excipients, or combinations thereof are selected from lactose, sucrose, mannitol, sorbitol, cellulose preparations, calcium phosphates, tricalcium phosphate, calcium hydrogen phosphate, starch, maize starch, wheat starch, rice starch, potato starch, gelatin, tragacanth, methyl cellulose, hydroxypropylmethylcellulose, sodium carboxymethylcellulose, polyvinyl pyrrolidone, and combinations thereof.

9. The pharmaceutical composition of claim 1, further comprising one or more additives selected from binders, fillers, disintegrating agents, sweeteners, wetting agents, colorants, controlled release agents, sustained release agents, and combinations thereof.

10. The pharmaceutical composition of claim 1, wherein the one or more pharmaceutically acceptable carriers, excipients, or combinations thereof is microcrystalline cellulose.

11. The pharmaceutical composition of claim 1, wherein the one or more pharmaceutically acceptable carriers, excipients, or combinations thereof is a starch.

12. The pharmaceutical composition of claim 1, further comprising at least one of magnesium stearate or stearic acid.

13. The pharmaceutical composition of claim 1, wherein the enteric coating is selected from cellulose acetate phthalate (CAP), methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate, stearic acid, and combinations thereof.

14. The pharmaceutical composition of claim 1, wherein the enteric coating is formulated to release the small molecule at a pH of from about 8.0 to about 10.

15. A pharmacaeutical composition for topical administration comprising:
a pharmaceutically effective amount of a small molecule that binds to the Pleckstrin Homology domain (PH) of AKT protein kinases and inhibits AKT protein kinase activity; and
one or more of pharmaceutically acceptable lipophilic bases, cosolvents, cosurfactants, or combinations thereof.

16. The pharmaceutical composition of claim 15, wherein the small molecule is a compound of formula IV: 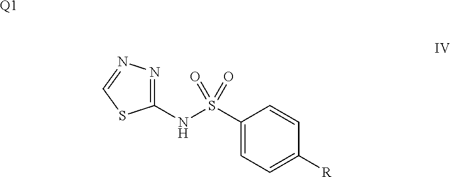
or pharmaceutically acceptable salt thereof wherein R is an amine, methyl, alkyl, alkene, alkyne, aminoalkyl, alkyl carbamate, alkyl acetamide, alkyl sulfonyl, alkyl sulfonic acid ester, or alkyl sulfonamide.

17. The pharmaceutical composition of claim 16, wherein R is a linear or branched C₂-C₂₀ alkyl, linear or branched C₂-C₂₀ alkene, linear or branched C₂-C₂₀ alkyne, linear or branched C₂-C₂₀ aminoalkyl, linear or branched C₂-C₂₀ alkyl carbamate branched C₂-C₂₀ alkyl acetamide, linear or branched C₂-C₂₀ sulfonyl, linear or branched C₂-C₂₀ sulfonic acid ester, or linear or branched C₂-C₂₀ sulfonamide.

18. The pharmaceutical composition of claim 16 or pharmaceutically acceptable salt thereof wherein, wherein R is a linear C₂-C₂₀ alkyl.

19. The pharmaceutical composition of claim 16, wherein R is alkyl acetamide of formula —NHC(O)CH₁₁CH₃ wherein n is 0 to 20.

20. The pharmaceutical composition of claim 16, wherein R is selected from —CH₁₁CH₃ and —NHC(O)CH₁₁CH₃.

21. The pharmaceutical composition of claim 15, wherein the compound of formula IV is: 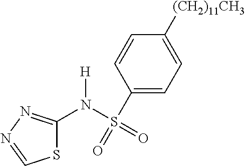
or a pharmaceutically acceptable salt thereof.

22. The pharmaceutical composition of claim 15, wherein the lipophilic base is selected from the group consisting of, White Ointment USP, Yellow Ointment NF, Oleic Acid USP, Olive Oil USP, Paraffin USP, Petrolatum NF, White Petrolatum USP, Spermaceti Wax USP, Synthetic Spermaceti NF, Starch Glycerite NF, White Wax USP, Yellow Wax USP, Cetearyl Alcohol, Behentrimonium Methylsulfate, Propylene Glycol, Dimethicone, Hydroxyethylcellulose, Stearalkonium Chloride, Fragrance, Methylparaben, Amodimethicone, Panthenol, Alcohol Denatured, Propylparaben, Hexylcinnamal, Linalool, Cetrimonium Chloride, Butyrospermum Parkii (Shea Butter), Cyclotetrasiloxane, Trideceth 12, and combinations thereof.

23. The pharmaceutical composition of claim 15, wherein the composition comprises water, arnica montana flower extract, calendula officinalis flower extract, chamomilla recutita (matricaria) flower extract, prunus serotina (wild cherry) bark extract, lavandula angustifolia (lavender) flower extract, cymbopogon schoenanthus extract, rosmarinus officinalis (rosemary) flower extract, passiflora incarnata extract, passiflora incarnata fruit extract (passion flower), cetyl alcohol, stearyl alcohol, cetrimonium chloride, glycerin, lupin amino acids, hydrolyzed soy protein, hydrolyzed wheat protein, hydrolyzed wheat starch, tocopherol acetate, aloe barbadensis leaf juice, algin, citric acid, limonene, methylparaben, propylparaben, and diazolidinyl urea.

24. The pharmaceutical composition of claim 15, wherein the cosolvent is selected from the group consisting of caprylocaproyl macrogolglycerides, polyglyceryl-6-distearate, and combinations thereof.

25. The pharmaceutical composition of claim 15, wherein the cosurfactant is selected from the group consisting of capryol 90, lauroglycol 90, and combinations thereof.

26. The pharmaceutical composition of claim 15, further comprising a penetration enhancer.

27. The pharmaceutical composition of claim 26, wherein the penetration enhancer is selected from the group consisting of methanol, ethanol 2-propanol, alkyl methyl sulfoxides such as dimethyl sulfoxide, decylmethyl sulfoxide, tetradecylmethyl sulfoxide, pyrrolidones, acetone, dimethyl acetamide, dimethyl formamide, and tetrahyrdofurfuryl alcohol, niacin, niacinamide, and combinations thereof.

28. The pharmaceutical composition of claim 26, wherein the penetration enhancer is selected from the group consisting of 2-pyrrolidone, N-methyl-2-pyrrolidone, N-(2-hydroxyethyl)pyrrolidone, laurocapram, and combinations thereof.
